SEQUENCE LISTING (248 sequences)

SEQ ID NO: 1 - protein, 118 aa, synthetic construct
  REGION 1..118: note = Gen1_5072 Humanized Anti-P VH
QVQLVQSAPEVAKPGTSVKMSCKASGYIFTNYPIHWVKQAPGQGLEWIGFIDPGGGYDEPDERFRDRATLTADKSTSTAYMELSSLRSEDTAIYYCARRGGGYYLDYWGQGTLVTVSS

SEQ ID NO: 2 - protein, 118 aa, synthetic construct
  REGION 1..118: note = Gen1_5072 Humanized Anti-P VH
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSS

SEQ ID NO: 3 - protein, 118 aa, synthetic construct; identical to SEQ ID NO: 2
  REGION 1..118: note = Gen1_5072 Humanized Anti-P VH

SEQ ID NO: 4 - protein, 118 aa, synthetic construct; identical to SEQ ID NO: 2
  REGION 1..118: note = Gen1_5072 Humanized Anti-P VH

SEQ ID NO: 5 - protein, 118 aa, synthetic construct
  REGION 1..118: note = Gen1_5072 Humanized Anti-P VH
EVQLVQSGAEVKKPGASVKVSCKASGHIFTNYPIHWVRQAPGQGLEWMGFIDPGGGHDEPDERFRDRVTMTADTSTSTAYMELSSLRSEDTAVYYCARRGGGYYLDYWGQGTTVTVSS

SEQ ID NO: 6 - protein, 118 aa, synthetic construct; identical to SEQ ID NO: 2
  REGION 1..118: note = Gen1_5072 Humanized Anti-P VH

SEQ ID NO: 7 - protein, 118 aa, synthetic construct
  REGION 1..118: note = Gen1_5072 Humanized Anti-P VH
EVQLVQSGAEVKKPGASVKVSCKASGYIFTNYPIHWVRQAPGQGLEWMGFIDPGGGYDEYAQKFQGRVTLTADTSTSTAYMELSSLRSEDTAVYYCARRGGGYYLDYWGQGTTLTVSS

SEQ ID NO: 8 - protein, 118 aa, synthetic construct
  REGION 1..118: note = Gen1_5072 Humanized Anti-P VH
EVQLVQSGAEVKKPGASVKVSCKASGYIFTNYPIHWVRQAPGQGLEWMGFIDPGGGYDESAQKFRGRVTLTADTSTSTVYMELSSLRSEDTAVYYCARRGGGYYLDYWGQGTTLTVSS

SEQ ID NO: 9 - protein, 118 aa, synthetic construct
  REGION 1..118: note = Gen2_5072 Humanized Anti-P VH
EVQLVQSGAEVKKPGASVKVSCKASGYIFTNYPIHWVRQAPGKGLEWMGFIDPGGGHDEYADSVKGRVTMTRDTSTSTVYMELSSLRSEDTAVYYCARRGGGYYLDYWGQGTLVTVSS

SEQ ID NO: 10 - protein, 118 aa, synthetic construct; identical to SEQ ID NO: 9
  REGION 1..118: note = Gen2_5072 Humanized Anti-P VH

SEQ ID NO: 11 - protein, 118 aa, synthetic construct
  REGION 1..118: note = Gen2_5072 Humanized Anti-P VH
EVQLLESGGGLVQPGGSLRLSCAASGYIFTNYPIHWVRQAPGKGLEWVSFIDPGGGYDEYADSVKGRFTISRDNSKNTLYLQMNSLRAEDTAVYYCAKRGGGYYLDYWGQGTLVTVSS

SEQ ID NO: 12 - protein, 118 aa, synthetic construct
  REGION 1..118: note = Gen2_5072 Humanized Anti-P VH
EVQLVESGGGLVQPGRSLRLSCAASGYIFTNYPIHWVRQAPGKGLEWVSFIDPGGGHIDYADSVEGRFTISRDNAKNSLYLQMNSLRAEDTAVYYCAKRGGGYYLDYWGQGTLVTVSS

SEQ ID NO: 13 - protein, 118 aa, synthetic construct
  REGION 1..118: note = Gen2_5072 Humanized Anti-P VH
EVQLVQSGAEVKKPGASVKVSCKASGYIFTNYPIHWVRQAPGKGLEWMGFIDPGGGYDEPDERFRDRVTMTRDTSTSTVYMELSSLRSEDTAVYYCARRGGGYYLDYWGQGTLVTVSS

SEQ ID NO: 14 - protein, 118 aa, synthetic construct
  REGION 1..118: note = Gen2_5072 Humanized Anti-P VH
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTLVTVSS

SEQ ID NO: 15 - protein, 118 aa, synthetic construct
  REGION 1..118: note = h9401-H1 Humanized Anti-P VH
QVQLVQSGAEVKKPGASVKVSCKASGYIFTNYPIHWVRQAPGQGLEWMGFIDPGGGYDEPDERFRDRVTMTRDTSTSTVYMELSSLRSEDTAVYYCARRGGGYYLDYWGQGTTVTVSS

SEQ ID NO: 16 - protein, 118 aa, synthetic construct
  REGION 1..118: note = h9401-H2 Humanized Anti-P VH
QVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSS

SEQ ID NO: 17 - protein, 118 aa, synthetic construct
  REGION 1..118: note = h9401-H3 Humanized Anti-P VH
QVQLVQSGAEVKKPGASVKMSCKASGYIFTNYPIHWVRQAPGQGLEWMGFIDPGGGYDEPDERFRDRATLTADTSTSTAYMELSSLRSEDTAVYYCARRGGGYYLDYWGQGTTLTVSS

SEQ ID NO: 18 - protein, 118 aa, synthetic construct
  REGION 1..118: note = h9401-H2b Humanized Anti-P VH
QVQLVQSGAEVKKPGASVKVSCKASGYIFTNYPIHWVRQAPGQGLEWMGFIDPGGGYDEPDERFRDRVTMTADKSTSTAYMELSSLRSEDTAVYYCARRGGGYYLDYWGQGTTVTVSS

SEQ ID NO: 19 - protein, 118 aa, synthetic construct
  REGION 1..118: note = h9401-H3b Humanized Anti-P VH
QVQLVQSGAEVKKPGASVKMSCKASGYIFTNYPIHWVRQAPGQGLEWMGFIDPGGGYDEPDERFRDRATLTADKSTSTAYMELSSLRSEDTAVYYCARRGGGYYLDYWGQGTTLTVSS

SEQ ID NO: 20 - protein, 118 aa, synthetic construct
  REGION 1..118: note = h9401-H2c Humanized Anti-P VH
QVQLVQSGAEVKKPGASVKVSCKASGYIFTNYPIHWVRQAPGQGLEWMGFIDPGGGYDEYAQKFQGRVTMTADTSTSTAYMELSSLRSEDTAVYYCARRGGGYYLDYWGQGTTVTVSS

SEQ ID NO: 21 - protein, 118 aa, synthetic construct
  REGION 1..118: note = h9401-H3c Humanized Anti-P VH
QVQLVQSGAEVKKPGASVKMSCKASGYIFTNYPIHWVRQAPGQGLEWMGFIDPGGGYDEYAQKFQGRATLTADTSTSTAYMELSSLRSEDTAVYYCARRGGGYYLDYWGQGTTLTVSS

SEQ ID NO: 22 - protein, 118 aa, synthetic construct
  REGION 1..118: note = h9401-H2d Humanized Anti-P VH
QVQLVQSGAEVKKPGASVKVSCKASGYIFTNYPIHWVRQAPGQGLEWMGFIDPGGGYDEYAQKFQGRVTMTADKSTSTAYMELSSLRSEDTAVYYCARRGGGYYLDYWGQGTTVTVSS

SEQ ID NO: 23 - protein, 122 aa, synthetic construct
  REGION 1..122: note = h9401-H3d Humanized Anti-P VH
QVQLVQSGAEVKKPGASVKMSCKASGYIFTNYPIHWVRQAPGQGLEWMGFIDPGGGYDEYAQKFQGRATLTADKSTSTAYMELSSLRSEDTAVYYCARRGGGYYLDYWGQGTTLTVSSASTK

SEQ ID NO: 24 - protein, 107 aa, synthetic construct
  REGION 1..107: note = Gen1_5072 Humanized Anti-P VL
DIQMTQSPSSLSASLGDRVTITCRASQDISFFLNWYQQKPDGTVKLLIYYTSRYHSGVPSRFSGSGSGTDFTLTISSLQPEDFATYFCQHGNTLPWTFGQGTKLEIK

SEQ ID NO: 25 - protein, 107 aa, synthetic construct
  REGION 1..107: note = Gen1_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGNTLPWTFGQGTKLEIK

SEQ ID NO: 26 - protein, 107 aa, synthetic construct
  REGION 1..107: note = Gen1_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGSTLPWTFGQGTKLEIK

SEQ ID NO: 27 - protein, 107 aa, synthetic construct
  REGION 1..107: note = Gen1_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGQTLPWTFGQGTKLEIK

SEQ ID NO: 28 - protein, 107 aa, synthetic construct; identical to SEQ ID NO: 27
  REGION 1..107: note = Gen1_5072 Humanized Anti-P VL

SEQ ID NO: 29 - protein, 107 aa, synthetic construct
  REGION 1..107: note = Gen1_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGSTLPYTFGQGTKLEIK

SEQ ID NO: 30 - protein, 107 aa, synthetic construct; identical to SEQ ID NO: 27
  REGION 1..107: note = Gen1_5072 Humanized Anti-P VL

SEQ ID NO: 31 - protein, 107 aa, synthetic construct; identical to SEQ ID NO: 29
  REGION 1..107: note = Gen1_5072 Humanized Anti-P VL

SEQ ID NO: 32 - protein, 107 aa, synthetic construct
  REGION 1..107: note = Gen2_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGSTLPWTFGQGTKVEIK

SEQ ID NO: 33 - protein, 107 aa, synthetic construct
  REGION 1..107: note = Gen2_5072 Humanized Anti-P VL
DIQMTQSPSSLSASVGDRVTITCRASQDISFFLNWYQQKPGKAPKLLIYGASSYHSGVPSRFSGSGSGTDFTLTISSLQPEDFATYYCQHGQTLPRTFGQGTKVEIK

SEQ ID NO: 34 - protein, 107 aa, synthetic construct
  REGION 1..107: note = Gen2_5072 Humanized Anti-P VL
EIVLTQSPGTLSLSPGERATLSCRASQDISFFLNWYQQKPGQAPRLLIYYTSRYHSGIPDRFSGSGSGTDFTLTISRLEPEDFAVFYCQHGSTLPRTFGQGTKVEIK

SEQ ID NO: 35 - protein, 107 aa, synthetic construct
  REGION 1..107: note = Gen2_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDVATYYCQHGSTLPYTFGQGTKVEIK

SEQ ID NO: 36 - protein, 107 aa, synthetic construct
  REGION 1..107: note = Gen2_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGSTLPRTFGQGTKVEIK

SEQ ID NO: 37 - protein, 107 aa, synthetic construct
  REGION 1..107: note = Gen2_5072 Humanized Anti-P VL
DIQMTQSPSSLSASVGDRVTITCRASQDISFFLNWYQQKPGKAPKLLIYYTSRYHSGVPSRFSGSGSGTDFTLTISSLQPEDFATYYCQHGQTLPWTFGQGTKVEIK

SEQ ID NO: 38 - protein, 107 aa, synthetic construct; identical to SEQ ID NO: 25
  REGION 1..107: note = h9401-L1 Humanized Anti-P VL

SEQ ID NO: 39 - protein, 107 aa, synthetic construct
  REGION 1..107: note = h9401-L2 Humanized Anti-P VL
DIQMTQSPSSLSASVGDRVTITCRASQDISFFLNWYQQKPGKAVKLLIYYTSRYHSGVPSRFSGSGSGTDFTLTISSLQPEDFATYYCQHGNTLPWTFGQGTKLEIK

SEQ ID NO: 40 - protein, 120 aa, synthetic construct
  REGION 1..120: note = 3196 Humanized Anti-P VH
EVTLKESGPTLVKPTQTLTLTCTFSGFSLSTSGMGVGWIRQPPGKALEWLAHIWWDDVKSYNPALKSRLTITKDTSKNQVVLTMTNMDPVDTATYYCARIGDGYYSFDYWGQGTTVTVSS

SEQ ID NO: 41 - protein, 120 aa, synthetic construct
  REGION 1..120: note = 3196 Humanized Anti-P VH
EVTLKESGPTLVKPTQTLTLTCTFSGFSLSTSGMGVGWIRQPPGKALEWLAHIWWDDVKSYNPALKSRLTITKDTSKNQVVLTITNVDPVDTATYYCARIGDGYYSFDYWGQGTTLTVSS

SEQ ID NO: 42 - protein, 120 aa, synthetic construct; identical to SEQ ID NO: 40
  REGION 1..120: note = 3196 Humanized Anti-P VH

SEQ ID NO: 43 - protein, 120 aa, synthetic construct
  REGION 1..120: note = h3196-H1 Humanized Anti-P VH
QVTLKESGPTLVKPTQTLTLTCTFSGFSLSTSGMGVGWIRQPPGKALEWLAHIWWDDVKSYNPALKSRLTITKDTSKNQVVLTMTNMDPVDTATYYCARIGDGYYSFDYWGQGTTVTVSS

SEQ ID NO: 44 - protein, 120 aa, synthetic construct
  REGION 1..120: note = h3196-H2 Humanized Anti-P VH
QVTLKESGPTLVKPTQTLTLTCTFSGFSLSTSGMGVGWIRQPPGKALEWLAHIWWDDVKSYNPALKSRLTITKDTSKNQVVLTITNVDPVDTATYYCARIGDGYYSFDYWGQGTTLTVSS

SEQ ID NO: 45 - protein, 107 aa, synthetic construct
  REGION 1..107: note = 3196 Humanized Anti-P VL
DIQMTQSPSSLSASVGDRVTITCRASQDVSDAVAWYQQKPGKAPKLLIYSPSYRYTGVPSRFSGSGSGTDFTLTISSLQPEDFATYYCQQHYSTPWTFGQGTKLEIK

SEQ ID NO: 46 - protein, 107 aa, synthetic construct
  REGION 1..107: note = 3196 Humanized Anti-P VL
DIQMTQSPSSLSASVGDRVTITCRASQDVSDAVAWFQQKPGKAPKLLIYSPSYRYTGVPSRFSGSGSGTDFTFTISSLQPEDFATYYCQQHYSTPWTFGQGTKLEIK

SEQ ID NO: 47 - protein, 107 aa, synthetic construct
  REGION 1..107: note = 3196 Humanized Anti-P VL
DIQMTQSPSSLSASVGDRVTITCRASQDVSDAVAWFQQKPGKAPKLLIYSPSYRYTGVPSRFSGSGSGTDFTFTISSLQPEDLATYYCQQHYSTPWTFGQGTKLEIK

SEQ ID NO: 48 - protein, 107 aa, synthetic construct; identical to SEQ ID NO: 45
  REGION 1..107: note = h3196-L1 Humanized Anti-P VL

SEQ ID NO: 49 - protein, 107 aa, synthetic construct; identical to SEQ ID NO: 46
  REGION 1..107: note = h3196-L2 Humanized Anti-P VL

SEQ ID NO: 50 - protein, 107 aa, synthetic construct; identical to SEQ ID NO: 47
  REGION 1..107: note = h3196-L3 Humanized Anti-P VL

SEQ ID NO: 51 - protein, 125 aa, synthetic construct
  REGION 1..125: note = Humanized Anti-P VH
EVQLLESGGGLVQPGGSLRLSCAASGFSFSSGYWIFWVRQAPGKGLEWVSGIYSGSSGTTYYADSVKGRFTISRDNSKNTLYLQMNSLRAEDTAVYYCAKSVDGIDSYDAAFNLWGQGTLVTVSS

SEQ ID NO: 52 - protein, 125 aa, synthetic construct
  REGION 1..125: note = Humanized Anti-P VH
EVQLLESGGGLVQPGGSLRLSCAASGFSFSSGYWIFWVRQAPGKGLELVGGIYSGSSGTTYYADSVKGRFTISKDNSKNTVYLQMNSLRAEDTAVYYCARSVDGIDSYDAAFNLWGQGTLVTVSS

SEQ ID NO: 53 - protein, 125 aa, synthetic construct
  REGION 1..125: note = Humanized Anti-P VH
EVQLLESGGGLVQPGGSLRLSCAASGFSFSSGYWIFWVRQAPGKGLELVGGIYSGSSGTTYYADWAKGRFTISKDNSKNTVYLQMNSLRAEDTAVYYCARSVDGIDSYDAAFNLWGQGTLVTVSS

SEQ ID NO: 54 - protein, 125 aa, synthetic construct
  REGION 1..125: note = Humanized Anti-P VH
EVQLLESGGGLVQPGGSLRLSCAASGFSFSSGYWIFWVRQAPGKGLELVGGIYSGSSGTTYYADSVKGRFTISKDSSKNTVYLQMNSLRAEDTAVYYCARSVDGIDSYDAAFNLWGQGTLVTVSS

SEQ ID NO: 55 - protein, 125 aa, synthetic construct
  REGION 1..125: note = Humanized Anti-P VH
EVQLLESGGGLVQPGGSLRLSCAASGFSFSSGYWIFWVRQAPGKGLELVGGIYSGSSGTTYYADWAKGRFTISKDSSKNTVYLQMNSLRAEDTAVYYCARSVDGIDSYDAAFNLWGQGTLVTVSS

SEQ ID NO: 56 - protein, 125 aa, synthetic construct
  REGION 1..125: note = Humanized Anti-P VH
EVQLLESGGGLVQPGGSLRLSCAASGFSFSSSYWIFWVRQAPGKGLEWVSGIYSSSGRMYYADSVKGRFTISRDNSKNTLYLQMNSLRAEDTAVYYCAKSADGSDSYDAYFTLWGQGTLVTVSSS

SEQ ID NO: 57 - protein, 125 aa, synthetic construct
  REGION 1..125: note = Humanized Anti-P VH
EVQLLESGGGLVQPGGSLRISCAASGFSFSSSYWIFWVRQAPGKGLELIGGIYSSSGRMYYADSVKGRFTISKDNSKNTMYLQMNSLRAEDTAVYYCARSADGSDSYDAYFTLWGQGTLVTVSSS

SEQ ID NO: 58 - protein, 125 aa, synthetic construct
  REGION 1..125: note = Humanized Anti-P VH
EVQLLESGGGLVQPGGSLRISCAASGFSFSSSYWIFWVRQAPGKGLELIGGIYSSSGRMYYADWAKGRFTISKDNSKNTMYLQMNSLRAEDTAVYYCARSADGSDSYDAYFTLWGQGTLVTVSSS

SEQ ID NO: 59 - protein, 125 aa, synthetic construct
  REGION 1..125: note = Humanized Anti-P VH
EVQLLESGGGLVQPGGSLRISCAASGFSFSSSYWIFWVRQAPGKGLELIGGIYSSSGRMYYADSVKGRFTISKDSSKNTMYLQMNSLRAEDTAVYYCARSADGSDSYDAYFTLWGQGTLVTVSSS

SEQ ID NO: 60 - protein, 125 aa, synthetic construct
  REGION 1..125: note = Humanized Anti-P VH
EVQLLESGGGLVQPGGSLRISCAASGFSFSSSYWIFWVRQAPGKGLELIGGIYSSSGRMYYADWAKGRFTISKDSSKNTMYLQMNSLRAEDTAVYYCARSADGSDSYDAYFTLWGQGTLVTVSSS

SEQ ID NO: 61 - protein, 110 aa, synthetic construct
  REGION 1..110: note = Humanized Anti-P VL
DIQMTQSPSSLSASVGDRVTITCRASDNIYSLLAWYQQKPGKAPKLLIYRASTLASGVPSRFSGSGSGTDFTLTISSLQPEDFATYYCQQHYDYNYLDVAFGGGTKVEIK

SEQ ID NO: 62 - protein, 110 aa, synthetic construct
  REGION 1..110: note = Humanized Anti-P VL
DIQLTQSPSSLSASVGDRVTITCRASDNIYSLLAWYQQKPGKAPKLLIYRASTLASGVPSRFSGSGSGTDFTLTISSVQPEDFATYYCQQHYDYNYLDVAFGGGTKVEIK

SEQ ID NO: 63 - protein, 110 aa, synthetic construct
  REGION 1..110: note = Humanized Anti-P VL
DYQLTQSPSSLSASVGDRVTITCRASDNIYSLLAWYQQKPGKAPKLLIYRASTLASGVPSRFSGSGSGTDFTLTISSVQPEDFATYYCQQHYDYNYLDVAFGGGTKVEIK

SEQ ID NO: 64 - protein, 110 aa, synthetic construct
  REGION 1..110: note = Humanized Anti-P VL
DIQLTQSPSSLSASVGDRVTITCRASDNIYSLLAWYQQKPGKAPKLLIYRASTLASGVPSRFSGSGSGTDFTLTISSVQPEDAATYYCQQHYDYNYLDVAFGGGTKVEIK

SEQ ID NO: 65 - protein, 110 aa, synthetic construct
  REGION 1..110: note = Humanized Anti-P VL
DYQLTQSPSSLSASVGDRVTITCRASDNIYSLLAWYQQKPGKAPKLLIYRASTLASGVPSRFSGSGSGTDFTLTISSVQPEDAATYYCQQHYDYNYLDVAFGGGTKVEIK

SEQ ID NO: 66 - protein, 110 aa, synthetic construct
  REGION 1..110: note = Humanized Anti-P VL
DIQMTQSPSSLSASVGDRVTITCRASQNIYSNLAWYQQKPGKAPKLLIYRASTLASGVPSRFSGSGSGTDFTLTISSLQPEDFATYYCQQHWDYDYIDVAFGGGTKVEIK

SEQ ID NO: 67 - protein, 110 aa, synthetic construct
  REGION 1..110: note = Humanized Anti-P VL
DIQMTQSPSSLSASVGDRVTITCRASQNIYSNLAWYQQKPGKAPKLLIYRASTLASGVPSRFSGSGSGTDFTLTISSVQPEDFATYYCQQHWDYDYIDVAFGGGTKVEIK

SEQ ID NO: 68 - protein, 110 aa, synthetic construct
  REGION 1..110: note = Humanized Anti-P VL
DYQMTQSPSSLSASVGDRVTITCRASQNIYSNLAWYQQKPGKAPKLLIYRASTLASGVPSRFSGSGSGTDFTLTISSVQPEDFATYYCQQHWDYDYIDVAFGGGTKVEIK

SEQ ID NO: 69 - protein, 110 aa, synthetic construct
  REGION 1..110: note = Humanized Anti-P VL
DIQMTQSPSSLSASVGDRVTITCRASQNIYSNLAWYQQKPGKAPKLLIYRASTLASGVPSRFSGSGSGTDFTLTISSVQPEDAATYYCQQHWDYDYIDVAFGGGTKVEIK

SEQ ID NO: 70 - protein, 110 aa, synthetic construct
  REGION 1..110: note = Humanized Anti-P VL
DYQMTQSPSSLSASVGDRVTITCRASQNIYSNLAWYQQKPGKAPKLLIYRASTLASGVPSRFSGSGSGTDFTLTISSVQPEDAATYYCQQHWDYDYIDVAFGGGTKVEIK

SEQ ID NO: 71 - protein, 125 aa, synthetic construct
  REGION 1..125: note = Humanized Anti-ALB VH
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTK

SEQ ID NO: 72 - protein, 116 aa, synthetic construct
  REGION 1..116: note = Humanized Anti-ALB VL
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAP

SEQ ID NO: 73 - protein, 119 aa, synthetic construct
  REGION 1..119: note = Humanized Lama Glama LVP058 Anti-P HC
EVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 74 - protein, 119 aa, synthetic construct
  REGION 1..119: note = Humanized LVP058 Anti-P HC
EVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 75 - protein, 119 aa, synthetic construct
  REGION 1..119: note = Humanized LVP058 Anti-P HC
EVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 76 - protein, 119 aa, synthetic construct
  REGION 1..119: note = Humanized Anti-Properdin Vhh
EVQLLESGGGLVQAGGSLRLSCAASGRISSIIHMAWYRQAPGKQRELVSEISRVGTTVYADSVKGRFTISRDDAKNTVTLQMNSLKPEDTAVYYCNALQYEAHGGASYWGQGTQVTVSS

SEQ ID NO: 77 - protein, 119 aa, synthetic construct
  REGION 1..119: note = Humanized Vicugna pacos Anti-Properdin Vhh
QVQLVESGGGLVQAGGSLRLSCAASGRISSIIHMAWYRQAPGKQRELVAEISRIGTTVYAESVAGRFTISRDDAKNTVTLQMNSLKPEDTAVYYCNALQYEKHGGASYWGQGTQVTVSG

SEQ ID NO: 78 - protein, 119 aa, synthetic construct
  REGION 1..119: note = Humanized Vicugna pacos Anti-Properdin Vhh
QVQVVESGGGLRQTGGSLRLSCTASGRIFEVNMMAWYRQAPGKQRELVAEISRVGTTVYADSVKGRFTISRDSAKNTVTLQMNSLKSEDTAVYYCNALQYDRYGGAEYWGQGTQVTVSS

SEQ ID NO: 79 - protein, 119 aa, synthetic construct
  REGION 1..119: note = Humanized Vicugna pacos Anti-Properdin Vhh
QVQLVESGGGLRQTGESLRLSCTASGRIFEVNMMAWYRQAPGKQRELVAEISRVGTTTYADSVKGRFTISRDSAKNTVTLQMNSLKSEDTAVYYCNALQYDRYGGAEYWGQGTQVTVSG

SEQ ID NO: 80 - protein, 119 aa, synthetic construct
  REGION 1..119: note = Humanized Vicugna pacos Anti-P Vhh
QVQLAESGGGLVQAGDSLKLSCTASGRIFEVNMMAWYRQAPGKDRELVAEISRVGTTTYADSVKGRFTISRDSAKNTVTLQMNSLKSEDTAVYYCNALQYSRYGGAEYWGQGTQVTVSG

SEQ ID NO: 81 - protein, 129 aa, synthetic construct
  REGION 1..129: note = Humanized Camelidae Anti-P Fab - Vhh Domain
QVQLVESGGGLVQAGGSLRLSCAASGGTFSSYSMGWFRQAPGKEREFVAAITWNGVSTYYADSVKGRFTISRDNAKNTVYLQMNSLKPTDTAVYYCAAEITTRYSGFYYYEDNKSYDYWGQGTQVTVSS

SEQ ID NO: 82 - protein, 129 aa, synthetic construct
  REGION 1..129: note = Humanized Lama glama Anti-P Fab - Vhh Domain
QVQLIESGGGLVQAGDSLRLSCATSEGTFSRFAMGWFRQAPGKEREFVAAINWSGGITYYADSIAGRFTISRDNAKNTVYLQMNSLKPEDTADYYCAAETTTRYSGYYYYEDNKSYDYWGQGTLVTVSG

SEQ ID NO: 83 - protein, 129 aa, synthetic construct
  REGION 1..129: note = Humanized Lama glama Anti-P Fab - Vhh Domain
QVQLVESGGGLVQAGGSLRLSCAASGRTFSTLGMGWFRQAPGKERQFVAAINWSGSSTYYANSVKGRFTISRDNAQSTMYLQMNSLKPEDTAVYYCAADLDSRYSAYYYYSDESQYDYWGQGTLVTVSG

SEQ ID NO: 84 - protein, 129 aa, synthetic construct
  REGION 1..129: note = Humanized Lama glama Anti-P Fab - Vhh Domain
QVQLVESGGGLVQAGGSLRLSCAASGRTFSSYAMGWFRQPPGKEREFVAAITWRGASTYYADPIKGRFTISRDNAKNTVYLQMSSLKPEDTAVYYCAAEEPSYYSGSYYYMMGESYAYWGQGTLVTVSG

SEQ ID NO: 85 - protein, 129 aa, synthetic construct
  REGION 1..129: note = Humanized Lama glama Anti-P Fab - Vhh Domain
QVHLVESGGGLVQAGGSLRLSCAASGRTFSSYAMGWFRQPPGKERQFVAAITWSGSSIYYADSIKGRFTISRDNAKNTVYLQMNSLKPEDTAVYYCAAEETSAYSGSYYYMMGDSYSYWGQGTQVTVSG

SEQ ID NO: 86 - protein, 129 aa, synthetic construct
  REGION 1..129: note = Humanized Lama glama Anti-P Fab - Vhh Domain
QVQLVESGGGLVQPGGSLSLSCAASGRTFSSYAMGWFRQAPGKEREWVAAITWSGANIYYADSIKGRFTLSRDNAENTVWLQLNSLKPEDTAVYYCAAAESGRYSGRAYYSAPGVYLYWGQGTLVTVSG

SEQ ID NO: 87 - protein, 129 aa, synthetic construct
  REGION 1..129: note = Humanized Lama glama Anti-P Fab - Vhh Domain
QVQLVESGGGLVQAGGSLRLSCAASGRTFSNYAMAWFRQAPGKEREFVASISGSGDSRYYADSVKGRFTISRDNAKNTVYLQTNSPKPEDTAVYYCAAVLPTRYSGFYYYSDGTQYHYWGQGTQVTVSS

SEQ ID NO: 88 - protein, 129 aa, synthetic construct
  REGION 1..129: note = Humanized Lama glama Anti-P Fab - Vhh Domain
QVRLVESGGGLVQAGDSLRLSCATSGRTLSSYAMGWFRQAPGKEREFVAATTWRDTSTYYADSVKGRFTISRDNAKNTVYLQTNSLKPEDTAAYYCAAEEPSKYSGRSYYMMGASYDYWGQGTQVTVSS

SEQ ID NO: 89 - protein, 129 aa, synthetic construct
  REGION 1..129: note = Humanized Lama glama Anti-P Fab - Vhh Domain
QVQLVESGGGLVQPGGSLRLSCATSGGTFSSYAMGWFRQAPGKEREFVAATTWQGSNRYYAESVAGRFTISRDNAKSTVWLQMNSLKPEDTAVYYCAAEHSTRYSGFYYYTRGETYHYWGQGTQVTVSG

SEQ ID NO: 90 - protein, 129 aa, synthetic construct
  REGION 1..129: note = Humanized Lama glama Anti-P Fab - Vhh Domain
QVQLVESGGGLVQPGGSLRLSCAASGFPLNSYAIGWFRQAPGKEREGVSCISVSDDSTYYTDSVKGRFTISRDNAKNTVYLQMNSLKPEDTAVYYCAVDSAPLYGDYVCKPLENEYDFWGQGTQVTVSG

SEQ ID NO: 91 - protein, 129 aa, synthetic construct
  REGION 1..129: note = Humanized Lama glama Anti-P Fab - Vhh Domain
QVQLVESGGGLVQAGDSLRFSCAASGFTFSSYAMGWFRQAPGKEREFVAAITWSGVSTYYADSIAGRFTISRDNAKNRVYLQMNSLKPEDTADYSCAADGSGRYSGMEYYNRDWVYDYWGQGTQVTVSS

SEQ ID NO: 92 - protein, 128 aa, synthetic construct
  REGION 1..128: note = Humanized Lama glama Anti-P Fab - Vhh Domain
QVRLTESGGGLVQYGTNLTLTCVASGLISTRNKMGWFRRRSGGQREFVASSTVLSDDVIQDDIAETVKGRFAVARNDYKNILYLQMTAVKPEDTGFYWCASGTSLFGASRREDDFNAWGVGTQVTVSA

SEQ ID NO: 93 - protein, 119 aa, synthetic construct
  REGION 1..119: note = Humanized Lama glama Fab - Vhh Domain
EVQLVESGGGLVQPGGSLRLSCAASGFTFGSADMSWVRQAPGKGPEWVSAINSNGGSTYYAASVKGRFTISRDNAKNTLYLQMNSLKPEDTAVYYCAQGNWYTEEYHYWGQGTQVTVSG

SEQ ID NO: 94 - protein, 122 aa, synthetic construct
  REGION 1..122: note = Humanized Vicugna pacos Fab HC Anti-Properdin
QVQLVESGGGLVQAGGSLRLSCAASGRTFSSYAMGWFRQAPGKEREFVAAIGWNGEGIYYADSVKGRFTISRDNAKNTGYLQMNSLKPEDTAVYYCAADSEGVVPGFPIAYWGQGTQVTVSG

SEQ ID NO: 95 - protein, 122 aa, synthetic construct
  REGION 1..122: note = Humanized Lama glama Anti-P Fab - Vhh Domain
EVQLVESGGGLVQPGGSLRLSCATSGRPFSSYAMGWFRQAPGKEREIVAGLSWSGGQIYYADSVKGRFTISRDNAKNTGDLQMNSLKPEDTAVYYCAIGPALTTGPTAYRYWGQGTQVTVSS

SEQ ID NO: 96 - protein, 122 aa, synthetic construct
  REGION 1..122: note = Humanized Lama glama Anti-P Fab - Vhh Domain
QVHLVESGGGLVQAGGSLRLSCTASGGTVGDYAVGWFRQAPGKERELIGVVSRLGARTGYADSVLGRFTISRDDVKNTVFLQMDSVKPEDTAVYYCAARRDYSFEVVPYDYWGQGTQVTVSG

SEQ ID NO: 97 - protein, 124 aa, synthetic construct
  REGION 1..124: note = Humanized Lama glama Anti-P Fab - Vhh Domain
QVQLVESGGGLVQPGGSLRLSCAASGRTFSSYSMGWFRQRHGNEREFVAAISWSGRSTYYAESVKGRFAISRDNANTTVYLQMNSLKPEDSAVYYCAASTRGWYGTQESDYNFWGQGTQVTVSG

SEQ ID NO: 98 - protein, 125 aa, synthetic construct
  REGION 1..125: note = Humanized Lama glama Anti-P Fab - Vhh Domain
QVQLIESGGGLVQAGGSLRLSCTASGRTFSNYAMGWFRQAPGKEREFLAAISRSGESTQYATFVKGRFAIARDNAKNTVSLQMNSLKPEDTAVYFCAAKIAVLVSTTYNSQYEYWGQGTLVTVSS

SEQ ID NO: 99 - protein, 126 aa, synthetic construct
  REGION 1..126: note = Humanized Lama glama Anti-P Fab - Vhh Domain
QVQLIESGGGLVQEGASLRLSCAGSGPMFSRLAVGWFRQAPGKEREFVAVINWSGSADFYTNSVKGRFTISRDNAKNTVYLEMNTLKPEDSAVYYCAADQNPLTLRTGVRDVGRQWGQGTEVTVSS

SEQ ID NO: 100 - protein, 127 aa, synthetic construct
  REGION 1..127: note = Humanized Lama glama Anti-P Fab - Vhh Domain
EVQLVESGGGLVQPGGSLRLSCAASGFTLDYYAIGWFRQAPGKEREGVSCISRTDGSTYYADSVKGRFTISRDNAKNTVYLQMNSLKPEDTAVYYCAVDDSYPTGGISCLFGHFGSWGQGTQVTVSS

SEQ ID NO: 101 - protein, 130 aa, synthetic construct
  REGION 1..130: note = Humanized Vicugna pacos Anti-P Fab - Vhh Domain
QVQLVESGGGLVQAGGSLRLSCAASGRTFSSYAMGWFRQAPGKEREFVAAVPWTYGSKYYADSVKGRFTISRDDAKNTVYLQMNNLKPEDTAVYYCAADSSAGYYSGFDYYSAATPYDLWGQGTQVTVSG

SEQ ID NO: 102 - protein, 116 aa, synthetic construct
  REGION 1..116: note = Humanized Lama glama Anti-P Fab - Vhh Domain
QVQMVESGGGLVQAGGSLRLSCAASGLTNRIRIMGWYRQAPGKLRELVATITNDGSTHYADSVKGRFTISTDNAKNTVFLQMNSLKPEDTAVYICNVGENWGPAYWGQGTQVTVSG

SEQ ID NO: 103 - protein, 118 aa, synthetic construct
  REGION 1..118: note = Humanized Lama glama Anti-P Fab - Vhh Domain
  VAR_SEQ 19: note = Xaa can be any naturally occurring amino acid
QVQLVESGGGLVQAGGSLXLSCAASGSDRRINGMGWYRHPPGKQRELVAAITSGGSTNYADSVKGRFTISTNNANNMMYLQMNSLKPEDTAVYYCAIDEFGTGWLDYCGQGTQVTVSG

SEQ ID NO: 104 - protein, 120 aa, synthetic construct
  REGION 1..120: note = Humanized Lama glama Anti-P Fab - Vhh Domain
QVLLEESGGGLERTGGSLRLSCAASGSIFSVNSMTWYRQAPGKRREFLGTITEEGRTNYADSVKGRFTISRDNAKNTMYLQMNSLKPEDTAVYYCYANLISSEDRTFGVWGQGTQVTVSS

SEQ ID NO: 105 - protein, 121 aa, synthetic construct
  REGION 1..121: note = Humanized Lama glama Anti-P Fab - Vhh Domain
QVHMVESGGGLVQAGGSLRFSCAASGNIFTISTLDWYRQAPGEQRELVATLTPDGITDYAGSVKGRFTISRDNAKNTVYLQMNSLKPEDTAVYYCNAWRYSDDYRGRVDYWGQGTQVTVSG

SEQ ID NO: 106 - protein, 129 aa, synthetic construct
  REGION 1..129: note = Humanized Lama glama Anti-P Fab - Vhh Domain
QVQLMESGGGEVQAGGSLRLSCAASGSIFDISAMGWYRQAPGKQRELVADITSSGSTQYADSVKGRFTISRDNAKNTVYLQMNSLKPEDTAVYTCAAESIRESQNRHQLGYMGPLYDYWGQGTQVTVSG

SEQ ID NO: 107 - protein, 125 aa, synthetic construct
  REGION 1..125: note = Humanized Lama glama Anti-P Fab - Vhh Domain
QVQLVESGGGLVQPGGSLRLSCAASGSDYYAIGWFRQAPGKEREGVSCMSRTDGSTYYADSVKDRFTISRDYAKNTVYLQMNSLKPEDTAVYYCGLDRSYPTGGISCLFGDFGSWGQGTQVTVSG

SEQ ID NO: 108 - protein, 123 aa, synthetic construct
  REGION 1..123: note = Humanized Anti-Albumin Fab-Vhh
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 109 - protein, 115 aa, synthetic construct [sequence omitted]

SEQ ID NO: 110 - protein, 115 aa, synthetic construct [sequence omitted]

SEQ ID NO: 111 - protein, 115 aa, synthetic construct [sequence omitted]

SEQ ID NO: 112 - protein, 115 aa, synthetic construct [sequence omitted]

SEQ ID NO: 113 - protein, 115 aa, synthetic construct [sequence omitted]

SEQ ID NO: 114 - protein, 115 aa, synthetic construct [sequence omitted]

SEQ ID NO: 115 - protein, 115 aa, synthetic construct
  REGION 1..115: note = Humanized Anti-Albumin
EVQLVESGGGLVQPGNSLRLSCAASGFTFSSFGMSWVRQAPGKGLEWVSSISGSGSDTLYADSVKGRETISRDNAKTTLYLQMNSLRPEDTAVYYCTIGGSLSRSSQGTLVTVSS

SEQ ID NO: 116 - protein, 115 aa, synthetic construct
  REGION 1..115: note = Humanized Anti-Albumin
EVQLVESGGGLVQPGNSLRLSCAASGFTFSSFGMSWVRQAPGKGLEWVSSISGSGSDTLYADSVKGRETISRDNAKNTLYLQMNSLRPEDTAVYYCTIGGSLSRSSQGTLVTVSS

SEQ ID NO: 117 - protein, 115 aa, synthetic construct [sequence omitted]

SEQ ID NO: 118 - protein, 115 aa, synthetic construct [sequence omitted]

SEQ ID NO: 119 - protein, 115 aa, synthetic construct [sequence omitted]

SEQ ID NO: 120 - protein, 115 aa, synthetic construct
  REGION 1..115: note = Humanized Anti-Albumin
AWQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 121 - protein, 115 aa, synthetic construct
  REGION 1..115: note = Humanized Anti-Albumin
AWQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTQWTVSS

SEQ ID NO: 122 - protein, 115 aa, synthetic construct
  REGION 1..115: note = Humanized Anti-Albumin
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTQVTVSS

SEQ ID NO: 123 - protein, 115 aa, synthetic construct [sequence omitted]

SEQ ID NO: 124 - protein, 115 aa, synthetic construct
  REGION 1..115: note = Humanized Anti-Albumin
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLWTVSS

SEQ ID NO: 125 - protein, 115 aa, synthetic construct; identical to SEQ ID NO: 124
  REGION 1..115: note = Humanized Anti-Albumin

SEQ ID NO: 126 - protein, 115 aa, synthetic construct [sequence omitted]

SEQ ID NO: 127 - protein, 115 aa, synthetic construct [sequence omitted]

SEQ ID NO: 128 - protein, 115 aa, synthetic construct; identical to SEQ ID NO: 124
  REGION 1..115: note = Humanized Anti-Albumin

SEQ ID NO: 129 - protein, 326 aa, synthetic construct
  REGION 1..326: note = Humanized IgG1 Fc
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 326 aa, synthetic construct
  REGION 1..326: note = Humanized N297 Fc HC
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 326 aa, synthetic construct
  REGION 1..326: note = Humanized N297 YTE Mutation Fc HC
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 322 aa, synthetic construct
  REGION 1..322: note = Humanized Ultomiris Fc HC
GPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 133 - protein, 322 aa, synthetic construct
  REGION 1..322: note = Humanized Soliris Fc HC
GPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 232 aa, synthetic construct
  REGION 1..232: note = Humanized IgG1 Fc (No CH1)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 228 aa, synthetic construct
  REGION 1..228: note = Humanized IgG2 Fc (No CH1)
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 136 - protein, 229 aa, synthetic construct
  REGION 1..229: note = Humanized IgG4 Fc (No CH1)
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - protein, 229 aa, synthetic construct
  REGION 1..229: note = Humanized IgG4 Fc (No CH1)
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 138 - protein, 100 aa, synthetic construct
  REGION 1..100: note = Humanized Fc (CH1 Only)
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 139 - protein, 101 aa, synthetic construct
  REGION 1..101: note = Humanized Fc LC
SVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 5 aa, synthetic construct
  REGION 1..5: note = Humanized Linker
GGGGS

SEQ ID NO: 141 - protein, 5 aa, synthetic construct
  REGION 1..5: note = Humanized Linker
GGGSG

SEQ ID NO: 142 - protein, 4 aa, synthetic construct
  REGION 1..4: note = Humanized Linker
SGGG

SEQ ID NO: 143 - protein, 15 aa, synthetic construct
  REGION 1..15: note = Humanized Linker
GGGGSGGGGSGGGGS

SEQ ID NO: 144 - protein, 14 aa, synthetic construct
  REGION 1..14: note = Humanized Linker
GGGGDGGGGDGGGG

SEQ ID NO: 145 - protein, 14 aa, synthetic construct
  REGION 1..14: note = Humanized Linker
GGGGEGGGGEGGGG

SEQ ID NO: 146 - protein, 4 aa, synthetic construct
  REGION 1..4: note = Humanized Linker
GGGE

SEQ ID NO: 147 - protein, 4 aa, synthetic construct
  REGION 1..4: note = Humanized Linker
GGGD

SEQ ID NO: 148 - protein, 5 aa, synthetic construct
  REGION 1..5: note = Humanized Linker
GGGGA

SEQ ID NO: 149 - protein, 15 aa, synthetic construct
  REGION 1..15: note = Humanized Linker
GGGGAGGGGAGGGGS

SEQ ID NO: 150 - protein, 125 aa, synthetic construct
  REGION 1..125: note = Humanized Anti-TNF VH
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTK

SEQ ID NO: 151 - protein, 113 aa, synthetic construct
  REGION 1..113: note = Humanized Anti-TNF VL
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAP

SEQ ID NO: 152 - protein, 127 aa, synthetic construct
  REGION 1..127: note = Humanized Anti-VEGF VH
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTK

SEQ ID NO: 153 - protein, 113 aa, synthetic construct
  REGION 1..113: note = Humanized Anti-VEGF VL
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAP

SEQ ID NO: 154 - protein, 254 aa, synthetic construct
  REGION 1..254: note = Humanized Camelid Anti-Alb & Anti-P mAb
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGSGGGSGGGSEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 155 - protein, 246 aa, synthetic construct
  REGION 1..246: note = Humanized Camelid Anti-Alb & Anti-P mAb
EVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGSGGGSGGGSEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 156 - protein, 222 aa, synthetic construct
  REGION 1..222: note = Humanized Anti-P HC
QVQLVQSAPEVAKPGTSVKMSCKASGYIFTNYPIHWVKQAPGQGLEWIGFIDPGGGYDEPDERFRDRATLTADKSTSTAYMELSSLRSEDTAIYYCARRGGGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 157 - protein, 214 aa, synthetic construct
  REGION 1..214: note = Humanized Anti-P LC
DIQMTQSPSSLSASLGDRVTITCRASQDISFFLNWYQQKPDGTVKLLIYYTSRYHSGVPSRFSGSGSGTDFTLTISSLQPEDFATYFCQHGNTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 360 aa, synthetic construct
  REGION 1..360: note = Humanized Anti-Alb & Anti-P - HC
QVQLVQSAPEVAKPGTSVKMSCKASGYIFTNYPIHWVKQAPGQGLEWIGFIDPGGGYDEPDERFRDRATLTADKSTSTAYMELSSLRSEDTAIYYCARRGGGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 159 - protein, 214 aa, synthetic construct; identical to SEQ ID NO: 157
  REGION 1..214: note = Humanized Anti-P LC

SEQ ID NO: 160 - protein, 352 aa, synthetic construct
  REGION 1..352: note = Humanized Anti-Alb & Anti-P - HC
QVQLVQSAPEVAKPGTSVKMSCKASGYIFTNYPIHWVKQAPGQGLEWIGFIDPGGGYDEPDERFRDRATLTADKSTSTAYMELSSLRSEDTAIYYCARRGGGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSEVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 161 - protein, 214 aa, synthetic construct; identical to SEQ ID NO: 157
  REGION 1..214: note = Humanized Anti-P LC

SEQ ID NO: 162 - protein, 363 aa, synthetic construct
  REGION 1..363: note = Humanized Camelid Anti-P Full IgG4 HC
EVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSSGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 163 - protein, 367 aa, synthetic construct
  REGION 1..367: note = Humanized Anti-Alb Full IgG4 HC
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 164 - protein, 366 aa, synthetic construct
  REGION 1..366: note = Humanized Camelid Anti-P Full IgG1 HC
EVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 370 aa, synthetic construct
  REGION 1..370: note = Humanized Anti-Alb Full IgG1 HC
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 448 aa, synthetic construct
  REGION 1..448: note = Humanized Anti-P Full IgG1 HC
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 214 aa, synthetic construct
  REGION 1..214: note = Humanized Anti-P Full LC
DIQMTQSPSSLSASVGDRVTITCRASQDISFFLNWYQQKPGKAPKLLIYYTSRYHSGVPSRFSGSGSGTDFTLTISSLQPEDFATYYCQHGNTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 451 aa, synthetic construct
  REGION 1..451: note = Humanized Anti-Alb Full IgG1 HC
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 217 aa, synthetic construct
  REGION 1..217: note = Humanized Anti-Alb Full LC
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 455 aa, synthetic construct
  REGION 1..455: note = Humanized Anti-P Full IgG1 HC
EVQLLESGGGLVQPGGSLRLSCAASGFSFSSGYWIFWVRQAPGKGLELVGGIYSGSSGTTYYADSVKGRFTISKDNSKNTVYLQMNSLRAEDTAVYYCARSVDGIDSYDAAFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 217 aa, synthetic construct
  REGION 1..217: note = Humanized Anti-P Full LC
DIQLTQSPSSLSASVGDRVTITCRASDNIYSLLAWYQQKPGKAPKLLIYRASTLASGVPSRFSGSGSGTDFTLTISSVQPEDFATYYCQQHYDYNYLDVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 451 aa, synthetic construct
  REGION 1..451: note = Humanized Anti-Alb Full IgG1 HC
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 217 aa, synthetic construct; identical to SEQ ID NO: 169
  REGION 1..217: note = Humanized Anti-Alb Full LC

SEQ ID NO: 174 - protein, 448 aa, synthetic construct
  REGION 1..448: note = Humanized Anti-P Full HC (IgG1)
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 214 aa, synthetic construct; identical to SEQ ID NO: 167
  REGION 1..214: note = Humanized Anti-P Full LC

SEQ ID NO: 176 - protein, 451 aa, synthetic construct
  REGION 1..451: note = Humanized Anti-TNF Full HC (IgG1)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 214 aa, synthetic construct
  REGION 1..214: note = Humanized Anti-TNF Full LC
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 448 aa, synthetic construct
  REGION 1..448: note = Humanized Anti-P Full HC
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 214 aa, synthetic construct; identical to SEQ ID NO: 167
  REGION 1..214: note = Humanized Anti-P Full LC

SEQ ID NO: 180 - protein, 453 aa, synthetic construct
  REGION 1..453: note = Humanized Anti-VEGF Full HC
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 214 aa, synthetic construct
  REGION 1..214: note = Humanized Anti-VEGF Full LC
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 460 aa, synthetic construct
  REGION 1..460: note = Humanized Camelid Anti-P w/ IgG1 N297A Fc
EVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSSGGGGSGGGGSGGGGSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 464 aa, synthetic construct
  REGION 1..464: note = Humanized Camelid Anti-Alb w/ IgG1 N297A Fc
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 244 aa, synthetic construct
  REGION 1..244: note = Humanized Anti-P Full HC (IgG1 YTE Fc)
EVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSSASTKEVQLVQSGAEVKKPGASVKVSCKASGYIFTNYPIHWVRQAPGQGLEWMGFIDPGGGYDEPDERFRDRVTMTADTSTSTAYMELSSLRSEDTAVYYCARRGGGYYLDYWGQGTTVTVSSASTK

SEQ ID NO: 185 - protein, 214 aa, synthetic construct; identical to SEQ ID NO: 167
  REGION 1..214: note = Humanized Anti-P Full LC

SEQ ID NO: 186 - protein, 456 aa, synthetic construct
  REGION 1..456: note = Humanized Camelid Anti-Alb Full HC (IgG1 YTE Fc)
EVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGGSGGGGSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 448 aa, synthetic construct; identical to SEQ ID NO: 178
  REGION 1..448: note = Humanized Anti-P Full HC (IgG1 N297A Fc)

SEQ ID NO: 188 - protein, 214 aa, synthetic construct; identical to SEQ ID NO: 167
  REGION 1..214: note = Humanized Anti-P Full LC

SEQ ID NO: 189 - protein, 451 aa, synthetic construct
  REGION 1..451: note = Humanized Anti-Alb Full HC (IgG1 N297A Fc)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 217 aa, synthetic construct; identical to SEQ ID NO: 169
  REGION 1..217: note = Humanized Anti-Alb Full LC

SEQ ID NO: 191 - protein, 363 aa, synthetic construct
  REGION 1..363: note = Humanized Camelid Anti-P IgG4 Fc (CH2/CH3 Only)
EVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSSGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 192 - protein, 367 aa, synthetic construct; identical to SEQ ID NO: 163
  REGION 1..367: note = Humanized Camelid Anti-Alb IgG4 Fc (CH2/CH3 Only)

SEQ ID NO: 193 - protein, 244 aa, synthetic construct; identical to SEQ ID NO: 184
  REGION 1..244: note = Humanized Anti-P Full HC (IgG1 YTE Fc)

SEQ ID NO: 194 - protein, 214 aa, synthetic construct; identical to SEQ ID NO: 167
  REGION 1..214: note = Humanized Anti-P Full LC

SEQ ID NO: 195 - protein, 451 aa, synthetic construct
  REGION 1..451: note = Humanized Anti-TNF Full HC (IgG1 YTE Fc)
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 214 aa, synthetic construct; identical to SEQ ID NO: 177
  REGION 1..214: note = Humanized Anti-TNF Full LC

SEQ ID NO: 197 - protein, 244 aa, synthetic construct; identical to SEQ ID NO: 184
  REGION 1..244: note = Humanized Anti-P Full HC (IgG1 YTE Fc)

SEQ ID NO: 198 - protein, 214 aa, synthetic construct; identical to SEQ ID NO: 167
  REGION 1..214: note = Humanized Anti-P Full LC

SEQ ID NO: 199 - protein, 453 aa, synthetic construct
  REGION 1..453: note = Humanized Anti-VEGF Full HC (IgG1 YTE Fc)
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 214 aa, synthetic construct; identical to SEQ ID NO: 181
  REGION 1..214: note = Humanized Anti-VEGF Full LC

SEQ ID NO: 201 - protein, 10 aa, synthetic construct
  REGION 1..10: note = Synthetic Construct
  VARIANT 6: note = Xaa is Asn, Gln, Ser, Ala, or Asp
  VARIANT 6: note = Xaa is Asn, Gln, Ser, Ala, or Asp
GYIFTXYPIH

SEQ ID NO: 202 - protein, 17 aa, synthetic construct
  REGION 1..17: note = Synthetic Construct
  VARIANT 3: note = Xaa is Asp, Glu, Ser, or Ala
  VARIANT 8: note = Xaa is His or Tyr
  VARIANT 11: note = Xaa is Pro, Ser, or Tyr
  VARIANT 12: note = Xaa is Ala or Asp
  VARIANT 13: note = Xaa is Asp, Arg, or Gln
  VARIANT 14: note = Xaa is Lys, Arg, or Ser
  VARIANT 15: note = Xaa is Phe or Val
  VARIANT 16: note = Xaa is Glu, Lys, Gln, or Arg
  VARIANT 17: note = Xaa is Asp or Gly
FIXPGGGXDEXXXXXXX

SEQ ID NO: 203 - protein, 9 aa, synthetic construct
  REGION 1..9: note = Synthetic Construct
RGGGYYLDY

SEQ ID NO: 204 - protein, 118 aa, synthetic construct
  REGION 1..118: note = Synthetic Construct
  VARIANT 31: note = Xaa is Asn, Gln, Ser, Ala, or Asp
  VARIANT 52: note = Xaa is Asp, Glu, Ser, or Ala
  VARIANT 57: note = Xaa is His or Tyr
  VARIANT 60: note = Xaa is Pro, Ser, or Tyr
  VARIANT 61: note = Xaa is Ala or Asp
  VARIANT 62: note = Xaa is Asp, Arg, or Gln
  VARIANT 63: note = Xaa is Lys, Arg, or Ser
  VARIANT 64: note = Xaa is Phe or Val
  VARIANT 65: note = Xaa is Glu, Lys, Gln, or Arg
  VARIANT 66: note = Xaa is Asp or Gly
EVQLVQSGAEVKKPGASVKVSCKASGYIFTXYPIHWVRQAPGQGLEWMGFIXPGGGXDEXXXXXXXRVTMTADTSTSTAYMELSSLRSEDTAVYYCARRGGGYYLDYWGQGTTVTVSS

SEQ ID NO: 205 - protein, 118 aa, synthetic construct
  REGION 1..118: note = Synthetic Construct
  VARIANT 31: note = Xaa is Asn, Gln, Ser, Ala, or Asp
  VARIANT 52: note = Xaa is Asp, Glu, Ser, or Ala
  VARIANT 57: note = Xaa is His or Tyr
  VARIANT 60: note = Xaa is Pro, Ser, or Tyr
  VARIANT 61: note = Xaa is Ala or Asp
  VARIANT 62: note = Xaa is Asp, Arg, or Gln
  VARIANT 63: note = Xaa is Lys, Arg, or Ser
  VARIANT 64: note = Xaa is Phe or Val
  VARIANT 65: note = Xaa is Glu, Lys, Gln, or Arg
  VARIANT 66: note = Xaa is Asp or Gly
QVQLVQSGAEVKKPGASVKVSCKASGYIFTXYPIHWVRQAPGQGLEWMGFIXPGGGXDEXXXXXXXRVTMTRDTSTSTVYMELSSLRSEDTAVYYCARRGGGYYLDYWGQGTTVTVSS

SEQ ID NO: 206 - protein, 11 aa, synthetic construct
  REGION 1..11: note = Synthetic Construct
RASQDISFFLN

SEQ ID NO: 207 - protein, 7 aa, synthetic construct
  REGION 1..7: note = Synthetic Construct
  VARIANT 1: note = Xaa is Gly or Tyr
  VARIANT 2: note = Xaa is Ala or Thr
  VARIANT 4: note = Xaa is Arg or Ser
XXSXYHS

SEQ ID NO: 208 - protein, 9 aa, synthetic construct
  REGION 1..9: note = Synthetic Construct
  VARIANT 4: note = Xaa is Ala, Asp, Asn, Gln, or Ser
  VARIANT 8: note = Xaa is Phe, His, Arg, Trp, or Tyr
QHGXTLPXT

SEQ ID NO: 209 - protein, 107 aa, synthetic construct
  REGION 1..107: note = Synthetic Construct
  VARIANT 49: note = Xaa is Gly or Tyr
  VARIANT 50: note = Xaa is Ala or Thr
  VARIANT 51: note = Xaa can be any naturally occurring amino acid
  VARIANT 52: note = Xaa is Arg or Ser
  VARIANT 53: note = Xaa can be any naturally occurring amino acid
  VARIANT 92: note = Xaa is Ala, Asp, Asn, Gln, or Ser
  VARIANT 96: note = Xaa is Phe, His, Arg, Trp, or Tyr
DIQMTQSPSSLSASVGDRVTITCRASQDISFFLNWYQQKPGKAPKLLIYXXSXYHSGVPSRFSGSGSGTDFTLTISSLQPEDFATYYCQHGXTLPXTFGQGTKLEIK

SEQ ID NO: 210 - protein, 107 aa, synthetic construct; identical to SEQ ID NO: 209
  REGION 1..107: note = Synthetic Construct
  VARIANT 50: note = Xaa is Gly or Tyr
  VARIANT 51: note = Xaa is Ala or Thr
  VARIANT 53: note = Xaa is Arg or Ser
  VARIANT 91: note = Xaa is Ala, Asp, Asn, Gln, or Ser
  VARIANT 92: note = Xaa can be any naturally occurring amino acid
  VARIANT 95: note = Xaa is Phe, His, Arg, Trp, or Tyr
  VARIANT 96: note = Xaa can be any naturally occurring amino acid

SEQ ID NO: 211 - protein, 12 aa, synthetic construct
  REGION 1..12: note = Synthetic Construct
  VARIANT 9: note = Xaa is Ile, Lys, Met, or Val
GFSLSTSGXGVG

SEQ ID NO: 212 - protein, 16 aa, synthetic construct
  REGION 1..16: note = Synthetic Construct
  VARIANT 3: note = Xaa is Phe, His, Trp, or Tyr
  VARIANT 4: note = Xaa is Phe, His, Trp, or Tyr
  VARIANT 11: note = Xaa is Ala, Asn, Gln, or Ser
HIXXDDVKSYXPALKS

SEQ ID NO: 213 - protein, 10 aa, synthetic construct
  REGION 1..10: note = Synthetic Construct
  VARIANT 3: note = Xaa is Ala, Asn, Glu, or Ser
IGXGYYSFDY

SEQ ID NO: 214 - protein, 120 aa, synthetic construct
  REGION 1..120: note = Synthetic Construct
  VARIANT 34: note = Xaa is Ile, Lys, Met, or Val
  VARIANT 54: note = Xaa is Phe, His, Trp, or Tyr
  VARIANT 55: note = Xaa is Phe, His, Trp, or Tyr
  VARIANT 62: note = Xaa is Ala, Asn, Gln, or Ser
  VARIANT 102: note = Xaa is Ala, Asp, Glu, or Ser
EVTLKESGPTLVKPTQTLTLTCTFSGFSLSTSGXGVGWIRQPPGKALEWLAHIXXDDVKSYXPALKSRLTITKDTSKNQVVLTMTNMDPVDTATYYCARIGXGYYSFDYWGQGTTVTVSS

SEQ ID NO: 215 - protein, 120 aa, synthetic construct
  REGION 1..120: note = Synthetic Construct
  VARIANT 34: note = Xaa is Ile, Lys, Met, or Val
  VARIANT 54: note = Xaa is Phe, His, Trp, or Tyr
  VARIANT 55: note = Xaa is Phe, His, Trp, or Tyr
  VARIANT 62: note = Xaa is Ala, Asn, Gln, or Ser
  VARIANT 102: note = Xaa is Ala, Asp, Glu, or Ser
QVTLKESGPTLVKPTQTLTLTCTFSGFSLSTSGXGVGWIRQPPGKALEWLAHIXXDDVKSYXPALKSRLTITKDTSKNQVVLTITNVDPVDTATYYCARIGXGYYSFDYWGQGTTLTVSS

SEQ ID NO: 216 - protein, 11 aa, synthetic construct
  REGION 1..11: note = Synthetic Construct
  VARIANT 1: note = Xaa can be any naturally occurring amino acid
XASQDVSDAVA

SEQ ID NO: 217 - protein, 7 aa, synthetic construct
  REGION 1..7: note = Synthetic Construct
SPSYRYT

SEQ ID NO: 218 - protein, 10 aa, synthetic construct
  REGION 1..10: note = Synthetic Construct
  VARIANT 8: note = Xaa is Phe, His, Trp, or Tyr
QQHYSTPXTF

SEQ ID NO: 219 - protein, 107 aa, synthetic construct
  REGION 1..107: note = Synthetic Construct
  VARIANT 24: note = Xaa is Lys or Arg
  VARIANT 96: note = Xaa is Phe, His, Trp, or Tyr
DIQMTQSPSSLSASVGDRVTITCXASQDVSDAVAWYQQKPGKAPKLLIYSPSYRYTGVPSRFSGSGSGTDFTLTISSLQPEDFATYYCQQHYSTPXTFGQGTKLEIK

SEQ ID NO: 220 - protein, 107 aa, synthetic construct
  REGION 1..107: note = Synthetic Construct
  VARIANT 24: note = Xaa is Lys or Arg
  VARIANT 96: note = Xaa is Phe, His, Trp, or Tyr
DIQMTQSPSSLSASVGDRVTITCXASQDVSDAVAWFQQKPGKAPKLLIYSPSYRYTGVPSRFSGSGSGTDFTFTISSLQPEDLATYYCQQHYSTPXTFGQGTKLEIK

SEQ ID NO: 221 - protein, 11 aa, synthetic construct
  REGION 1..11: note = Synthetic Construct
  VARIANT 9: note = Xaa is Phe, His, Trp, or Tyr
  VARIANT 9: note = Xaa is Phe, His, Trp, or Tyr
GFSFSSGYXIF

SEQ ID NO: 222 - protein, 11 aa, synthetic construct
  REGION 1..11: note = Synthetic Construct
GIYSGSSGTTY

SEQ ID NO: 223 - protein, 13 aa, synthetic construct
  REGION 1..13: note = Synthetic Construct
  VARIANT 2: note = Xaa is Ala, Asp, Glu, or Ser
  VARIANT 5: note = Xaa is Ala, Asp, Glu, or Ser
  VARIANT 8: note = Xaa is Ala, Asp, Glu, or Ser
  VARIANT 12: note = Xaa is Ala, Asn, Gln, or Ser
VXGIXSYXAAFXL

SEQ ID NO: 224 - protein, 125 aa, synthetic construct
  REGION 1..125: note = Synthetic Construct
  VARIANT 34: note = Xaa is Phe, His, Trp, or Tyr
  VARIANT 103: note = Xaa is Ala, Asp, Glu, or Ser
  VARIANT 106: note = Xaa is Ala, Asp, Glu, or Ser
  VARIANT 109: note = Xaa is Ala, Asp, Glu, or Ser
  VARIANT 113: note = Xaa is Ala, Asn, Gln, or Ser
EVQLLESGGGLVQPGGSLRLSCAASGFSFSSGYXIFWVRQAPGKGLEWVSGIYSGSSGTTYYADSVKGRFTISRDNSKNTLYLQMNSLRAEDTAVYYCAKSVXGIXSYXAAFXLWGQGTLVTVSS

SEQ ID NO: 225 - protein, 11 aa, synthetic construct
  REGION 1..11: note = Synthetic Construct
  VARIANT 9: note = Xaa is Phe, His, Trp, or Tyr
GFSFSSSYXIF

SEQ ID NO: 226 - protein, 10 aa, synthetic construct
  REGION 1..10: note = Synthetic Construct
  VARIANT 9: note = Xaa is Ile, Lys, Leu, or Met
GIYSSSGRXY

SEQ ID NO: 227 - protein, 14 aa, synthetic construct
  REGION 1..14: note = Synthetic Construct
  VARIANT 3: note = Xaa is Ala, Asp, Glu, or Ser
  VARIANT 6: note = Xaa is Ala, Asp, Glu, or Ser
  VARIANT 9: note = Xaa is Ala, Asp, Glu, or Ser
SAXGSXSYXAYFTL

SEQ ID NO: 228 - protein, 124 aa, synthetic construct
  REGION 1..124: note = Synthetic Construct
  VARIANT 34: note = Xaa is Phe, His, Trp, or Tyr
  VARIANT 59: note = Xaa is Phe, His, Trp, or Tyr
  VARIANT 102: note = Xaa can be any naturally occurring amino acid
  VARIANT 105: note = Xaa can be any naturally occurring amino acid
  VARIANT 108: note = Xaa can be any naturally occurring amino acid
  VARIANT 112: note = Xaa is Ala, Asp, Glu, or Ser
  VARIANT 115: note = Xaa is Ala, Asp, Glu, or Ser
  VARIANT 118: note = Xaa is Ala, Asp, Glu, or Ser
EVQLLESGGGLVQPGGSLRISCAASGFSFSSSYXIFWVRQAPGKGLELIGGIYSSSGRXYYADSVKGRFTISKDNSKNTMYLQMNSLRAEDTAVYYCARSAXGSXSYXAYFTLWGQGTLVTVSS

SEQ ID NO: 229 - protein, 11 aa, synthetic construct
  REGION 1..11: note = Synthetic Construct
  VARIANT 1: note = Xaa is Gln or Arg
  VARIANT 5: note = Xaa is Ala, Asn, Gln, or Ser
XASDXIYSLLA

SEQ ID NO: 230 - protein, 7 aa, synthetic construct
  REGION 1..7: note = Synthetic Construct
RASTLAS

SEQ ID NO: 231 - protein, 12 aa, synthetic construct
  REGION 1..12: note = Synthetic Construct
  VARIANT 7: note = Xaa is Ala, Asn, Gln, or Ser
QQHYDYXYLDVA

SEQ ID NO: 232 - protein, 110 aa, synthetic construct
  REGION 1..110: note = Synthetic Construct
  VARIANT 24: note = Xaa is Gln or Arg
  VARIANT 28: note = Xaa is Ala, Asn, Gln, or Ser
  VARIANT 95: note = Xaa is Ala, Asn, Gln, or Ser
DIQMTQSPSSLSASVGDRVTITCXASDXIYSLLAWYQQKPGKAPKLLIYRASTLASGVPSRFSGSGSGTDFTLTISSLQPEDFATYYCQQHYDYXYLDVAFGGGTKVEIK

SEQ ID NO: 233 - protein, 11 aa, synthetic construct
  REGION 1..11: note = Synthetic Construct
  VARIANT 1: note = Xaa is Gln or Arg
  VARIANT 5: note = Xaa is Ala, Asn, Gln, or Ser
  VARIANT 9: note = Xaa is Ala, Asn, Gln, or Ser
XASDXIYSXLA

SEQ ID NO: 234 - protein, 7 aa, synthetic construct
  REGION 1..7: note = Synthetic Construct
RASTLAS

SEQ ID NO: 235 - protein, 11 aa, synthetic construct
  REGION 1..11: note = Synthetic Construct
  VARIANT 4: note = Xaa is Phe, His, Trp, or Tyr
QQHXDYDYIDV

SEQ ID NO: 236 - protein, 110 aa, synthetic construct
  REGION 1..110: note = Synthetic Construct
  VARIANT 24: note = Xaa is Gln or Arg
  VARIANT 28: note = Xaa is Ala, Asn, Gln, or Ser
  VARIANT 32: note = Xaa is Ala, Asn, Gln, or Ser
  VARIANT 92: note = Xaa is Phe, His, Trp, or Tyr
DYQMTQSPSSLSASVGDRVTITCXASDXIYSXLAWYQQKPGKAPKLLIYRASTLASGVPSRFSGSGSGTDFTLTISSVQPEDFATYYCQQHXDYDYIDVAFGGGTKVEIK

SEQ ID NO: 237 - protein, 10 aa, synthetic construct
  REGION 1..10: note = Synthetic Construct
GRISSIIHMA

SEQ ID NO: 238 - protein, 13 aa, synthetic construct
  REGION 1..13: note = Synthetic Construct
  VARIANT 2: note = Xaa is Ile or Val
  VARIANT 6: note = Xaa is Ile or Val
  VARIANT 9: note = Xaa is Ala, Asp, Glu, or Ser
  VARIANT 11: note = Xaa is Ile or Val
  VARIANT 12: note = Xaa is Ala or Lys
RXGTTXYAXSXXG

SEQ ID NO: 239 - protein, 11 aa, synthetic construct
  REGION 1..11: note = Synthetic Construct
  VARIANT 5: note = Xaa is Ala or Lys
  VARIANT 10: note = Xaa is Ala, Asp, Glu, or Ser
LQYEXHGGAXY

SEQ ID NO: 240 - protein, 119 aa, synthetic construct
  REGION 1..119: note = Synthetic Construct
  VARIANT 54: note = Xaa is Ile or Val
  VARIANT 58: note = Xaa is Ile or Val
  VARIANT 61: note = Xaa is Ala, Asp, Glu, or Ser
  VARIANT 63: note = Xaa is Ile or Val
  VARIANT 64: note = Xaa is Ala or Lys
  VARIANT 102: note = Xaa is Ala or Lys
  VARIANT 107: note = Xaa is Ala, Asp, Glu, or Ser
EVQLLESGGGLVQPGGSLRLSCAASGRISSIIHMAWFRQAPGKERELVSEISRXGTTXYAXSXXGRFTISRDNSKNTLYLQMNSLKPEDTAVYYCNALQYEXHGGAXYWGQGTLVTVSS

SEQ ID NO: 241 - protein, 10 aa, synthetic construct
  REGION 1..10: note = Synthetic Construct
  VARIANT 6: note = Xaa is Ile or Val
  VARIANT 7: note = Xaa is Ala, Asp, Asn, Gln, or Ser
GRIFEXXMMA

SEQ ID NO: 242 - protein, 13 aa, synthetic construct
  REGION 1..13: note = Synthetic Construct
  VARIANT 2: note = Xaa is Ile or Val
  VARIANT 9: note = Xaa is Ala, Asp, Glu, or Ser
  VARIANT 11
  VARIANT 12: note = Xaa is Ala or Lys
RXGTTTYAXSXXG

SEQ ID NO: 243 - protein, 11 aa, synthetic construct
  REGION 1..11: note = Synthetic Construct
  VARIANT 5: note = Xaa is Ala or Lys
  VARIANT 10: note = Xaa is Ala, Asp, Glu, or Ser
LQYEXHGGAXY

SEQ ID NO: 244 - protein, 119 aa, synthetic construct
  REGION 1..119: note = Synthetic Construct
  VARIANT 31: note = Xaa is Ile or Val
  VARIANT 32: note = Xaa is Ala, Asp, Asn, Gln, or Ser
  VARIANT 54: note = Xaa is Ile or Val
  VARIANT 61: note = Xaa is Ala, Asp, Glu, or Ser
  VARIANT 63: note = Xaa is Ile or Val
  VARIANT 64: note = Xaa is Ala or Lys
  VARIANT 102: note = Xaa is Ala or Lys
  VARIANT 107: note = Xaa is Ala, Asp, Glu, or Ser
QVQLAESGGGLVQAGDSLKLSCTASGRIFEXXMMAWYRQAPGKDRELVAEISRXGTTTYAXSXXGRFTISRDSAKNTVTLQMNSLKSEDTAVYYCNALQYEXHGGAXYWGQGTQVTVSG

SEQ ID NO: 245 - protein, 4 aa, synthetic construct
  REGION 1..4: note = Synthetic Construct
ASTK

SEQ ID NO: 246 - protein, 6 aa, synthetic construct
  REGION 1..6: note = Synthetic Construct
RTVAAP

SEQ ID NO: 247 - protein, 120 aa, synthetic construct
  REGION 1..120: note = Synthetic Construct
  VARIANT 31: note = Xaa is Gln, Ser, Ala, or Asp
  VARIANT 53: note = Xaa is Glu, Ser, or Ala
EVQLVQSGAEVKKPGASVKVSCKASGYIFTXNYPIHWVRQAPGQGLEWMGFIXDPGGGYDEPDERFRDRVTMTADTSTSTAYMELSSLRSEDTAVYYCARRGGGYYLDYWGQGTTVTVSS

SEQ ID NO: 248 - protein, 109 aa, synthetic construct
  REGION 1..109: note = Synthetic Construct
  VARIANT 92: note = Xaa is Gln, Ser, Ala, or Asp
  VARIANT 96: note = Xaa is His, Tyr, or Phe
DIQMTQSPSSLSASVGDRVTITCRASQDISFFLNWYQQKPGKAPKLLIYYTSRYHSGVPSRFSGSGSGTDFTLTISSLQPEDFATYYCQHGXNTLXPWTFGQGTKLEIK

(19) United States
(12) Patent Application Publication
(10) Pub. No.: US 2025/0042981 A1
(43) Pub. Date: Feb. 6, 2025

(54) MONOSPECIFIC AND BISPECIFIC ANTIBODIES AND ANTIGEN BINDING FRAGMENTS THEREOF

(71) Applicant: NovelMed Therapeutics, Inc., Cleveland, OH (US)

(72) Inventor: Rekha Bansal, Solon, OH (US)

(21) Appl. No.: 18/663,175

(22) Filed: May 14, 2024

Related U.S. Application Data
(63) Continuation of application No. 17/462,795, filed on Aug. 31, 2021, now Pat. No. 11,981,727.

(51) Int. Cl. C07K 16/18; C07K 16/22; C07K 16/24

(52) U.S. Cl. CPC C07K 16/18; C07K 16/22; C07K 16/241; C07K 2317/24; C07K 2317/31; C07K 2317/52; C07K 2317/522; C07K 2317/55; C07K 2317/565; C07K 2317/569; C07K 2317/622; C07K 2317/76; C07K 2317/92; C07K 2317/94

(57) ABSTRACT

Isolated monospecific and bispecific anti-properdin antibodies or antigen binding fragments thereof for use in treating complement mediated disorders.

### RELATED APPLICATION

This application is a Continuation of U.S. patent application Ser. No. 17/462,795, filed Aug. 31, 2021 (now U.S. patent Ser. No. 11/981,727), the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 18, 2024, is named NMT-028649WO ORD.st.26 and is 369,057 bytes in size.

### FIELD OF THE INVENTION

This disclosure relates to monospecific and bispecific antibodies and antigen-binding fragments thereof that can bind to properdin and selectively inhibit the alternative complement pathway in disease conditions where the alternative pathway contributes to disease pathology.

### BACKGROUND

The complement system is important for clearance of pathogens and host defense against pathogens. The complement system is activated via three distinct complement pathways; the classical, the lectin and the alternative pathways. The classical pathway is activated via antigen-antibody complexes. The lectin pathway is a variation of the classical pathway. The alternative pathway (AP) is activated by foreign material, artificial surfaces, dead tissues, bacteria, and dead yeast cells. In disease conditions, AP activation generates C3a, C5a, and C5b-9 (also known as the MAC complex). Elevated levels of C3a, C5a, and C5b-9 have been found to be associated with multiple acute and chronic disease conditions. These inflammatory molecules activate leukocytes, neutrophils, monocytes, platelets, mast cells, and endothelial cells as well as induce vascular permeability, cytolysis, and tissue injury. Activated cells release inflammatory mediators, such as TNF-α, TL-10B, TL-6, TL-8, VEGF, neutrophil elastase, and peroxides. Therefore, inhibition of disease-induced AP activation is important for clinical benefit in diseases where complement activation plays a role in disease pathology.

The initiation of the alternative complement pathway requires the binding of properdin to C3b, which occurs with high affinity. Properdin-bound C3b (PC3b) associates with factor B to form the PC3bB complex, which is then cleaved by factor D into PC3bBb and Ba, in which Ba is released. Properdin-depleted serum completely lacks AP activation activity, showing that properdin is essential for this initiation process to occur. Properdin concentration in blood is nearly 5 μg/ml, and consequently, it is the only non-protease molecule present at much lower concentration than other non-protease molecules.

Inhibiting AP activation is an important therapeutic strategy to mitigate symptoms and slow or prevent disease progression. Depleting, neutralizing, or inactivating properdin can block AP activation without inhibiting the classical complement pathway and, thus, is a viable and promising therapeutic strategy. The benefit of leaving the classical pathway intact is increased protection against infection.

### SUMMARY

Embodiments described herein relate to isolated monospecific or bispecific antibodies or antigen binding fragments thereof. The isolated monospecific or bispecific antibodies or antigen binding fragments thereof specifically bind properdin and can selectively block the alternative complement pathway. The isolated monospecific or bispecific antibodies or antigen binding fragments thereof described herein can neutralize properdin functional activity and prevent AP induced production of C3a, C5a, C3b, and Mac complex (C5b-9). As a result, cellular activation, inflammation, and release of inflammatory mediators can also be prevented. Since AP activation is linked to various acute and chronic human diseases, inhibition of AP activation with the isolated monospecific or bispecific antibodies or antigen binding fragments thereof can also inhibit inflammation, providing clinical benefits to human beings treated with the isolated monospecific or bispecific antibodies or antigen binding fragments thereof.

In some embodiments, an isolated monospecific or bispecific antibody or antigen binding fragment can include at least one of:
a)
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEX₃X₄X₅X₆X₇X₈X₉ (SEQ ID NO: 202), wherein X₁ is D, E, S, or A; X₂ is H or Y; X₃ is P, S, or Y; X₄ is A or D; X₅ is D, E, or Q; X₆ is K, R, or S; X₇ is F or V; X₈ is E, K, Q, or R; and X₉ is D or G; and
a CDR-H3 comprising the amino acid sequence of RGGGYYLDY (SEQ ID NO: 203);
b)
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y;
c)
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX₂PALKS (SEQ ID NO: 212), wherein X₁ is F, H, W, or Y; and X₂ is A, N, Q, or S; and
a CDR-H3 comprising the amino acid sequence of IGX₁GYYSFDY (SEQ ID NO: 213), wherein X₁ is A, D, E, or S;
d)
a CDR-L1 comprising the amino acid sequence of X₁ASQDVSDAVA (SEQ ID NO: 216), wherein X₁ is K or R;
a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y;
e)
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222); and
a CDR-H3 comprising the amino acid sequence of SVX₁GIX₁SYX₁ AAFX₂L (SEQ ID NO: 223), wherein X₁ is A, D, E, or S; and X₂ is A, N, Q, or S; f)
a CDR-L1 comprising the amino acid sequence of X₁ASDX₂IYSLLA (SEQ ID NO: 229), wherein X₁ is Q or R; X₂ is A, N, Q, or S;
a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S;
g)
a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M; and
a CDR-H3 comprising the amino acid sequence of SAX₁GSX₁SYX₁AYFTL (SEQ ID NO: 227), wherein X₁ is A, D, E, or S;
h)
a CDR-L1 comprising the amino acid sequence of X₁ASDX₂IYSX₂LA (SEQ ID NO: 233), wherein X₁ is Q or R; X₂ is A, N, Q, or S;
a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y;
i)
a CDR-H1 comprising the amino acid sequence of GRISSIIHMA (SEQ ID NO: 237), wherein X₁ is F, H, W, or Y,
a CDR-H2 comprising the amino acid sequence of RX₁GTTX₁YAX₂SX₁X₃G (SEQ ID NO: 238), wherein X₁ is I or V; X₂ is A, D, E, or S; and X₃ is A or K; and
a CDR-H3 comprising the amino acid sequence of LQYEX₁HGGAX₂Y (SEQ ID NO: 239), wherein X₁ is A or K; and X₂ is A, D, E, or S;
j)
a CDR-H1 comprising the amino acid sequence of GRIFEX₁X₂MMA (SEQ ID NO: 241), wherein X₁ is I or V; and X₂ is A, D, N, Q, or S,
a CDR-H2 comprising the amino acid sequence of RX₁GTTTYAX₂SX₁X₃G (SEQ ID NO: 242), wherein X₁ is I or V; X₂ is A, D, E, or S; and X₃ is A or K; and
a CDR-H3 comprising the amino acid sequence of LQYX₁RYGGAEY (SEQ ID NO: 243), wherein X₁ is A, D, E, or S; or
k)
or a heavy chain variable region and/or light chain variable region that competitively inhibits binding of an isolated monospecific or bispecific antibody or antigen binding fragment thereof comprising at least one of a), b), c), d), e), f), g), h), i), or j) to monomeric properdin.

In some embodiments, the isolated monospecific or bispecific antibody or antigen binding fragment, can include:
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEX₃X₄X₅X₆X₇X₈X₉ (SEQ ID NO: 202), wherein X₁ is D, E, S, or A; X₂ is H or Y; X₃ is P, S, or Y; X₄ is A or D; X₅ is D, E, or Q; X₆ is K, R, or S; X₇ is F or V; X₈ is E, K, Q, or R; and X₉ is D or G; and
a CDR-H3 comprising the amino acid sequence of RGGGYYLDY (SEQ ID NO: 203);
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y.

In some embodiments, the isolated monospecific or bispecific antibody or antigen binding fragment can include:
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX₂PALKS (SEQ ID NO: 212), wherein X₁ is F, H, W, or Y; and X₂ is A, N, Q, or S;
a CDR-H3 comprising the amino acid sequence of IGX₁GYYSFDY (SEQ ID NO: 213), wherein X₁ is A, D, E, or S;
a CDR-L1 comprising the amino acid sequence of X₁ASQDVSDAVA (SEQ ID NO: 216), wherein X₁ is K or R;
a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y.

In some embodiments, the isolated monospecific or bispecific antibody or antigen binding fragment thereof can include:
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222);
a CDR-H3 comprising the amino acid sequence of SVX₁GIX₁SYX₁ AAFX₂L (SEQ ID NO: 223), wherein X₁ is A, D, E, or S; and X₂ is A, N, Q, or S;
a CDR-L1 comprising the amino acid sequence of X₁ASDX₂IYSLLA (SEQ ID NO: 229), wherein X₁ is Q or R; X₂ is A, N, Q, or S;
a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S.

In some embodiments, the isolated monospecific or bispecific antibody or antigen binding fragment thereof can include a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M;
a CDR-H3 comprising the amino acid sequence of SAX₁GSX₁SYX₁AYFTL (SEQ ID NO: 227), wherein X₁ is A, D, E, or S;
a CDR-L1 comprising the amino acid sequence of X₁ASDX₂IYSX₂LA (SEQ ID NO: 233), wherein X₁ is Q or R; X₂ is A, N, Q, or S;
a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y.

In some embodiments, the isolated monospecific or bispecific antibody or antigen binding fragment thereof can include a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107; or a heavy chain variable region that competitively inhibits binding of an isolated antibody or antigen binding fragment comprising at least one of a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

In other embodiments, the isolated monospecific or bispecific antibody or antigen binding fragment thereof can include a heavy chain variable region that includes an amino acid sequence at least about 70%, at least about 80%, or at least about 90% identical to the amino acid sequence of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

In still other embodiments, the isolated monospecific or bispecific antibody or antigen binding fragment thereof can include a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72; or a light chain variable region that competitively inhibits binding of an isolated antibody or antigen binding fragment comprising at least one of a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

In some embodiments, the isolated monospecific or bispecific antibody or antigen binding fragment thereof can include a light chain variable region that includes an amino acid sequence at least about 70%, at least about 80%, or at least about 90% identical to the amino acid sequence of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

In some embodiments, the isolated monospecific or bispecific antibody or antigen binding fragment thereof can include at least one of the following:
a) a heavy chain variable region that includes the 3 CDRs of SEQ ID NO: 1 and a light chain variable region that includes the 3 CDRs of SEQ ID NO: 24;
b) a heavy chain variable region that includes the 3 CDRs of SEQ ID NO: 2 and a light chain variable region that includes the 3 CDRs of SEQ ID NO: 25;
c) a heavy chain variable region that includes the 3 CDRs of SEQ ID NO: 3 and a light chain variable region that includes the 3 CDRs of SEQ ID NO: 26;
d) a heavy chain variable region that includes the 3 CDRs of SEQ ID NO: 4 and a light chain variable region that includes the 3 CDRs of SEQ ID NO: 27;
e) a heavy chain variable region that includes the 3 CDRs of SEQ ID NO: 5 and a light chain variable region that includes the 3 CDRs of SEQ ID NO: 28;
f) a heavy chain variable region that includes the 3 CDRs of SEQ ID NO: 6 and a light chain variable region that includes the 3 CDRs of SEQ ID NO: 29;
g) a heavy chain variable region that includes the 3 CDRs of SEQ ID NO: 7 and a light chain variable region that includes the 3 CDRs of SEQ ID NO: 30;
h) a heavy chain variable region that includes the 3 CDRs of SEQ ID NO: 8 and a light chain variable region that includes the 3 CDRs of SEQ ID NO: 31;
i) a heavy chain variable region that includes the 3 CDRs of SEQ ID NO: 9 and a light chain variable region that includes the 3 CDRs of SEQ ID NO: 32;
j) a heavy chain variable region that includes the 3 CDRs of SEQ ID NO: 10 and a light chain variable region that includes the 3 CDRs of SEQ ID NO: 33;
k) a heavy chain variable region that includes the 3 CDRs of SEQ ID NO: 11 and a light chain variable region that includes the 3 CDRs of SEQ ID NO: 34;
l) a heavy chain variable region that includes the 3 CDRs of SEQ ID NO: 12 and a light chain variable region that includes the 3 CDRs of SEQ ID NO: 35;
m) a heavy chain variable region that includes the 3 CDRs of SEQ ID NO: 13 and a light chain variable region that includes the 3 CDRs of SEQ ID NO: 36;
n) a heavy chain variable region that includes the 3 CDRs of SEQ ID NO: 14 and a light chain variable region that includes the 3 CDRs of SEQ ID NO: 37;
o) a heavy chain variable region that includes the 3 CDRs of SEQ ID NO: 15 and a light chain variable region that includes the 3 CDRs of SEQ ID NO: 38 or SEQ ID NO: 39;
p) a heavy chain variable region that includes the 3 CDRs of SEQ ID NO: 16 and a light chain variable region that includes the 3 CDRs of SEQ ID NO: 38 or SEQ ID NO: 39;
q) a heavy chain variable region that includes the 3 CDRs of SEQ ID NO: 17 and a light chain variable region that includes the 3 CDRs of SEQ ID NO: 38 or SEQ ID NO: 39;
r) a heavy chain variable region that includes the 3 CDRs of SEQ ID NO: 18 and a light chain variable region that includes the 3 CDRs of SEQ ID NO: 38 or SEQ ID NO: 39;
s) a heavy chain variable region that includes the 3 CDRs of SEQ ID NO: 19 and a light chain variable region that includes the 3 CDRs of SEQ ID NO: 38 or SEQ ID NO: 39;
t) a heavy chain variable region that includes the 3 CDRs of SEQ ID NO: 20 and a light chain variable region that includes the 3 CDRs of SEQ ID NO: 38 or SEQ ID NO: 39;
u) a heavy chain variable region that includes the 3 CDRs of SEQ ID NO: 21 and a light chain variable region that includes the 3 CDRs of SEQ ID NO: 38 or SEQ ID NO: 39;
v) a heavy chain variable region that includes the 3 CDRs of SEQ ID NO: 22 and a light chain variable region that includes the 3 CDRs of SEQ ID NO: 38 or SEQ ID NO: 39;
w) a heavy chain variable region that includes the 3 CDRs of SEQ ID NO: 23 and a light chain variable region that includes the 3 CDRs of SEQ ID NO: 38 or SEQ ID NO: 39;
x) a heavy chain variable region that includes the 3 CDRs of SEQ ID NO: 40 and a light chain variable region that includes the 3 CDRs of SEQ ID NO: 45;
y) a heavy chain variable region that includes the 3 CDRs of SEQ ID NO: 41 and a light chain variable region that includes the 3 CDRs of SEQ ID NO: 46;
z) a heavy chain variable region that includes the 3 CDRs of SEQ ID NO: 42 and a light chain variable region that includes the 3 CDRs of SEQ ID NO: 47;
aa) a heavy chain variable region that includes the 3 CDRs of SEQ ID NO: 43 and a light chain variable region that includes the 3 CDRs of SEQ ID NO: 48, SEQ ID NO: 49, or SEQ ID NO: 50;
bb) a heavy chain variable region that includes the 3 CDRs of SEQ ID NO: 44 and a light chain variable region that includes the 3 CDRs of SEQ ID NO: 48, SEQ ID NO: 49, or SEQ ID NO: 50;
cc) a heavy chain variable region that includes the 3 CDRs of SEQ ID NO: 51 and a light chain variable region that includes the 3 CDRs of SEQ ID NO: 61, SEQ ID NO: 62, SEQ ID NO: 63, SEQ ID NO: 64, or SEQ ID NO: 65;
dd) a heavy chain variable region that includes the 3 CDRs of SEQ ID NO: 52 and a light chain variable region that includes the 3 CDRs of SEQ ID NO: 61, SEQ ID NO: 62, SEQ ID NO: 63, SEQ ID NO: 64, or SEQ ID NO: 65;
ee) a heavy chain variable region that includes the 3 CDRs of SEQ ID NO: 53 and a light chain variable region that includes the 3 CDRs of SEQ ID NO: 61, SEQ ID NO: 62, SEQ ID NO: 63, SEQ ID NO: 64, or SEQ ID NO: 65;
ff) a heavy chain variable region that includes the 3 CDRs of SEQ ID NO: 54 and a light chain variable region that includes the 3 CDRs of SEQ ID NO: 61, SEQ ID NO: 62, SEQ ID NO: 63, SEQ ID NO: 64, or SEQ ID NO: 65;
gg) a heavy chain variable region that includes the 3 CDRs of SEQ ID NO: 55 and a light chain variable region that includes the 3 CDRs of SEQ ID NO: 61, SEQ ID NO: 62, SEQ ID NO: 63, SEQ ID NO: 64, or SEQ ID NO: 65;
hh) a heavy chain variable region that includes the 3 CDRs of SEQ ID NO: 56 and a light chain variable region that includes the 3 CDRs of SEQ ID NO: 66, SEQ ID NO: 67, SEQ ID NO: 68, SEQ ID NO: 69, or SEQ ID NO: 70;
ii) a heavy chain variable region that includes the 3 CDRs of SEQ ID NO: 57 and a light chain variable region that includes the 3 CDRs of SEQ ID NO: 66, SEQ ID NO: 67, SEQ ID NO: 68, SEQ ID NO: 69, or SEQ ID NO: 70;
jj) a heavy chain variable region that includes the 3 CDRs of SEQ ID NO: 58 and a light chain variable region that includes the 3 CDRs of SEQ ID NO: 66, SEQ ID NO: 67, SEQ ID NO: 68, SEQ ID NO: 69, or SEQ ID NO: 70;
kk) a heavy chain variable region that includes the 3 CDRs of SEQ ID NO: 59 and a light chain variable region that includes the 3 CDRs of SEQ ID NO: 66, SEQ ID NO: 67, SEQ ID NO: 68, SEQ ID NO: 69, or SEQ ID NO: 70; or
ll) a heavy chain variable region that includes the 3 CDRs of SEQ ID NO: 60 and a light chain variable region that includes the 3 CDRs of SEQ ID NO: 66, SEQ ID NO: 67, SEQ ID NO: 68, SEQ ID NO: 69, or SEQ ID NO: 70.

In some embodiments, the isolated monospecific or bispecific antibody or antigen binding fragment thereof can include at least one of the following:
a) a heavy chain that includes an amino acid sequence at least about 70%, at least about 80%, or at least about 90% identical to the amino acid sequence of SEQ ID NO: 1 and a light chain that includes an amino acid sequence at least about 70%, at least about 80%, or at least about 90% identical to the amino acid sequence of SEQ ID NO: 24;
b) a heavy chain that includes an amino acid sequence at least about 70%, at least about 80%, or at least about 90% identical to the amino acid sequence of SEQ ID NO: 2 and a light chain that includes an amino acid sequence at least about 70%, at least about 80%, or at least about 90% identical to the amino acid sequence of SEQ ID NO: 25;
c) a heavy chain variable that includes an amino acid sequence at least about 70%, at least about 80%, or at least about 90% identical to the amino acid sequence of SEQ ID NO: 3 and a light chain that includes an amino acid sequence at least about 70%, at least about 80%, or at least about 90% identical to the amino acid sequence of SEQ ID NO: 26;
d) a heavy chain that includes an amino acid sequence at least about 70%, at least about 80%, or at least about 90% identical to the amino acid sequence of SEQ ID NO: 4 and a light chain that includes an amino acid sequence at least about 70%, at least about 80%, or at least about 90% identical to the amino acid sequence of SEQ ID NO: 27;
e) a heavy chain that includes an amino acid sequence at least about 70%, at least about 80%, or at least about 90% identical to the amino acid sequence of SEQ ID NO: 5 and a light chain that includes an amino acid sequence at least about 70%, at least about 80%, or at least about 90% identical to the amino acid sequence of SEQ ID NO: 28;
f) a heavy chain that includes an amino acid sequence at least about 70%, at least about 80%, or at least about 90% identical to the amino acid sequence of SEQ ID NO: 6 and a light chain that includes an amino acid sequence at least about 70%, at least about 80%, or at least about 90% identical to the amino acid sequence of SEQ ID NO: 29;
g) a heavy chain that includes an amino acid sequence at least about 70%, at least about 80%, or at least about 90% identical to the amino acid sequence of SEQ ID NO: 7 and a light chain that includes an amino acid sequence at least about 70%, at least about 80%, or at least about 90% identical to the amino acid sequence of SEQ ID NO: 30;
h) a heavy chain that includes an amino acid sequence at least about 70%, at least about 80%, or at least about 90% identical to the amino acid sequence of SEQ ID NO: 8 and a light chain that includes an amino acid sequence at least about 70%, at least about 80%, or at least about 90% identical to the amino acid sequence of SEQ ID NO: 31;
i) a heavy chain that includes an amino acid sequence at least about 70%, at least about 80%, or at least about 90% identical to the amino acid sequence of SEQ ID NO: 9 and a light chain that includes an amino acid sequence at least about 70%, at least about 80%, or at least about 90% identical to the amino acid sequence of SEQ ID NO: 32;
j) a heavy chain that includes an amino acid sequence at least about 70%, at least about 80%, or at least about 90% identical to the amino acid sequence of SEQ ID NO: 10 and a light chain that includes an amino acid sequence at least about 70%, at least about 80%, or at least about 90% identical to the amino acid sequence of SEQ ID NO: 33;
k) a heavy chain that includes an amino acid sequence at least about 70%, at least about 80%, or at least about 90% identical to the amino acid sequence of SEQ ID NO: 11 and a light chain that includes an amino acid sequence at least about 70%, at least about 80%, or at least about 90% identical to the amino acid sequence of SEQ ID NO: 34;
l) a heavy chain that includes an amino acid sequence at least about 70%, at least about 80%, or at least about 90% identical to the amino acid sequence of SEQ ID NO: 12 and a light chain that includes an amino acid sequence at least about 70%, at least about 80%, or at least about 90% identical to the amino acid sequence of SEQ ID NO: 35;
m) a heavy chain that includes an amino acid sequence at least about 70%, at least about 80%, or at least about 90% identical to the amino acid sequence of SEQ ID NO: 13 and a light chain that includes an amino acid sequence at least about 70%, at least about 80%, or at least about 90% identical to the amino acid sequence of SEQ ID NO: 36;
n) a heavy chain that includes an amino acid sequence at least about 70%, at least about 80%, or at least about 90% identical to the amino acid sequence of SEQ ID NO: 14 and a light chain that includes an amino acid sequence at least about 70%, at least about 80%, or at least about 90% identical to the amino acid sequence of SEQ ID NO: 37;
o) a heavy chain that includes an amino acid sequence at least about 70%, at least about 80%, or at least about 90% identical to the amino acid sequence of SEQ ID NO: 15 and a light chain that includes an amino acid sequence at least about 70%, at least about 80%, or at least about 90% identical to the amino acid sequence of SEQ ID NO: 38 or SEQ ID NO: 39;
p) a heavy chain that includes an amino acid sequence at least about 70%, at least about 80%, or at least about 90% identical to the amino acid sequence of SEQ ID NO: 16 and a light chain that includes an amino acid sequence at least about 70%, at least about 80%, or at least about 90% identical to the amino acid sequence of SEQ ID NO: 38 or SEQ ID NO: 39;
q) a heavy chain that includes an amino acid sequence at least about 70%, at least about 80%, or at least about 90% identical to the amino acid sequence of SEQ ID NO: 17 and a light chain that includes an amino acid sequence at least about 70%, at least about 80%, or at least about 90% identical to the amino acid sequence of SEQ ID NO: 38 or SEQ ID NO: 39;
r) a heavy chain that includes an amino acid sequence at least about 70%, at least about 80%, or at least about 90% identical to the amino acid sequence of SEQ ID NO: 18 and a light chain that includes an amino acid sequence at least about 70%, at least about 80%, or at least about 90% identical to the amino acid sequence of SEQ ID NO: 38 or SEQ ID NO: 39;
s) a heavy chain that includes an amino acid sequence at least about 70%, at least about 80%, or at least about 90% identical to the amino acid sequence of SEQ ID NO: 19 and a light chain that includes an amino acid sequence at least about 70%, at least about 80%, or at least about 90% identical to the amino acid sequence of SEQ ID NO: 38 or SEQ ID NO: 39;
t) a heavy chain that includes an amino acid sequence at least about 70%, at least about 80%, or at least about 90% identical to the amino acid sequence of SEQ ID NO: 20 and a light chain that includes an amino acid sequence at least about 70%, at least about 80%, or at least about 90% identical to the amino acid sequence of SEQ ID NO: 38 or SEQ ID NO: 39;
u) a heavy chain that includes an amino acid sequence at least about 70%, at least about 80%, or at least about 90% identical to the amino acid sequence of SEQ ID NO: 21 and a light chain that includes an amino acid sequence at least about 70%, at least about 80%, or at least about 90% identical to the amino acid sequence of SEQ ID NO: 38 or SEQ ID NO: 39;
v) a heavy chain that includes an amino acid sequence at least about 70%, at least about 80%, or at least about 90% identical to the amino acid sequence of SEQ ID NO: 22 and a light chain that includes an amino acid sequence at least about 70%, at least about 80%, or at least about 90% identical to the amino acid sequence of SEQ ID NO: 38 or SEQ ID NO: 39;
w) a heavy chain that includes an amino acid sequence at least about 70%, at least about 80%, or at least about 90% identical to the amino acid sequence of SEQ ID NO: 23 and a light chain that includes an amino acid sequence at least about 70%, at least about 80%, or at least about 90% identical to the amino acid sequence of SEQ ID NO: 38 or SEQ ID NO: 39;
x) a heavy chain that includes an amino acid sequence at least about 70%, at least about 80%, or at least about 90% identical to the amino acid sequence of SEQ ID NO: 40 and a light chain that includes an amino acid sequence at least about 70%, at least about 80%, or at least about 90% identical to the amino acid sequence of SEQ ID NO: 45;
y) a heavy chain that includes an amino acid sequence at least about 70%, at least about 80%, or at least about 90% identical to the amino acid sequence of SEQ ID NO: 41 and a light chain that includes an amino acid sequence at least about 70%, at least about 80%, or at least about 90% identical to the amino acid sequence of SEQ ID NO: 46;
z) a heavy chain that includes an amino acid sequence at least about 70%, at least about 80%, or at least about 90% identical to the amino acid sequence of SEQ ID NO: 42 and a light chain that includes an amino acid sequence at least about 70%, at least about 80%, or at least about 90% identical to the amino acid sequence of SEQ ID NO: 47;
aa) a heavy chain that includes an amino acid sequence at least about 70%, at least about 80%, or at least about 90% identical to the amino acid sequence of SEQ ID NO: 43 and a light chain that includes an amino acid sequence at least about 70%, at least about 80%, or at least about 90% identical to the amino acid sequence of SEQ ID NO: 48, SEQ ID NO: 49, or SEQ ID NO: 50;
bb) a heavy chain that includes an amino acid sequence at least about 70%, at least about 80%, or at least about 90% identical to the amino acid sequence of SEQ ID NO: 44 and a light chain that includes an amino acid sequence at least about 70%, at least about 80%, or at least about 90% identical to the amino acid sequence of SEQ ID NO: 48, SEQ ID NO: 49, or SEQ ID NO: 50;
cc) a heavy chain that includes an amino acid sequence at least about 70%, at least about 80%, or at least about 90% identical to the amino acid sequence of SEQ ID NO: 51 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
dd) a heavy chain that includes an amino acid sequence at least about 70%, at least about 80%, or at least about 90% identical to the amino acid sequence of SEQ ID NO: 52 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ee) a heavy chain that includes an amino acid sequence at least about 70%, at least about 80%, or at least about 90% identical to the amino acid sequence of SEQ ID NO: 53 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ff) a heavy chain that includes an amino acid sequence at least about 70%, at least about 80%, or at least about 90% identical to the amino acid sequence of SEQ ID NO: 54 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
gg) a heavy chain that includes an amino acid sequence at least about 70%, at least about 80%, or at least about 90% identical to the amino acid sequence of SEQ ID NO: 55 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
hh) a heavy chain that includes an amino acid sequence at least about 70%, at least about 80%, or at least about 90% identical to the amino acid sequence of SEQ ID NO: 56 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
ii) a heavy chain that includes an amino acid sequence at least about 70%, at least about 80%, or at least about 90% identical to the amino acid sequence of SEQ ID NO: 57 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
jj) a heavy chain that includes an amino acid sequence at least about 70%, at least about 80%, or at least about 90% identical to the amino acid sequence of SEQ ID NO: 58 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
kk) a heavy chain that includes an amino acid sequence at least about 70%, at least about 80%, or at least about 90% identical to the amino acid sequence of SEQ ID NO: 59 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70; or
ll) a heavy chain that includes an amino acid sequence at least about 70%, at least about 80%, or at least about 90% identical to the amino acid sequence of SEQ ID NO: 60 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70.

In some embodiments, the isolated monospecific or bispecific antibody or antigen binding fragment thereof can include a constant chain region and wherein the antibody or antigen binding fragment thereof including the constant chain region has enhanced in vivo half-live and/or reduced immunogenicity compared to the antibody or antigen binding fragment thereof without the constant chain region.

In some embodiments, the constant chain region includes an amino acid sequence at least about 70%, at least about 80%, or at least about 90% identical to the amino acid sequence of SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, or SEQ ID NO: 139.

In some embodiments, the isolated monospecific or bispecific antibody or antigen binding fragment thereof can include at least one of the following:
a heavy chain that includes an amino acid sequence at least about 70%, at least about 80%, or at least about 90% identical to the amino acid sequence of SEQ ID NO: 156 and a light chain that includes an amino acid sequence at least about 70%, at least about 80%, or at least about 90% identical to the amino acid sequence of SEQ ID NO: 157;
a heavy chain that includes an amino acid sequence at least about 70%, at least about 80%, or at least about 90% identical to the amino acid sequence of SEQ ID NO: 166 and a light chain that includes an amino acid sequence at least about 70%, at least about 80%, or at least about 90% identical to the amino acid sequence of SEQ ID NO: 167;
a heavy chain that includes an amino acid sequence at least about 70%, at least about 80%, or at least about 90% identical to the amino acid sequence of SEQ ID NO: 170 and a light chain that includes an amino acid sequence at least about 70%, at least about 80%, or at least about 90% identical to the amino acid sequence of SEQ ID NO: 171;
a heavy chain that includes an amino acid sequence at least about 70%, at least about 80%, or at least about 90% identical to the amino acid sequence of SEQ ID NO: 178 and a light chain that includes an amino acid sequence at least about 70%, at least about 80%, or at least about 90% identical to the amino acid sequence of SEQ ID NO: 179;
a heavy chain that includes an amino acid sequence at least about 70%, at least about 80%, or at least about 90% identical to the amino acid sequence of SEQ ID NO: 182;
a heavy chain that includes an amino acid sequence at least about 70%, at least about 80%, or at least about 90% identical to the amino acid sequence of SEQ ID NO: 184 and a light chain that includes an amino acid sequence at least about 70%, at least about 80%, or at least about 90% identical to the amino acid sequence of SEQ ID NO: 185;
a heavy chain that includes an amino acid sequence at least about 70%, at least about 80%, or at least about 90% identical to the amino acid sequence of SEQ ID NO: 187 and a light chain that includes an amino acid sequence at least about 70%, at least about 80%, or at least about 90% identical to the amino acid sequence of SEQ ID NO: 188;
a heavy chain that includes an amino acid sequence at least about 70%, at least about 80%, or at least about 90% identical to the amino acid sequence of SEQ ID NO: 191;
a heavy chain that includes an amino acid sequence at least about 70%, at least about 80%, or at least about 90% identical to the amino acid sequence of SEQ ID NO: 193 and a light chain that includes an amino acid sequence at least about 70%, at least about 80%, or at least about 90% identical to the amino acid sequence of SEQ ID NO: 194; or
a heavy chain that includes an amino acid sequence at least about 70%, at least about 80%, or at least about 90% identical to the amino acid sequence of SEQ ID NO: 197 and a light chain that includes an amino acid sequence at least about 70%, at least about 80%, or at least about 90% identical to the amino acid sequence of SEQ ID NO: 198.

In some embodiments, the isolated monospecific or bispecific antibody or antigen binding fragment thereof can include a peptide linker. The peptide linker can include the amino acid sequence of SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 245, or SEQ ID NO: 246.

In some embodiments, the antibody or antigen binding fragment thereof is humanized.

In other embodiments, the antibody or antigen binding fragment thereof binds to human properdin.

In some embodiments, the antibody or antigen binding fragment thereof inhibits alternative complement pathway activation in a mammal without inhibiting classical complement pathway activation.

In some embodiments, the isolated monospecific or bispecific antibody or antigen binding fragment thereof can include a first heavy chain variable region that binds to properdin and a second heavy chain variable region that binds to a different epitope than the first heavy chain variable region.

In some embodiments, the second heavy chain variable region increases the in vivo half-life of the antibody or antigen binding fragment thereof to about 3 week to about 8 weeks.

In other embodiments, the second heavy chain variable region binds to one of albumin, TNF, or VEGF.

In some embodiments, the second antibody or antigen binding fragment thereof comprises an amino acid sequence at least about 70%, at least about 80%, or at least about 90% identical to the amino acid sequence of SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 150, or SEQ ID NO: 152.

In some embodiments, the isolated monospecific or bispecific antibody or antigen binding fragment thereof can inhibit C3b and Mac complex (C5b-9) formation in vivo.

Other embodiments described herein relate to an isolated monospecific or bispecific anti-properdin antibody or antigen binding fragment that includes:
an anti-properdin heavy chain variable region that includes an amino acid sequence at least about 70%, at least about 80%, or at least about 90% identical to the amino acid sequence of SEQ ID NO: 2 and an anti-properdin light chain variable region that includes an amino acid sequence at least about 70%, at least about 80%, or at least about 90% identical to the amino acid sequence of SEQ ID NO: 25 and
wherein the heavy chain variable region includes:
a CDR-H1 comprising the amino acid sequence of GYIFTX₁YPIH (SEQ ID NO: 201), wherein X₁ is N, Q, S, A, or D (e.g., X₁ is Q, S, A, or D)
a CDR-H2 comprising the amino acid sequence of FIX₁PGGGX₂DEX₃X₄X₅X₆X₇X₈X₉ (SEQ ID NO: 202), wherein X₁ is D, E, S (e.g., E or S), or A; X₂ is H or Y; X₃ is P, S, or Y; X₄ is A or D; X₅ is D, E, or Q; X₆ is K, R, or S; X₇ is F or V; X₈ is E, K, Q, or R; and X₉ is D or G; and
a CDR-H3 comprising the amino acid sequence of RGGGYYLDY (SEQ ID NO: 203); and the light chain variable region includes:
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is N, A, D, Q, or S (e.g., A, D, Q, or S); and X₂ is F, H, R, W, or Y (e.g., F, H, R, or Y).

In some embodiments, the isolated monospecific or bispecific antibody or antigen binding fragment thereof can include at least one of a constant chain region or a second heavy chain variable region binds that binds to a different epitope than the anti-properdin heavy chain region and wherein the antibody or antigen binding fragment including the constant chain region or second heavy chain variable region has enhanced in vivo half-live and/or reduced immunogenicity compared to the antibody or antigen binding fragment thereof without the constant chain region or second heavy chain variable region.

In some embodiments, the constant chain region includes an amino acid sequence at least about 70%, at least about 80%, or at least about 90% identical to the amino acid sequence of SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, or SEQ ID NO: 139; and the second heavy chain variable region includes an amino acid sequence at least about 70%, at least about 80%, or at least about 90% identical to the amino acid sequence of SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 150, or SEQ ID NO: 152.

In some embodiments, the isolated monospecific or bispecific antibody or antigen binding fragment thereof can include a heavy chain having an amino acid sequence at least 90% identical to SEQ ID NO: 247, and a light chain having an amino acid sequence at least 90% identical to SEQ ID NO: 248.

Other embodiments described herein relate to a method of treating a complement mediated disease or disorder in a subject in need thereof. The method includes administering a therapeutically effective amount of the antibody or antigen binding fragment thereof as described herein to a subject in need thereof.

In some embodiments, the subject has a disease or disorder in which activation of the alternative complement pathway plays a role, and wherein the step of administering the antibody or antigen binding fragment thereof treats the disease or disorder.

In another aspect, the method includes the step of treating a disease or disorder in which activation of the alternative complement pathway plays a role, comprising administering antibody or antigen binding fragment thereof described herein to an individual that has, or is at risk of developing, said disease or disorder.

In a further aspect, the method includes the step of treating a disease or disorder selected from the group consisting of inflammatory diseases and inflammatory disorders.

In another aspect, the method includes the step of treating a disease or disorder selected from the group consisting of autoimmune diseases and autoimmune disorders.

In a further aspect, the method includes the step of treating an autoimmune disease or autoimmune disorder selected from the group consisting of systemic lupus erythematosus, myasthenia gravis, arthritis condition, Alzheimer's disease and multiple sclerosis.

In another aspect, the method includes the step of treating an arthritis condition. The arthritis condition can be selected from the group consisting of rheumatoid arthritis, osteo-arthritis, and juvenile arthritis.

In a further aspect, the method includes the step of treating a complement-associated disease or disorder selected from a group consisting of ocular diseases and ocular disorders. The ocular disease or ocular disorder can be selected from the group consisting of diabetic retinopathy, histoplasmosis of the eye, age-related macular degeneration, diabetic retinopathy, choroidal neo-vascularization (CNV), uveitis, diabetic macular edema, pathological myopia, von Hippel-Lindau disease, histoplasmosis of the eye, Central Retinal Vein Occlusion (CRVO), corneal neo-vascularization, and retinal neovascularization. The age-related macular degeneration can be selected from the group consisting of intermediate dry AMD and geographic atrophy.

In another aspect, the step of treating a complement-associated disorder is selected from the group consisting of asthmatic disorders and airway inflammation disorders. The airway inflammation disorder can be selected from the group consisting of: asthma, chronic obstructive pulmonary disease (“COPD”), allergic broncho-pulmonary aspergillosis, hypersensitivity pneumonia, eosinophilic pneumonia, emphysema, bronchitis, allergic bronchitis bronchiecstasis, cyctic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma, cough variant asthma, parasitic lung disease, respiratory syncytial virus (“RSV”) infection, parainfluenza virus (“PIV”) infection, rhinovirus (“RV”) infection, and adenovirus infection.

In another aspect, the step of treating a complement-associated disorder is selected Central and Peripheral Nervous System/Neurological diseases and disorders including multiple sclerosis (MS), myasthenia gravis (MG), myasthenia gravis, multiple sclerosis, Guillain Barre syndrome, Miller-Fisher syndrome, stroke, reperfusion following stroke, Alzheimer's disease, multifocal motor neuropathy (MMN), demyelination, Huntington's disease, amyotrophic lateral sclerosis (ALS), Parkinson's disease, degenerative disc disease (DDD), meningitis, cranial nerve damage from meningitis, variant Creutzfeldt-Jakob Disease (vCJD), idiopathic polyneuropathy, brain/cerebral trauma (including, but not limited to, hemorrhage, inflammation, and edema), neuromyelitis optica (NMO), including those serologically positive for aquaporin-4 (AQP4)-IgG autoantibody, and neuropathic pain.

### BRIEF DESCRIPTION OF THE DRAWINGS

FIG. 1 illustrates a schematic showing the structure of a monospecific anti-properdin antibody and a table listing the amino acid sequences of the V_{H} regions (SEQ ID NOs: 204 and 205) and V_{L} regions (SEQ ID NOs: 209 and 210) of the monospecific anti-properdin antibody in accordance with an embodiment.

FIG. 2 illustrates a schematic showing the structure of a monospecific anti-properdin antibody and a table listing the amino acid sequences of the V_{H} regions (SEQ ID NOs: 214 and 215) and V_{L} regions (SEQ ID NOs: 219 and 220) of the monospecific anti-properdin antibody in accordance with another embodiment.

FIG. 3 illustrates a schematic showing the structure of a monospecific anti-properdin antibody and a table listing the amino acid sequences of the V_{H} regions (SEQ ID NOs: 224 and 228) and V_{L} regions (SEQ ID NOs: 232 and 236) of the monospecific anti-properdin antibody in accordance with another embodiment.

FIG. 4 illustrates a table showing variable and constant chain linkers (SEQ ID NOs: 140-149).

FIG. 5 illustrates a schematic showing the structure of a bispecific anti-properdin/anti-albumin antibody and a table listing the amino acid sequences of the camelid anti-properdin portion (SEQ ID NOs: 240 and 244) and anti-albumin portion (SEQ ID NOs: 108 and 119) of the bispecific anti-properdin/anti-albumin antibody in accordance with another embodiment.

FIG. 6 illustrates a schematic showing the structure of single chain camelid anti-properdin/anti-albumin antibodies (NMT1001 and NMT1002) and a table listing the amino acid sequences of the camelid anti-properdin V_{HH} region (SEQ ID NO: 108), linker (SEQ ID NO: 143), and anti-albumin region (SEQ ID NO: 75) of NMT1001 (SEQ ID NOs: 108, 143 and 75) and the camelid anti-properdin V_{HH} region (SEQ ID NO: 110), linker (SEQ ID NO: 143), and anti-albumin region (SEQ ID NO: 75) of NMT 1002.

FIG. 7 illustrates a schematic showing the structure of a monospecific anti-properdin (Fab)2 (NMT1003) and a table listing the amino acid sequences of the V_{H} and V_{L} regions (SEQ ID NOs: 1 and 24), Fc-CH1 (SEQ ID NO: 138), Fc-LC (SEQ ID NO: 139) of NMT1003.

FIG. 8 illustrates a schematic showing the structure of bispecific anti-albumin/anti-properdin Fabs (NMT1004 and NMT1005) and a table listing the amino acid sequences of the anti-properdin VH (SEQ ID NO: 1), Fc-CH1 (SEQ ID NO: 138), VL (SEQ ID NO: 24), and Fc-LC (SEQ ID NO: 139), linker (SEQ ID NO: 143), and anti-albumin region (SEQ ID NO: 110) of NMT1004 and the anti-properdin VH (SEQ ID NO: 1), Fc-CH1 (SEQ ID NO: 138), VL (SEQ ID NO: 24), and Fc-LC (SEQ ID NO: 139), linker (SEQ ID NO: 143), and anti-albumin region (SEQ ID NO: 108) of NMT1005.

FIG. 9 illustrates a schematic showing the structure of a monospecific camelid anti-properdin antibody and a table listing the amino acid sequences of the camelid anti-properdin V_{HH} (SEQ ID NO: 74), linker (SEQ ID NO: 143), and IgG4 Fc with hinge region (SEQ ID NO: 137) of the monospecific camelid anti-properdin antibody (SEQ ID NO: 162) in accordance with another embodiment.

FIG. 10 illustrates a schematic showing the structure of a bispecific anti-properdin/anti-albumin antibody and a table listing the amino acid sequences of anti-properdin V_{H} (SEQ ID NO: 2), IG1 Fc (SEQ ID NO: 129), VL (SEQ ID NO: 25), and Fc-LC (SEQ ID NO: 139) and anti-albumin V_{H} (SEQ ID NO: 71), IG1 Fe (SEQ ID NO: 129), VL (SEQ ID NO: 72), and Fc-LC (SEQ ID NO: 139) of the bispecific anti-properdin/anti-albumin antibody in accordance with another embodiment.

FIG. 11 illustrates a schematic showing the structure of a bispecific anti-properdin/anti-albumin antibody and a table listing the amino acid sequences of anti-properdin V_{H} (SEQ ID NO: 52), IG1 Fc (SEQ ID NO: 131), VL (SEQ ID NO: 62), and Fc-LC (SEQ ID NO: 139) and anti-albumin V_{H} (SEQ ID NO: 71), IG1 Fc (SEQ ID NO: 131), VL (SEQ ID NO: 72), and Fc-LC (SEQ ID NO: 139) of the bispecific anti-properdin/anti-albumin antibody in accordance with another embodiment.

FIG. 12 illustrates a schematic showing the structure of a bispecific anti-properdin/anti-TNF antibody and a table listing the amino acid sequences of anti-properdin V_{H}(SEQ ID NO: 2), IG1 Fc (SEQ ID NO: 129), VL (SEQ ID NO: 25), and Fc-LC (SEQ ID NO: 139) and anti-TNF V_{H} (SEQ ID NO: 150), IG1 Fc (SEQ ID NO: 129), VL (SEQ ID NO: 151), and Fc-LC (SEQ ID NO: 139) of the bispecific anti-properdin/anti-TNF antibody in accordance with another embodiment.

FIG. 13 illustrates a schematic showing the structure of a bispecific anti-properdin/anti-VEGF antibody and a table listing the amino acid sequences of anti-properdin V_{H}(SEQ ID NO: 2), IG1 Fc (SEQ ID NO: 130), VL (SEQ ID NO: 25), and Fc-LC (SEQ ID NO: 139) and anti-VEGF V_{H} (SEQ ID NO: 152), IG1 Fc (SEQ ID NO: 130), VL (SEQ ID NO: 153), and Fc-LC (SEQ ID NO: 139) of the bispecific anti-properdin/anti-VEGF antibody in accordance with another embodiment.

FIG. 14 illustrates a schematic showing the structure of a bispecific camelid anti-properdin/anti-albumin antibody and a table listing the amino acid sequences of the camelid anti-properdin V_{HH} (SEQ ID NO: 74), linker (SEQ ID NO: 143), and IgG1 Fc with hinge region (SEQ ID NO: 130) and anti-albumin V_{H} (SEQ ID NO: 108), linker (SEQ ID NO: 143), and IgG1 Fc (SEQ ID NO: 143) of the bispecific camelid anti-properdin/anti-albumin antibody in accordance with another embodiment.

FIG. 15 illustrates a schematic showing the structure of a bispecific anti-properdin/anti-albumin antibody and a table listing the amino acid sequences of anti-properdin V_{H} (SEQ ID NO: 2), IG1 Fe (SEQ ID NO: 131), VL (SEQ ID NO: 25), and Fc-LC (SEQ ID NO: 139) and anti-albumin V_{H} (SEQ ID NO: 110), linker (SEQ ID NO: 143), and IgG1 Fe (SEQ ID NO: 131) of the bispecific anti-properdin/anti-albumin antibody in accordance with another embodiment.

FIG. 16 illustrates a schematic showing the structure of a bispecific anti-properdin/anti-albumin antibody and a table listing the amino acid sequences of anti-properdin V_{H} (SEQ ID NO: 2), IG1 Fc (SEQ ID NO: 130), VL (SEQ ID NO: 25), and Fc-LC (SEQ ID NO: 139) and anti-albumin V_{H} (SEQ ID NO: 71), IG1 Fc (SEQ ID NO: 130), VL (SEQ ID NO: 72), and Fc-LC (SEQ ID NO: 139) of the bispecific anti-properdin/anti-albumin antibody in accordance with another embodiment.

FIG. 17 illustrates a schematic showing the structure of a bispecific camelid anti-properdin/anti-albumin antibody and a table listing the amino acid sequences of the camelid anti-properdin V_{HH} (SEQ ID NO: 75), linker (SEQ ID NO: 143), and IgG4 Fc with hinge region (SEQ ID NO: 137) and anti-albumin V_{H} (SEQ ID NO: 108), linker (SEQ ID NO: 143), and IgG4 Fc (SEQ ID NO: 137) of the bispecific camelid anti-properdin/anti-albumin antibody in accordance with another embodiment.

FIG. 18 illustrates a schematic showing the structure of a bispecific anti-properdin/anti-TNF antibody and a table listing the amino acid sequences of anti-properdin V_{H}(SEQ ID NO: 2), IG1 Fc (SEQ ID NO: 131), VL (SEQ ID NO: 25), and Fc-LC (SEQ ID NO: 139) and anti-TNF V_{H} (SEQ ID NO: 150), IG1 Fc (SEQ ID NO: 131), VL (SEQ ID NO: 151), and Fc-LC (SEQ ID NO: 139) of the bispecific anti-properdin/anti-TNF antibody in accordance with another embodiment.

FIG. 19 illustrates a schematic showing the structure of a bispecific anti-properdin/anti-VEGF antibody and a table listing the amino acid sequences of anti-properdin V_{H}(SEQ ID NO: 2), IG1 Fc (SEQ ID NO: 131), VL (SEQ ID NO: 25), and Fc-LC (SEQ ID NO: 139) and anti-VEGF V_{H} (SEQ ID NO: 152), IG1 Fc (SEQ ID NO: 131), VL (SEQ ID NO: 153), and Fc-LC (SEQ ID NO: 139) of the bispecific anti-properdin/anti-VEGF antibody in accordance with another embodiment.

FIG. 20 illustrates plots showing binding affinity of NMT15, NMT16, NMT17, and NMT18 to properdin.

FIG. 21 illustrates plots showing binding affinity of NMT19, NMT20, NMT21, and NMT22 to properdin.

FIG. 22 illustrates plots showing inhibition of AP Hemolysis by NMT15, NMT16, NMT17, and NMT18.

FIG. 23. Illustrates plots showing inhibition of AP Hemolysis by NMT19, NMT20, NMT21, and NMT22.

FIG. 24 illustrates plots showing inhibition of MAC Formation by NMT15, NMT16, NMT17, and NMT18.

FIG. 25 illustrates plots showing inhibition of MAC Formation by NMT19, NMT20, NMT21, and NMT22.

FIG. 26 illustrates plots showing inhibition of Properdin-C3b Binding by NMT15, NMT16, NMT17, and NMT18.

FIG. 27 illustrates plots showing inhibition of Properdin-C3b Binding by NMT19, NMT20, NMT21, and NMT22.

FIG. 28 illustrates plots showing inhibition of AP mediated C3b Formation and Deposition by NMT15, NMT17, NMT18, NMT20, and NMT21.

FIG. 29 illustrates plots showing CP mediated MAC Formation and Deposition by NMT15, NMT17, NMT18, NMT20, and NMT21.

FIG. 30 illustrates plots showing CP mediated C3b Formation and Deposition by NMT15, NMT17, NMT18, NMT20, and NMT21.

FIG. 31 illustrates plots showing binding affinity of NMT23, NMT24, NMT25, NMT26, NMT27, and NMT28 to properdin.

FIG. 32 illustrates plots showing inhibition of MAC Formation and Deposition by NMT23, NMT24, NMT25, NMT26, NMT27, and NMT28.

FIG. 33 illustrates plots showing inhibition of AP Hemolysis by NMT23, NMT24, NMT26, and NMT27.

FIG. 34. illustrates plots showing inhibition of AP mediated C3 convertase formation and Deposition by NMT28.

FIG. 35 illustrates plots showing CP mediated C3b Formation and Deposition by NMT28.

FIG. 36 illustrates plots showing binding affinity of NMT17-100, NMT17-101, NMT18-100, NMT18-101, NMT28-100, and NMT28-101 to Properdin.

FIG. 37 illustrates plots showing inhibition of AP Hemolysis by NMT17-100, NMT17-101, NMT18-100, NMT18-101, NMT28-100, and NMT28-101.

FIG. 38 illustrates plots showing inhibition of Mac Formation by NMT17-100, NMT17-101, NMT18-100, NMT18-101, NMT28-100, and NMT28-101.

FIG. 39 illustrates plots showing inhibition of AP mediated C3b Formation and Deposition by NMT17-100, NMT17-101, NMT18-100, NMT18-101, NMT28-100, and NMT28-101.

FIG. 40 illustrates plots showing binding Affinity Comparison of N297 and Xtend Fc regions—NMT17, NMT18, NMT28, NMT17-100, NMT17-101, NMT18-100, NMT18-101, NMT28-100, and NMT28-101 to Properdin.

FIG. 41 illustrates plots showing binding Affinity Comparison of N297 and Xtend Fc regions—NMT17, NMT18, NMT28, NMT17-100, NMT17-101, NMT18-100, NMT18-101, NMT28-100, and NMT28-101 to Properdin.

FIG. 42 illustrates plots showing inhibition of AP-Hemolysis Comparison of N297 and Xtend Fc regions—NMT17, NMT18, NMT28, NMT17-100, NMT17-101, NMT18-100, NMT18-101, NMT28-100, and NMT28-101.

FIG. 43 illustrates plots showing inhibition of C3b Formation and Deposition—Comparison of N297 and Xtend Fc regions—NMT17, NMT18, NMT28, NMT17-100, NMT17-101, NMT18-100, NMT18-101, NMT28-100, and NMT28-101.

FIG. 44 illustrates plots showing binding Potency to Properdin—Comparison of N297 and Xtend Fc regions—NMT29, NMT30, NMT31.

FIG. 45 illustrates plots showing formation of AP mediated MAC—Comparison of N297 and Xtend Fe regions—NMT29, NMT30, NMT31.

FIG. 46 illustrates plots showing inhibition of AP-mediated Hemolysis—Comparison of N297 and Xtend Fc regions—NMT29, NMT30, NMT31.

FIG. 47 illustrates plots showing inhibition of AP-mediated C3b Formation & Deposition—Comparison of N297 and Xtend Fc regions—NMT29, NMT30, NMT31.

FIG. 48 illustrates plots showing binding Potency to Properdin—Comparison of N297 and Xtend Fc regions—NMT30-100, NMT30-101, NMT31-100, and NMT31-101.

FIG. 49 illustrates plots showing inhibition of AP Hemolysis—Comparison of N297 and Xtend Fc regions—NMT30-100, NMT30-101, NMT31-100, and NMT31-101.

FIG. 50 illustrates plots showing inhibition of AP mediated MAC Formation—Comparison of N297 and Xtend Fc regions—NMT30-100, NMT30-101, NMT31-100, and NMT31-101.

FIG. 51 illustrates plots showing inhibition of AP mediated C3b Formation and Deposition—Comparison of N297 and Xtend Fc regions—NMT30-100, NMT30-101, NMT31-100, and NMT31-101.

FIG. 52 illustrates plots showing potency Binding to Properdin—Comparison of N297 and Xtend Fc regions—NMT30, NMT31, NMT30-100, NMT30-101, NMT31-100, and NMT31-101.

FIG. 53 illustrates plots showing inhibition of AP Hemolysis—Comparison of N297 and Xtend Fc regions—NMT30, NMT31, NMT30-100, NMT30-101, NMT31-100, and NMT31-101.

FIG. 54 illustrates plots showing inhibition of MAC Formation—Comparison of N297 and Xtend Fc regions—NMT30, NMT31, NMT30-100, NMT30-101, NMT31-100, and NMT31-101.

FIG. 55 illustrates plots showing inhibition of C3b Formation and Deposition—Comparison of N297 and Xtend Fc regions—NMT30, NMT31, NMT30-100, NMT30-101, NMT31-100, and NMT31-101.

### DETAILED DESCRIPTION OF THE INVENTION

As used herein, the term “acceptor human framework” refers to a framework comprising the amino acid sequence of a VL or VH framework derived from a human immunoglobulin framework, or from a human consensus framework.

As used herein, the term “antibody” covers full length monoclonal antibodies, polyclonal antibodies, nanobodies and multi-specific antibodies. Biological antibodies are usually hetero-tetrameric glycoproteins of about 150,000 Daltons, composed of two identical light (L) chains and two identical heavy (H) chains. The two heavy chains are linked together by disulfide bonds, and each heavy chain is linked to a light chain by a disulfide bond. Each full-length IgG molecule contains at least two binding sites for a specific target or antigen. Light chains are either kappa or the lambda. Both light chains contain a domain of variable amino acid sequences, called the variable region (variously referred to as a “V_{L},” “Vₖₐₚₚₐ,” or “V_{lambda}-region”) and a domain of relatively conserved amino acid sequences, called the constant region (“CL-region”). Similarly, each heavy chain contains a variable region (“V_{H}-region”) and three constant domains (“C_{H1}-,” “C_{H2}-,” and “C_{H3}-regions”) and a hinge region.

As used herein, the term “antibody fragment” refers to a segment of a full-length antibody, generally called as the target binding or variable region. Other antibodies include diabodies, linear antibodies, single-chain antibody molecules and multispecific antibodies formed from antibody fragments. Examples include Fab, Fab′, F(ab′)2, Fv, or scFv fragments. An “Fv” fragment is the minimum antibody fragment which contains a complete target recognition and binding site.

As used herein, the term “antigen binding fragment” refers to a fragment or fragments of an antibody molecule that contain the antibody variable regions responsible for antigen binding.

Antigen-binding fragments can be prepared from full-length antibody by protease digestion. Antigen-binding fragments may be produced using standard recombinant DNA methodology by those skilled in the art. Examples of antigen binding fragments:
“Fab” fragments (single chain variable regions with VH and VL)
“Monovalent Fragments” (antibody fragments consisting of the VL, VH, CL and CH1 domains)
“F(ab′)2” fragments (bivalent fragments comprising two Fab fragments linked by a disulfide bridge at the hinge region);
“Fd” fragments (which consist of the VH and CH1 domains of an antibody);
“Fv” fragment (which consist of the VL and VH domains of a single arm of an antibody);
single domain antibody (“dAb”), which consist of a VH domain or a VL domain; and
an isolated Complementarity Determining Region (“CDR”).

As used herein, complementarity-determining region (“CDR”) are the key binding regions of the antibody. CDR refers to a specific region within variable regions of the heavy and the light chain. Generally, the variable region consists of four framework regions (FR1, FR2, FR3, FR4) and three CDRs arranged in the following manner: NH₂—FR1-CDR1-FR2-CDR2-FR3-CDR3-FR4-COOH. The term “framework regions” refers to those variable domain residues other than the CDR residues herein defined.

CDRs, as antigen binding fragments, can also be incorporated into single domain antibodies, maxi bodies, mini bodies, intrabodies, diabodies, triabodies, tetra bodies, v-NAR and bis-scFv. Antigen binding fragments of antibodies can be grafted into scaffolds based on polypeptides. Antigen binding fragments can be incorporated into single chain molecules comprising a pair of tandem Fv segments (VH-CH1-VH-CH1) which, together with complementary light chain polypeptides, form a pair of antigen binding regions.

As used herein, a “single chain Fv” or “scFv” antibody fragment comprises the VH and VL domains of an antibody, wherein these domains are present in a single polypeptide chain.

As used herein “effector functions” refer to those biological activities attributable to the native Fc region of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); lack of activation of platelets that express Fc receptor; and B cell activation. In order to minimize or eliminate side effects of a therapeutic antibody, it may be preferable to minimize or eliminate effector functions.

In the context of this application “undesired effector functions” include ADCC, CDC, classical pathway activation, cell activation, and antibody mediated inflammation.

An “engineered antibody” is an antibody that is not naturally produced, and which has been altered or created to achieve a specific purpose or to have a specific characteristic. For example, antibodies which have undergone deliberate modifications to their wild type forms, to have reduced effector functions, are engineered antibodies.

As used herein, the term “Fc region” refers to the region of the antibody that provides defense to a given antigen.

As used herein, the term “first portion of the antibody” refers to a portion of a whole antibody, a portion less than the whole, which contains the antigen binding regions of the antibody. “Second portion of the antibody” refers to a portion of a whole antibody, a portion less than the whole, which consists of the portion of the antibody which is not included in the first portion.

The terms “Fc receptor” or “FcγR” describe a receptor that binds to the Fc region of an IgG. “FcγRI,” “FcγRII,” and “FcγRIII” are subclasses of FcγRs.

As used herein, the term “reduced Fc effector function(s)” refers to the function(s) of an antibody wherein the antibody does not act against an antigen that recognizes the Fc region of the antibody. Examples of reduced Fc effector functions can include, but are not limited to, reduced Fc binding to the antigen, lack of Fc activation against an antigen, an Fc region that contains mutations to prevent normal Fc effector functions, or prevention of the activation of platelets and other cells that have Fc receptors.

A “modification” to an antibody, antibody fragment, and/or Fc region of an antibody, refers to a substitution, insertion, or deletion of one or more amino acids in the protein's wild type polypeptide sequence. A modified antibody, antibody fragment, and/or Fc region is one in which a modification has been artificially made.

As used herein, “competitively inhibits” refers to competitive inhibition of binding of a isolated antibody or antigen binding portion thereof to properdin by any other molecule.

As used herein, the term “epitope” refers to a site on properdin to which antibody and fragments thereof bind and perform the functional activity. The term epitope is the same as “antigenic site”, and “antibody binding site,”. One skilled in the art can align the sequence of properdin of a human with the sequence of properdin from another animal species and determine the positions of the epitope.

As used herein, “Fab fragment” refers to the constant domain of the light chain and the first constant domain of the heavy chain. Fab′ fragments differ from Fab fragments by the few extra residues at the carboxyl terminus of the heavy chain CH1 domain including one or more cysteines from the antibody hinge region. F(ab′) fragments are produced by cleavage of the disulfide bond at the hinge cysteines of the F(ab′)2pepsin digestion product.

As used herein, the term “antigen binding fragment thereof” of an antibody refers to an antibody fragment having qualitative biological activity in common with a full-length antibody. For example, an antigen binding fragment thereof of an antibody is one which can bind to properdin in such a manner so as to prevent or substantially reduce the alternative complement activation.

As used herein, the term “human consensus framework” refers to a framework which represents the most commonly occurring amino acid residue in a selection of human immunoglobulin VL or VH framework sequences. Generally, the selection of human immunoglobulin VL or VH sequences is from a subgroup of variable domain sequences.

As used herein, a “humanized antibody” refers to an antibody consisting of mostly human sequences, except for CDR1, CDR2, and CDR3. All framework regions are also humanized. A chimeric antibody comprises murine CDRs, murine framework regions, and human constant regions. Collectively, chimeric antibodies contain murine both variable regions and human constant regions.

As used herein, the term “monovalent antibody or antigen binding fragment thereof” refers to an antibody or antigen binding fragment thereof comprising a single binding domain, e.g., V_{H} or V_{HH}, for an antigen, e.g., a single properdin molecule. In one embodiment, the bound antigen molecule is part of a multimer, e.g., a trimer or higher order multimer of properdin monomers. Antibodies generally, including monovalent antibodies or antibody fragments thereof, bind with a high degree of specificity to a particular antigen.

As used herein, the term “single domain antibody” defines molecules where the antigen binding site is present on, and formed by, a single immunoglobulin domain. Generally, the antigen binding site of an immunoglobulin single variable domain is formed by no more than three CDRs. The single variable domain may, for example, include a light chain variable domain sequence (a V_{L} sequence) or a suitable fragment thereof; or a heavy chain variable domain sequence (e.g., a V_{H} sequence or V_{HH} sequence) or a suitable fragment thereof; as long as it is capable of forming a single antigen binding unit (i.e., a functional antigen binding unit that essentially is the single variable domain, such that the single antigen binding domain does not need to interact with another variable domain to form a functional antigen binding unit).

As used herein, the term “camelid antibody” refers to an antibody derived from a camelid species, for example, in a camel, dromedary, llama, alpaca or guanaco. Camelid antibodies differ from those of most other mammals in that they lack a light chain, and thus include only heavy chains with complete and diverse antigen binding capabilities (Hamers-Casterman, C. et al., Nature, 363:446-8, 1993).

As used herein, the term “V_{HH}” refers to a single heavy chain variable domain antibody devoid of light chains. V_{HH} chains, for example, can be of the type that can be found in Camelidae or cartilaginous fish that are naturally devoid of light chains or to a synthetic and non-immunized V_{HH} that can be constructed accordingly. Each heavy chain includes a variable region encoded by V-, D- and J-exons. A V_{HH} may be a natural V_{HH} antibody, e.g., a camelid antibody, or a recombinant protein including a heavy chain variable domain.

As used herein, the term an “isolated antibody” refers to an antibody that is substantially free of other antibodies having different antigenic specificities (e.g., an isolated antibody that binds to properdin is substantially free of contaminants, e.g., antibodies that do not bind to properdin). In addition, an “isolated” antibody is one that has been identified and separated and/or recovered from a component of its natural environment. Contaminant components of its natural environment are materials that could interfere with diagnostic or therapeutic uses for the antibody, and may include enzymes, hormones, and other proteinaceous or nonproteinaceous solutes.

As used herein, the term “specific binding” of an antibody or fragment thereof, polypeptide, or peptidomimetic is binding to a target molecule that is measurably different from binding to molecules that are not target molecules. As used herein, specific binding refers to a greater than 95% preference for binding a particular antigen versus background (“non-specific”) binding. “Substantially specific” binding refers to a greater than about 80% preference for binding a particular antigen versus background. Binding can be measured using a variety of methods including, but not limited to, Western blot, immunoblot, enzyme-linked immunosorbant assay (“ELISA”), radioimmunoassay (“RIA”), immunoprecipitation, surface plasmon resonance, bio-layer interferometry, chemiluminescence, fluorescent polarization, phosphorescence, immunohistochemical analysis, matrix-assisted laser desorption/ionization time-of-flight (“MALDI-TOF”) mass spectrometry, microcytometry, microarray, microscopy, fluorescence activated cell sorting (“FACS”) and flow cytometry.

As used herein, the term “human properdin” refers to a 469 amino acid soluble glycoprotein found in plasma that has seven thrombospondin type I repeats (TSR) with the N-terminal domain, TSR0, being a truncated domain. Human properdin, a 53 kDa protein, includes a signal peptide (amino acids 1-28), and six, non-identical TSR repeats about 60 amino acids each, as follows: amino acids 80-134 (TSR1), amino acids 139-191 (TSR2), amino acids 196-255 (TSR3), amino acids 260-313 (TSR4), amino acids 318-377 (TSR5), and amino acids 382-462 (TSR6). Properdin is formed by oligomerization of a rod-like monomer into cyclic dimers, trimers, and tetramers. The amino acid sequence of human properdin is found in the GenBank database under the following accession numbers: for human properdin, see, e.g., GenBank Accession Nos. AAA36489, NP_002612, AAH15756, AAP43692, S29126 and CAA40914. Properdin is a positive regulator of the alternative complement activation cascade. Known binding ligands for properdin include C3b, C3bB and C3bBb (Blatt, A. et al., Immunol. Rev., 274:172-90, 2016).

As used herein, the term “mouse properdin” refers to a 457 amino acid soluble glycoprotein found in plasma that has seven TSRs with the N-terminal domain, TSR0, being truncated. Mouse properdin, a 50 kDa protein, includes a signal peptide (amino acids 1-24), and six, non-identical TSRs of about 60 amino acids each, as follows: amino acids 73-130 (TSR1), amino acids 132-187 (TSR2), amino acids 189-251 (TSR3), amino acids 253-309 (TSR4), amino acids 311-372 (TSR5), and amino acids 374-457 (TSR6). Mouse properdin is formed by oligomerization of a rod-like monomer into cyclic dimers, trimers, and tetramers. The amino acid sequence of mouse properdin is found, for example, in the GenBank database (Gen Bank Accession Nos. P11680 and 505478).

As used herein, the term “TSR0 domain” refers to the truncated domain of properdin that precedes the TSR1 domain of properdin. For example, the TSR0 domain of human properdin includes amino acids 28-76.

As used herein, the term “TSR1 domain” refers to the domain of properdin adjacent to the TSR0 domain of properdin. For example, the TSR0 domain of human properdin includes amino acids 77-134.

As used herein, the term “an activity of properdin” refers to the biological activity of properdin including, but not limited to, binding interactions that lead to the stability of the C3/C5 convertase. Properdin binds most avidly to C3b,Bb—the alternative pathway C3/C5 convertase, but also binds to C3b; C3b,B and C3b,Bb. One function is to stabilize the C3b,Bb complex allowing increased alternative pathway activation (Pangburn, M., Methods Enzymol., 162:639-53, 1988; Nolan, K. & Reid, K., Methods Enzymol., 223:35-46, 1993). Properdin enhances formation of the alternative pathway C3 convertase by increasing binding of factor B to P,C3b complexes. Thus, properdin is an accelerator (positive regulator) of complement activation. Properdin also has been implicated in initiating activation of the alternative pathway by binding to the target surface and initiating C3/C5 convertase formation (Kemper C. & Hourcade, D., Mol. Immunol., 45:4048-56, 2008).

As used herein, the term “alternative complement pathway” refers to one of three pathways of complement activation (the others being the classical pathway and the lectin pathway). The alternative complement pathway is typically activated by bacteria, parasites, viruses or fungi, although IgA Abs and certain IgL chains have also been reported to activate this pathway.

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur both in fluid phase as well as at cell surface and can lead to excessive complement activation or insufficient regulation, both causing tissue injury.

As used herein, the term “a disease mediated by alternative complement pathway dysregulation” refers to an interruption, cessation or disorder of body functions, systems or organs caused by alternative complement pathway dysregulation. Such diseases would benefit from treatment with a composition or formulation described herein. In some embodiments, the disease is caused by any aberration in the ability of the alternative complement pathway to provide host defense against pathogens and clear immune complexes and damaged cells, and for immunoregulation. Also encompassed herein are diseases, directly or indirectly, mediated by dysregulation of one or more components of the alternative complement pathway, or a product generated by the alternative complement pathway.

As used herein, the term “alternative complement pathway-dependent membrane attack complex assembly” refers to a terminal complex formed as a result of alternative complement pathway activation and includes complement components C5, C6, C7, C8 and C9. Assembly of the membrane attack complex (MAC) leads to cell lysis.

As used herein, the term “alternative complement pathway dependent hemolysis” refers to the lysis of red blood cells mediated by increased alternative complement pathway-dependent MAC assembly and/or deposition on red blood cells.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. A linker may refer to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer may be part of the primary sequence of a polypeptide (e.g., joined to the spaced polypeptides or polypeptide domains via the polypeptide backbone). A linker may comprise one or more glycine and serine residues.

As used herein, the term “identical” or “substantially identical” with respect to an antibody chain polypeptide sequence may be construed as an antibody chain exhibiting at least 65%, 70%, 80%, 90% or 95% sequence identity to the reference polypeptide sequence present in the variable region of the antigen binding fragment. The term with respect to a nucleic acid sequence may be construed as a sequence of nucleotides exhibiting at least about 65%, 75%, 85%, 90%, 95% or 97% sequence identity to the reference nucleic acid sequence.

As used herein, the term “individual” refers to a vertebrate, preferably a mammal and more preferably a human. Individuals amenable to treatment include those who are presently asymptomatic, but who are at risk of developing a symptomatic disorder in which the alternative complement pathway plays a role, or in which activation of the alternative complement pathway plays a role.

As used herein, the term “mammal” refers to any animal classified as a mammal includes humans, higher primates, domestic and farm animals, horses, pigs, cattle, dogs, cats and ferrets, etc. In one embodiment of the invention, the mammal is a human.

As used herein, “monoclonal antibody” refers to a homogeneous population of antibodies. Such antibodies are highly specific and are directed against a single target antigen. These monoclonal antibodies are homogeneously produced by the hybridoma culture, uncontaminated by other immunoglobulins. Monoclonal antibodies can also be produced by other procedures such as phase display by well known methods.

As used herein, the term “selectively inhibit the alternative complement pathway” refers to preferentially and exclusively inhibits the alternative complement pathway, but does not inhibit other pathways for complement activation, including the classical complement pathway. For example, the humanized and chimerized antibodies and their antigen-binding fragments selectively inhibits the alternative complement pathway. This definition applies to other methods described herein wherein the alternative complement pathway is selectively inhibited.

As used herein, the term “therapeutically effective amount” refers to the amount of a “properdin antagonist” which is required to achieve a measurable improvement in the state, for example, pathology, of the target disease or condition, such as, for example, a complement-associated eye condition.

As used herein, the term “treatment” refers to both therapeutic treatment and prophylactic or preventative measures.

Described herein are novel anti-properdin antibodies and antigen binding fragments thereof that are useful for the prevention and treatment of complement-mediated and/or associated conditions. These anti-properdin antibodies and antigen binding fragments can include, but are not limited to, anti-properdin antibodies and antibody variants thereof, antigen-binding fragments thereof, other binding polypeptides, and/or peptides. These anti-properdin antibodies and antigen binding fragments can bind to properdin and can be capable of neutralizing, blocking, partially or fully inhibiting, abrogating, reducing or interfering with properdin functional activities, for example the ability of properdin to participate in the pathology of any complement-associated inflammatory disease or disorder.

### Anti-Properdin Antibodies and Antigen Binding Fragments Thereof

The anti-properdin antibodies and antigen binding fragments thereof can prevent the binding of properdin to C3b to form the PC3b complex by selectively binding to properdin. As a result, the PC3b complex and the PC3bBb complex will not form. Since the PC3bBb complex cleaves C5 into C5a and C5b, the MAC complex (C5b-9) also will not form. Thus, by inhibiting the binding of properdin to C3b, the anti-properdin antibodies and antigen binding fragments thereof will inhibit the formation of the MAC complex. Elevated levels of the MAC complex have been found to be associated with multiple acute and chronic disease conditions. Therefore, inhibition of the MAC complex via the anti-properdin antibodies and antigen binding fragments thereof is important for clinical benefit in the diseases where complement activation plays a role in disease pathology.

The PC3b complex, the PC3bB complex, and the PC3bBb complex can all be polymerized. The anti-properdin antibodies and antigen binding fragments thereof described herein can inhibit the polymerization of each of these complexes, where the molar ratio of properdin to each of C3b, factor B, or factor Bb can be 1:1. The anti-properdin antibodies and antigen binding fragments thereof can inhibit the polymerization of each of these complexes, where each of these complexes comprises at least one more mole properdin than to each of, C3b, factor B, and factor Bb in each complex respectively. In one example, for the PC3b complex, the molar ratio between properdin and C3b can be expressed as (P)x(C3b)_{y}, where X=Y+1. In another example, for the PC3bB complex, the molar ratio between properdin, C, C3b, and factor B can be expressed as (P)x(C3b)_{y}(B)_{z}, where X=Y+Z. This example also can express the molar ratio of properdin to C3b and factor Bb in the PC3bBb complex.

The anti-properdin antibodies and antigen binding fragments thereof can have the ability to inhibit any biological activity of properdin. Such activity can bring a measurable improvement in the state of pathology of a properdin-associated disease or condition, for example, a complement-associated inflammatory disease or disorder. The activity can be evaluated in in vitro or in vivo tests, including, but not limited to, binding assays, alternative pathway hemolysis assays using a relevant animal model, or human clinical trials.

In another embodiment, anti-properdin antibodies and antigen binding fragments thereof can bind to a specific epitope located on properdin to inhibit AP activation. In one example, the anti-properdin agent can bind to the N-terminal domain of properdin to inhibit the binding of properdin to C3b.

The anti-properdin antibodies or antigen binding fragments thereof described herein can be produced by using full-length properdin, properdin polypeptides, and/or using antigenic properdin epitope-bearing peptides, for example, a fragment of the properdin polypeptide. Properdin peptide and polypeptides can be isolated and used to generate antibodies as natural polypeptides, recombinant or synthetic recombinant polypeptides. All antigens useful for producing anti-properdin antibodies can be used to generate monospecific and bispecific antibodies.

The anti-properdin antibody may be a monoclonal antibody or derived from a monoclonal antibody. Suitable monoclonal antibodies to selected antigens may be prepared by known techniques (“Monoclonal Antibodies: A manual of techniques,” Zola (CRC Press, 1988); “Monoclonal Hybridoma Antibodies: Techniques and Applications,” Hurrell (CRC Press, 1982), the entire contents of which are incorporated herein by reference).

The anti-properdin antibodies and antigen binding fragments thereof can include humanized monoclonal anti-properdin antibodies or antigen-binding fragments thereof that selectively bind to properdin and selectively inhibit activation of the alternative complement pathway. The anti-properdin antibodies and antigen binding fragments thereof can be used to treat any alternative pathway associated inflammatory diseases or disorders in humans or other mammals.

Methods for making humanized non-human antibodies are well known in the art. Humanization is essentially performed by substituting rodent CDRs or CDR sequences for the corresponding sequences of a human antibody. The choice of human variable domains, both light and heavy, to be used in making the humanized antibodies can, in some instances, be important to reduce antigenicity and/or human anti-mouse antibody (HAMA) response. In some embodiments, the anti-properdin antibodies and antigen binding fragments thereof are humanized such that HAMA response is reduced or eliminated.

Ordinarily, properdin can have a range of percentages of amino acid sequence identity, ranging from at least about 60%, to at least about 70%, to at least about 80%, to at least about 85%, to at least about least about 90%, to at least about 95%, to at least about 98%, to at least about 99% amino acid sequence identity with the mature human amino acid sequence.

The variable domain of the antibodies refers to certain portions of the variable domains that differ in sequence among antibodies. The variability in the anti-properdin antibodies and antigen binding fragments thereof can be concentrated in three CDR segments, located in both the light chain and the heavy chain variable domains. The highly conserved portions of variable domains are called framework (FR) regions. In the anti-properdin antibodies described herein, there are four FR regions, connected by three CDRs, that can comprise a variable chain. The CDRs in each of the light and heavy chains are held together in close proximity by the FR regions and, with the CDRs from the other chain, can contribute to the formation of the target binding site of antibodies.

Antibody Humanization is a process that can generate engineered human antibodies with variable region (“V-region”) sequences that are substantially similar to actual human germ-line sequences, while retaining the binding specificity and affinity of a reference antibody. This process can graft, for example, the CDR1, CDR2, and CDR3 regions of the heavy and the light chain sequences into humanized human framework that is both optimized and previously identified prior to the start of the grafting process. The variable region containing humanized framework can be produced into Fab, Fab′, Fab2, or single chain antigen binding fragments thereof. The resulting engineered humanized antibody fragments can retain the binding specificity of the parent murine antibody for the antigen properdin, and can have an equivalent or higher binding affinity for a specific antigen than the parent antibody. The engineered antigen binding fragments can have heavy and light chain V-regions with a high degree of amino acid sequence identity compared to the closest human germline antibody genes. For example, additional maturational changes can be introduced in the CDR3 regions of each chain during construction in order to identify antibodies with optimal binding kinetics.

In some embodiments, the antibody may be a single-domain antibody, such as a V_{HH}. Such antibodies exist naturally in camelids and sharks (Saerens, D. et al., Curr. Opin. Pharmacol., 8:600-8, 2008). Camelid antibodies are described in, for example. U.S. Pat. Nos. 5,759,808; 5,800,988; 5,840,526; 5,874,541; 6,005,079; and 6,015,695, the entire contents of each of which are incorporated herein by reference. The cloned and isolated V_{HH} domain is a stable polypeptide that features the full antigen-binding capacity of the original heavy-chain antibody. V_{HH} domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture.

In some embodiments, the anti-properdin antibodies and antigen binding fragments thereof can bind to the same epitope on properdin as the antibodies recited in this application. Such antibodies can be identified based on their ability to cross-compete with or competitively inhibit the anti-properdin antibodies and antigen binding fragments thereof in standard properdin binding assays. Thus, all anti-properdin antibodies and antigen binding fragments thereof that competitively inhibit the binding of anti-properdin antibodies and the antigen binding fragments thereof are encompassed by this disclosure.

The antigen binding fragments of antibodies can be identified following protease digestion. These include, for example, the “Fab fragment”, “Fab′ fragment” (a Fab with the heavy chain hinge region), and “F(ab′)₂ fragment” (a dimer of Fab′ fragments joined by the heavy chain hinge region). Recombinant methods have been used to generate such fragments and to generate even smaller antibody fragments, e.g., those referred to as “single chain Fv” (variable fragment) or “scFv,” consisting of V_{L} and V_{H} joined by a synthetic peptide linker (V_{L}-linker-V_{H}). Fab fragments, Fab′ fragments and scFv fragments are monovalent or monospecific for antigen binding, as they each include only one antigen binding domain including one V_{H}/V_{L} dimer. Even smaller monovalent antibody fragments are the dAbs, which include only a single immunoglobulin variable domain, e.g., V_{H} or V_{L}, that alone specifically binds antigen, i.e., without the need for a complementary V_{L} or V_{H} domain, respectively. A dAb binds antigen independently of other V domains; however, a dAb can be present in a homo- or hetero-multimer with other V_{H} or V_{L} domains where the other domains are not required for antigen binding by the dAb, i.e., where the dAb binds antigen independently of the additional V_{H} or V_{L} domains.

In some embodiments, the anti-properdin antibody or antigen binding fragment thereof can be a monospecific antibody or antigen binding fragment thereof that includes at least one of:
a)
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEX₃X₄X₅X₆X₇X₈X₉ (SEQ ID NO: 202), wherein X₁ is D, E, S, or A; X₂ is H or Y; X₃ is P, S, or Y; X₄ is A or D; X₅ is D, R, or Q; X₆ is K, E, or S; X₇ is F or V; X₈ is E, K, Q, or R; and X₉ is D or G; and
a CDR-H3 comprising the amino acid sequence of RGGGYYLDY (SEQ ID NO: 203);
b)
   a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y;
c)
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX₂PALKS (SEQ ID NO: 212), wherein X₁ is F, H, W, or Y; and X₂ is A, N, Q, or S; and
a CDR-H3 comprising the amino acid sequence of IGX₁GYYSFDY (SEQ ID NO: 213), wherein X₁ is A, D, E, or S;
d)
a CDR-L1 comprising the amino acid sequence of X₁ASQDVSDAVA (SEQ ID NO: 216), wherein X₁ is K or R;
   a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y;
e)
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222); and
a CDR-H3 comprising the amino acid sequence of SVX₁GIX₁SYX₁ AAFX₂L (SEQ ID NO: 223), wherein X₁ is A, D, E, or S; and X₂ is A, N, Q, or S;
f)
a CDR-L1 comprising the amino acid sequence of X₁ASDX₂IYSLLA (SEQ ID NO: 229), wherein X₁ is Q or R; X₂ is A, N, Q, or S;
   a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S;
g)
a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M; and
a CDR-H3 comprising the amino acid sequence of SAX₁GSX₁SYX₁AYFTL (SEQ ID NO: 227), wherein X₁ is A, D, E, or S;
h)
a CDR-L1 comprising the amino acid sequence of X₁ASDX₂IYSX₂LA (SEQ ID NO: 233), wherein X₁ is Q or R; X₂ is A, N, Q, or S;
   a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y;
i)
a CDR-H1 comprising the amino acid sequence of GRISSIIHMA (SEQ ID NO: 237), wherein X₁ is F, H, W, or Y,
a CDR-H2 comprising the amino acid sequence of RX₁GTTX₁YAX₂SX₁X₃G (SEQ ID NO: 238), wherein X₁ is I or V; X₂ is A, D, E, or S; and X₃ is A or K; and
a CDR-H3 comprising the amino acid sequence of LQYEX₁HGGAX₂Y (SEQ ID NO: 239), wherein X₁ is A or K; and X₂ is A, D, E, or S;
j)
a CDR-H1 comprising the amino acid sequence of GRIFEX₁X₂MMA (SEQ ID NO: 241), wherein X₁ is I or V; and X₂ is A, D, N, Q, or S,
a CDR-H2 comprising the amino acid sequence of RX₁GTTTYAX₂SX₁X₃G (SEQ ID NO: 242), wherein X₁ is I or V; X₂ is A, D, E, or S; and X₃ is A or K; and
a CDR-H3 comprising the amino acid sequence of LQYX₁RYGGAEY (SEQ ID NO: 243), wherein X₁ is A, D, E, or S; or
k)
or a heavy chain variable region and/or light chain variable region that competitively inhibits binding of an isolated monospecific or bispecific antibody or antigen binding fragment thereof comprising at least one of a), b), c), d), e), f), g), h), i), or j) to monomeric properdin.

In other embodiments, the anti-properdin antibody or antigen binding fragment thereof, can include:
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEX₃X₄X₅X₆X₇X₈X₉ (SEQ ID NO: 202), wherein X₁ is D, E, S, or A; X₂ is H or Y; X₃ is P, S, or Y; X₄ is A or D; X₅ is D, R, or Q; X₆ is K, E, or S; X₇ is F or V; X₈ is E, K, Q, or R; and X₉ is D or G; and
a CDR-H3 comprising the amino acid sequence of RGGGYYLDY (SEQ ID NO: 203);
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y.

In some embodiments, the anti-properdin antibody or antigen binding fragment thereof can include:
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX₂PALKS (SEQ ID NO: 212), wherein X₁ is F, H, W, or Y; and X₂ is A, N, Q, or S;
a CDR-H3 comprising the amino acid sequence of IGX₁GYYSFDY (SEQ ID NO: 213), wherein X₁ is A, D, E, or S;
a CDR-L1 comprising the amino acid sequence of X₁ASQDVSDAVA (SEQ ID NO: 216), wherein X₁ is K or R;
a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y.

In some embodiments, the anti-properdin antibody or antigen binding fragment thereof can include:
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222);
a CDR-H3 comprising the amino acid sequence of SVX₁GIX₁SYX₁ AAFX₂L (SEQ ID NO: 223), wherein X₁ is A, D, E, or S; and X₂ is A, N, Q, or S;
a CDR-L1 comprising the amino acid sequence of X₁ASDX₂IYSLLA (SEQ ID NO: 229), wherein X₁ is Q or R; X₂ is A, N, Q, or S;
a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S.

In some embodiments, the anti-properdin antibody or antigen binding fragment thereof can include:
a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M;
a CDR-H3 comprising the amino acid sequence of SAX₁GSX₁SYX₁AYFTL (SEQ ID NO: 227), wherein X₁ is A, D, E, or S;
a CDR-L1 comprising the amino acid sequence of X₁ASDX₂IYSX₂LA (SEQ ID NO: 233), wherein X₁ is Q or R; X₂ is A, N, Q, or S;
a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y.

In some embodiments, the anti-properdin antibody or antigen binding fragment thereof can include a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107; or a heavy chain variable region that competitively inhibits binding of an isolated antibody or antigen binding fragment comprising at least one of a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

In other embodiments, the anti-properdin antibody or antigen binding fragment thereof can include a heavy chain variable region that includes an amino acid sequence at least about 70%, at least about 80%, or at least about 90% identical to the amino acid sequence of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

In still other embodiments, the anti-properdin antibody or antigen binding fragment thereof can include a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72; or a light chain variable region that competitively inhibits binding of an isolated antibody or antigen binding fragment comprising at least one of a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

In some embodiments, the anti-properdin antibody or antigen binding fragment thereof can include a light chain variable region that includes an amino acid sequence at least about 70%, at least about 80%, or at least about 90% identical to the amino acid sequence of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

In some embodiments, the anti-properdin antibody or antigen binding fragment thereof can include at least one of the following:
a) a heavy chain variable region that includes the 3 CDRs of SEQ ID NO: 1 and a light chain variable region that includes the 3 CDRs of SEQ ID NO: 24;
b) a heavy chain variable region that includes the 3 CDRs of SEQ ID NO: 2 and a light chain variable region that includes the 3 CDRs of SEQ ID NO: 25;
c) a heavy chain variable region that includes the 3 CDRs of SEQ ID NO: 3 and a light chain variable region that includes the 3 CDRs of SEQ ID NO: 26;
d) a heavy chain variable region that includes the 3 CDRs of SEQ ID NO: 4 and a light chain variable region that includes the 3 CDRs of SEQ ID NO: 27;
e) a heavy chain variable region that includes the 3 CDRs of SEQ ID NO: 5 and a light chain variable region that includes the 3 CDRs of SEQ ID NO: 28;
f) a heavy chain variable region that includes the 3 CDRs of SEQ ID NO: 6 and a light chain variable region that includes the 3 CDRs of SEQ ID NO: 29;
g) a heavy chain variable region that includes the 3 CDRs of SEQ ID NO: 7 and a light chain variable region that includes the 3 CDRs of SEQ ID NO: 30;
h) a heavy chain variable region that includes the 3 CDRs of SEQ ID NO: 8 and a light chain variable region that includes the 3 CDRs of SEQ ID NO: 31;
i) a heavy chain variable region that includes the 3 CDRs of SEQ ID NO: 9 and a light chain variable region that includes the 3 CDRs of SEQ ID NO: 32;
j) a heavy chain variable region that includes the 3 CDRs of SEQ ID NO: 10 and a light chain variable region that includes the 3 CDRs of SEQ ID NO: 33;
k) a heavy chain variable region that includes the 3 CDRs of SEQ ID NO: 11 and a light chain variable region that includes the 3 CDRs of SEQ ID NO: 34;
l) a heavy chain variable region that includes the 3 CDRs of SEQ ID NO: 12 and a light chain variable region that includes the 3 CDRs of SEQ ID NO: 35;
m) a heavy chain variable region that includes the 3 CDRs of SEQ ID NO: 13 and a light chain variable region that includes the 3 CDRs of SEQ ID NO: 36;
n) a heavy chain variable region that includes the 3 CDRs of SEQ ID NO: 14 and a light chain variable region that includes the 3 CDRs of SEQ ID NO: 37;
o) a heavy chain variable region that includes the 3 CDRs of SEQ ID NO: 15 and a light chain variable region that includes the 3 CDRs of SEQ ID NO: 38 or SEQ ID NO: 39;
p) a heavy chain variable region that includes the 3 CDRs of SEQ ID NO: 16 and a light chain variable region that includes the 3 CDRs of SEQ ID NO: 38 or SEQ ID NO: 39;
q) a heavy chain variable region that includes the 3 CDRs of SEQ ID NO: 17 and a light chain variable region that includes the 3 CDRs of SEQ ID NO: 38 or SEQ ID NO: 39;
r) a heavy chain variable region that includes the 3 CDRs of SEQ ID NO: 18 and a light chain variable region that includes the 3 CDRs of SEQ ID NO: 38 or SEQ ID NO: 39;
s) a heavy chain variable region that includes the 3 CDRs of SEQ ID NO: 19 and a light chain variable region that includes the 3 CDRs of SEQ ID NO: 38 or SEQ ID NO: 39;
t) a heavy chain variable region that includes the 3 CDRs of SEQ ID NO: 20 and a light chain variable region that includes the 3 CDRs of SEQ ID NO: 38 or SEQ ID NO: 39;
u) a heavy chain variable region that includes the 3 CDRs of SEQ ID NO: 21 and a light chain variable region that includes the 3 CDRs of SEQ ID NO: 38 or SEQ ID NO: 39;
v) a heavy chain variable region that includes the 3 CDRs of SEQ ID NO: 22 and a light chain variable region that includes the 3 CDRs of SEQ ID NO: 38 or SEQ ID NO: 39;
w) a heavy chain variable region that includes the 3 CDRs of SEQ ID NO: 23 and a light chain variable region that includes the 3 CDRs of SEQ ID NO: 38 or SEQ ID NO: 39;
x) a heavy chain variable region that includes the 3 CDRs of SEQ ID NO: 40 and a light chain variable region that includes the 3 CDRs of SEQ ID NO: 45;
y) a heavy chain variable region that includes the 3 CDRs of SEQ ID NO: 41 and a light chain variable region that includes the 3 CDRs of SEQ ID NO: 46;
z) a heavy chain variable region that includes the 3 CDRs of SEQ ID NO: 42 and a light chain variable region that includes the 3 CDRs of SEQ ID NO: 47;
aa) a heavy chain variable region that includes the 3 CDRs of SEQ ID NO: 43 and a light chain variable region that includes the 3 CDRs of SEQ ID NO: 48, SEQ ID NO: 49, or SEQ ID NO: 50;
bb) a heavy chain variable region that includes the 3 CDRs of SEQ ID NO: 44 and a light chain variable region that includes the 3 CDRs of SEQ ID NO: 48, SEQ ID NO: 49, or SEQ ID NO: 50;
cc) a heavy chain variable region that includes the 3 CDRs of SEQ ID NO: 51 and a light chain variable region that includes the 3 CDRs of SEQ ID NO: 61, SEQ ID NO: 62, SEQ ID NO: 63, SEQ ID NO: 64, or SEQ ID NO: 65;
dd) a heavy chain variable region that includes the 3 CDRs of SEQ ID NO: 52 and a light chain variable region that includes the 3 CDRs of SEQ ID NO: 61, SEQ ID NO: 62, SEQ ID NO: 63, SEQ ID NO: 64, or SEQ ID NO: 65;
ee) a heavy chain variable region that includes the 3 CDRs of SEQ ID NO: 53 and a light chain variable region that includes the 3 CDRs of SEQ ID NO: 61, SEQ ID NO: 62, SEQ ID NO: 63, SEQ ID NO: 64, or SEQ ID NO: 65;
ff) a heavy chain variable region that includes the 3 CDRs of SEQ ID NO: 54 and a light chain variable region that includes the 3 CDRs of SEQ ID NO: 61, SEQ ID NO: 62, SEQ ID NO: 63, SEQ ID NO: 64, or SEQ ID NO: 65;
gg) a heavy chain variable region that includes the 3 CDRs of SEQ ID NO: 55 and a light chain variable region that includes the 3 CDRs of SEQ ID NO: 61, SEQ ID NO: 62, SEQ ID NO: 63, SEQ ID NO: 64, or SEQ ID NO: 65;
hh) a heavy chain variable region that includes the 3 CDRs of SEQ ID NO: 56 and a light chain variable region that includes the 3 CDRs of SEQ ID NO: 66, SEQ ID NO: 67, SEQ ID NO: 68, SEQ ID NO: 69, or SEQ ID NO: 70;
ii) a heavy chain variable region that includes the 3 CDRs of SEQ ID NO: 57 and a light chain variable region that includes the 3 CDRs of SEQ ID NO: 66, SEQ ID NO: 67, SEQ ID NO: 68, SEQ ID NO: 69, or SEQ ID NO: 70;
jj) a heavy chain variable region that includes the 3 CDRs of SEQ ID NO: 58 and a light chain variable region that includes the 3 CDRs of SEQ ID NO: 66, SEQ ID NO: 67, SEQ ID NO: 68, SEQ ID NO: 69, or SEQ ID NO: 70;
kk) a heavy chain variable region that includes the 3 CDRs of SEQ ID NO: 59 and a light chain variable region that includes the 3 CDRs of SEQ ID NO: 66, SEQ ID NO: 67, SEQ ID NO: 68, SEQ ID NO: 69, or SEQ ID NO: 70; or
ll) a heavy chain variable region that includes the 3 CDRs of SEQ ID NO: 60 and a light chain variable region that includes the 3 CDRs of SEQ ID NO: 66, SEQ ID NO: 67, SEQ ID NO: 68, SEQ ID NO: 69, or SEQ ID NO: 70.

In some embodiments, the anti-properdin antibody or antigen binding fragment thereof can include at least one of the following:
a) a heavy chain that includes an amino acid sequence at least about 70%, at least about 80%, or at least about 90% identical to the amino acid sequence of SEQ ID NO: 1 and a light chain that includes an amino acid sequence at least about 70%, at least about 80%, or at least about 90% identical to the amino acid sequence of SEQ ID NO: 24;
b) a heavy chain that includes an amino acid sequence at least about 70%, at least about 80%, or at least about 90% identical to the amino acid sequence of SEQ ID NO: 2 and a light chain that includes an amino acid sequence at least about 70%, at least about 80%, or at least about 90% identical to the amino acid sequence of SEQ ID NO: 25;
c) a heavy chain variable that includes an amino acid sequence at least about 70%, at least about 80%, or at least about 90% identical to the amino acid sequence of SEQ ID NO: 3 and a light chain that includes an amino acid sequence at least about 70%, at least about 80%, or at least about 90% identical to the amino acid sequence of SEQ ID NO: 26;
d) a heavy chain that includes an amino acid sequence at least about 70%, at least about 80%, or at least about 90% identical to the amino acid sequence of SEQ ID NO: 4 and a light chain that includes an amino acid sequence at least about 70%, at least about 80%, or at least about 90% identical to the amino acid sequence of SEQ ID NO: 27;
e) a heavy chain that includes an amino acid sequence at least about 70%, at least about 80%, or at least about 90% identical to the amino acid sequence of SEQ ID NO: 5 and a light chain that includes an amino acid sequence at least about 70%, at least about 80%, or at least about 90% identical to the amino acid sequence of SEQ ID NO: 28;
f) a heavy chain that includes an amino acid sequence at least about 70%, at least about 80%, or at least about 90% identical to the amino acid sequence of SEQ ID NO: 6 and a light chain that includes an amino acid sequence at least about 70%, at least about 80%, or at least about 90% identical to the amino acid sequence of SEQ ID NO: 29;
g) a heavy chain that includes an amino acid sequence at least about 70%, at least about 80%, or at least about 90% identical to the amino acid sequence of SEQ ID NO: 7 and a light chain that includes an amino acid sequence at least about 70%, at least about 80%, or at least about 90% identical to the amino acid sequence of SEQ ID NO: 30;
h) a heavy chain that includes an amino acid sequence at least about 70%, at least about 80%, or at least about 90% identical to the amino acid sequence of SEQ ID NO: 8 and a light chain that includes an amino acid sequence at least about 70%, at least about 80%, or at least about 90% identical to the amino acid sequence of SEQ ID NO: 31;
i) a heavy chain that includes an amino acid sequence at least about 70%, at least about 80%, or at least about 90% identical to the amino acid sequence of SEQ ID NO: 9 and a light chain that includes an amino acid sequence at least about 70%, at least about 80%, or at least about 90% identical to the amino acid sequence of SEQ ID NO: 32;
j) a heavy chain that includes an amino acid sequence at least about 70%, at least about 80%, or at least about 90% identical to the amino acid sequence of SEQ ID NO: 10 and a light chain that includes an amino acid sequence at least about 70%, at least about 80%, or at least about 90% identical to the amino acid sequence of SEQ ID NO: 33;
k) a heavy chain that includes an amino acid sequence at least about 70%, at least about 80%, or at least about 90% identical to the amino acid sequence of SEQ ID NO: 11 and a light chain that includes an amino acid sequence at least about 70%, at least about 80%, or at least about 90% identical to the amino acid sequence of SEQ ID NO: 34;
l) a heavy chain that includes an amino acid sequence at least about 70%, at least about 80%, or at least about 90% identical to the amino acid sequence of SEQ ID NO: 12 and a light chain that includes an amino acid sequence at least about 70%, at least about 80%, or at least about 90% identical to the amino acid sequence of SEQ ID NO: 35;
m) a heavy chain that includes an amino acid sequence at least about 70%, at least about 80%, or at least about 90% identical to the amino acid sequence of SEQ ID NO: 13 and a light chain that includes an amino acid sequence at least about 70%, at least about 80%, or at least about 90% identical to the amino acid sequence of SEQ ID NO: 36;
n) a heavy chain that includes an amino acid sequence at least about 70%, at least about 80%, or at least about 90% identical to the amino acid sequence of SEQ ID NO: 14 and a light chain that includes an amino acid sequence at least about 70%, at least about 80%, or at least about 90% identical to the amino acid sequence of SEQ ID NO: 37;
o) a heavy chain that includes an amino acid sequence at least about 70%, at least about 80%, or at least about 90% identical to the amino acid sequence of SEQ ID NO: 15 and a light chain that includes an amino acid sequence at least about 70%, at least about 80%, or at least about 90% identical to the amino acid sequence of SEQ ID NO: 38 or SEQ ID NO: 39;
p) a heavy chain that includes an amino acid sequence at least about 70%, at least about 80%, or at least about 90% identical to the amino acid sequence of SEQ ID NO: 16 and a light chain that includes an amino acid sequence at least about 70%, at least about 80%, or at least about 90% identical to the amino acid sequence of SEQ ID NO: 38 or SEQ ID NO: 39;
q) a heavy chain that includes an amino acid sequence at least about 70%, at least about 80%, or at least about 90% identical to the amino acid sequence of SEQ ID NO: 17 and a light chain that includes an amino acid sequence at least about 70%, at least about 80%, or at least about 90% identical to the amino acid sequence of SEQ ID NO: 38 or SEQ ID NO: 39;
r) a heavy chain that includes an amino acid sequence at least about 70%, at least about 80%, or at least about 90% identical to the amino acid sequence of SEQ ID NO: 18 and a light chain that includes an amino acid sequence at least about 70%, at least about 80%, or at least about 90% identical to the amino acid sequence of SEQ ID NO: 38 or SEQ ID NO: 39;
s) a heavy chain that includes an amino acid sequence at least about 70%, at least about 80%, or at least about 90% identical to the amino acid sequence of SEQ ID NO: 19 and a light chain that includes an amino acid sequence at least about 70%, at least about 80%, or at least about 90% identical to the amino acid sequence of SEQ ID NO: 38 or SEQ ID NO: 39;
t) a heavy chain that includes an amino acid sequence at least about 70%, at least about 80%, or at least about 90% identical to the amino acid sequence of SEQ ID NO: 20 and a light chain that includes an amino acid sequence at least about 70%, at least about 80%, or at least about 90% identical to the amino acid sequence of SEQ ID NO: 38 or SEQ ID NO: 39;
u) a heavy chain that includes an amino acid sequence at least about 70%, at least about 80%, or at least about 90% identical to the amino acid sequence of SEQ ID NO: 21 and a light chain that includes an amino acid sequence at least about 70%, at least about 80%, or at least about 90% identical to the amino acid sequence of SEQ ID NO: 38 or SEQ ID NO: 39;
v) a heavy chain that includes an amino acid sequence at least about 70%, at least about 80%, or at least about 90% identical to the amino acid sequence of SEQ ID NO: 22 and a light chain that includes an amino acid sequence at least about 70%, at least about 80%, or at least about 90% identical to the amino acid sequence of SEQ ID NO: 38 or SEQ ID NO: 39;
w) a heavy chain that includes an amino acid sequence at least about 70%, at least about 80%, or at least about 90% identical to the amino acid sequence of SEQ ID NO: 23 and a light chain that includes an amino acid sequence at least about 70%, at least about 80%, or at least about 90% identical to the amino acid sequence of SEQ ID NO: 38 or SEQ ID NO: 39;
x) a heavy chain that includes an amino acid sequence at least about 70%, at least about 80%, or at least about 90% identical to the amino acid sequence of SEQ ID NO: 40 and a light chain that includes an amino acid sequence at least about 70%, at least about 80%, or at least about 90% identical to the amino acid sequence of SEQ ID NO: 45;
y) a heavy chain that includes an amino acid sequence at least about 70%, at least about 80%, or at least about 90% identical to the amino acid sequence of SEQ ID NO: 41 and a light chain that includes an amino acid sequence at least about 70%, at least about 80%, or at least about 90% identical to the amino acid sequence of SEQ ID NO: 46;
z) a heavy chain that includes an amino acid sequence at least about 70%, at least about 80%, or at least about 90% identical to the amino acid sequence of SEQ ID NO: 42 and a light chain that includes an amino acid sequence at least about 70%, at least about 80%, or at least about 90% identical to the amino acid sequence of SEQ ID NO: 47;
aa) a heavy chain that includes an amino acid sequence at least about 70%, at least about 80%, or at least about 90% identical to the amino acid sequence of SEQ ID NO: 43 and a light chain that includes an amino acid sequence at least about 70%, at least about 80%, or at least about 90% identical to the amino acid sequence of SEQ ID NO: 48, SEQ ID NO: 49, or SEQ ID NO: 50;
bb) a heavy chain that includes an amino acid sequence at least about 70%, at least about 80%, or at least about 90% identical to the amino acid sequence of SEQ ID NO: 44 and a light chain that includes an amino acid sequence at least about 70%, at least about 80%, or at least about 90% identical to the amino acid sequence of SEQ ID NO: 48, SEQ ID NO: 49, or SEQ ID NO: 50;
cc) a heavy chain that includes an amino acid sequence at least about 70%, at least about 80%, or at least about 90% identical to the amino acid sequence of SEQ ID NO: 51 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
dd) a heavy chain that includes an amino acid sequence at least about 70%, at least about 80%, or at least about 90% identical to the amino acid sequence of SEQ ID NO: 52 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ee) a heavy chain that includes an amino acid sequence at least about 70%, at least about 80%, or at least about 90% identical to the amino acid sequence of SEQ ID NO: 53 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ff) a heavy chain that includes an amino acid sequence at least about 70%, at least about 80%, or at least about 90% identical to the amino acid sequence of SEQ ID NO: 54 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
gg) a heavy chain that includes an amino acid sequence at least about 70%, at least about 80%, or at least about 90% identical to the amino acid sequence of SEQ ID NO: 55 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
hh) a heavy chain that includes an amino acid sequence at least about 70%, at least about 80%, or at least about 90% identical to the amino acid sequence of SEQ ID NO: 56 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
ii) a heavy chain that includes an amino acid sequence at least about 70%, at least about 80%, or at least about 90% identical to the amino acid sequence of SEQ ID NO: 57 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
jj) a heavy chain that includes an amino acid sequence at least about 70%, at least about 80%, or at least about 90% identical to the amino acid sequence of SEQ ID NO: 58 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
kk) a heavy chain that includes an amino acid sequence at least about 70%, at least about 80%, or at least about 90% identical to the amino acid sequence of SEQ ID NO: 59 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70; or
ll) a heavy chain that includes an amino acid sequence at least about 70%, at least about 80%, or at least about 90% identical to the amino acid sequence of SEQ ID NO: 60 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70.

In some embodiments, the antibody or antigen binding fragment thereof can include a constant chain region and wherein the antibody or antigen binding fragment including the constant chain region has enhanced in vivo half-live and/or reduced immunogenicity compared to the antibody or antigen binding fragment thereof without the constant chain region.

In some embodiments, the constant chain region includes an amino acid sequence at least about 70%, at least about 80%, or at least about 90% identical to the amino acid sequence of SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, or SEQ ID NO: 139.

In some embodiments, the anti-properdin antibody or antigen binding fragment thereof can include at least one of the following:
a heavy chain that includes an amino acid sequence at least about 70%, at least about 80%, or at least about 90% identical to the amino acid sequence of SEQ ID NO: 156 and a light chain that includes an amino acid sequence at least about 70%, at least about 80%, or at least about 90% identical to the amino acid sequence of SEQ ID NO: 157;
a heavy chain that includes an amino acid sequence at least about 70%, at least about 80%, or at least about 90% identical to the amino acid sequence of SEQ ID NO: 166 and a light chain that includes an amino acid sequence at least about 70%, at least about 80%, or at least about 90% identical to the amino acid sequence of SEQ ID NO: 167;
a heavy chain that includes an amino acid sequence at least about 70%, at least about 80%, or at least about 90% identical to the amino acid sequence of SEQ ID NO: 170 and a light chain that includes an amino acid sequence at least about 70%, at least about 80%, or at least about 90% identical to the amino acid sequence of SEQ ID NO: 171;
a heavy chain that includes an amino acid sequence at least about 70%, at least about 80%, or at least about 90% identical to the amino acid sequence of SEQ ID NO: 178 and a light chain that includes an amino acid sequence at least about 70%, at least about 80%, or at least about 90% identical to the amino acid sequence of SEQ ID NO: 179;
a heavy chain that includes an amino acid sequence at least about 70%, at least about 80%, or at least about 90% identical to the amino acid sequence of SEQ ID NO: 182;
a heavy chain that includes an amino acid sequence at least about 70%, at least about 80%, or at least about 90% identical to the amino acid sequence of SEQ ID NO: 184 and a light chain that includes an amino acid sequence at least about 70%, at least about 80%, or at least about 90% identical to the amino acid sequence of SEQ ID NO: 185;
a heavy chain that includes an amino acid sequence at least about 70%, at least about 80%, or at least about 90% identical to the amino acid sequence of SEQ ID NO: 187 and a light chain that includes an amino acid sequence at least about 70%, at least about 80%, or at least about 90% identical to the amino acid sequence of SEQ ID NO: 188;
a heavy chain that includes an amino acid sequence at least about 70%, at least about 80%, or at least about 90% identical to the amino acid sequence of SEQ ID NO: 191;
a heavy chain that includes an amino acid sequence at least about 70%, at least about 80%, or at least about 90% identical to the amino acid sequence of SEQ ID NO: 193 and a light chain that includes an amino acid sequence at least about 70%, at least about 80%, or at least about 90% identical to the amino acid sequence of SEQ ID NO: 194; or
a heavy chain that includes an amino acid sequence at least about 70%, at least about 80%, or at least about 90% identical to the amino acid sequence of SEQ ID NO: 197 and a light chain that includes an amino acid sequence at least about 70%, at least about 80%, or at least about 90% identical to the amino acid sequence of SEQ ID NO: 198.

### Linkers

In some embodiments, the anti-properdin antibody or antigen binding fragment thereof can include a linker. The linker can be used as a linkage or connection between polypeptides or protein domains and/or associated non-protein moieties of the anti-properdin antibody or antigen binding fragment thereof. In some embodiments, a linker is a linkage or connection between at least two polypeptide constructs, e.g., such that the two polypeptide constructs are joined to each other in tandem series (e.g., an antibody or antigen binding fragment thereof linked to a second polypeptide or antibody). A linker can attach the N-terminus or C-terminus of one antibody construct to the N-terminus or C-terminus of a second polypeptide construct.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

A linker between two peptide constructs can be an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine. In certain embodiments, the peptide linker can include the amino acid sequence of SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 245, or SEQ ID NO: 246.

### Bispecific, Enhanced Stability, and Modified Antibodies or Antigen Binding Fragments Thereof

In some embodiments, the antibody or antigen binding fragment thereof can include a first heavy chain variable region that binds to properdin and a second polypeptide, such as a second antibody or antigen binding fragment thereof having second heavy chain variable region that binds to a different epitope than the first heavy chain variable region so as to form a bispecific antibody or antigen binding fragment thereof.

In some embodiments, the second heavy chain variable region increases the in vivo half-life of the antibody or antigen binding fragment thereof to about 3 week to about 8 weeks.

In other embodiments, the second heavy chain variable region binds to one of albumin, TNF, or VEGF.

In some embodiments, the second polypeptide is an albumin binding molecule, an albumin binding peptide, or an anti-albumin antibody (e.g., a monovalent antibody), or a modified form thereof. Albumin binding peptides are known in the art and are described, for example, in WO 2007/106120 (see Tables 1 to 9) and Dennis et al., 2002, J Biol. Chem. 277: 35035-35043, the disclosures of which are hereby incorporated by reference.

In some embodiments, the second polypeptide is a Fc domain that enhances in vivo stability of the construct.

In some embodiments, the second antibody or antigen binding fragment thereof comprises an amino acid sequence at least about 70%, at least about 80%, or at least about 90% identical to the amino acid sequence of SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 150, or SEQ ID NO: 152.

In some embodiments, the isolated monospecific or bispecific antibody or antigen binding fragment thereof can inhibit C3b and Mac complex (C5b-9) formation in vivo.

Other embodiments described herein relate to an isolated monospecific or bispecific antibody or antigen binding fragment that includes:
an anti-properdin heavy chain variable region that includes an amino acid sequence at least about 70%, at least about 80%, or at least about 90% identical to the amino acid sequence of SEQ ID NO: 2 and anti-properdin light chain variable region that includes an amino acid sequence at least about 70%, at least about 80%, or at least about 90% identical to the amino acid sequence of SEQ ID NO: 25 and
wherein the heavy chain variable region includes:
a CDR-H1 comprising the amino acid sequence of GYIFTX₁YPIH (SEQ ID NO: 201), wherein X₁ is N, Q, S, A, or D (e.g., X₁ is Q, S, A, or D)
a CDR-H2 comprising the amino acid sequence of FIX₁PGGGX₂DEX₃X₄X₅X₆X₇X₈X₉ (SEQ ID NO: 202), wherein X₁ is D, E, S (e.g., E or S), or A; X₂ is H or Y; X₃ is P, S, or Y; X₄ is A or D; X₅ is D, E, or Q; X₆ is K, R, or S; X₇ is F or V; X₈ is E, K, Q, or R; and X₉ is D or G; and
a CDR-H3 comprising the amino acid sequence of RGGGYYLDY (SEQ ID NO: 203); and
the light chain variable region includes:
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is N, A, D, Q, or S (e.g., A, D, Q, or S); and X₂ is F, H, R, W, or Y (e.g., F, H, R, or Y).

In some embodiments, the antibody or antigen binding fragment thereof can include at least one of a constant chain region or a second heavy chain variable region binds that binds to a different epitope than the anti-properdin heavy chain region and wherein the antibody or antigen binding fragment including the constant chain region or second heavy chain variable region has enhanced in vivo half-live and/or reduced immunogenicity compared to the antibody or antigen binding fragment thereof without the constant chain region or second heavy chain variable region.

In some embodiments, the constant chain region includes an amino acid sequence at least about 70%, at least about 80%, or at least about 90% identical to the amino acid sequence of SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, or SEQ ID NO: 139; and the second heavy chain variable region includes an amino acid sequence at least about 70%, at least about 80%, or at least about 90% identical to the amino acid sequence of SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 150, or SEQ ID NO: 152.

In some embodiments, the isolated monospecific or bispecific antibody or antigen binding fragment thereof can include a heavy chain having an amino acid sequence at least 90% identical to SEQ ID NO: 247, and light chain having an amino acid sequence at least 90% identical to SEQ ID NO: 248.

### Generation of Single Domain Antibodies

In some embodiments, an isolated monospecific or bispecific antibody or antigen binding fragment thereof use a single domain antibody that is a heavy chain variable domain (V_{H}, e.g., V_{HH}) or a light chain domain (V_{L}). Thus, one means of generating single domain antibodies specific for properdin is to amplify and express the V_{H} and V_{L} regions of the heavy chain and light chain gene sequences isolated, for example, from a hybridoma (e.g., a mouse hybridoma) that expresses anti-properdin monoclonal antibody. The boundaries of V_{H} and V_{L} domains are set out, for example, by Kabat et al. (Sequences of Proteins of Immunological Interest (National Institutes of Health, Bethesda, Md., 1991). The information regarding the boundaries of the V_{H} and V_{L} domains of heavy and light chain genes is used to design PCR primers that amplify the V domain from a heavy or light chain coding sequence encoding an antibody known to bind properdin. The amplified V domains are inserted into a suitable expression vector, e.g., pHEN-1 (Hoogenboom, H. et al., Nucleic Acids Res., 19:4133-7, 1991) and expressed, for example, as a fusion of the V.sub.H and V.sub.L in a scFv or other suitable monovalent format. The resulting polypeptide can then be screened for high affinity monovalent binding to properdin. Screening for binding can be performed by methods known in the art. Single domain antibodies can be generated using methods known in the art (WO2005118642; Ward, E. et al., Nature, 341:544-6, 1989; Holt, L. et al., Trends Biotechnol., 21:484-90, 2003). Each light chain domain may be either of the kappa or lambda subgroup. Methods for isolating V_{H} and V_{L} domains have been described in the art (EP0368684).

In one embodiment, the antibody or antigen binding fragment thereof can include a single domain antibody that is obtained from a human, humanized rodent, camelid or shark. Any such single domain antibody can be optionally humanized. Humanization of camelid single domain antibodies requires the introduction and mutagenesis of a limited number of amino acids in a single polypeptide chain. This is in contrast to humanization of scFv, Fab, (Fab′)2 and IgG, which requires the introduction of amino acid changes in two chains, the light and the heavy chain and the preservation of the assembly of both chains. In some embodiments, the single domain antibody includes V_{HH} domains. In some embodiments, the V_{HH} domains correspond to the V_{HH} domains of naturally occurring heavy chain antibodies directed against properdin. Such V_{HH} sequences can be generated, for example, by suitably immunizing a species of camelid with properdin (i.e., so as to raise an immune response and/or heavy chain antibodies directed against properdin), by obtaining a suitable biological sample from said camelid (such as a blood sample, serum sample or sample of B-cells), and by generating V_{HH} sequences directed against properdin, starting from said sample, using any suitable technique known in the art (e.g., the gene encoding the single domain antibody may be cloned by single cell PCR, or the B-cell(s) encoding the single domain antibody may be immortalized by EBV transformation, or by fusion to an immortal cell line).

Alternatively, such naturally occurring V_{HH} domains against properdin, can be obtained from naive libraries of camelid V_{HH} sequences, for example by screening such a library using properdin, or at least one part, fragment, antigenic determinant or epitope thereof using one or more screening techniques known in the art (WO 99/37681, WO 01/90190, WO 03/025020 and WO 03/035694). Alternatively, improved synthetic or semi-synthetic libraries derived from naive V_{HH} libraries may be used, such as V_{HH} libraries obtained from naive V_{HH} libraries by techniques such as random mutagenesis and/or CDR shuffling (WO 00/43507). In a certain embodiment, a V_{HH} library is constructed and expressed on phages after infection with helper phages. After several rounds of bio-panning, single domain antibodies against human properdin can be isolated and efficiently expressed.

A library of fusion proteins including V_{HH} or V_{HH} fragments can be displayed on a phage, phagemid, ribosome or suitable microorganism (such as yeast), to facilitate screening. Suitable methods, techniques and host organisms for displaying and screening (a set, collection or library of) fusion proteins including V_{HH} or V_{HH} fragments are known in the art (WO 03/054016; Hoogenboom, H., Nat. Biotechnol., 23:1105-16, 2005).

In an additional embodiment, a method for generating fusion proteins including V_{HH} or V_{HH} fragment sequences includes at least the steps of: a) providing a collection or sample of cells derived from a species of camelid that express immunoglobulin sequences; b) screening the collection or sample of cells for (i) cells that express an immunoglobulin sequence that can bind to and/or have affinity for properdin; and (ii) cells that express heavy chain antibodies, in which substeps (i) and (ii) can be performed essentially as a single screening step or in any suitable order as two separate screening steps, to provide at least one cell that expresses a heavy chain antibody that can bind to and/or has affinity for properdin; and c) either (i) isolating from the cell the V_{HH} sequence present in the heavy chain antibody; or (ii) isolating from the cell a nucleic acid sequence that encodes the V_{HH} sequence present in the heavy chain antibody, followed by expressing the V_{HH} domain.

The method for generating an amino acid sequence directed against properdin can include at least the steps of: a) providing a set, collection or library of nucleic acid sequences encoding heavy chain antibodies or V_{HH} sequences; b) screening the set, collection or library of nucleic acid sequences for nucleic acid sequences that encode a heavy chain antibody or a fusion protein including the V_{HH} sequence that can bind to and/or has affinity for properdin; and c) isolating the nucleic acid sequence, followed by expressing the V_{HH} sequence present in the heavy chain antibody or by expressing the fusion protein including the V_{HH} sequence, respectively.

Other suitable methods and techniques for obtaining the single domain antibodies and/or nucleic acids encoding the same, starting from naturally occurring V_{H} sequences or V_{HH} sequences may, for example, include combining one or more parts of one or more naturally occurring V_{HH} sequences (such as one or more framework region (FR) sequences and/or CDR sequences), one or more parts of one or more naturally occurring V_{HH} sequences (such as one or more framework region sequences or CDR sequences), and/or one or more synthetic or semi-synthetic sequences, in a suitable manner, so as to provide a monovalent anti-properdin single domain antibody or a nucleotide sequence or nucleic acid encoding the same. Nucleotide sequences encoding framework sequences of V_{HH} or single domain antibodies are known in the art and may alternatively be obtained polymerase chain reaction (PCR) starting from the nucleotide sequences obtained using the methods described herein. Such compositions can be suitably combined with nucleotide sequences that encode the desired CDRs (for example, by PCR assembly using overlapping primers), to provide a single domain antibody, or antibody fragment fused with a regulator of the alternative complement pathway or fragment thereof.

Antibody antigen binding fragments that recognize the same epitope as a parent antibody can be generated by known techniques. For example, antibody antigen binding fragments can be prepared by proteolytic hydrolysis of an antibody or by expression in E. coli of the DNA coding for the fragment. The antibody antigen binding fragments are antigen binding portions of an antibody, such as Fab, F(ab′)2, and scFV can be obtained by pepsin or papain digestion of whole antibodies by conventional methods or by genetic engineering techniques.

An antibody antigen binding fragment can be produced by enzymatic cleavage of antibodies with pepsin to provide a 100 kDa fragment denoted F(ab′)2. This fragment can be further cleaved using a thiol reducing agent, and optionally a blocking group for the sulfhydryl groups resulting from cleavage of disulfide linkages, to produce 50 kDa Fab′ monovalent fragments. Alternatively, an enzymatic cleavage using papain produces two monovalent Fab fragments and an Fc fragment directly (U.S. Pat. Nos. 4,036,945 and 4,331,647; Nisonoff, A. et al., Arch. Biochem. Biophys., 89:230-44, 1960; Porter, R., Biochem. J., 73:119-26, 1959; Edelman et al., in Methods in Enzymology Vol. I, page 422 (Academic Press 1967), and Coligan el al., Current Protocols in Immunology, Vol. 1, pages 2.8.1-2.8.10 and 2.10.-2.10.4 (John Wiley & Sons 1991).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

Another form of an antibody antigen binding fragment is a peptide coding for a single complementarity-determining region (CDR). CDR peptides can be obtained by constructing genes encoding the CDR of an antibody of interest. Such genes are prepared, for example, by using the polymerase chain reaction to synthesize the variable region from RNA of antibody producing cells (Larrick, J & Fry, K. METHODS—a companion to Methods in Enzymology Volume: New Techniques in Antibody Generation, 2:106-110, 1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles And Applications, Birch et al., (eds.), pages 137-185 (Wiley-Liss, Inc. 1995)).

Other antibody antigen binding fragments, for example single domain antibody fragments, are known in the art and may be used in the claimed constructs (Muyldermans, S. et al., Trends Biochem. Sci., 26:230-5, 2001; Yau, K. et al., J. Immunol. Methods, 281:161-75, 2003; Maass, D. et al., J. Immunol. Methods, 324:13-25, 2007). The V_{HH} may have potent antigen binding capacity and can interact with novel epitopes that are inaccessible to conventional V_{H}-V_{L} pairs. Camelidae may be immunized with known antigens, such as properdin, and V_{HH}S can be isolated that bind to and neutralize the target antigen.

### Screening of Antibodies or Antigen Binding Fragments Thereof for Properdin Binding

Library screening methods can be used to identify properdin-specific binding antibodies or antigen binding fragments thereof. Phage display technology provides an approach for the selection of antibodies that bind a desired target (e.g., human properdin) from among large, diverse repertoires of antibodies (Smith, G., Science, 228:1315-7, 1985; Scott, J. & Smith, G., Science, 249:386-90, 1990; McCafferty, J. et al., Nature, 348:552-4, 1990). These phage-antibody libraries can be grouped into two categories: natural libraries that use rearranged V genes harvested from human B-cells (Marks, J. et al., J. Mol. Biol., 222:581-97, 1991; Vaughan, T. et al., Nat. Biotechnol., 14:309-14, 1996) or synthetic libraries whereby germline V gene segments or other antibody polypeptide coding sequences are ‘rearranged’ in vitro (Hoogenboom, H. & Winter, G., J. Mol. Biol., 227:381-8, 1992; Nissim, A. et al., EMBO J., 13:692-8, 1994; Griffiths, A. et al., EMBO J., 13:3245-60, 1994; de Kruif, J. et al., J. Mol. Biol., 248:97-105, 1995) or where synthetic CDRs are incorporated into a single rearranged V gene (Barbas, C. et al., Proc. Natl. Acad. Sci. USA, 89:4457-61, 1992). Methods involving genetic display packages (e.g., phage display, polysome display) are suited for the selection of properdin-specific antibody constructs because they generally express only fragments, rather than whole, antibodies, on the display packages. Methods for the preparation of phage display libraries displaying various antibody fragments are described in the preceding references and, for example, in U.S. Pat. No. 6,696,245, which is incorporated herein by reference in its entirety.

Following expression of a repertoire of antibodies on the surface of phage, selection is performed by contacting the phage repertoire with immobilized target antigen (e.g., properdin), washing to remove unbound phage, and propagation of the bound phage, the whole process frequently referred to as “panning.” This process is applicable to the screening of antibodies and antigen binding fragments thereof that can be expressed on a display library (e.g., scFv, Fab, (Fab′)2, and V_{HH}; Harrison, J. et al., Meth. Enzymol., 267:83-109, 1996). Alternatively, phages are pre-selected for the expression of properly folded member variants by panning against an immobilized generic ligand (e.g., protein A or protein L) that is only bound by folded members (WO 99/20749). This has the advantage of reducing the proportion of non-functional members, thereby increasing the proportion of members likely to bind a target antigen. The screening of phage antibody libraries is generally described, for example, by.

Screening is commonly performed using purified antigen immobilized on a solid support, for example, plastic tubes or wells, or on a chromatography matrix, for example Sepharose™ (Pharmacia). Screening or selection can also be performed on complex antigens, such as the surface of cells (Marks, J. et al., Biotechnology (NY), 11:1145-9, 1993; de Kruif, J. et al., Proc. Natl. Acad. Sci. USA, 92:3938-42, 1995). Another alternative involves selection by binding biotinylated antigen in solution, followed by capture on streptavidin-coated beads.

### Pharmaceutical Compositions

Other embodiments relate to formulations or compositions comprising an inhibitor of the alternative complement pathway and a selective inhibitor including, but not limited to, a murine, chimeric, or human antibody that prevents alternative pathway activation in a mammal. The formulation comprises: (a) an inhibitor of the alternative complement pathway as described herein; and (b) a pharmaceutically acceptable carrier. In one embodiment, the formulation or composition can include one or more additional agents, such as an anti-inflammatory agent suitable for reducing inflammation in a mammal that has, or is at risk of developing, an inflammatory disorder. In another embodiment, the formulation or composition can include one or more additional agents, such as an additional agent suitable for preventing or reducing ischemia-reperfusion injury in a mammal. In yet another embodiment, the formulation or composition can include one or more additional agents, such as an additional agent suitable for treatment of another disease or condition associated with activation of the alternative complement pathway.

A monospecific or bispecific anti-properdin antibody or antigen binding fragment thereof described herein can be included with a pharmaceutically acceptable carrier, including, but not limited to, pharmaceutically acceptable excipients and/or pharmaceutically acceptable delivery vehicles, which are suitable for use in the administration of a formulation or composition to a suitable in vivo site.

One type of pharmaceutically acceptable carrier can include a controlled-release formulation that is capable of slowly releasing a composition of the present invention into a mammal. As used herein, a controlled-release formulation comprises an agent of the present invention in a controlled-release vehicle. Suitable controlled-release vehicles can include, but are not limited to, biocompatible polymers, other polymeric matrices, capsules, microcapsules, microparticles, bolus preparations, osmotic pumps, diffusion devices, liposomes, lipospheres, and transdermal delivery systems. Other suitable carriers can include any carrier that can be bound to or incorporated with the monospecific or bispecific anti-properdin antibody or antigen binding fragment thereof described herein that extends that half-life of the monospecific or bispecific anti-properdin antibody or antigen binding fragment thereof described herein to be delivered. Such a carrier can include any suitable protein carrier or a fusion segment that extends the half-life of a protein when delivered in vivo. Suitable delivery vehicles can include, but are not limited to liposomes, viral vectors or other delivery vehicles, including ribozymes, and natural lipid-containing delivery vehicles such as cells and cellular membranes.

Intravenous, intraperitoneal, intramuscular and intramuscular administrations can be performed using methods standard in the art. Aerosol delivery can be performed using methods standard in the art. Devices for delivery of aerosolized formulations can include, but are not limited to, pressurized metered dose inhalers (“MDI”), dry powder inhalers (“DPI”), and metered solution devices (“MSI”), and include devices that are nebulizers and inhalers.

Another type of dose of a monospecific or bispecific anti-properdin antibody or antigen binding fragment thereof described herein or a particularly when the antibody formulation is delivered by nebulization, comprises a collection of ranges between about 200 ng/kg and about 600 μg/kg body weight of the mammal, between about 200 ng/kg and about 500 g/kg, between about 200 ng/kg and about 400 μg/kg, between about 200 ng/kg and about 300 g/kg, between about 200 ng/kg and about 200 μg/kg, between about 200 ng/kg and about 100 μg/kg, and preferably, between about 200 ng/kg and about 50 μg/kg body weight of the mammal.

The monospecific or bispecific anti-properdin antibody or antigen binding fragment thereof described herein can be conjugated with a synthetic or biological entity at the —SH group, or any other position which does not interfere with the binding. Such conjugates can also be covered in the present invention.

### Uses of Anti-Properdin Antibody or Antigen Binding Fragment

In some embodiments, the anti-properdin antibody or antigen binding fragment can be used to inhibit complement activation via the alternative pathway in vivo in subjects, including humans, suffering from an acute or chronic pathological injury. For example, anti-properdin antibody or antigen binding fragment can be used to treat the following complement mediated or associated diseases, disorders, or conditions, including but not limited to:

Extracorporeal circulation diseases and disorders: Post-cardiopulmonary bypass inflammation, post-operative pulmonary dysfunction, cardiopulmonary bypass, hemodialysis, leukopheresis, plasmapheresis, plateletpheresis, heparin-induced extracorporeal LDL precipitation (HELP), postperfusion syndrome, extracorporeal membrane oxygenation (ECMO), cardiopulmonary bypass (CPB), post-perfusion syndrome, systemic inflammatory response, and multiple organ failure.

Cardiovascular diseases and disorders: acute coronary syndromes, Kawaski disease (arteritis), Takayasu's arteritis, Henoch-Schonlein purpura nephritis, vascular leakage syndrome, percutaneous coronary intervention (PCI), myocardial infarction, ischemia-reperfusion injury following acute myocardial infarction, atherosclerosis, vasculitis, immune complex vasculitis, vasculitis associated with rheumatoid arthritis (also called malignant rheumatoid arthritis), systemic lupus erythematosus-associated vasculitis, sepsis, arteritis, aneurysm, cardiomyopathy, dilated cardiomyopathy, cardiac surgery, peripheral vascular conditions, renovascular conditions, cardiovascular conditions, cerebrovascular conditions, mesenteric/enteric vascular conditions, diabetic angiopathy, venous gas embolus (VGE), Wegener's granulomatosis, heparin-induced extracorporeal membrane oxygenation, and Behcet's syndrome.

Bone/Musculoskeletal diseases and disorders: arthritis, inflammatory arthritis, non-inflammatory arthritis, rheumatoid arthritis, juvenile rheumatoid arthritis, systemic juvenile rheumatoid arthritis, osteoarthritis, osteoporosis, systemic lupus erythematosus (SLE), Behcet's syndrome, and Sjogren's syndrome.

Transplantation diseases and disorders: transplant rejection, xenograft rejection, graft versus host disease, xenotransplantation of organs or grafts, allotransplantation of organs or grafts, and hyperacute rejection.

Eye/Ocular diseases and disorders: wet and dry age-related macular degeneration (AMD), choroidal neurovascularization (CNV), retinal damage, diabetic retinopathy, diabetic retinal microangiopathy, histoplasmosis of the eye, uveitis, diabetic macular edema, diabetic retinopathy, diabetic retinal microangiopathy, pathological myopia, central retinal vein occlusion (CRVO), corneal neovascularization, retinal neovascularization, retinal pigment epithelium (RPE), histoplasmosis of the eye, and Purtscher's retinopathy.

Hemolytic/Blood diseases and disorders: sepsis, systemic inflammatory response syndrome” (SIRS), hemorrhagic shock, acute respiratory distress syndrome (ARDS), catastrophic anti-phospholipid syndrome (CAPS), cold agglutinin disease (CAD), autoimmune thrombotic thrombocytopenic purpura (TTP), endotoxemia, hemolytic uremic syndrome (HUS), atypical hemolytic uremic syndrome (aHUS), paroxysmal nocturnal hemoglobinuria (PNH), sepsis, septic shock, sickle cell anemia, hemolytic anemia, hypereosinophilic syndrome, and anti-phospholipid syndrome (APLS).

Respiratory/Pulmonary diseases and disorders: asthma, Wegener's granulomatosis, transfusion-related acute lung injury (TRALI), antiglomerular basement membrane disease (Goodpasture's disease), eosinophilic pneumonia, hypersensitivity pneumonia, allergic bronchitis bronchiecstasis, reactive airway disease syndrome, respiratory syncytial virus (RSV) infection, parainfluenza virus infection, rhinovirus infection, adenovirus infection, allergic bronchopulmonary aspergillosis (ABPA), tuberculosis, parasitic lung disease, adult respiratory distress syndrome, chronic obstructive pulmonary disease (COPD), sarcoidosis, emphysema, bronchitis, cystic fibrosis, interstitial lung disease, acute respiratory distress syndrome (ARDS), transfusion-related acute lung injury, ischemia/reperfusion acute lung injury, byssinosis, heparin-induced extracorporeal membrane oxygenation, anaphylactic shock, and asbestos-induced inflammation.

Central and Peripheral Nervous System/Neurological diseases and disorders: multiple sclerosis (MS), myasthenia gravis (MG), myasthenia gravis, multiple sclerosis, Guillain Barre syndrome, Miller-Fisher syndrome, stroke, reperfusion following stroke, Alzheimer's disease, multifocal motor neuropathy (MMN), demyelination, Huntington's disease, amyotrophic lateral sclerosis (ALS), Parkinson's disease, degenerative disc disease (DDD), meningitis, cranial nerve damage from meningitis, variant Creutzfeldt-Jakob Disease (vCJD), idiopathic polyneuropathy, brain/cerebral trauma (including, but not limited to, hemorrhage, inflammation, and edema), neuromyelitis optica (NMO), including those serologically positive for aquaporin-4 (AQP4)-IgG autoantibody, and neuropathic pain.

Trauma-induced injuries and disorders: hemorrhagic shock, hypovolemic shock, spinal cord injury, neuronal injury, cerebral trauma, cerebral ischemia reperfusion, crush injury, wound healing, severe burns, and frostbite.

Renal diseases and disorders: renal reperfusion injury, poststreptococcal glomerulonephritis (PSGN), Goodpasture's disease, membranous nephritis, Berger's Disease/IgA nephropathy, mesangioproliferative glomerulonephritis, membranous glomerulonephritis, membranoproliferative glomerulonephritis (mesangiocapillary glomerulonephritis), acute postinfectious glomerulonephritis, cryoglobulinemic glomerulonephritis, lupus nephritis, Henoch-Schonlein purpura nephritis, and renal cortical necrosis (RCN).

Reperfusion injuries and disorders of organs: including but not limited to heart, brain, kidney, and liver.

Reproduction and urogenital diseases and disorders: painful bladder diseases and disorders, sensory bladder diseases and disorders, spontaneous abortion, male and female diseases from infertility, diseases from pregnancy, fetomaternal tolerance, pre-eclampsia, urogenital inflammatory diseases, diseases and disorders from placental dysfunction, diseases and disorders from miscarriage, chronic abacterial cystitis, and interstitial cystitis.

Skin/Dermatologic diseases and disorders: burn injuries, psoriasis, atopic dermatitis (AD), eosinophilic spongiosis, urticaria, thermal injuries, pemphigoid, epidermolysis bullosa acquisita, autoimmune bullous dermatoses, bullous pemphigoid, scleroderma, angioedema, hereditary angioneurotic edema (HAE), erythema multiforme, herpes gestationis, Sjogren's syndrome, dermatomyositis, and dermatitis herpetiformis.

Gastrointestinal diseases and disorders: Crohn's disease, Celiac Disease/gluten-sensitive enteropathy, Whipple's disease, intestinal ischemia, inflammatory bowel disease, and ulcerative colitis.

Endocrine diseases and disorders: Hashimoto's thyroiditis, juvenile lymphocytic thyroiditis, stress anxiety, and other diseases affecting prolactin, growth or insulin-like growth factor, adrenocorticotropin release, pancreatitis, Addison's disease, diabetic conditions including, but not limited to, type 1 and type 2 diabetes, type I diabetes mellitus, sarcoidosis, diabetic retinal microangiopathy, non-obese diabetes (IDDM), angiopathy, neuropathy or retinopathy complications of IDDM or Type-2 diabetes, and insulin resistance.

Treatment of Malignancies: diseases and disorders arising from chemotherapeutics and radiation therapy.

### EXAMPLES

Properdin is an activator of the complement system. It is the only endogenous activator of the alternative pathway (AP). Additionally, properdin has no effect on the classical pathway (CP); therefore, blocking the formation of properdin still leaves many essential mechanisms of the CP intact.

The following anti-properdin antibodies and antigen binding fragments thereof were constructed using variable chains, constant chains, and linkers. Substitutions were made in each of these sequences and linkers in order to generate multiple variants and embodiments of Anti-properdin antibodies.

### Example 1

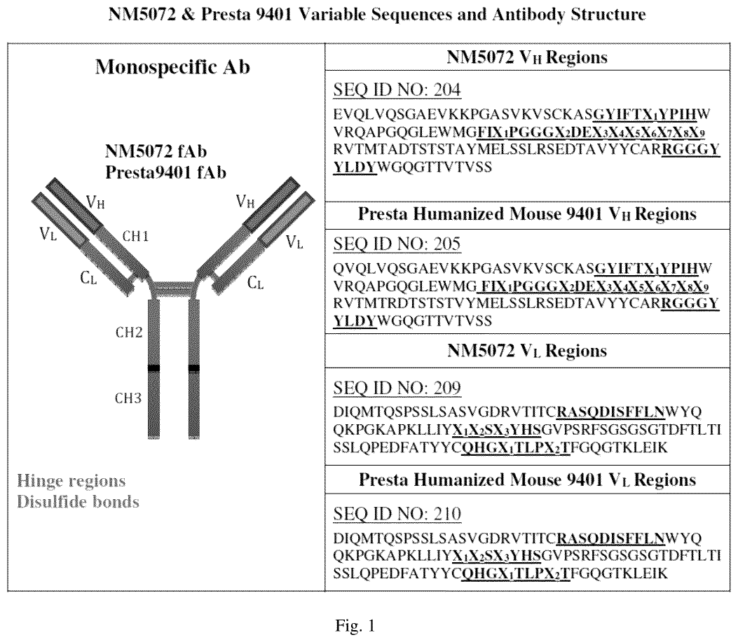

Example 1 describes the structure and amino acid sequences of anti-properdin antibodies NMT16-NMT28. The CDR regions of the variable light and heavy chain sequences for NMT16-NMT28 (SEQ ID NOs: 2-14 & 24-37) are shown in FIG. 1 and Table 1. The framework regions are from NMT16's heavy chain (SEQ ID NO: 2) and light chain (SEQ ID NO: 24) sequences. The CDR regions for the V_{H} Region (SEQ ID NO: 204) and V_{L} region (SEQ ID NOs: 209) contain multiple substitutions across variants of the anti-properdin antibody NM5072 where the amino acids that can be substituted in the CDR regions are marked as X.

The Presta humanized mouse variable sequences anti-properdin antibody 9401 (SEQ ID NOs: 15-23 & 38-39) are presented in the same manner. The CDR regions contain multiple substitutions across all humanized 9401 sequences, and the framework regions used are from Presta h9401-H1 heavy chain (SEQ ID NO: 15) and h9401-L1 light chain (SEQ ID NO: 38). The CDR regions for the V_{H} Region (SEQ ID NO: 205) and V_{L} region (SEQ ID NO: 210) contain multiple substitutions where the amino acids that can be substituted in the CDR regions are marked as X. The full list of amino acid substitutions for NM5072 and Presta 9401 can be found in the table below.

NM5072 Example 1 (NMT16-NMT28)
and Presta Mouse 9401
CDR Regions for SEQ ID NOs: 204 and 209

| | CDR1 | CDR2 | CDR3 |
|---|---|---|---|
| Heavy | GYIFTX₁YPIH | FIX₁PGGGX₂DEX₃ | RGGGYYLDY |
| Chain | (SEQ ID | X₄X₅X₆X₇X₈X₉ | (SEQ ID |
| | NO: 201) | (SEQ ID NO: 202) | NO: 203) |
| | Wherein | Wherein | No |
| | X₁ is | X₁ is D, E, S, | substitutions |
| | N, Q, S, | or A; | |
| | A, D | X₂ is H or Y; | |
| | | X₃ is P, S, or Y; | |
| | | X₄ is A or D; | |
| | | X₅ is D, R, or Q; | |
| | | X₆ is K, R, or S; | |
| | | X₇ is F or V; | |
| | | X₈ is E, | |
| | | K, Q, or R; | |
| | | X₉ is D or G | |
| Light | RASQDISFFLN | X₁X₂SX₃YHS | QHGX₁TLPX₂T |
| Chain | (SEQ ID | (SEQ ID NO: 207) | (SEQ ID |
| | NO: 206) | Wherein | NO: 208) |
| | No | X₁ is G or Y; | Wherein |
| | substitutions | X₂ is A or T; | X₁ is A, |
| | | or S | D, N, Q, |
| | | X₃ is R | or S; X₂ is F, |
| | | | H, R, W, |
| | | | or Y |

The Ab structure of FIG. 1 is a monospecific antibody that contains fAb regions from the V_{H} and V_{L} of NM5072 or Presta humanized mouse 9401. Different combinations of the variable chain sequences can be utilized with different amino acid CDR substitutions and framework variations. Further combinations can be implemented when linking the V_{H} to an Fc region. The heavy chain can be linked to any of the Fc's present in Table 6 using any of the linkers from Table 7 with the most common being the ASTK linker (SEQ ID NO: 245), whereas the V_{L} will be linked to the CL (SEQ ID NO: 246) using the RTVAAP linker (SEQ ID NO 247). The heavy and light chain sequences are joined together by a disulfide bond that occurs between the CH1 and CL chains at the Cystine residues. Similarly, disulfide bonds are formed between the hinge regions of the two fAbs creating a full monoclonal antibody. The antibody can be made bispecific by replacing one of the NM5072 or Presta fAbs with an Anti-Alb sequence or a fAb that binds to a different target.

For NM5072, the most important factor was identifying substitutions in the CDR regions of the sequences. The CDR region allows the antibody to recognize its specific binding site, in this case Properdin, in order to block the activation of the Alternative Pathway. Four main heavy chain substitutions were made. One substitution had the replacement of amino acid V with I; another substitution had the replacement of amino acid K with A; the third substitution had the replacement of amino acid D with E, S, or A also known as a Asn/Pro fragmentation substitution; the final substitution had the replacement of amino acid N with Q, S, A, or D also known as a deamidation substitution.

The light chain had three main substitutions. One substitution had the replacement of amino acid A with T; another substitution had the deamidation substitution; the final substitution had the replacement of amino acid W with H, Y, or F also known as a tryptophan oxidation substitution.

All other substitutions are based off of slight changes in the CDR regions when comparing all NM5072 sequences (NMT16-NMT28) with respect to each other. Differences in the framework regions are listed as separate sequences.

Additional CDR variants and differences in the framework regions are listed as separate sequences in Table 1.

TABLE 1
HEAVY CHAIN

| | |
|---|---|
| NMT15-HC | QVQLVQSAPEVAKPGTSVKMSCKASGYIFTNYPIHWVKQAPGQ |
| | GLEWIGFIDPGGGYDEPDERFRDRATLTADKSTSTAYMELSSLR |
| | SEDTAIYYCARRGGGYYLDYWGQGTLVTVSS (SEQ ID NO: 1) |
| NMT16-HC | EVQLVQSGAEVKKPGASVKVSCKASGYIFT NYPIHWVRQA |
| | PGQGLEWMGF IDPGGGYDEPDERFRDRVTM TADTSTSTAY |
| | MELSSLRSED TAVYYCARRG GGYYLDYWGQ GTTVTVSS |
| | (SEQ ID NO: 2) |
| NMT17-HC | EVQLVQSGAE VKKPGASVKV SCKASGYIFT NYPIHWVRQA |
| | PGQGLEWMGF IDPGGGYDEP DERFRDRVTM TADTSTSTAY |
| | MELSSLRSED TAVYYCARRG GGYYLDYWGQ GTTVTVSS |
| | (SEQ ID NO: 3) |
| NMT18-HC | EVQLVQSGAE VKKPGASVKV SCKASGYIFT NYPIHWVRQA |
| | PGQGLEWMGF IDPGGGYDEP DERFRDRVTM TADTSTSTAY |
| | MELSSLRSED TAVYYCARRG GGYYLDYWGQ GTTVTVSS |
| | (SEQ ID NO: 4) |
| NMT19-HC | EVQLVQSGAE VKKPGASVKV SCKASGHIFT NYPIHWVRQA |
| | PGQGLEWMGF IDPGGGHDEP DERFRDRVTM TADTSTSTAY |
| | MELSSLRSED TAVYYCARRG GGYYLDYWGQ GTTVTVSS |
| | (SEQ ID NO: 5) |
| NMT20-HC | EVQLVQSGAE VKKPGASVKV SCKASGYIFT NYPIHWVRQA |
| | PGQGLEWMGF IDPGGGYDEP DERFRDRVTM TADTSTSTAY |
| | MELSSLRSED TAVYYCARRG GGYYLDYWGQ GTTVTVSS |
| | (SEQ ID NO: 6) |
| NMT21-HC | EVQLVQSGAE VKKPGASVKV SCKASGYIFT NYPIHWVRQA |
| | PGQGLEWMGF IDPGGGYDEY AQKFQGRVTL TADTSTSTAY |
| | MELSSLRSED TAVYYCARRG GGYYLDYWGQ GTTLTVSS |
| | (SEQ ID NO: 7) |
| NMT22-HC | EVQLVQSGAE VKKPGASVKV SCKASGYIFT NYPIHWVRQA |
| | PGQGLEWMGF IDPGGGYDES AQKFRGRVTL TADTSTSTVY |
| | MELSSLRSED TAVYYCARRG GGYYLDYWGQ GTTLTVSS |
| | (SEQ ID NO: 8) |
| NMT23-HC | EVQLVQSGAE VKKPGASVKV SCKASGYIFT NYPIHWVRQA |
| | PGKGLEWMGF IDPGGGHDEY ADSVKGRVTM TRDTSTSTVY |
| | MELSSLRSED TAVYYCARRG GGYYLDYWGQ GTLVTVSS |
| | (SEQ ID NO: 9) |
| NMT24-HC | EVQLVQSGAE VKKPGASVKV SCKASGYIFT NYPIHWVRQA |
| | PGKGLEWMGF IDPGGGHDEY ADSVKGRVTM TRDTSTSTVY |
| | MELSSLRSED TAVYYCARRG GGYYLDYWGQ GTLVTVSS |
| | (SEQ ID NO: 10) |
| NMT25-HC | EVQLLESGGG LVQPGGSLRL SCAASGYIFT NYPIHWVRQA |
| | PGKGLEWVSF IDPGGGYDEY ADSVKGRFTI SRDNSKNTLY |
| | LQMNSLRAED TAVYYCAKRG GGYYLDYWGQ GTLVTVSS |
| | (SEQ ID NO: 11) |
| NMT26-HC | EVQLVESGGG LVQPGRSLRL SCAASGYIFT NYPIHWVRQA |
| | PGKGLEWVSF IDPGGGHIDY ADSVEGRFTI SRDNAKNSLY |
| | LQMNSLRAED TAVYYCAKRG GGYYLDYWGQ GTLVTVSS |
| | (SEQ ID NO: 12) |
| NMT27-HC | EVQLVQSGAE VKKPGASVKV SCKASGYIFT NYPIHWVRQA |
| | PGKGLEWMGF IDPGGGYDEP DERFRDRVTM TRDTSTSTVY |
| | MELSSLRSED TAVYYCARRG GGYYLDYWGQ GTLVTVSS |
| | (SEQ ID NO: 13) |
| NMT28-HC | EVQLVQSGAE VKKPGASVKV SCKASGYIFT NYPIHWVRQA |
| | PGQGLEWMGF IDPGGGYDEP DERFRDRVTM TADTSTSTAY |
| | MELSSLRSED TAVYYCARRG GGYYLDYWGQ GTLVTVSS |
| | (SEQ ID NO: 14) |
| h9401-H1_HC | QVQLVQSGAE VKKPGASVKV SCKASGYIFT NYPIHWVRQA |
| | PGQGLEWMGF IDPGGGYDEP DERFRDRVTM TRDTSTSTVY |
| | MELSSLRSED TAVYYCARRG GGYYLDYWGQ GTTVTVSS |
| | (SEQ ID NO: 15) |
| h9401-H2_HC | QVQLVQSGAE VKKPGASVKV SCKASGYIFT NYPIHWVRQA |
| | PGQGLEWMGF IDPGGGYDEP DERFRDRVTM TADTSTSTAY |
| | MELSSLRSED TAVYYCARRG GGYYLDYWGQ GTTVTVSS |
| | (SEQ ID NO: 16) |
| h9401-H3_HC | QVQLVQSGAE VKKPGASVKM SCKASGYIFT NYPIHWVRQA |
| | PGQGLEWMGF IDPGGGYDEP DERFRDRATL TADTSTSTAY |
| | MELSSLRSED TAVYYCARRG GGYYLDYWGQ GTTLTVSS |
| | (SEQ ID NO: 17) |
| h9401-H2b_HC | QVQLVQSGAE VKKPGASVKV SCKASGYIFT NYPIHWVRQA |
| | PGQGLEWMGF IDPGGGYDEP DERFRDRVTM TADKSTSTAY |
| | MELSSLRSED TAVYYCARRG GGYYLDYWGQ GTTVTVSS |
| | (SEQ ID NO: 18) |
| h9401-H3b_HC | QVQLVQSGAE VKKPGASVKM SCKASGYIFT NYPIHWVRQA |
| | PGQGLEWMGF IDPGGGYDEP DERFRDRATL TADKSTSTAY |
| | MELSSLRSED TAVYYCARRG GGYYLDYWGQ GTTLTVSS |
| | (SEQ ID NO: 19) |
| h9401-H2c_HC | QVQLVQSGAE VKKPGASVKV SCKASGYIFT NYPIHWVRQA |
| | PGQGLEWMGF IDPGGGYDEY AQKFQGRVTM TADTSTSTAY |
| | MELSSLRSED TAVYYCARRG GGYYLDYWGQ GTTVTVSS |
| | (SEQ ID NO: 20) |
| h9401-H3c_HC | QVQLVQSGAE VKKPGASVKM SCKASGYIFT NYPIHWVRQA |
| | PGQGLEWMGF IDPGGGYDEY AQKFQGRATL TADTSTSTAY |
| | MELSSLRSED TAVYYCARRG GGYYLDYWGQ GTTLTVSS |
| | (SEQ ID NO: 21) |
| h9401-H2d_HC | QVQLVQSGAE VKKPGASVKV SCKASGYIFT NYPIHWVRQA |
| | PGQGLEWMGF IDPGGGYDEY AQKFQGRVTM TADKSTSTAY |
| | MELSSLRSED TAVYYCARRG GGYYLDYWGQ GTTVTVSS |
| | (SEQ ID NO: 22) |
| h9401-H3d_HC | QVQLVQSGAE VKKPGASVKM SCKASGYIFT NYPIHWVRQA |
| | PGQGLEWMGF IDPGGGYDEY AQKFQGRATL TADKSTSTAY |
| | MELSSLRSED TAVYYCARRG GGYYLDYWGQ GTTLTVSS |
| | (SEQ ID NO: 23) |

LIGHT CHAIN

| | |
|---|---|
| NMT15-LC | DIQMTQSPSS LSASLGDRVT ITCRASQDIS FFLNWYQQKP |
| | DGTVKLLIYY TSRYHSGVPS RFSGSGSGTD FTLTISSLQP |
| | EDFATYFCQH GNTLPWTFGQ GTKLEIK (SEQ ID NO: 24) |
| NMT16-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GNTLPWTFGQ GTKLEIK (SEQ ID NO: 25) |
| NMT17-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GSTLPWTFGQ GTKLEIK (SEQ ID NO: 26) |
| NMT18-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GQTLPWTFGQ GTKLEIK (SEQ ID NO: 27) |
| NMT19-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GQTLPWTFGQ GTKLEIK (SEQ ID NO: 28) |
| NMT20-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GSTLPYTFGQ GTKLEIK (SEQ ID NO: 29) |
| NMT21-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GQTLPWTFGQ GTKLEIK (SEQ ID NO: 30) |
| NMT22-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GSTLPYTFGQ GTKLEIK (SEQ ID NO: 31) |
| NMT23-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GSTLPWTFGQ GTKVEIK (SEQ ID NO: 32) |
| NMT24-LC | DIQMTQSPSS LSASVGDRVT ITCRASQDIS FFLNWYQQKP |
| | GKAPKLLIYG ASSYHSGVPS RFSGSGSGTD FTLTISSLQP |
| | EDFATYYCQH GQTLPRTFGQ GTKVEIK (SEQ ID NO: 33) |
| NMT25-LC | EIVLTQSPGT LSLSPGERAT LSCRASQDIS FFLNWYQQKP |
| | GQAPRLLIYY TSRYHSGIPD RFSGSGSGTD FTLTISRLEP |
| | EDFAVFYCQH GSTLPRTFGQ GTKVEIK (SEQ ID NO: 34) |
| NMT26-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDVATYYCQH GSTLPYTFGQ GTKVEIK (SEQ ID NO: 35) |
| NMT27-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GSTLPRTFGQ GTKVEIK (SEQ ID NO: 36) |
| NMT28-LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GQTLPWTFGQ GTKVEIK (SEQ ID NO: 37) |
| h9401-L1_LC | DIQMTQSPSS LSASVGDRVT ITCRASQDIS FFLNWYQQKP |
| | GKAPKLLIYY TSRYHSGVPS RFSGSGSGTD FTLTISSLQP |
| | EDFATYYCQH GNTLPWTFGQ GTKLEIKR (SEQ ID NO: 38) |
| h9401-L2_LC | DIQMTQSPSS LSASVGDRVT ITCRASQDIS FFLNWYQQKP |
| | GKAVKLLIYY TSRYHSGVPS RFSGSGSGTD FTLTISSLQP |
| | EDFATYYCQH GNTLPWTFGQ GTKLEIKR (SEQ ID NO: 39) |

### Example 2

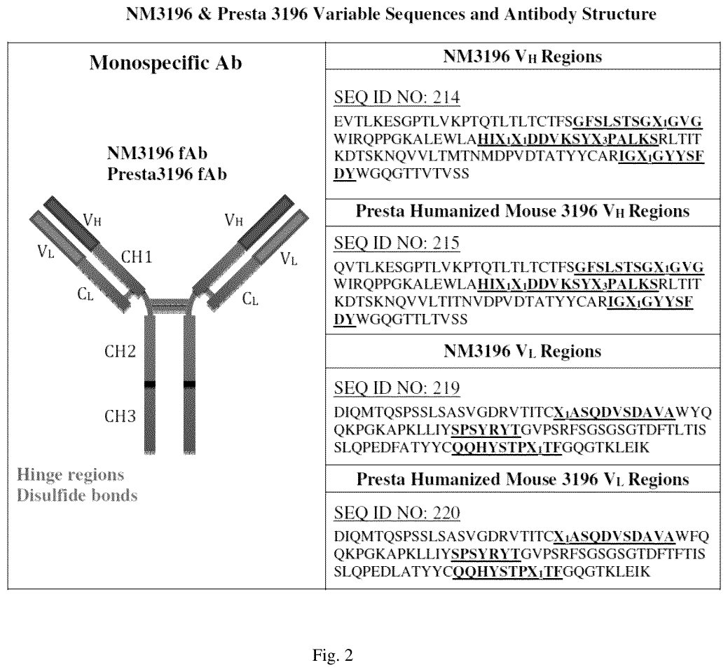

Example 2 describes structure and amino acid sequences of anti-properdin antibodies NMT-29-NMT31. The CDR substitutions for the variable light and heavy chain regions of NMT29-NMT31 (SEQ ID NO: 40-42 & 45-47) are listed in FIG. 2. The framework regions are from NMT29's heavy chain (SEQ ID NO: 40) and light chain (SEQ ID NO: 45) sequences. The CDR regions for the V_{H} Region (SEQ ID NO: 214) and V_{L} region (SEQ ID NOs: 219) contain multiple substitutions across variants of the anti-properdin antibody, where X represents the positions in the CDR's that are prone to substitutions.

The Presta humanized mouse 3196 variable light and heavy chain sequence CDR's are presented in the same manner. The framework regions are Presta h3196-H2 heavy chain (SEQ ID NO: 43) and h3196-L3 light chain (SEQ ID NO: 48). The CDR regions for the V_{H} Region (SEQ ID NO: 215) and V_{L} region (SEQ ID NOs: 220) contain multiple substitutions across variants of the anti-properdin antibody, where X represents the positions in the CDR's that are prone to substitutions. A full list of substitutions for NM3196 and Presta 3196 can be found below.

NM3196 (NMT29-NMT31) and Presta Mouse 3196
CDR Regions for SEQ ID NOs: 214 and 219

| | CDR1 | CDR2 | CDR3 |
|---|---|---|---|
| Heavy | GFSLSTSGX₁GVG | HIX₁X₁DDVKSYX₂PALKS | IGX₁GYYSFDY |
| Chain | (SEQ ID NO: 211) | (SEQ ID NO: 212) | (SEQ ID NO: 213) |
| | Wherein X₁ is 1, | Wherein X₁ is F, H, | Wherein X₁ is A, D, |
| | K, M, or V | W, or Y; X₂ is A, | E, or S |
| | | N, Q, or S | |
| Light | X₁ASQDVSDAVA | SPSYRYT | QQHYSTPX₁TF |
| Chain | (SEQ ID NO: 216) | (SEQ ID NO: 217) | (SEQ ID NO: 218) |
| | Wherein X₁ is K | No substitutions | Wherein X₁ is F, H, |
| | or R | | W, or Y |

The Ab structure is the same as the one presented in FIG. 1. In this case the fAb regions are made from the variable chains from NM3196 and Presta 3196. Multiple combinations of this antibody can be formed by using the CDR substitutions listed above. The V_{H} can be linked to any of the constant heavy chain Fc regions found in Table 6 using any of the linkers found in Table 7, the most common being the ASTK linker (SEQ ID NO: 245). The V_{L} is usually linked to the CL (SEQ ID NO: 246) with the RTVAAP (SEQ ID NO: 247) linker. A bispecific antibody can be generated by replacing a 3196 fAb with anti-Alb or a fAb that binds to a different target.

For NM3196, the CDR substitutions were based off of Presta's humanized mouse 3196. In the heavy chain sequence 5 main substitutions were presented. One substitution had the replacement of amino acid V with L; another substitution had the replacement of amino acid M with V, I, or K also known as a methionine substitution; another substitution had the same tryptophan substitution present in NM5072; the fourth substitution had the replacement of N with Q, 5, or A also known as the Asn/Pro Fragmentation substitution for NM3196; the final substitution had the replacement of D with E, 5, or A also known as an isoaspartate substitution for NM3196.

For the light chain sequence four main substitutions were presented. One substitution had the replacement of amino acid L with M or V; another substitution had the replacement of amino acid A with S or T; the third substitution had the replacement of amino acid R with K; the final substitution was a tryptophan substitution.

Additional CDR variants and differences in the framework regions are listed as separate sequences in Table 2.

TABLE 2
HEAVY CHAIN

| | |
|---|---|
| NMT29-HC | EVTLKESGPT LVKPTQTLTL TCTFSGFSLS TSGMGVGWIR |
| | QPPGKALEWL AHIWWDDVKS YNPALKSRLT ITKDTSKNQV |
| | VLTMTNMDPV DTATYYCARI GDGYYSFDYW GQGTTVTVSS |
| | (SEQ ID NO: 40) |
| NMT30-HC | EVTLKESGPT LVKPTQTLTL TCTFSGFSLS TSGMGVGWIR |
| | QPPGKALEWL AHIWWDDVKS YNPALKSRLT ITKDTSKNQV |
| | VLTITNVDPV DTATYYCARI GDGYYSFDYW GQGTTLTVSS |
| | (SEQ ID NO: 41) |
| NMT31-HC | EVTLKESGPT LVKPTQTLTL TCTFSGFSLS TSGMGVGWIR |
| | QPPGKALEWL AHIWWDDVKS YNPALKSRLT ITKDTSKNQV |
| | VLTMTNMDPV DTATYYCARI GDGYYSFDYW GQGTTVTVSS |
| | (SEQ ID NO: 42) |
| Prest_h3196-H1_HC | QVTLKESGPT LVKPTQTLTL TCTFSGFSLS TSGMGVGWIR |
| | QPPGKALEWL AHIWWDDVKS YNPALKSRLT ITKDTSKNQV |
| | VLTMTNMDPV DTATYYCARI GDGYYSFDYW GQGTTVTVSS |
| | (SEQ ID NO: 43) |
| Prest_h3196-H2_HC | QVTLKESGPT LVKPTQTLTL TCTFSGFSLS TSGMGVGWIR |
| | QPPGKALEWL AHIWWDDVKS YNPALKSRLT ITKDTSKNQV |
| | VLTITNVDPV DTATYYCARI GDGYYSFDYW GQGTTLTVSS |
| | (SEQ ID NO: 44) |

LIGHT CHAIN

| | |
|---|---|
| NMT29-LC | DIQMTQSPSS LSASVGDRVT ITCRASQDVS DAVAWYQQKP |
| | GKAPKLLIYS PSYRYTGVPS RFSGSGSGTD FTLTISSLQP |
| | EDFATYYCQQ HYSTPWTFGQ GTKLEIK (SEQ ID NO: 45) |
| NMT30-LC | DIQMTQSPSS LSASVGDRVT ITCRASQDVS DAVAWFQQKP |
| | GKAPKLLIYS PSYRYTGVPS RFSGSGSGTD FTFTISSLQP |
| | EDFATYYCQQ HYSTPWTFGQ GTKLEIK (SEQ ID NO: 46) |
| NMT31-LC | DIQMTQSPSS LSASVGDRVT ITCRASQDVS DAVAWFQQKP |
| | GKAPKLLIYS PSYRYTGVPS RFSGSGSGTD FTFTISSLQP |
| | EDLATYYCQQ HYSTPWTFGQ GTKLEIK (SEQ ID NO: 47) |
| Prest_h3196-L1_LC | DIQMTQSPSS LSASVGDRVT ITCRASQDVS DAVAWYQQKP |
| | GKAPKLLIYS PSYRYTGVPS RFSGSGSGTD FTLTISSLQP |
| | EDFATYYCQQ HYSTPWTFGQ GTKLEIK (SEQ ID NO: 48) |
| Prest_h3196-L2_LC | DIQMTQSPSS LSASVGDRVT ITCRASQDVS DAVAWFQQKP |
| | GKAPKLLIYS PSYRYTGVPS RFSGSGSGTD FTFTISSLQP |
| | EDFATYYCQQ HYSTPWTFGQ GTKLEIK (SEQ ID NO: 49) |
| Prest_h3196-L3_LC | DIQMTQSPSS LSASVGDRVT ITCRASQDVS DAVAWFQQKP |
| | GKAPKLLIYS PSYRYTGVPS RFSGSGSGTD FTFTISSLQP |
| | EDLATYYCQQ HYSTPWTFGQ GTKLEIK (SEQ ID NO: 50) |

### Example 3

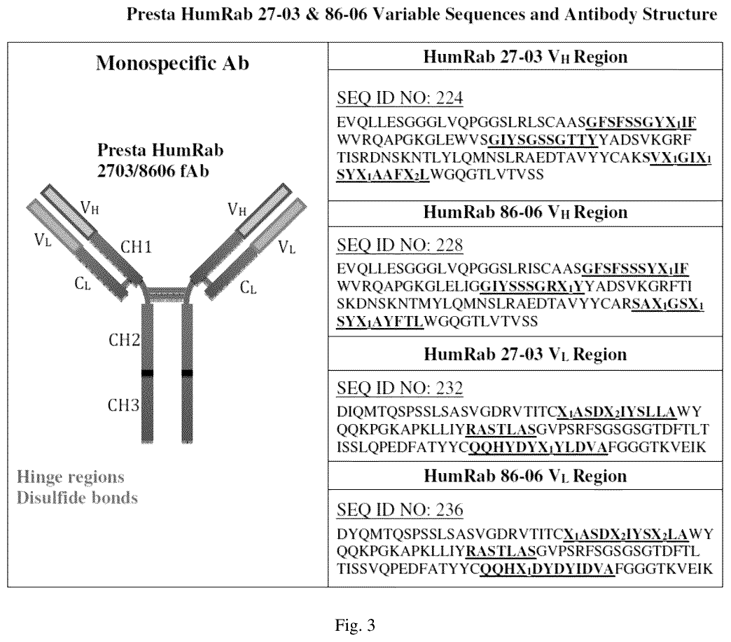

Example 3 describes the structure and amino acid sequences of anti-Properdin antibodies that include CDR's for two different humanized rabbit variable light and heavy chain sequences. The humanized rabbit variable light and heavy chain sequences are listed in FIG. 3 (Rabbit 27-03 and Rabbit 86-06). For rabbit 27-03, the framework regions were from Presta report's h2703-H1 heavy chain (SEQ ID NO: 51) and h2703-L1 light chain (SEQ ID NO: 61). For rabbit 86-06, the framework regions were from Presta report's h8606-H2 heavy chain (SEQ ID NO: 56) and h8606-L3 light chain (SEQ ID NO: 66). The CDR regions for the V_{H} Regions (SEQ ID NO: 224 and 232) and V_{L} regions (SEQ ID NOs: 228 and 236) contain multiple substitutions across variants of the anti-properdin antibody, where X represents the positions in the CDR's that are prone to substitutions. A full list of CDR substitutions for both 27-03 and 86-06 can be found in the table below.

Presta Humanized Rabbit Anti-P Sequences

| | | |
|---|---|---|
| CDR1 | CDR2 | CDR3 |

CDR Regions for 27-03 SEQ ID NOs: 221-223 (VH)
& SEQ ID NO: 229-231 (VL)

| | | | |
|---|---|---|---|
| Heavy | GFSFSSGYX₁IF | GIYSGSSGTTY | SVX₁GIX₁SYX₁ AAFX₂L |
| Chain | (SEQ ID NO: 221) | (SEQ ID NO: 222) | (SEQ ID NO: 223) |
| | Wherein X₁ is F, | No substitutions | Wherein X₁ is A, D, E, |
| | H, W, or Y | | or S; X₂ is A, N, Q, |
| | | | or S |
| Light | X₁ASDX₂IYSLLA | RASTLAS | QQHYDYX₁YLDVA |
| Chain | (SEQ ID NO: 229) | (SEQ ID NO: 230) | (SEQ ID NO: 231) |
| | Wherein X₁ is Q or R; | No substitutions | Wherein X₁ is A, N, Q, |
| | X₂ is A, N, Q, or S | | or S |

CDR Regions for 86-06 SEQ ID NOs: 225-227 (VH)
& SEQ ID NO: 233-235 (VL)

| | | | |
|---|---|---|---|
| Heavy | GFSFSSSYX₁IF | GIYSSSGRXiY | SAX₁GSX₁SYX₁AYFTL |
| Chain | (SEQ ID NO: 225) | (SEQ ID NO: 226) | (SEQ ID NO: 227) |
| | Wherein X₁ is F, H, | Wherein X₁ is I, | Wherein X₁ is A, D, E, |
| | W, or Y | K, L, or M | or S |
| Light | X₁ASDX₂IYSX₂LA | RASTLAS | QQHX₁DYDYIDVA |
| Chain | (SEQ ID NO: 233) | (SEQ ID NO: 234) | (SEQ ID NO: 235) |
| | Wherein X₁ is Q or R; | No substitutions | Wherein X₁ is F, H, W, |
| | X₂ is A, N, Q, or S | | or Y |

The Ab structure is a monospecific antibody much like NM5072 and NM3196. Different antibody sequence combinations can be made using the CDR substitutions. The V_{H} can be linked to a variation of different IgG Fc's found in Table 6. The linkers that can be used to join these two regions are in Table 7 with the most common being the ASTK (SEQ ID NO: 245) linker. The V_{L} is usually joined to the CL (SEQ ID NO: 246) using the RTVAAP (SEQ ID NO: 247) linker. The antibody can be made bispecific by replacing one of the fAbs with anti-albumin or a fAb with a different target.

For rabbit 27-03, the heavy chain CDR's had 6 main substitutions. One substitution had the replacement of amino acid D with T; another substitution had the replacement of N with T; yet another substitution had the replacement of V with T; one substitution was the tryptophan substitution; one substitution was the isoaspartate formation substitution present in NM3196; the final substitution was the replacement of N with Q, S, or A also known as a deamidation substitution for rabbit 27-03.

Humanized rabbit 27-03 light chain CDR's had 6 main substitutions. One substitution had the replacement of amino acid L with V; one substitution had the replacement of R with Q; another substitution had the replacement of A with P; one substitution was the deamidation substitution; one substitution had the replacement of S with K; the final substitution had the replacement of I with V.

For rabbit 86-06, the heavy chain CDR's had 6 main substitutions as well. One substitution was the replacement of amino acid V with A or T; another was the tryptophan substitution; one was the replacement of M with L, I, or K also known as a methionine substitution for rabbit 86-06; another substitution had the replacement of D with T; yet another substitution had the replacement of N with S, or T; the final substitution was an isoaspartate substitution present in NM3196.

Humanized rabbit 86-06 light chain CDR's also contained 6 main substitutions. L can be replaced by V; R can be replaced by Q; one substitution was the deamidation substitution; A can be replaced by R; one substitution was the tryptophan substitution; and the final substitution had the replacement of I with V.

Additional CDR variants and differences in the framework regions are listed as separate sequences in Table 3.

TABLE 3
Humanized RabMab-HC

| | |
|---|---|
| h2703-H1 | EVQLLESGGG LVQPGGSLRL SCAASGFSFS SGYWIFWVRQ |
| | APGKGLEWVS GIYSGSSGTT YYADSVKGRF TISRDNSKNT |
| | LYLQMNSLRA EDTAVYYCAK SVDGIDSYDA AFNLWGQGTL |
| | VTVSS (SEQ ID NO: 51) |
| h2703-H2 | EVQLLESGGG LVQPGGSLRL SCAASGFSFS SGYWIFWVRQ |
| | APGKGLELVG GIYSGSSGTT YYADSVKGRF TISKDNSKNT |
| | VYLQMNSLRA EDTAVYYCAR SVDGIDSYDA AFNLWGQGTL |
| | VTVSS (SEQ ID NO: 52) |
| h2703-H3 | EVQLLESGGG LVQPGGSLRL SCAASGFSFS SGYWIFWVRQ |
| | APGKGLELVG GIYSGSSGTT YYADWAKGRF TISKDNSKNT |
| | VYLQMNSLRA EDTAVYYCAR SVDGIDSYDA AFNLWGQGTL |
| | VTVSS (SEQ ID NO: 53) |
| h2703-H2a | EVQLLESGGG LVQPGGSLRL SCAASGFSFS SGYWIFWVRQ |
| | APGKGLELVG GIYSGSSGTT YYADSVKGRF TISKDSSKNT |
| | VYLQMNSLRA EDTAVYYCAR SVDGIDSYDA AFNLWGQGTL |
| | VTVSS (SEQ ID NO: 54) |
| h2703-H3a | EVQLLESGGG LVQPGGSLRL SCAASGFSFS SGYWIFWVRQ |
| | APGKGLELVG GIYSGSSGTT YYADWAKGRF TISKDSSKNT |
| | VYLQMNSLRA EDTAVYYCAR SVDGIDSYDA AFNLWGQGTL |
| | VTVSS (SEQ ID NO: 55) |
| h8606-H1 | EVQLLESGGG LVQPGGSLRL SCAASGFSFS SSYWIFWVRQ |
| | APGKGLEWVS GIYSSSGRMY YADSVKGRFT ISRDNSKNTL |
| | YLQMNSLRAE DTAVYYCAKS ADGSDSYDAY FTLWGQGTLV |
| | TVSSS (SEQ ID NO: 56) |
| h8606-H2 | EVQLLESGGG LVQPGGSLRI SCAASGFSFS SSYWIFWVRQ |
| | APGKGLELIG GIYSSSGRMY YADSVKGRFT ISKDNSKNTM |
| | YLQMNSLRAE DTAVYYCARS ADGSDSYDAY FTLWGQGTLV |
| | TVSSS (SEQ ID NO: 57) |
| h8606-H3 | EVQLLESGGG LVQPGGSLRI SCAASGFSFS SSYWIFWVRQ |
| | APGKGLELIG GIYSSSGRMY YADWAKGRFT ISKDNSKNTM |
| | YLQMNSLRAE DTAVYYCARS ADGSDSYDAY FTLWGQGTLV |
| | TVSSS (SEQ ID NO: 58) |
| h8606-H2a | EVQLLESGGG LVQPGGSLRI SCAASGFSFS SSYWIFWVRQ |
| | APGKGLELIG GIYSSSGRMY YADSVKGRFT ISKDSSKNTM |
| | YLQMNSLRAE DTAVYYCARS ADGSDSYDAY FTLWGQGTLV |
| | TVSSS (SEQ ID NO: 59) |
| h8606-H3a | EVQLLESGGG LVQPGGSLRI SCAASGFSFS SSYWIFWVRQ |
| | APGKGLELIG GIYSSSGRMY YADWAKGRFT ISKDSSKNTM |
| | YLQMNSLRAE DTAVYYCARS ADGSDSYDAY FTLWGQGTLV |
| | TVSSS (SEQ ID NO: 60) |

Rabbit mAb-LC

| | |
|---|---|
| h2703-L1 | DIQMTQSPSS LSASVGDRVT ITCRASDNIY SLLAWYQQKP |
| | GKAPKLLIYR ASTLASGVPS RFSGSGSGTD FTLTISSLQP |
| | EDFATYYCQQ HYDYNYLDVA FGGGTKVEIK (SEQ ID NO: 61) |
| h2703-L2 | DIQLTQSPSS LSASVGDRVT ITCRASDNIY SLLAWYQQKP |
| | GKAPKLLIYR ASTLASGVPS RFSGSGSGTD FTLTISSVQP |
| | EDFATYYCQQ HYDYNYLDVA FGGGTKVEIK (SEQ ID NO: 62) |
| h2703-L3 | DYQLTQSPSS LSASVGDRVT ITCRASDNIY SLLAWYQQKP |
| | GKAPKLLIYR ASTLASGVPS RFSGSGSGTD FTLTISSVQP |
| | EDFATYYCQQ HYDYNYLDVA FGGGTKVEIK (SEQ ID NO: 63) |
| h2703-L2a | DIQLTQSPSS LSASVGDRVT ITCRASDNIY SLLAWYQQKP |
| | GKAPKLLIYR ASTLASGVPS RFSGSGSGTD FTLTISSVQP |
| | EDAATYYCQQ HYDYNYLDVA FGGGTKVEIK (SEQ ID NO: 64) |
| h2703-L3a | DYQLTQSPSS LSASVGDRVT ITCRASDNIY SLLAWYQQKP |
| | GKAPKLLIYR ASTLASGVPS RFSGSGSGTD FTLTISSVQP |
| | EDAATYYCQQ HYDYNYLDVA FGGGTKVEIK (SEQ ID NO: 65) |
| h8606-L1 | DIQMTQSPSS LSASVGDRVT ITCRASQNIY SNLAWYQQKP |
| | GKAPKLLIYR ASTLASGVPS RFSGSGSGTD FTLTISSLQP |
| | EDFATYYCQQ HWDYDYIDVA FGGGTKVEIK (SEQ ID NO: 66) |
| h8606-L2 | DIQMTQSPSS LSASVGDRVT ITCRASQNIY SNLAWYQQKP |
| | GKAPKLLIYR ASTLASGVPS RFSGSGSGTD FTLTISSVQP |
| | EDFATYYCQQ HWDYDYIDVA FGGGTKVEIK (SEQ ID NO: 67) |
| h8606-L3 | DYQMTQSPSS LSASVGDRVT ITCRASQNIY SNLAWYQQKP |
| | GKAPKLLIYR ASTLASGVPS RFSGSGSGTD FTLTISSVQP |
| | EDFATYYCQQ HWDYDYIDVA FGGGTKVEIK (SEQ ID NO: 68) |
| h8606-L2a | DIQMTQSPSS LSASVGDRVT ITCRASQNIY SNLAWYQQKP |
| | GKAPKLLIYR ASTLASGVPS RFSGSGSGTD FTLTISSVQP |
| | EDAATYYCQQ HWDYDYIDVA FGGGTKVEIK (SEQ ID NO: 69) |
| h8606-L3a | DYQMTQSPSS LSASVGDRVT ITCRASQNIY SNLAWYQQKP |
| | GKAPKLLIYR ASTLASGVPS RFSGSGSGTD FTLTISSVQP |
| | EDAATYYCQQ HWDYDYIDVA FGGGTKVEIK (SEQ ID NO: 70) |
| Fv-(VH HumRab | EVQLLESGGG LVQPGGSLRL SCAVSGIDLS NYAINWVRQA |
| Anti-ALB) | PGKGLEWIGIIWASGTTFYA TWAKGRFTIS RDNSKNTVYL |
| | QMNSLRAEDT AVYYCARTVP GYSTAPYFDL WGQGTLVTVS |
| | SASTK (SEQ ID NO: 71) |
| Fv-(VL HumRab | DIQMTQSPSS VSASVGDRVT ITCQSSPSVW SNFLSWYQQK |
| Anti-ALB) | PGKAPKLLIY EASKLTSGVP SRFSGSGSGT DFTLTISSLQ |
| | PEDFATYYCG GGYSSISDTT FGGGTKVEIK RTVAAP (SEQ ID |
| | NO: 72) |

### IgG Fe Constant Chain Variations

The variable chains in FIG. 1, FIG. 2, and FIG. 3 can be combined into full fAb regions and linked to full IgG constant chain regions. Full constant chain regions (Fc's) are listed in Table 6. The CH regions contain variations that are present in their sequences. The IgG N298A Fc, as described in the name, has a point mutation at position 298, which replaces amino acid N with A. The mutation does not always occur at position 298 in the sequence. It is dependent on the length of the V_{H} and the linker.

The YTE Fc contains three point mutations: M253Y, S255T, and T257E. Like the N298A Fc, the mutations are not always guaranteed to occur at the listed positions. The length of the V_{H} and linker will influence the location.

IgG2 is a commonly used Fc sequence for monospecific and bispecific complement targeting antibodies.

There is no variation in the IgG constant light chain. The C_{L} is almost always the same for every antibody.

The common linkers used to join the variable chain regions to the constant chain regions are listed in Table 7. The most commonly used variable heavy chain linker is the ASTK linker (SEQ ID NO: 245) and the most commonly used variable light chain linker is the RTVAAP linker (SEQ ID NO: 247). All other linkers are normally used to link the variable heavy chain to the constant heavy chain. In some embodiments the linkers can be used to link two variable heavy chains together directly with a CH1 region separating them.

### Example 4

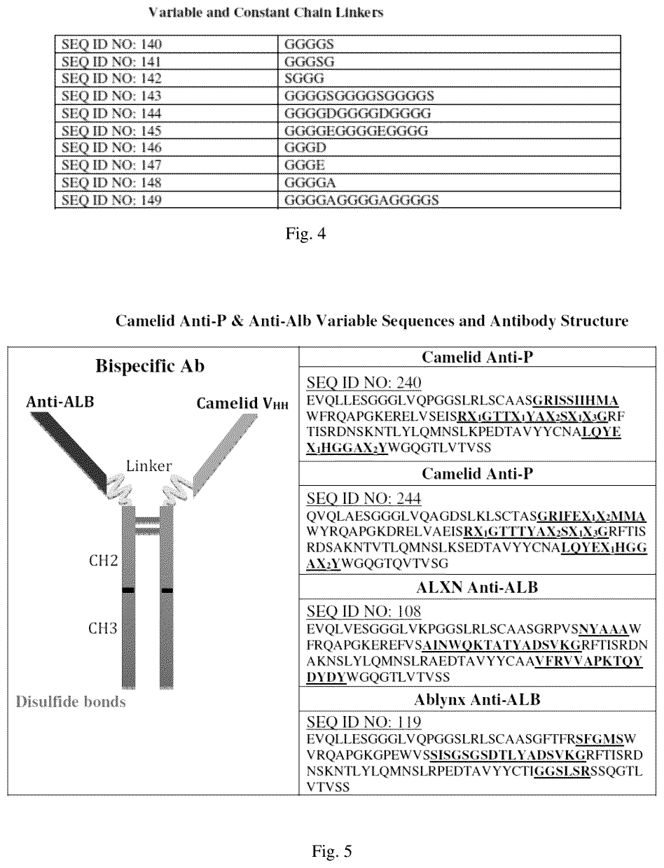

Example 4 describes the structure and amino acid sequences of monospecific and bispecific camelid anti-properdin antibodies. CDR substitutions for the camelid sequences (LVP058 and V_{HH}) along with selected framework regions are shown in FIG. 4. The framework regions for the sequences are from SEQ ID NOs: 45-51, 59, and 61 of U.S. Patent Application Publication No US20190352381A1, which is herein incorporate by reference in its entirety. The CDR regions for the V_{HH} Regions (SEQ ID NO: 240 and 244) contain multiple substitutions across variants of the camelid anti-properdin antibody, where X represents the positions in the CDR's that are prone to substitutions. Also listed are the humanized Anti-ALB sequences. A full list of CDR substitutions for the camelid anti-properdin sequences can be found in the table below.

Humanized Camelid Heavy Chain Fv

| | | |
|---|---|---|
| CD1 | CDR2 | CDR3 |

SEQ ID NO: 237-239

| | | | |
|---|---|---|---|
| Heavy | GRISSIIHMA | RX₁GTTX₁YAX₂SX₁X₃G | LQYEX₁HGGAX₂Y |
| Chain | (SEQ ID NO: 237) | (SEQ ID NO: 238) | (SEQ ID NO: 239) |
| | No substitutions | Wherein X₁ is 1 | Wherein X₁ is |
| | | or V; X₂ is A, D, | A or K; X₂ is A, |
| | | E, or S; X₃ is A | D, E, or S |
| | | or K | |

SEQ ID NO: 241-243

| | | | |
|---|---|---|---|
| Heavy | GRIFEX₁X₂MMA | RX₁GTTTYAX₂SX₁X₃G | LQYX₁RYGGAEY |
| Chain | (SEQ ID NO: 241) | (SEQ ID NO: 242) | (SEQ ID NO: 243) |
| | Wherein X₁ is I or | Wherein X₁ is 1 or | X₁ is A, D, E, |
| | V; X₂ is A, D, N, | V; X₂ is A, D, E, | or S |
| | Q, or S | or S; X₃ is A or K | |

The structure in FIG. 5 is a bispecific antibody that binds both properdin and Albumin. This embodiment has the fAb regions directly linked to the CH2 region of the Fc. The CH1 region is not present due to the fact that a V_{L} is not required for camelid species. The addition of the Alb will help increase the half-life and decrease the clearance rate of this antibody in the body. Using the Presta substitutions, different combinations of camelid heavy chain sequences can be attached to the Fc region.

Additionally, these heavy chain regions can be linked to any of the heavy chain Fc's shown in Table 6 with the CH1 region removed. For these embodiments, any linker can be utilized from Table 7 with the most common being the (G₄S)₃ linker.

For the camelid anti-properdin sequences, there is no light chain region. Camelid species contains only the heavy chain domain (V_{HH}). There were four main substitutions that were given to the CDR regions. The substitutions were based off of Presta substitutions found in humanized mouse 9401 and 3196. One substitution had the replacement of amino acid V with I; another substitution had the replacement of amino acid K with A; one substitution was the presence of the deamidation substitution from Presta report 9401; the final substitution was the isoaspartate formation substitution from Presta report 3196.

These substitutions do not cover all the changes in the CDR's for the camelid heavy chain sequences. Additional CDR variants and differences in the framework regions are listed as separate sequences in Table 4 below.

TABLE 4
Camelid Anti-P

| | |
|---|---|
| Anti-P (from LPV058) | EVQLVESGGG LVQAGGSLRL SCAASGRISS IIHMAWYRQA |
| | PGKQRELVAE ISRVGTTVYA DSVKGRFTIS RDDAKNTVTL |
| | QMNSLKPEDT AVYYCNALQY EKHGGADYWG |
| | QGTQVTVSS (SEQ ID NO: 73) |
| Anti-P (from LPV058) | EVQLLESGGG LVQPGGSLRL SCAASGRISS IIHMAWFRQA |
| | PGKERELVSE ISRVGTTVYA DSVKGRFTIS RDNSKNTLYL |
| | QMNSLKPEDT AVYYCNALQY EKHGGADYWG |
| | QGTLVTVSS (SEQ ID NO: 74) |
| Anti-P (from LPV058) | EVQLVESGGG LVQPGGSLRL SCAASGRISS IIHMAWVRQA |
| | PGKQRELVSE ISRVGTTVYA DSVKGRFTIS RDNSKNTLYL |
| | QMNSLRAEDT AVYYCNALQY EKHGGADYWG |
| | QGTLVTVSS (SEQ ID NO: 75) |
| V_{HH} on Silent | EVQLLESGGG LVQAGGSLRL SCAASGRISS IIHMAWYRQA |
| Human Fc | PGKQRELVSE ISRVGTTVYA DSVKGRFTIS RDDAKNTVTL |
| | QMNSLKPEDT AVYYCNALQY EAHGGASYWG QGTQVTVSS |
| | (SEQ ID NO: 76) |
| Anti-Properdin-V_{HH} | QVQLVESGGG LVQAGGSLRL SCAASGRISS IIHMAWYRQA |
| | PGKQRELVAE ISRIGTTVYA ESVAGRFTIS RDDAKNTVTL |
| | QMNSLKPEDT AVYYCNALQY EKHGGASYWG |
| | QGTQVTVSG (SEQ ID NO: 77) |
| Anti-Properdin-V_{HH} | QVQVVESGGG LRQTGGSLRL SCTASGRIFE |
| | VNMMAWYRQA PGKQRELVAE ISRVGTTVYA |
| | DSVKGRFTIS RDSAKNTVTL QMNSLKSEDT AVYYCNALQY |
| | DRYGGAEYWG QGTQVTVSS (SEQ ID NO: 78) |
| Anti-Properdin-V_{HH} | QVQLVESGGG LRQTGESLRL SCTASGRIFE |
| | VNMMAWYRQA PGKQRELVAE ISRVGTTTYA DSVKGRFTIS |
| | RDSAKNTVTL QMNSLKSEDT AVYYCNALQY |
| | DRYGGAEYWG QGTQVTVSG (SEQ ID NO: 79) |
| Anti-Properdin-V_{HH} | QVQLAESGGG LVQAGDSLKL SCTASGRIFE |
| | VNMMAWYRQA PGKDRELVAE ISRVGTTTYA DSVKGRFTIS |
| | RDSAKNTVTL QMNSLKSEDT AVYYCNALQY |
| | SRYGGAEYWG QGTQVTVSG (SEQ ID NO: 80) |
| Fab-Vhh Domain | QVQLVESGGG LVQAGGSLRL SCAASGGTFS |
| | SYSMGWFRQA PGKEREFVAA ITWNGVSTYY ADSVKGRFTI |
| | SRDNAKNTVY LQMNSLKPTD TAVYYCAAEI TTRYSGFYYY |
| | EDNKSYDYWG QGTQVTVSS (SEQ ID NO: 81) |
| Fab-Vhh Domain | QVQLIESGGG LVQAGDSLRL SCATSEGTFS RFAMGWFRQA |
| | PGKEREFVAA INWSGGITYY ADSIAGRFTI SRDNAKNTVY |
| | LQMNSLKPED TADYYCAAET TTRYSGYYYY |
| | EDNKSYDYWG QGTLVTVSG (SEQ ID NO: 82) |
| Fab-Vhh Domain | QVQLVESGGG LVQAGGSLRL SCAASGRTFS |
| | TLGMGWFRQA PGKERQFVAA INWSGSSTYY ANSVKGRFTI |
| | SRDNAQSTMY LQMNSLKPED TAVYYCAADL |
| | DSRYSAYYYY SDESQYDYWG QGTLVTVSG (SEQ ID NO: 83) |
| Fab-Vhh Domain | QVQLVESGGG LVQAGGSLRL SCAASGRTFS SYAMGWFRQP |
| | PGKEREFVAA ITWRGASTYY ADPIKGRFTI SRDNAKNTVY |
| | LQMSSLKPED TAVYYCAAEE PSYYSGSYYY |
| | MMGESYAYWG QGTLVTVSG (SEQ ID NO: 84) |
| Fab-Vhh Domain | QVHLVESGGG LVQAGGSLRL SCAASGRTFS SYAMGWFRQP |
| | PGKERQFVAA ITWSGSSIYY ADSIKGRFTI SRDNAKNTVY |
| | LQMNSLKPED TAVYYCAAEE TSAYSGSYYY |
| | MMGDSYSYWG QGTQVTVSG (SEQ ID NO: 85) |
| Fab-Vhh Domain | QVQLVESGGG LVQPGGSLSL SCAASGRTFS SYAMGWFRQA |
| | PGKEREWVAA ITWSGANIYY ADSIKGRFTL SRDNAENTVW |
| | LQLNSLKPED TAVYYCAAAE SGRYSGRAYY |
| | SAPGVYLYWG QGTLVTVSG (SEQ ID NO: 86) |
| Fab-Vhh Domain | QVQLVESGGG LVQAGGSLRL SCAASGRTFS |
| | NYAMAWFRQA PGKEREFVAS ISGSGDSRYY ADSVKGRFTI |
| | SRDNAKNTVY LQTNSPKPED TAVYYCAAVL PTRYSGFYYY |
| | SDGTQYHYWG QGTQVTVSS (SEQ ID NO: 87) |
| Fab-Vhh Domain | QVRLVESGGG LVQAGDSLRL SCATSGRTLS |
| | SYAMGWFRQA PGKEREFVAA TTWRDTSTYY |
| | ADSVKGRFTI SRDNAKNTVY LQTNSLKPED TAAYYCAAEE |
| | PSKYSGRSYY MMGASYDYWG QGTQVTVSS (SEQ ID NO: |
| | 88) |
| Fab-Vhh Domain | QVQLVESGGG LVQPGGSLRL SCATSGGTFS SYAMGWFRQA |
| | PGKEREFVAA TTWQGSNRYY AESVAGRFTI SRDNAKSTVW |
| | LQMNSLKPED TAVYYCAAEH STRYSGFYYY |
| | TRGETYHYWG QGTQVTVSG (SEQ ID NO: 89) |
| Fab-Vhh Domain | QVQLVESGGG LVQPGGSLRL SCATSGGTFS SYAMGWFRQA |
| | PGKEREFVAA TTWQGSNRYY AESVAGRFTI SRDNAKSTVW |
| | LQMNSLKPED TAVYYCAAEH STRYSGFYYY |
| | TRGETYHYWG QGTQVTVSG (SEQ ID NO: 90) |
| Fab-Vhh Domain | QVQLVESGGG LVQAGDSLRF SCAASGFTFS SYAMGWFRQA |
| | PGKEREFVAA ITWSGVSTYY ADSIAGRFTI SRDNAKNRVY |
| | LQMNSLKPED TADYSCAADG SGRYSGMEYY |
| | NRDWVYDYWG QGTQVTVSS (SEQ ID NO: 91) |
| Fab-Vhh Domain | QVQLVESGGG LVQAGDSLRF SCAASGFTFS SYAMGWFRQA |
| | PGKEREFVAA ITWSGVSTYY ADSIAGRFTI SRDNAKNRVY |
| | LQMNSLKPED TADYSCAADG SGRYSGMEYY |
| | NRDWVYDYWG QGTQVTVSS (SEQ ID NO: 92) |
| Fab-Vhh Domain | EVQLVESGGG LVQPGGSLRL SCAASGFTFG SADMSWVRQA |
| | PGKGPEWVSA INSNGGSTYY AASVKGRFTI SRDNAKNTLY |
| | LQMNSLKPED TAVYYCAQGN WYTEEYHYWG |
| | QGTQVTVSG (SEQ ID NO: 93) |
| Fab-Vhh Domain | QVQLVESGGG LVQAGGSLRL SCAASGRTFS |
| | SYAMGWFRQA PGKEREFVAA IGWNGEGIYY ADSVKGRFTI |
| | SRDNAKNTGY LQMNSLKPED TAVYYCAADS EGVVPGFPIA |
| | YWGQGTQVTV SG (SEQ ID NO: 94) |
| Fab-Vhh Domain | EVQLVESGGG LVQPGGSLRL SCATSGRPFS SYAMGWFRQA |
| | PGKEREIVAG LSWSGGQIYY ADSVKGRFTI SRDNAKNTGD |
| | LQMNSLKPED TAVYYCAIGP ALTTGPTAYR |
| | YWGQGTQVTV SS (SEQ ID NO: 95) |
| Fab-Vhh Domain | QVHLVESGGG LVQAGGSLRL SCTASGGTVG |
| | DYAVGWFRQA PGKERELIGV VSRLGARTGY ADSVLGRFTI |
| | SRDDVKNTVF LQMDSVKPED TAVYYCAARR |
| | DYSFEVVPYD YWGQGTQVTV SG (SEQ ID NO: 96) |
| Fab-Vhh Domain | QVQLVESGGG LVQPGGSLRL SCAASGRTFS SYSMGWFRQR |
| | HGNEREFVAA ISWSGRSTYY AESVKGRFAI SRDNANTTVY |
| | LQMNSLKPED SAVYYCAAST RGWYGTQESD |
| | YNFWGQGTQV TVSG (SEQ ID NO: 97) |
| Fab-Vhh Domain | QVQLIESGGG LVQAGGSLRL SCTASGRTFS NYAMGWFRQA |
| | PGKEREFLAA ISRSGESTQY ATFVKGRFAI ARDNAKNTVS |
| | LQMNSLKPED TAVYFCAAKI AVLVSTTYNS QYEYWGQGTL |
| | VTVSS (SEQ ID NO: 98) |
| Fab-Vhh Domain | QVQLIESGGG LVQEGASLRL SCAGSGPMFS RLAVGWFRQA |
| | PGKEREFVAV INWSGSADFY TNSVKGRFTI SRDNAKNTVY |
| | LEMNTLKPED SAVYYCAADQ NPLTLRTGVR |
| | DVGRQWGQGT EVTVSS (SEQ ID NO: 99) |
| Fab-Vhh Domain | EVQLVESGGG LVQPGGSLRL SCAASGFTLD YYAIGWFRQA |
| | PGKEREGVSC ISRTDGSTYY ADSVKGRFTI SRDNAKNTVY |
| | LQMNSLKPED TAVYYCAVDD SYPTGGISCL FGHFGSWGQG |
| | TQVTVSS (SEQ ID NO: 100) |
| Fab-Vhh Domain | QVQLVESGGG LVQAGGSLRL SCAASGRTFS |
| | SYAMGWFRQA PGKEREFVAA VPWTYGSKYY |
| | ADSVKGRFTI SRDDAKNTVY LQMNNLKPED |
| | TAVYYCAADS SAGYYSGFDY YSAATPYDLW |
| | GQGTQVTVSG (SEQ ID NO: 101) |
| Fab-Vhh Domain | QVQMVESGGG LVQAGGSLRL SCAASGLTNR |
| | IRIMGWYRQA PGKLRELVAT ITNDGSTHYA DSVKGRFTIS |
| | TDNAKNTVFL QMNSLKPEDT AVYICNVGEN |
| | WGPAYWGQGT QVTVSG (SEQ ID NO: 102 |
| Fab-Vhh Domain | QVQLVESGGG LVQAGGSLXL SCAASGSDRR |
| | INGMGWYRHP PGKQRELVAA ITSGGSTNYA DSVKGRFTIS |
| | TNNANNMMYL QMNSLKPEDT AVYYCAIDEF |
| | GTGWLDYCGQ GTQVTVSG (SEQ ID NO: 103) |
| Fab-Vhh Domain | QVLLEESGGG LERTGGSLRL SCAASGSIFS VNSMTWYRQA |
| | PGKRREFLGT ITEEGRTNYA DSVKGRFTIS RDNAKNTMYL |
| | QMNSLKPEDT AVYYCYANLI SSEDRTFGVW GQGTQVTVSS |
| | (SEQ ID NO: 104) |
| Fab-Vhh Domain | QVHMVESGGG LVQAGGSLRF SCAASGNIFT ISTLDWYRQA |
| | PGEQRELVAT LTPDGITDYA GSVKGRFTIS RDNAKNTVYL |
| | QMNSLKPEDT AVYYCNAWRY SDDYRGRVDY |
| | WGQGTQVTVS G (SEQ ID NO: 105) |
| Fab-Vhh Domain | QVQLMESGGG EVQAGGSLRL SCAASGSIFD ISAMGWYRQA |
| | PGKQRELVAD ITSSGSTQYA DSVKGRFTIS RDNAKNTVYL |
| | QMNSLKPEDT AVYTCAAESI RESQNRHQLG |
| | YMGPLYDYWG QGTQVTVSG (SEQ ID NO: 106) |
| Fab-Vhh Domain | QVQLVESGGG LVQPGGSLRL SCAASGSDYY AIGWFRQAPG |
| | KEREGVSCMS RTDGSTYYAD SVKDRFTISR DYAKNTVYLQ |
| | MNSLKPEDTA VYYCGLDRSY PTGGISCLFG DFGSWGQGTQ |
| | VTVSG (SEQ ID NO: 107) |
| Human-Anti-Alb | EVQLVESGGG LVKPGGSLRL SCAASGRPVS NYAAAWFRQA |
| | PGKEREFVSA INWQKTATYA DSVKGRFTIS RDNAKNSLYL |
| | QMNSLRAEDT AVYYCAAVFR VVAPKTQYDY |
| | DYWGQGTLVT VSS (SEQ ID NO: 108) |

### Example 5

Example 5 describes the structure and amino acid sequences of additional bispecific anti-properdin antibodies. The sequences contain variations of an anti-albumin chain, camelid anti-properdin heavy chain, and NovelMed's NMT15 light and heavy chains.

### Anti-Properdin Antibodies

| Sequence | | |
|---|---|---|
| Number | mAb | Sequence Layout |
| 1 | Single Chain | Human anti-albumin with camelid |
| | | anti-properdin |
| 2 | Single Chain | Humanized anti-albumin with camelid |
| | | anti-properdin |
| 3 | (Fab)2 Linker | Two NMT15 light and heavy chain Fab |
| | | regions |
| 4 | Alb-Fab Linker | Human anti-albumin with NMT15 Fab |
| 5 | Alb-Fab Linker | Humanized anti-albumin with NMT15 |
| | | Fab region |

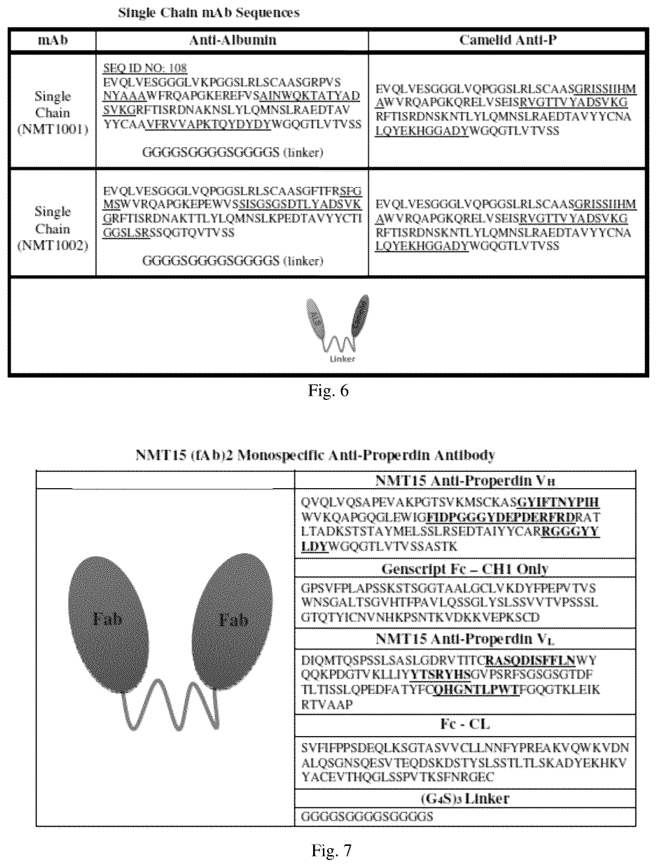

The camelid anti-properdin heavy chain sequence for these antibodies was modified from U.S. Patent Application Publication No. 20190352381A1 as well as the human anti-albumin sequence in the first and fourth constructs. The humanized anti-albumin sequence in constructs two and five were modified from U.S. Patent Application Publication No. 2007/0269422A1, which is incorporated by reference in its entirety. The NMT15 light and heavy chains were generated through humanization of a mouse anti-properdin Fab region of the original Presta sequences described above. The anti-albumin is used in the synthesis of these new construct to aid in extending the half-life and bioavailability of the drug. Examples of anti-albumin amino acid sequences are listed in Table 5. The full sequences of each construct can be found in FIGS. 6-8.

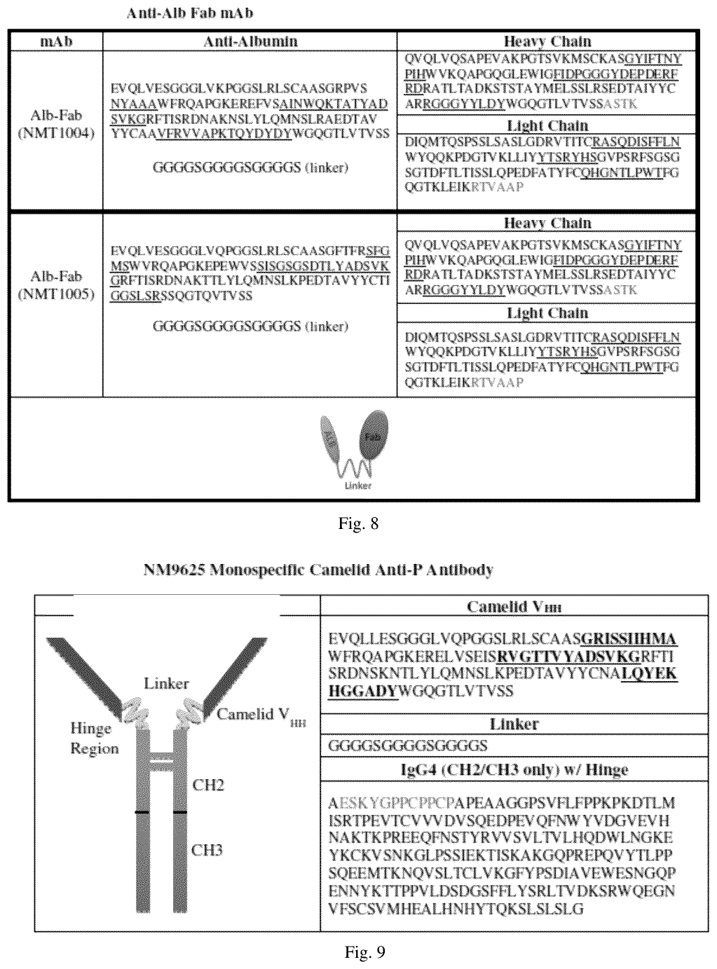

For all sequences in the tables above, the sequences were connected using a (G4S)3 linker (GGGSGGGSGGGS) (SEQ ID NO: 143). In FIG. 6, the anti-albumin is linked with the camelid anti-properdin. Similarly, in FIG. 7, two identical Fab regions (NMT15 HC/LC) are linked together to create a full mAb. In FIG. 8, the anti-albumin sequences from FIG. 6 are linked with the Fab region in FIG. 7 to form a full mAb. Specifically for sequence NMT1003-NMT1005, the heavy chain regions were connected to an Fc that only contained the CH1 region (SEQ ID NO 138) and the light chain regions were connected to a CL (SEQ ID NO 139).

TABLE 5
Anti-ALB

| | |
|---|---|
| ALB1 (PMP6A6) | AVQLVESGGG LVQPGNSLRL SCAASGFTFR SFGMSWVRQA |
| | PGKEPEWVSS ISGSGSDTLY ADSVKGRFTI SRDNAKTTLY |
| | LQMNSLKPED TAVYYCTIGG SLSRSSQGTQ VTVSS (SEQ ID |
| | NO: 109 |
| ALB3 (ALB1, HUM1) | EVQLVESGGG LVQPGGSLRL SCAASGFTFR SFGMSWVRQA |
| | PGKEPEWVSS ISGSGSDTLY ADSVKGRFTI SRDNAKTTLY |
| | LQMNSLKPED TAVYYCTIGG SLSRSSQGTQ VTVSS (SEQ ID |
| | NO: 110) |
| ALB4 (ALB1, HUM2) | EVQLVESGGG LVQPGGSLRL SCAASGFTFS SFGMSWVRQA |
| | PGKEPEWVSS ISGSGSDTLY ADSVKGRFTI SRDNAKTTLY |
| | LQMNSLKPED TAVYYCTIGG SLSRSSQGTQ VTVSS (SEQ ID |
| | NO: 111) |
| ALB5 (ALB1, HUM3) | EVQLVESGGG LVQPGGSLRL SCAASGFTFR SFGMSWVRQA |
| | PGKGLEWVSS ISGSGSDTLY ADSVKGRFTI SRDNAKTTLY |
| | LQMNSLKPED TAVYYCTIGG SLSRSSQGTQ VTVSS (SEQ ID |
| | NO: 112) |
| ALB6 (ALB1, HUM1) | EVQLVESGGG LVQPGNSLRL SCAASGFTFR SFGMSWVRQA |
| | PGKGLEWVSS ISGSGSDTLY ADSVKGRFTI SRDNAKTTLY |
| | LQMNSLKPED TAVYYCTIGG SLSRSSQGTL VTVSS (SEQ ID |
| | NO: 113) |
| ALB7 (ALB1, HUM2) | EVQLVESGGG LVQPGNSLRL SCAASGFTFR SFGMSWVRQA |
| | PGKGLEWVSS ISGSGSDTLY ADSVKGRFTI SRDNAKTTLY |
| | LQMNSLRPED TAVYYCTIGG SLSRSSQGTL VTVSS (SEQ ID |
| | NO: 114) |
| ALB8 (ALB1,HUM3) | EVQLVESGGG LVQPGNSLRL SCAASGFTFS SFGMSWVRQA |
| | PGKGLEWVSS ISGSGSDTLY ADSVKGRETI SRDNAKTTLY |
| | LQMNSLRPED TAVYYCTIGG SLSRSSQGTL VTVSS (SEQ ID |
| | NO: 115) |
| ALB9 (ALB1, HUM4) | EVQLVESGGG LVQPGNSLRL SCAASGFTFS SFGMSWVRQA |
| | PGKGLEWVSS ISGSGSDTLY ADSVKGRETI SRDNAKNTLY |
| | LQMNSLRPED TAVYYCTIGG SLSRSSQGTL VTVSS (SEQ ID |
| | NO: 116) |
| ALB10 (ALB1, HUM5) | EVQLVESGGG LVQPGNSLRL SCAASGFTFS SFGMSWVRQA |
| | PGKGLEWVSS ISGSGSDTLY ADSVKGRFTI SRDNAKNTLY |
| | LQMNSLRPED TAVYYCTIGG SLSRSGQGTL VTVSS (SEQ ID |
| | NO: 117) |
| ALB11 | EVQLVESGGG LVQPGNSLRL SCAASGFTFS SFGMSWVRQA |
| | PGKGLEWVSS ISGSGSDTLY ADSVKGRFTI SRDNAKTTLY |
| | LQMNSLRPED TAVYYCTIGG SLSRSSQGTL VTVSS (SEQ ID |
| | NO: 118) |
| ALB23 | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL VTVSS (SEQ ID |
| | NO: 119) |
| ALB23A | AWQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL VTVSS (SEQ ID |
| | NO: 120) |
| ALB23B | AWQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTQ WTVSS (SEQ ID |
| | NO: 121) |
| ALB23C | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTQ VTVSS (SEQ ID |
| | NO: 122) |
| ALB23D | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL VTVSS (SEQ ID |
| | NO: 123) |
| ALB23E | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL WTVSS (SEQ ID |
| | NO: 124) |
| ALB23F | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL WTVSS (SEQ ID |
| | NO: 125) |
| ALB23G | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL VTVSS (SEQ ID |
| | NO: 126) |
| ALB23H | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL VTVSS (SEQ ID |
| | NO: 127) |
| ALB23I | EVQLLESGGG LVQPGGSLRL SCAASGFTFR SFGMSWVRQA |
| | PGKGPEWVSS ISGSGSDTLY ADSVKGRFTI SRDNSKNTLY |
| | LQMNSLRPED TAVYYCTIGG SLSRSSQGTL WTVSS (SEQ ID |
| | NO: 128) |

### Example 6

Example 6 describes the structure and amino acid sequences of bispecific anti-properdin antibodies. The bispecific anti-properdin antibodies described in this example and the table below were designed to be more stable and longer lasting in circulation. Attaching an anti-albumin sequence to an anti-properdin antibody or antigen binding fragment thereof allowed the antibody to bind to serum albumin giving it a longer half-life and circulation time. Furthermore, the addition of the Fc region can also aid in increasing the half-life of the antibody and improves the stability of the overall structure. Multiple embodiments of the bispecific Anti-P antibody constructed using different Fc's listed in Table 6, linkers listed in Table 7, and fAB binding regions listed in Table 8, are described generally in the Table and embodiments below.

Monospecific LVP058 w/IgG4 Fc
Bispecific LVP058 - Anti-ALB w/IgG 4 Fc
Bispecific LVP058 - ALXN Anti-ALB w/(US20190002568A1)
IgG1 Fc
Bispecific NMT16 Anti-P Fab - HumRab Anti-ALB
w/(US20190002568A1) IgG1 Fc
Bispecific Presta HumRab Anti-P - HumRab Anti-ALB
w/(US20190002568A1) IgG2 Fc
Bispecific NMT16 Anti-P Fab - Anti-TNF Fab w/IgG Fc
Bispecific NMT16 Anti-P Fab - Anti-VEGF Fab w/N298A IgG Fc

NM9625 is a monospecific antibody that binds to properdin. Using a camelid Anti-P sequence, it is attached to a variation of the IgG4 Fc using a (G₄S)₃ linker. In this embodiment of the IgG4 Fe, there is no CH1 region, and the camelid variable heavy chain domain is directly linked to the hinge region of the Fc. The full sequence listing and the antibody structure can be found in FIG. 9.

NM9626 and NM9627 are bispecific antibodies that bind to both properdin and serum albumin. The sequence and structure of these antibodies in FIG. 4. A variation of the camelid Anti-P sequence is used along with a humanized camelid anti-albumin sequence. These variable heavy chain sequences are attached to a variation of the IgG Fc with a (G₄S)₃ linker. In these embodiments of the IgG Fc's, there is no CH1 region, and both the camelid Anti-P and Anti-Alb are directly linked to the hinge region of the IgG Fc. The major difference between these two antibodies is that NM9626 is formulated using the same IgG4 Fe as NM9625 (SEQ ID NO: 163 & 164) and NM9627 is formulated using an IgG1 Fe (SEQ ID NO: 165 & 166).

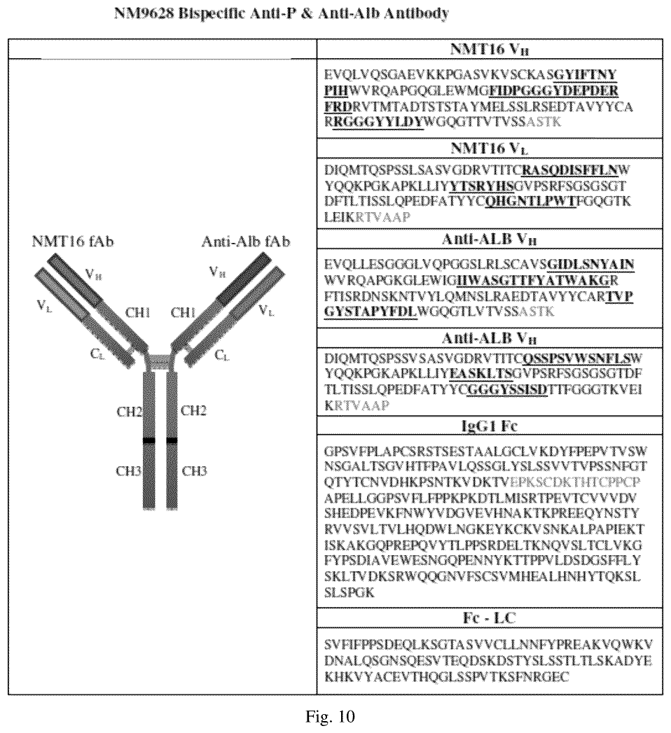

NM9628 is a bispecific antibody that binds to both properdin and serum albumin. The full sequence listing and the antibody structure can be found in FIG. 10. NovelMed's NMT16 Anti-P fAb region is combined with a humanized rabbit serum albumin fAb. The heavy chain regions for NMT16 and Anti-Alb are joined to a full IgG1 Fc. The light chain regions for NMT16 and Anti-ALB are joined to the CL.

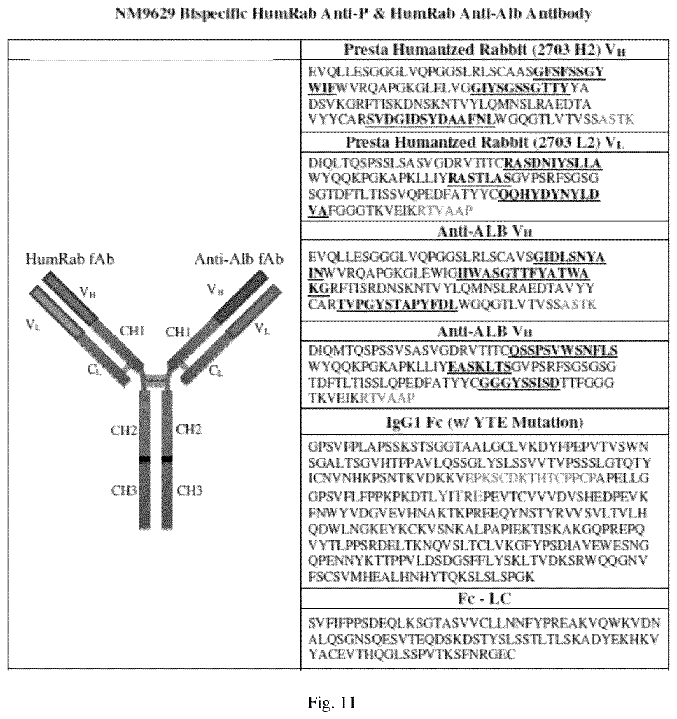

NM9629 is a bispecific antibody that binds both properdin and serum albumin. The full sequence listing and the antibody structure can be found in FIG. 11. Presta's humanized rabbit 27-03 Anti-P sequence is combined with a humanized rabbit anti-albumin sequence. The heavy chain regions for Presta humanized rabbit and anti-albumin are joined to the IgG1 Full Fe with the YTE mutation (SEQ ID NO: 131). The light chain regions for Presta humanized rabbit and Anti-Alb are joined to the CL.

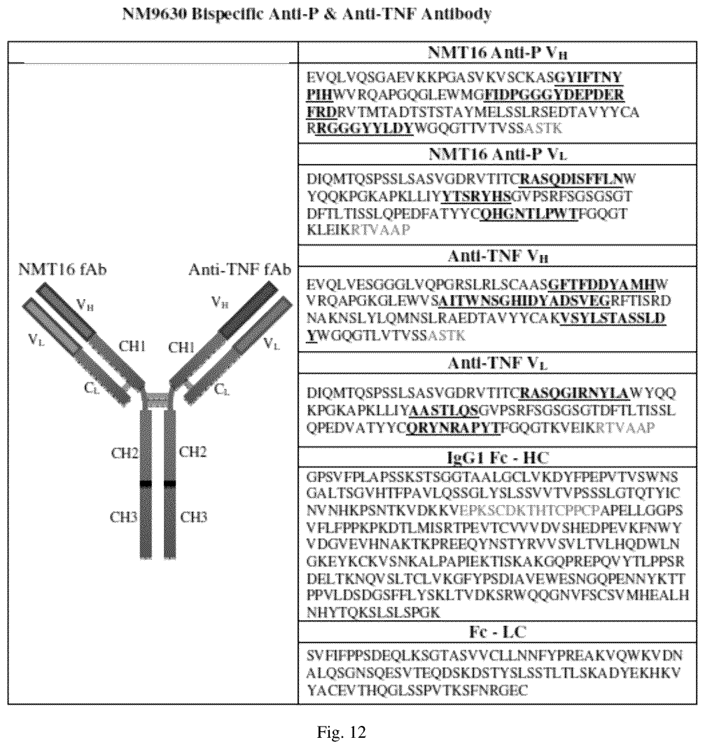

NM9630 is a bispecific antibody that binds both properdin and TNF. The full sequence listing and the antibody sequence can be found in FIG. 12. NovelMed's NMT16 fAb is combined with an anti-TNF fAb. The heavy chains are connected to an IgG1 Fc pulled from an anti-TNF antibody: Humira (SEQ ID NO: 129), and the light chains are connected to the CL.

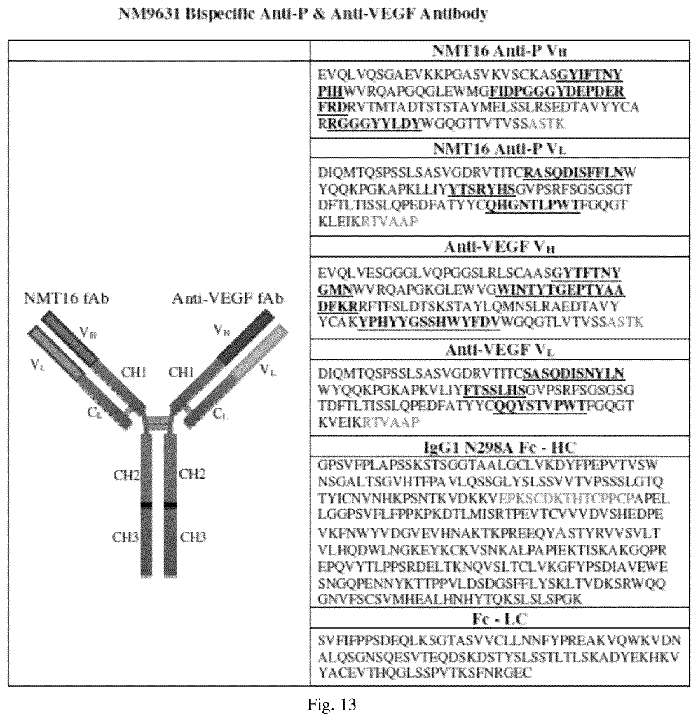

NM9631 is a bispecific antibody that binds both properdin and VEGF-A. A full sequence listing and the antibody structure can be found in FIG. 13.

TABLE 6
Half-Lif Fc NM5072
Heavy Chain

| | |
|---|---|
| Fc-CH | GPSVFPLAPS SKSTSGGTAA LGCLVKDYFP EPVTVSWNSG |
| | ALTSGVHTFP AVLQSSGLYS LSSVVTVPSS SLGTQTYICN |
| | VNHKPSNTKV DKKVEPKSCD KTHTCPPCPA PELLGGPSVF |
| | LFPPKPKDTL MISRTPEVTC VVVDVSHEDP EVKFNWYVDG |
| | VEVHNAKTKP REEQYNSTYR VVSVLTVLHQ |
| | DWLNGKEYKC KVSNKALPAP IEKTISKAKG QPREPQVYTL |
| | PPSRDELTKN QVSLTCLVKG FYPSDIAVEW ESNGQPENNY |
| | KTTPPVLDSD GSFFLYSKLT VDKSRWQQGN VFSCSVMHEA |
| | LHNHYTQKSL SLSPGK (SEQ ID NO: 129) |
| Fc-CH | GPSVFPLAPS SKSTSGGTAA LGCLVKDYFP EPVTVSWNSG |
| | ALTSGVHTFP AVLQSSGLYS LSSVVTVPSS SLGTQTYICN |
| | VNHKPSNTKV DKKVEPKSCD KTHTCPPCPA PELLGGPSVF |
| | LFPPKPKDTL MISRTPEVTC VVVDVSHEDP EVKFNWYVDG |
| | VEVHNAKTKP REEQYASTYR VVSVLTVLHQ |
| | DWLNGKEYKC KVSNKALPAP IEKTISKAKG QPREPQVYTL |
| | PPSRDELTKN QVSLTCLVKG FYPSDIAVEW ESNGQPENNY |
| | KTTPPVLDSD GSFFLYSKLT VDKSRWQQGN VFSCSVMHEA |
| | LHNHYTQKSL SLSPGK (SEQ ID NO: 130) |
| Fc-CH | GPSVFPLAPS SKSTSGGTAA LGCLVKDYFP EPVTVSWNSG |
| | ALTSGVHTFP AVLQSSGLYS LSSVVTVPSS SLGTQTYICN |
| | VNHKPSNTKV DKKVEPKSCD KTHTCPPCPA PELLGGPSVF |
| | LFPPKPKDTL YITREPEVTC VVVDVSHEDP EVKFNWYVDG |
| | VEVHNAKTKP REEQYASTYR VVSVLTVLHQ |
| | DWLNGKEYKC KVSNKALPAP IEKTISKAKG QPREPQVYTL |
| | PPSRDELTKN QVSLTCLVKG FYPSDIAVEW ESNGQPENNY |
| | KTTPPVLDSD GSFFLYSKLT VDKSRWQQGN VFSCSVMHEA |
| | LHNHYTQKSL SLSPGK (SEQ ID NO: 131) |
| Fc-CH | GPSVFPLAPC SRSTSESTAA LGCLVKDYFP EPVTVSWNSG |
| | ALTSGVHTFP AVLQSSGLYS LSSVVTVPSS NFGTQTYTCN |
| | VDHKPSNTKV DKTVERKCCV ECPPCPAPPV AGPSVFLFPP |
| | KPKDTLMISR TPEVTCVVVD VSQEDPEVQF NWYVDGVEVH |
| | NAKTKPREEQ FNSTYRVVSV LTVLHQDWLN |
| | GKEYKCKVSN KGLPSSIEKT ISKAKGQPRE PQVYTLPPSQ |
| | EEMTKNQVSL TCLVKGFYPS DIAVEWESNG QPENNYKTTP |
| | PVLDSDGSFF LYSRLTVDKS RWQEGNVFSC SVLHEALHSH |
| | YTQKSLSLSL GK (SEQ ID NO: 132) |
| Fc-CH | GPSVFPLAPC SRSTSESTAA LGCLVKDYFP EPVTVSWNSG |
| | ALTSGVHTFP AVLQSSGLYS LSSVVTVPSS NFGTQTYTCN |
| | VDHKPSNTKV DKTVERKCCV ECPPCPAPPV AGPSVFLFPP |
| | KPKDTLMISR TPEVTCVVVD VSQEDPEVQF NWYVDGVEVH |
| | NAKTKPREEQ FNSTYRVVSV LTVLHQDWLN |
| | GKEYKCKVSN KGLPSSIEKT ISKAKGQPRE PQVYTLPPSQ |
| | EEMTKNQVSL TCLVKGFYPS DIAVEWESNG QPENNYKTTP |
| | PVLDSDGSFF LYSRLTVDKS RWQEGNVFSC SVMHEALHNH |
| | YTQKSLSLSL GK (SEQ ID NO: 133) |
| IgG1 | EPKSCDKTHT CPPCPAPELL GGPSVFLFPP KPKDTLMISR |
| Fc-CH | TPEVTCVVVD VSHEDPEVKF NWYVDGVEVH |
| | NAKTKPREEQ YNSTYRVVSV LTVLHQDWLN |
| | GKEYKCKVSN KALPAPIEKT ISKAKGQPRE PQVYTLPPSR |
| | DELTKNQVSL TCLVKGFYPS DIAVEWESNG QPENNYKTTP |
| | PVLDSDGSFF LYSKLTVDKS RWQQGNVFSC SVMHEALHNH |
| | YTQKSLSLSP GK (SEQ ID NO: 134) |
| IgG2 | ERKCCVECPP CPAPPVAGPS VFLFPPKPKD TLMISRTPEV |
| Fc-CH | TCVVVDVSHE DPEVQFNWYV DGMEVHNAKT |
| | KPREEQFNST FRVVSVLTVV HQDWLNGKEY KCKVSNKGLP |
| | APIEKTISKT KGQPREPQVY TLPPSREEMT KNQVSLTCLV |
| | KGFYPSDIAV EWESNGQPEN NYKTTPPMLD SDGSFFLYSK |
| | LTVDKSRWQQ GNVFSCSVMH EALHNHYTQK SLSLSPGK |
| | (SEQ ID NO: 135) |
| IgG4 | ESKYGPPCPS CPAPEFLGGP SVFLFPPKPK DTLMISRTPE |
| Fc-CH | VTCVVVDVSQ EDPEVQFNWY VDGVEVHNAK |
| | TKPREEQFNS TYRVVSVLTV LHQDWLNGKE |
| | YKCKVSNKGL PSSIEKTISK AKGQPREPQV YTLPPSQEEM |
| | TKNQVSLTCL VKGFYPSDIA VEWESNGQPE NNYKTTPPVL |
| | DSDGSFFLYS RLTVDKSRWQ EGNVFSCSVM HEALHNHYTQ |
| | KSLSLSLGK (SEQ ID NO: 136) |
| Fc | AESKYGPPCP PCPAPEAAGG PSVFLFPPKP KDTLMISRTP |
| (IgG4) | EVTCVVVDVS QEDPEVQFNW YVDGVEVHNA |
| | KTKPREEQFN STYRVVSVLT VLHQDWLNGK |
| | EYKCKVSNKG LPSSIEKTIS KAKGQPREPQ VYTLPPSQEE |
| | MTKNQVSLTC LVKGFYPSDIAVEWESNGQP ENNYKTTPPV |
| | LDSDGSFFLY SRLTVDKSRW QEGNVFSCSV MHEALHNHYT |
| | QKSLSLSLG (SEQ ID NO: 137) |
| Fc-CH1 | GPSVFPLAPS SKSTSGGTAA LGCLVKDYFP EPVTVSWNSG |
| | ALTSGVHTFP AVLQSSGLYS LSSVVTVPSS SLGTQTYICN |
| | VNHKPSNTKV DKKVEPKSCD (SEQ ID NO: 138) |

Light Chain

| | |
|---|---|
| Fc-LC | SVFIFPPSDE QLKSGTASVV CLLNNFYPRE AKVQWKVDNA |
| | LQSGNSQESV TEQDSKDSTY SLSSTLTLSK ADYEKHKVYA |
| | CEVTHQGLSS PVTKSFNRGE C (SEQ ID NO: 139 |

TABLE 7
LINKERS

| | |
|---|---|
| G4S | GGGGS (SEQ ID NO: 140) |
| G3SG | GGGSG (SEQ ID NO: 141) |
| SG4 | SGGG (SEQ ID NO: 142) |
| (G4S)3 | GGGGSGGGGS GGGGS (SEQ ID NO: 143) |
| (G4D)3 | GGGGDGGGGD GGGG (SEQ ID NO: 144) |
| G4E | GGGGEGGGGE GGGG (SEQ ID NO: 145) |
| G3D | GGGE (SEQ ID NO: 146) |
| G3E | GGGD (SEQ ID NO: 147) |
| G4A | GGGGA (SEQ ID NO: 148) |
| G4A/S | GGGGAGGGGA GGGGS (SEQ ID NO: 149) |

TABLE 8
Anti-TNF Anti VEGF fAb

| | |
|---|---|
| Fv-(VH Anti-TNF) | EVQLVESGGG LVQPGRSLRL SCAASGFTFD |
| | DYAMHWVRQA PGKGLEWVSA ITWNSGHIDY |
| | ADSVEGRFTI SRDNAKNSLY LQMNSLRAED TAVYYCAKVS |
| | YLSTASSLDY WGQGTLVTVS SASTK (SEQ ID No: 150) |
| Fv-(VL Anti-TNF) | DIQMTQSPSS LSASVGDRVT ITCRASQGIR NYLAWYQQKP |
| | GKAPKLLIYA ASTLQSGVPS RFSGSGSGTD FTLTISSLQP |
| | EDVATYYCQR YNRAPYTFGQ GTKVEIKRTV AAP (SEQ ID |
| | NO: 151) |
| Fv-(VH Anti-VEGF) | EVQLVESGGG LVQPGGSLRL SCAASGYTFT |
| | NYGMNWVRQA PGKGLEWVGW INTYTGEPTY |
| | AADFKRRFTF SLDTSKSTAY LQMNSLRAED TAVYYCAKYP |
| | HYYGSSHWYF DVWGQGTLVT VSSASTK (SEQ ID NO: 152 |
| Fv-(VL Anti-VEGF) | DIQMTQSPSS LSASVGDRVT ITCSASQDIS NYLNWYQQKP |
| | GKAPKVLIYF TSSLHSGVPS RFSGSGSGTD FTLTISSLQP |
| | EDFATYYCQQ YSTVPWTFGQ GTKVEIKRTV AAP (SEQ ID |
| | NO: 153) |

### Example 7

Example 7 describes the structure and amino acid sequences of antibodies that were modified and reconstructed from Example 5 and Example 6. Specifically, NMT1003, the (fAb)2 monospecific antibody from Example 5 is listed along with a couple bispecific fAb regions from Example 6 (Anti-P/Anti-Alb, Anti-P/Anti-TNF, Anti-P/Anti-VEGF). The main difference is the variations of the Fc's used to attach to these heavy chain regions. By varying the Fc, the ability to get monospecific and bispecific antibodies with better binding, half-life, and circulation time can be generated. The table below shows the list of antibodies that have been developed for Example 7.

| | |
|---|---|
| 1 | Monospecific NMT15 Anti-P (fAb)₂ with CH1 Fc Only |
| 2 | Bispecific Camelid Anti-P - Anti-Alb w/IgG1 N297A Fc |
| 3 | Bispecific NMT16 Anti-P - Anti-Alb w/IgG1 YTE Fc |
| 4 | Bispecific NMT16 Anti-P - HumRab Anti-Alb w/IgG1 N297A Fc |
| 5 | Bispecific Camelid Anti-P - ALXN Anti-Alb w/IgG4 (CH2/CH3 |
| | only) |
| 6 | Bispecific NMT16 Anti-P - Anti-TNF w/IgG1 YTE Fc |
| 7 | Bispecific NMT16 Anti-P - Anti-VEGF w/IgG1 YTE Fc |

FIG. 7 shows a monospecific antibody that only binds to Properdin. This antibody contains NMT15 for its variable heavy and light chains (fAb). The variable heavy chain is attached to Fc CH1 region only. The light chain is attached to the Fc CL. The two identical NMT15 fAb regions will be joined together with a (G₄S)₃ linker. A structure of the antibody is presented as well with the two ovals representing the NMT15 fAb regions along with the CH1 and CL regions. The green line between the two represents the linker joining them together.

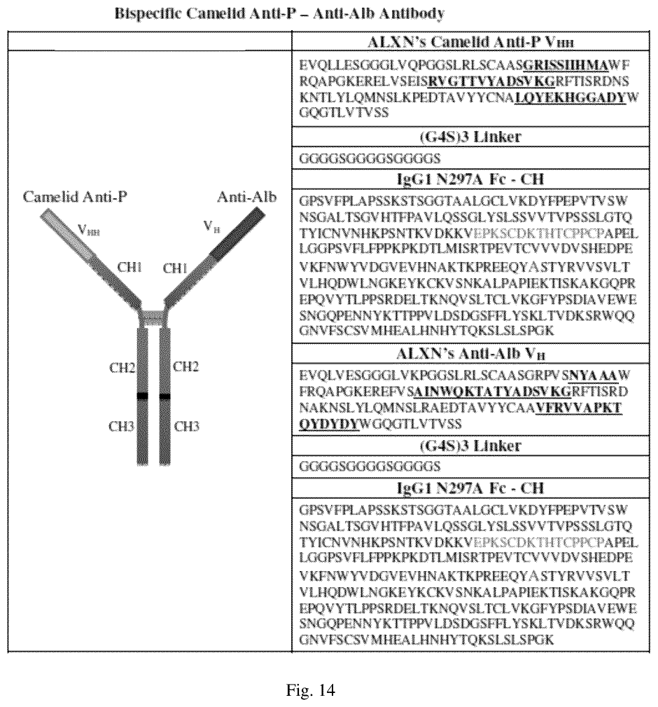

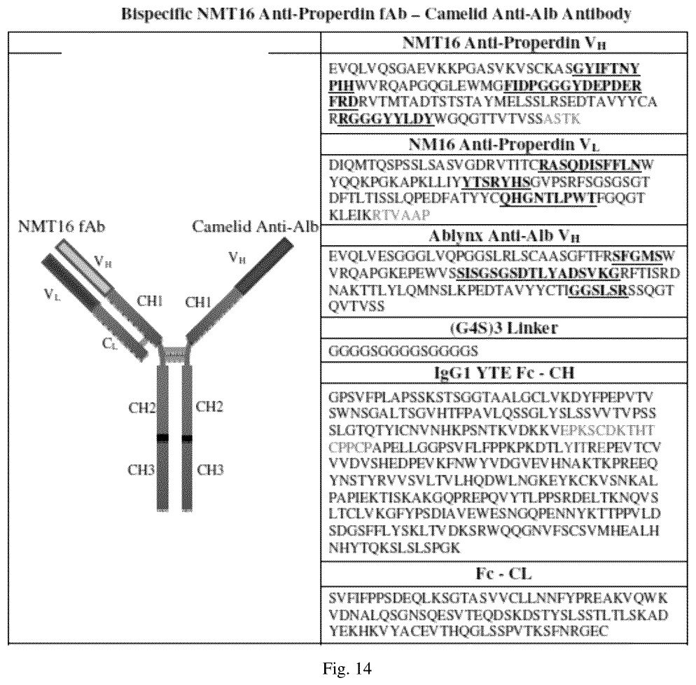

FIG. 14 illustrates the sequences and structure of a bispecific antibody that binds to both properdin and serum albumin. Both the anti-properdin and anti-albumin variable heavy chain sequences are derived from camelid species. Additionally, both are attached the an IgG1 full Fc with a N297A mutation (a mutation of Aparagine to Alanine at position 297). However, this position is subject to change depending on the variable heavy chain it is attached to. A (G4S)3 linker is used to connect the variable chains to the constant chains.

FIG. 15 shows the sequences and structure of a bispecific antibody that binds to both properdin and serum albumin. NMT16 variable heavy and light chains are combined with a camelid Anti-Alb single variable heavy chain. NMT16's variable heavy chain region is attached to an IgG1 Fc with a YTE mutation to help induce a longer half-life. Ablynx's camelid anti-alb is attached to the same Fc using a (G4S)3 linker. NMT16's light chain is attached to the CL Fc.

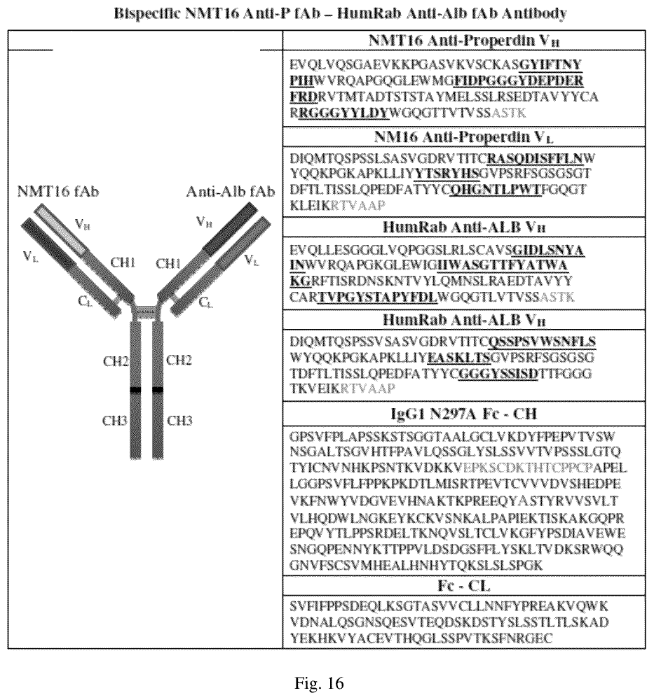

FIG. 16 shows the sequences and structure of a bispecific antibody that binds to both properdin and serum albumin. NMT16 Anti-P fAb region is combined with humanized rabbit serum albumin. This is similar to the NM9628 construct from Example 6. The difference is that the heavy chain regions for NMT16 and Anti-ALB are joined to a IgG1 N297A Fc (mutation might not always occur at position 297, it depends on the VH to which it is attached). The light chain regions for NMT16 and Anti-ALB are joined to the CL.

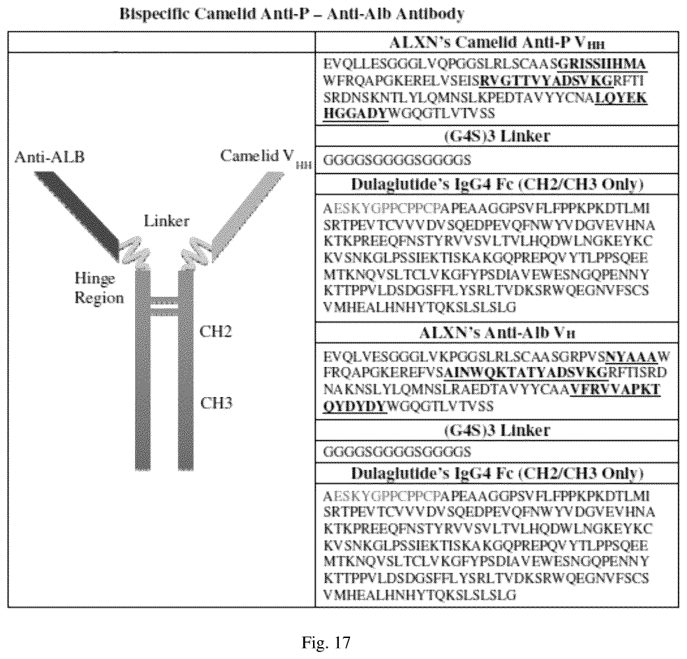

FIG. 17 shows the sequences and structure of a bispecific antibody that binds both properdin and serum albumin. A camelid anti-properdin single variable heavy chain is combined with camelid anti-alb single heavy chain. These two variable heavy chain regions are joined to Dulaglutide's IgG4 Fc (CH2/CH3 regions only) with a (G4S)3 linker.

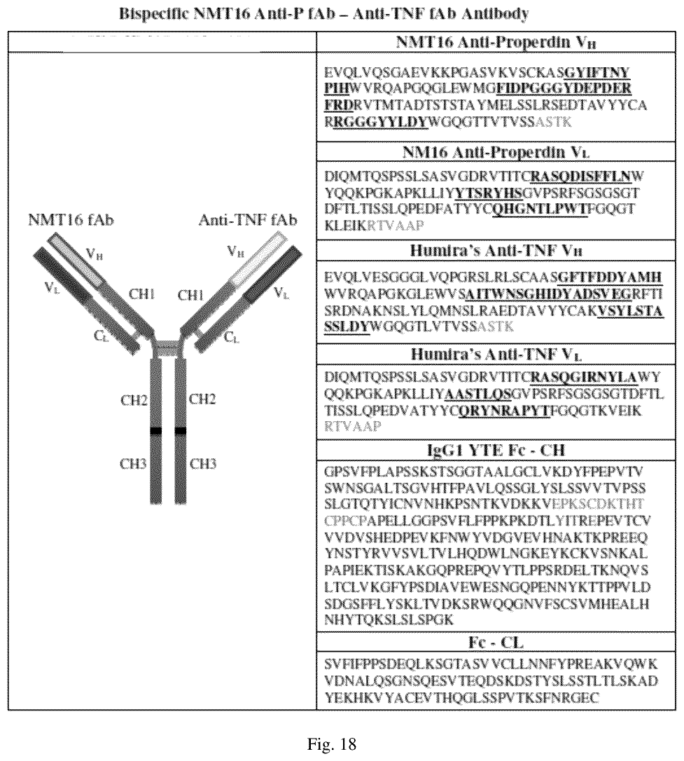

FIG. 18 shows the sequences and structure of a bispecific antibody that binds both properdin and TNF. This is similar to Example 6's NM9630. NMT16 fAb is combined with an anti-TNF fAb. The difference is that the variable heavy chain sequences are connected to an IgG1 YTE Fc. The light chains are attached to the CL.

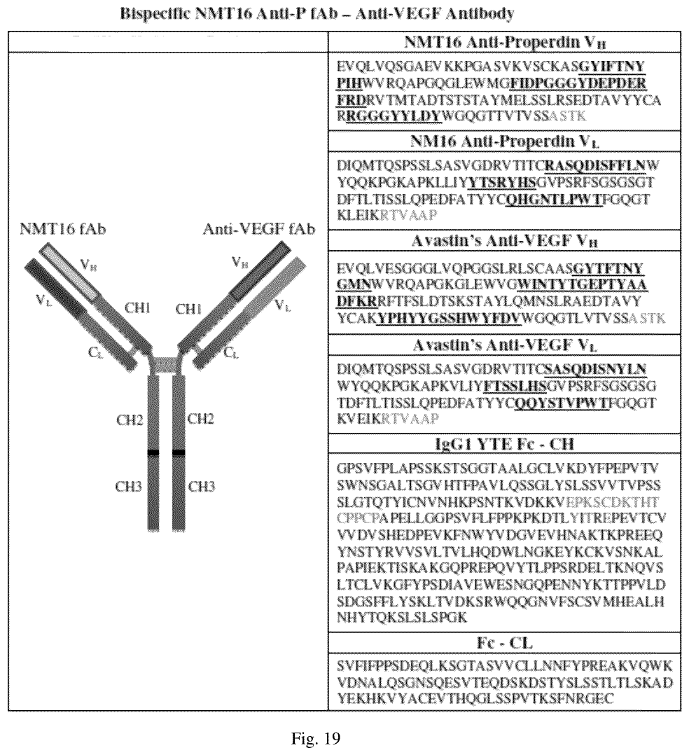

FIG. 19 shows the sequences and structure of a bispecific antibody that binds both properdin and VEGF-A. This is similar to Example 6's NM9631 construct. NMT16 fAb is combined with an anti-VEGF-A fAb. The difference is that the heavy chains are to an IgG1 YTE Fc, just like the bispecific construct above. The light chains are connected to the CL.

Using the Presta substitutions, multiple different combinations of variable chain sequences can be generated. These sequences can be utilized to formulate and test how different substitutions can affect the affinity, hemolysis, and potency of different antibody structures. Additionally, the ability to create bispecific molecules using anti-Alb, anti-TNF, anti-VEGF, and the mutation of the IgG Fc's can help increase the half-life and circulation time of these antibodies in vivo.

Amino acid sequences of these anti-properdin antibodies and other monospecific and bispecific anti-properdin antibodies are listed in Tables 9, 10, and 11.

TABLE 9
Full Chain SEQ ID NOs

| | |
|---|---|
| NMT 1001-Single | EVQLVESGGG LVKPGGSLRL SCAASGRPVS NYAAAWFRQA |
| Chain mAb | PGKEREFVSA INWQKTATYA DSVKGRFTIS RDNAKNSLYL |
| Anti-Alb/Camelid | QMNSLRAEDT AVYYCAAVFR VVAPKTQYDY |
| Anti-P (LPV058) | DYWGQGTLVT VSSGGGSGGG SGGGSEVQLV |
| | ESGGGLVQPG GSLRLSCAAS GRISSIIHMA WVRQAPGKQR |
| | ELVSEISRVG TTVYADSVKG RFTISRDNSK NTLYLQMNSL |
| | RAEDTAVYYC NALQYEKHGG ADYWGQGTLV TVSS (SEQ |
| | ID NO: 154) |
| NMT 1002-Single | EVQLVESGGG LVQPGGSLRL SCAASGFTFR SFGMSWVRQA |
| Chain mAb | PGKEPEWVSS ISGSGSDTLY ADSVKGRFTI SRDNAKTTLY |
| Anti-Alb/Camelid | LQMNSLKPED TAVYYCTIGG SLSRSSQGTQ VTVSSGGGSG |
| Anti-P (LPV058) | GGSGGGSEVQ LVESGGGLVQ PGGSLRLSCA ASGRISSIIH |
| | MAWVRQAPGK QRELVSEISR VGTTVYADSV KGRFTISRDN |
| | SKNTLYLQMN SLRAEDTAVY YCNALQYEKH |
| | GGADYWGQGT LVTVSS (SEQ ID NO: 155) |
| NMT 1003-(Fab)2 | QVQLVQSAPE VAKPGTSVKM SCKASGYIFT NYPIHWVKQA |
| Fv (VH NMT15) | PGQGLEWIGF IDPGGGYDEP DERFRDRATL TADKSTSTAY |
| Fc-CH1 | MELSSLRSED TAIYYCARRG GGYYLDYWGQ GTLVTVSSAS |
| | TKGPSVFPLA PSSKSTSGGT AALGCLVKDY FPEPVTVSWN |
| | SGALTSGVHT FPAVLQSSGL YSLSSVVTVP SSSLGTQTYI |
| | CNVNHKPSNT KVDKKVEPKS CD (SEQ ID NO: 156) |
| NMT-1003-(Fab)2 | DIQMTQSPSS LSASLGDRVT ITCRASQDIS FFLNWYQQKP |
| Fv (VL NMT15) | DGTVKLLIYY TSRYHSGVPS RFSGSGSGTD FTLTISSLQP |
| Fc-CL | EDFATYFCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 157) |
| NMT1004-NMT15 fAb- | QVQLVQSAPE VAKPGTSVKM SCKASGYIFT NYPIHWVKQA |
| Camelid Anti-Alb | PGQGLEWIGF IDPGGGYDEP DERFRDRATL TADKSTSTAY |
| Fv (VHNMT15) | MELSSLRSED TAIYYCARRG GGYYLDYWGQ GTLVTVSSAS |
| Fc-CH1 | TKGPSVFPLA PSSKSTSGGT AALGCLVKDY FPEPVTVSWN |
| (G4S)3 Linker | SGALTSGVHT FPAVLQSSGL YSLSSVVTVP SSSLGTQTYI |
| Hum Anti-Alb | CNVNHKPSNT KVDKKVEPKS CDGGGGSGGG |
| | GSGGGGSEVQ LVESGGGLVK PGGSLRLSCA ASGRPVSNYA |
| | AAWFRQAPGK EREFVSAINW QKTATYADSV KGRFTISRDN |
| | AKNSLYLQMN SLRAEDTAVY YCAAVFRVVA |
| | PKTQYDYDYW GQGTLVTVSS (SEQ ID NO: 158) |
| Fv (VL NMT15) | DIQMTQSPSS LSASLGDRVT ITCRASQDIS FFLNWYQQKP |
| Fc-CL | DGTVKLLIYY TSRYHSGVPS RFSGSGSGTD FTLTISSLQP |
| | EDFATYFCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 159) |
| NMT1005-NMT15 fAb- | QVQLVQSAPE VAKPGTSVKM SCKASGYIFT NYPIHWVKQA |
| Camelid Anti-Alb | PGQGLEWIGF IDPGGGYDEP DERFRDRATL TADKSTSTAY |
| Fv (VHNMT15) | MELSSLRSED TAIYYCARRG GGYYLDYWGQ GTLVTVSSAS |
| Fc-CH1 | TKGPSVFPLA PSSKSTSGGT AALGCLVKDY FPEPVTVSWN |
| (G4S)3 Linker | SGALTSGVHT FPAVLQSSGL YSLSSVVTVP SSSLGTQTYI |
| Human-Anti-Alb | CNVNHKPSNT KVDKKVEPKS CDGGGGSGGG |
| | GSGGGGSEVQ LVESGGGLVQ PGGSLRLSCA ASGFTFRSFG |
| | MSWVRQAPGK EPEWVSSISG SGSDTLYADS VKGRFTISRD |
| | NAKTTLYLQM NSLKPEDTAV YYCTIGGSLS RSSQGTQVTV |
| | SS (SEQ ID NO: 160) |
| Fv (VL NMT15) | DIQMTQSPSS LSASLGDRVT ITCRASQDIS FFLNWYQQKP |
| | DGTVKLLIYY TSRYHSGVPS RFSGSGSGTD FTLTISSLQP |
| | EDFATYFCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 161) |

TABLE 10
Full Chain SEQ ID NOs

| | |
|---|---|
| NM9625-Camelid | EVQLLESGGG LVQPGGSLRL SCAASGRISS IIHMAWFRQA |
| Anti-P w/ IgG4 | PGKERELVSE ISRVGTTVYA DSVKGRFTIS RDNSKNTLYL |
| (fAb)2 Camelid | QMNSLKPEDT AVYYCNALQY EKHGGADYWG |
| (Anti-P) Linker | QGTLVTVSSG GGGSGGGGSG GGGSAESKYG |
| Fc (IgG4) | PPCPPCPAPE AAGGPSVFLF PPKPKDTLMI SRTPEVTCVV |
| | VDVSQEDPEV QFNWYVDGVE VHNAKTKPRE |
| | EQFNSTYRVV SVLTVLHQDW LNGKEYKCKV |
| | SNKGLPSSIE KTISKAKGQP REPQVYTLPP SQEEMTKNQV |
| | SLTCLVKGFY PSDIAVEWES NGQPENNYKT TPPVLDSDGS |
| | FFLYSRLTVD KSRWQEGNVF SCSVMHEALH |
| | NHYTQKSLSL SLG (SEQ ID NO: 162) |
| NM9626-Camelid | EVQLLESGGG LVQPGGSLRL SCAASGRISS IIHMAWFRQA |
| Anti-P-Anti-Alb | PGKERELVSE ISRVGTTVYA DSVKGRFTIS RDNSKNTLYL |
| w/ IgG4 Camelid | QMNSLKPEDT AVYYCNALQY EKHGGADYWG |
| (Anti-P) | QGTLVTVSSG GGGSGGGGSG GGGSAESKYG |
| (G4S)3 Linker | PPCPPCPAPE AAGGPSVFLF PPKPKDTLMI SRTPEVTCVV |
| Fc (IgG4) | VDVSQEDPEV QFNWYVDGVE VHNAKTKPRE |
| | EQFNSTYRVV SVLTVLHQDW LNGKEYKCKV |
| | SNKGLPSSIE KTISKAKGQP REPQVYTLPP SQEEMTKNQV |
| | SLTCLVKGFY PSDIAVEWES NGQPENNYKT TPPVLDSDGS |
| | FFLYSRLTVD KSRWQEGNVF SCSVMHEALH |
| | NHYTQKSLSL SLG (SEQ ID NO: 162) |
| Anti-ALB | EVQLVESGGG LVKPGGSLRL SCAASGRPVS |
| (G4S)3 Linker | NYAAAWFRQA PGKEREFVSA INWQKTATYA |
| Fc (IgG4) | DSVKGRFTIS RDNAKNSLYL QMNSLRAEDT |
| | AVYYCAAVFR VVAPKTQYDY DYWGQGTLVT |
| | VSSGGGGSGG GGSGGGGSAE SKYGPPCPPC |
| | PAPEAAGGPS VFLFPPKPKD TLMISRTPEV TCVVVDVSQE |
| | DPEVQFNWYV DGVEVHNAKT KPREEQFNST |
| | YRVVSVLTVL HQDWLNGKEY KCKVSNKGLP |
| | SSIEKTISKA KGQPREPQVY TLPPSQEEMT KNQVSLTCLV |
| | KGFYPSDIAV EWESNGQPEN NYKTTPPVLD SDGSFFLYSR |
| | LTVDKSRWQE GNVFSCSVMH EALHNHYTQK SLSLSLG |
| | (SEQ ID NO: 163) |
| NM9627-Camelid | EVQLLESGGG LVQPGGSLRL SCAASGRISS IIHMAWFRQA |
| Anti-P-Anti-Alb | PGKERELVSE ISRVGTTVYA DSVKGRFTIS RDNSKNTLYL |
| w/ IgG1 Camelid | QMNSLKPEDT AVYYCNALQY EKHGGADYWG |
| (Anti-P) (G4S)3 | QGTLVTVSSG GGGSGGGGSG GGGSEPKSCD |
| Linker IgG1-Fc | KTHTCPPCPA PELLGGPSVF LFPPKPKDTL MISRTPEVTC |
| | VWDVSHEDP EVKFNWYVDG VEVHNAKTKP |
| | REEQYNSTYR VVSVLTVLHQ DWLNGKEYKC |
| | KVSNKALPAP IEKTISKAKG QPREPQVYTL PPSRDELTKN |
| | QVSLTCLVKG FYPSDIAVEW ESNGQPENNY KTTPPVLDSD |
| | GSFFLYSKLT VDKSRWQQGN VFSCSVMHEA |
| | LHNHYTQKSL SLSPGK (SEQ ID NO: 164) |
| Anti-ALB | EVQLVESGGG LVKPGGSLRL SCAASGRPVS |
| (G4S)3 Linker | NYAAAWFRQA PGKEREFVSA INWQKTATYA |
| IgG1-Fc | DSVKGRFTIS RDNAKNSLYL QMNSLRAEDT |
| | AVYYCAAVFR VVAPKTQYDY DYWGQGTLVT |
| | VSSGGGGSGG GGSGGGGSEP KSCDKTHTCP |
| | PCPAPELLGG PSVFLFPPKP KDTLMISRTP EVTCVVVDVS |
| | HEDPEVKFNW YVDGVEVHNA KTKPREEQYN |
| | STYRVVSVLT VLHQDWLNGK EYKCKVSNKA |
| | LPAPIEKTIS KAKGQPREPQ VYTLPPSRDE LTKNQVSLTC |
| | LVKGFYPSDI AVEWESNGQP ENNYKTTPPV LDSDGSFFLY |
| | SKLTVDKSRW QQGNVFSCSV MHEALHNHYT |
| | QKSLSLSPGK (SEQ ID NO: 165) |
| NM9628-NMT16 fAb- | EVQLVQSGAE VKKPGASVKV SCKASGYIFT |
| LIT HumRab Anti-ALB | NYPIHWVRQA PGQGLEWMGF IDPGGGYDEP |
| fAb w/ IgG1 | DERFRDRVTM TADTSTSTAY MELSSLRSED |
| Fv-(VH NMT16) | TAVYYCARRG GGYYLDYWGQ GTTVTVSSAS |
| Fc-IgG1 | TKGPSVFPLA PCSRSTSEST AALGCLVKDY FPEPVTVSWN |
| | SGALTSGVHT FPAVLQSSGL YSLSSVVTVP SSNFGTQTYT |
| | CNVDHKPSNT KVDKTVEPKS CDKTHTCPPC PAPELLGGPS |
| | VFLFPPKPKD TLMISRTPEV TCVVVDVSHE |
| | DPEVKFNWYV DGVEVHNAKT KPREEQYNST |
| | YRVVSVLTVL HQDWLNGKEY KCKVSNKALP |
| | APIEKTISKA KGQPREPQVY TLPPSRDELT KNQVSLTCLV |
| | KGFYPSDIAV EWESNGQPEN NYKTTPPVLD SDGSFFLYSK |
| | LTVDKSRWQQ GNVFSCSVMH EALHNHYTQK SLSLSPGK |
| | (SEQ ID NO: 166) |
| Fv-(VL NMT16) | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 167) |
| Fv-(VH HumRab | EVQLLESGGG LVQPGGSLRL SCAVSGIDLS |
| Anti-ALB) | NYAINWVRQA PGKGLEWIGIIWASGTTFYA |
| Fc-IgG1 | TWAKGRFTIS RDNSKNTVYL QMNSLRAEDT |
| | AVYYCARTVP GYSTAPYFDL WGQGTLVTVS |
| | SASTKGPSVF PLAPCSRSTS ESTAALGCLV KDYFPEPVTV |
| | SWNSGALTSG VHTFPAVLQS SGLYSLSSVV TVPSSNFGTQ |
| | TYTCNVDHKP SNTKVDKTVE PKSCDKTHTC |
| | PPCPAPELLG GPSVFLFPPK PKDTLMISRT PEVTCVVVDV |
| | SHEDPEVKFN WYVDGVEVHN AKTKPREEQY |
| | NSTYRVVSVL TVLHQDWLNG KEYKCKVSNK |
| | ALPAPIEKTI SKAKGQPREP QVYTLPPSRD ELTKNQVSLT |
| | CLVKGFYPSD IAVEWESNGQ PENNYKTTPP VLDSDGSFFL |
| | YSKLTVDKSR WQQGNVFSCS VMHEALHNHY |
| | TQKSLSLSPG K (SEQ ID NO: 168) |
| Fv-(VL HumRab | DIQMTQSPSS VSASVGDRVT ITCQSSPSVW SNFLSWYQQK |
| Anti-ALB) | PGKAPKLLIY EASKLTSGVP SRFSGSGSGT DFTLTISSLQ |
| Fc-CL | PEDFATYYCG GGYSSISDTT FGGGTKVEIK RTVAAPSVFI |
| | FPPSDEQLKS GTASVVCLLN NFYPREAKVQ |
| | WKVDNALQSG NSQESVTEQD SKDSTYSLSS |
| | TLTLSKADYE KHKVYACEVT HQGLSSPVTK SFNRGEC |
| | (SEQ ID NO: 169) |
| NM9629-NMT HumRab | EVQLLESGGG LVQPGGSLRL SCAASGFSFS SGYWIFWVRQ |
| Anti-P-LIT HumRab | APGKGLELVG GIYSGSSGTT YYADSVKGRF TISKDNSKNT |
| Anti-ALB fAb w/ | VYLQMNSLRA EDTAVYYCAR SVDGIDSYDA |
| IgG1 YTE | AFNLWGQGTL VTVSSASTKG PSVFPLAPSS KSTSGGTAAL |
| Fv-(VH HumRab | GCLVKDYFPE PVTVSWNSGA LTSGVHTFPA |
| 2703 H2) | VLQSSGLYSL SSVVTVPSSS LGTQTYICNV NHKPSNTKVD |
| Fc-IgG1 YTE | KKVEPKSCDK THTCPPCPAP ELLGGPSVFL FPPKPKDTLY |
| Mutation | ITREPEVTCV VVDVSHEDPE VKFNWYVDGV |
| | EVHNAKTKPR EEQYNSTYRV VSVLTVLHQD |
| | WLNGKEYKCK VSNKALPAPIEKTISKAKGQ PREPQVYTLP |
| | PSRDELTKNQ VSLTCLVKGF YPSDIAVEWE |
| | SNGQPENNYK TTPPVLDSDG SFFLYSKLTV |
| | DKSRWQQGNV FSCSVMHEAL HNHYTQKSLS LSPGK (SEQ |
| | ID NO: 170) |
| Fv-(VL HumRab | DIQLTQSPSS LSASVGDRVT ITCRASDNIY SLLAWYQQKP |
| 2703 L2) | GKAPKLLIYR ASTLASGVPS RFSGSGSGTD FTLTISSVQP |
| Fc-CL | EDFATYYCQQ HYDYNYLDVA FGGGTKVEIK |
| | RTVAAPSVFI FPPSDEQLKS GTASVVCLLN NFYPREAKVQ |
| | WKVDNALQSG NSQESVTEQD SKDSTYSLSS |
| | TLTLSKADYE KHKVYACEVT HQGLSSPVTK SFNRGEC |
| | (SEQ ID NO: 171) |
| Fv-(VH HumRab | DIQMTQSPSS VSASVGDRVT ITCQSSPSVW SNFLSWYQQK |
| Anti-ALB) | PGKAPKLLIY EASKLTSGVP SRFSGSGSGT DFTLTISSLQ |
| Fc-IgG1 YTE | PEDFATYYCG GGYSSISDTT FGGGTKVEIK RTVAAPSVFI |
| Mutation | FPPSDEQLKS GTASVVCLLN NFYPREAKVQ |
| | WKVDNALQSG NSQESVTEQD SKDSTYSLSS |
| | TLTLSKADYE KHKVYACEVT HQGLSSPVTK SFNRGEC |
| | (SEQ ID NO: 172) |
| Fv-(VL HumRab | DIQMTQSPSS VSASVGDRVT ITCQSSPSVW SNFLSWYQQK |
| Anti-ALB) | PGKAPKLLIY EASKLTSGVP SRFSGSGSGT DFTLTISSLQ |
| Fc-CL | PEDFATYYCG GGYSSISDTT FGGGTKVEIK RTVAAPSVFI |
| | FPPSDEQLKS GTASVVCLLN NFYPREAKVQ |
| | WKVDNALQSG NSQESVTEQD SKDSTYSLSS |
| | TLTLSKADYE KHKVYACEVT HQGLSSPVTK SFNRGEC |
| | (SEQ ID NO: 173) |
| NM9630-NMT16 fAb- | EVQLVQSGAE VKKPGASVKV SCKASGYIFT |
| Anti-TNF w/ IgG1 | NYPIHWVRQA PGQGLEWMGF IDPGGGYDEP |
| Fv-(VH NMT16) | DERFRDRVTM TADTSTSTAY MELSSLRSED |
| Fc-IgG1 | TAVYYCARRG GGYYLDYWGQ GTTVTVSSAS |
| | TKGPSVFPLA PSSKSTSGGT AALGCLVKDY FPEPVTVSWN |
| | SGALTSGVHT FPAVLQSSGL YSLSSVVTVP SSSLGTQTYI |
| | CNVNHKPSNT KVDKKVEPKS CDKTHTCPPC |
| | PAPELLGGPS VFLFPPKPKD TLMISRTPEV TCVVVDVSHE |
| | DPEVKFNWYV DGVEVHNAKT KPREEQYNST |
| | YRVVSVLTVL HQDWLNGKEY KCKVSNKALP |
| | APIEKTISKA KGQPREPQVY TLPPSRDELT KNQVSLTCLV |
| | KGFYPSDIAV EWESNGQPEN NYKTTPPVLD SDGSFFLYSK |
| | LTVDKSRWQQ GNVFSCSVMH EALHNHYTQK SLSLSPGK |
| | (SEQ ID NO: 174) |
| Fv-(VL NMT16) | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 175) |
| Fv-(VH Anti-TNF) | EVQLVESGGG LVQPGRSLRL SCAASGFTFD |
| Fc-IgG1 | DYAMHWVRQA PGKGLEWVSA ITWNSGHIDY |
| | ADSVEGRFTI SRDNAKNSLY LQMNSLRAED |
| | TAVYYCAKVS YLSTASSLDY WGQGTLVTVS |
| | SASTKGPSVF PLAPSSKSTS GGTAALGCLV KDYFPEPVTV |
| | SWNSGALTSG VHTFPAVLQS SGLYSLSSVV TVPSSSLGTQ |
| | TYICNVNHKP SNTKVDKKVE PKSCDKTHTC PPCPAPELLG |
| | GPSVFLFPPK PKDTLMISRT PEVTCVVVDV SHEDPEVKFN |
| | WYVDGVEVHN AKTKPREEQY NSTYRVVSVL |
| | TVLHQDWLNG KEYKCKVSNK ALPAPIEKTI |
| | SKAKGQPREP QVYTLPPSRD ELTKNQVSLT CLVKGFYPSD |
| | IAVEWESNGQ PENNYKTTPP VLDSDGSFFL YSKLTVDKSR |
| | WQQGNVFSCS VMHEALHNHY TQKSLSLSPG K (SEQ ID |
| | NO: 176) |
| Fv-(VL Anti-TNF) | DIQMTQSPSS LSASVGDRVT ITCRASQGIR NYLAWYQQKP |
| Fc-CL | GKAPKLLIYA ASTLQSGVPS RFSGSGSGTD FTLTISSLQP |
| | EDVATYYCQR YNRAPYTFGQ GTKVEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 177) |
| NM9631-NMT16fAb- | EVQLVQSGAE VKKPGASVKV SCKASGYIFT |
| Anti-VEGF w/ IgG1 | NYPIHWVRQA PGQGLEWMGF IDPGGGYDEP |
| N298A | DERFRDRVTM TADTSTSTAY MELSSLRSED |
| Fv-(VH NMT16) | TAVYYCARRG GGYYLDYWGQ GTTVTVSSAS |
| Fc-IgG1 N298A | TKGPSVFPLA PSSKSTSGGT AALGCLVKDY FPEPVTVSWN |
| | SGALTSGVHT FPAVLQSSGL YSLSSVVTVP SSSLGTQTYI |
| | CNVNHKPSNT KVDKKVEPKS CDKTHTCPPC |
| | PAPELLGGPS VFLFPPKPKD TLMISRTPEV TCVVVDVSHE |
| | DPEVKFNWYV DGVEVHNAKT KPREEQYAST |
| | YRVVSVLTVL HQDWLNGKEY KCKVSNKALP |
| | APIEKTISKA KGQPREPQVY TLPPSRDELT KNQVSLTCLV |
| | KGFYPSDIAV EWESNGQPEN NYKTTPPVLD SDGSFFLYSK |
| | LTVDKSRWQQ GNVFSCSVMH EALHNHYTQK SLSLSPGK |
| | (SEQ ID NO: 178) |
| Fv-(VL NMT16) | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 179) |
| Fv-(VH Anti-VEGF) | EVQLVESGGG LVQPGGSLRL SCAASGYTFT |
| Fc-IgG1 N298A | NYGMNWVRQA PGKGLEWVGW INTYTGEPTY |
| | AADFKRRFTF SLDTSKSTAY LQMNSLRAED |
| | TAVYYCAKYP HYYGSSHWYF DVWGQGTLVT |
| | VSSASTKGPS VFPLAPSSKS TSGGTAALGC LVKDYFPEPV |
| | TVSWNSGALT SGVHTFPAVL QSSGLYSLSS VVTVPSSSLG |
| | TQTYICNVNH KPSNTKVDKK VEPKSCDKTH TCPPCPAPEL |
| | LGGPSVFLFP PKPKDTLMIS RTPEVTCVVV DVSHEDPEVK |
| | FNWYVDGVEV HNAKTKPREE QYASTYRVVS |
| | VLTVLHQDWL NGKEYKCKVS NKALPAPIEK |
| | TISKAKGQPR EPQVYTLPPS RDELTKNQVS LTCLVKGFYP |
| | SDIAVEWESN GQPENNYKTT PPVLDSDGSF FLYSKLTVDK |
| | SRWQQGNVFS CSVMHEALHN HYTQKSLSLS PGK (SEQ ID |
| | NO: 180) |
| Fv-(VL Anti-VEGF) | DIQMTQSPSS LSASVGDRVT ITCSASQDIS NYLNWYQQKP |
| Fc-CL | GKAPKVLIYF TSSLHSGVPS RFSGSGSGTD FTLTISSLQP |
| | EDFATYYCQQ YSTVPWTFGQ GTKVEIKRTV AAPSVFIFPP |
| | SDEQLKSGTA SVVCLLNNFY PREAKVQWKV |
| | DNALQSGNSQ ESVTEQDSKD STYSLSSTLT LSKADYEKHK |
| | VYACEVTHQG LSSPVTKSFN RGEC (SEQ ID NO: 181) |

TABLE 11
Full Chain SEQ ID NOs

| | |
|---|---|
| Anti-P-Anti-Alb w/ | EVQLVESGGG LVQPGGSLRL SCAASGRISS |
| N297A Fc | IIHMAWVRQA PGKQRELVSE ISRVGTTVYA |
| Camelid Anti-P-VHH | DSVKGRFTIS RDNSKNTLYL QMNSLRAEDT |
| Linker | AVYYCNALQY EKHGGADYWG QGTLVTVSSG |
| IgG1 N297A Fc-Full CH | GGGSGGGGSG GGGSGPSVFP LAPSSKSTSG |
| | GTAALGCLVK DYFPEPVTVS WNSGALTSGV |
| | HTFPAVLQSS GLYSLSSVVT VPSSSLGTQT YICNVNHKPS |
| | NTKVDKKVEP KSCDKTHTCP PCPAPELLGG PSVFLFPPKP |
| | KDTLMISRTP EVTCVVVDVS HEDPEVKFNW |
| | YVDGVEVHNA KTKPREEQYA STYRVVSVLT |
| | VLHQDWLNGK EYKCKVSNKA LPAPIEKTIS |
| | KAKGQPREPQ VYTLPPSRDE LTKNQVSLTC |
| | LVKGFYPSDI AVEWESNGQP ENNYKTTPPV |
| | LDSDGSFFLY SKLTVDKSRW QQGNVFSCSV |
| | MHEALHNHYT QKSLSLSPGK (SEQ ID NO: 182) |
| Anti-Alb | EVQLVESGGG LVKPGGSLRL SCAASGRPVS |
| Linker | NYAAAWFRQA PGKEREFVSA INWQKTATYA |
| IgG1 N297A Fc-Full CH | DSVKGRFTIS RDNAKNSLYL QMNSLRAEDT |
| | AVYYCAAVFR VVAPKTQYDY DYWGQGTLVT |
| | VSSGGGGSGG GGSGGGGSGP SVFPLAPSSK |
| | STSGGTAALG CLVKDYFPEP VTVSWNSGAL |
| | TSGVHTFPAV LQSSGLYSLS SVVTVPSSSL GTQTYICNVN |
| | HKPSNTKVDK KVEPKSCDKT HTCPPCPAPE |
| | LLGGPSVFLF PPKPKDTLMI SRTPEVTCVV VDVSHEDPEV |
| | KFNWYVDGVE VHNAKTKPRE EQYASTYRVV |
| | SVLTVLHQDW LNGKEYKCKV SNKALPAPIE |
| | KTISKAKGQP REPQVYTLPP SRDELTKNQV |
| | SLTCLVKGFY PSDIAVEWES NGQPENNYKT |
| | TPPVLDSDGS FFLYSKLTVD KSRWQQGNVF |
| | SCSVMHEALH NHYTQKSLSL SPGK (SEQ ID NO: 183) |
| NMT16 fAb-Alb w/ YTE | EVQLVQSGAE VKKPGASVKV SCKASGYIFT |
| Fc | NYPIHWVRQA PGQGLEWMGF IDPGGGYDEP |
| NMT16 Fv-VH | DERFRDRVTM TADTSTSTAY MELSSLRSED |
| IgG1 YTE Fc-Full CH | TAVYYCARRG GGYYLDYWGQ GTTVTVSSAS |
| | TKEVQLVQSG AEVKKPGASV KVSCKASGYI |
| | FTNYPIHWVR QAPGQGLEWM GFIDPGGGYD |
| | EPDERFRDRV TMTADTSTST AYMELSSLRS |
| | EDTAVYYCAR RGGGYYLDYW GQGTTVTVSS ASTK |
| | (SEQ ID NO: 184) |
| NMT16 Fv-VL | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN RGEC (SEQ |
| | ID NO: 185) |
| Anti-Alb Fv-VH | EVQLVESGGG LVQPGGSLRL SCAASGFTFR |
| Linker | SFGMSWVRQA PGKEPEWVSS ISGSGSDTLY |
| IgG1 YTE Fc-Full CH | ADSVKGRFTI SRDNAKTTLY LQMNSLKPED |
| | TAVYYCTIGG SLSRSSQGTQ VTVSSGGGGS |
| | GGGGSGGGGS GPSVFPLAPS SKSTSGGTAA |
| | LGCLVKDYFP EPVTVSWNSG ALTSGVHTFP |
| | AVLQSSGLYS LSSVVTVPSS SLGTQTYICN VNHKPSNTKV |
| | DKKVEPKSCD KTHTCPPCPA PELLGGPSVF LFPPKPKDTL |
| | YITREPEVTC VVVDVSHEDP EVKFNWYVDG |
| | VEVHNAKTKP REEQYNSTYR VVSVLTVLHQ |
| | DWLNGKEYKC KVSNKALPAP IEKTISKAKG |
| | QPREPQVYTL PPSRDELTKN QVSLTCLVKG |
| | FYPSDIAVEW ESNGQPENNY KTTPPVLDSD GSFFLYSKLT |
| | VDKSRWQQGN VFSCSVMHEA LHNHYTQKSL SLSPGK |
| | (SEQ ID NO: 186) |
| NMT16 fAb-HumRab | EVQLVQSGAE VKKPGASVKV SCKASGYIFT |
| Anti-Alb w/ N297A | NYPIHWVRQA PGQGLEWMGF IDPGGGYDEP |
| NMT16 Fv-VH | DERFRDRVTM TADTSTSTAY MELSSLRSED |
| IgG1 N297A Fc-Full CH | TAVYYCARRG GGYYLDYWGQ GTTVTVSSAS |
| | TKGPSVFPLA PSSKSTSGGT AALGCLVKDY |
| | FPEPVTVSWN SGALTSGVHT FPAVLQSSGL YSLSSVVTVP |
| | SSSLGTQTYI CNVNHKPSNT KVDKKVEPKS |
| | CDKTHTCPPC PAPELLGGPS VFLFPPKPKD TLMISRTPEV |
| | TCVVVDVSHE DPEVKFNWYV DGVEVHNAKT |
| | KPREEQYAST YRVVSVLTVL HQDWLNGKEY |
| | KCKVSNKALP APIEKTISKA KGQPREPQVY TLPPSRDELT |
| | KNQVSLTCLV KGFYPSDIAV EWESNGQPEN |
| | NYKTTPPVLD SDGSFFLYSK LTVDKSRWQQ |
| | GNVFSCSVMH EALHNHYTQK SLSLSPGK (SEQ ID NO: |
| | 187) |
| NMT16 Fv-VL | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN RGEC (SEQ |
| | ID NO: 188) |
| HumRab Anti-Alb Fv-VH | EVQLLESGGG LVQPGGSLRL SCAVSGIDLS |
| IgG1 N297A Fc-Full CH | NYAINWVRQA PGKGLEWIGIIWASGTTFYA |
| | TWAKGRFTIS RDNSKNTVYL QMNSLRAEDT |
| | AVYYCARTVP GYSTAPYFDL WGQGTLVTVS |
| | SASTKGPSVF PLAPSSKSTS GGTAALGCLV KDYFPEPVTV |
| | SWNSGALTSG VHTFPAVLQS SGLYSLSSVV TVPSSSLGTQ |
| | TYICNVNHKP SNTKVDKKVE PKSCDKTHTC |
| | PPCPAPELLG GPSVFLFPPK PKDTLMISRT PEVTCVVVDV |
| | SHEDPEVKFN WYVDGVEVHN AKTKPREEQY |
| | ASTYRVVSVL TVLHQDWLNG KEYKCKVSNK |
| | ALPAPIEKTI SKAKGQPREP QVYTLPPSRD ELTKNQVSLT |
| | CLVKGFYPSD IAVEWESNGQ PENNYKTTPP |
| | VLDSDGSFFL YSKLTVDKSR WQQGNVFSCS |
| | VMHEALHNHY TQKSLSLSPG K (SEQ ID NO: 189) |
| HumRab Anti-Alb Fv-VL | DIQMTQSPSS VSASVGDRVT ITCQSSPSVW |
| Fc-CL | SNFLSWYQQK PGKAPKLLIY EASKLTSGVP SRFSGSGSGT |
| | DFTLTISSLQ PEDFATYYCG GGYSSISDTT FGGGTKVEIK |
| | RTVAAPSVFI FPPSDEQLKS GTASVVCLLN NFYPREAKVQ |
| | WKVDNALQSG NSQESVTEQD SKDSTYSLSS |
| | TLTLSKADYE KHKVYACEVT HQGLSSPVTK SFNRGEC |
| | (SEQ ID NO: 190) |
| ALXN Anti-P-Anti-Alb w/ | EVQLVESGGG LVQPGGSLRL SCAASGRISS |
| IgG4 | IIHMAWVRQA PGKQRELVSE ISRVGTTVYA |
| Camelid Anti-P VHH | DSVKGRFTIS RDNSKNTLYL QMNSLRAEDT |
| Linker | AVYYCNALQY EKHGGADYWG QGTLVTVSSG |
| IgG4 Fc-CH2/CH3 Only | GGGSGGGGSG GGGSAESKYG PPCPPCPAPE |
| | AAGGPSVFLF PPKPKDTLMI SRTPEVTCVV |
| | VDVSQEDPEV QFNWYVDGVE VHNAKTKPRE |
| | EQFNSTYRVV SVLTVLHQDW LNGKEYKCKV |
| | SNKGLPSSIE KTISKAKGQP REPQVYTLPP SQEEMTKNQV |
| | SLTCLVKGFY PSDIAVEWES NGQPENNYKT |
| | TPPVLDSDGS FFLYSRLTVD KSRWQEGNVF |
| | SCSVMHEALH NHYTQKSLSL SLG (SEQ ID NO: 191) |
| Anti-Alb | EVQLVESGGG LVKPGGSLRL SCAASGRPVS |
| Linker | NYAAAWFRQA PGKEREFVSA INWQKTATYA |
| IgG4 Fc-CH2/CH3 Only | DSVKGRFTIS RDNAKNSLYL QMNSLRAEDT |
| | AVYYCAAVFR VVAPKTQYDY DYWGQGTLVT |
| | VSSGGGGSGG GGSGGGGSAE SKYGPPCPPC |
| | PAPEAAGGPS VFLFPPKPKD TLMISRTPEV TCVVVDVSQE |
| | DPEVQFNWYV DGVEVHNAKT KPREEQFNST |
| | YRVVSVLTVL HQDWLNGKEY KCKVSNKGLP |
| | SSIEKTISKA KGQPREPQVY TLPPSQEEMT KNQVSLTCLV |
| | KGFYPSDIAV EWESNGQPEN NYKTTPPVLD |
| | SDGSFFLYSR LTVDKSRWQE GNVFSCSVMH |
| | EALHNHYTQK SLSLSLG (SEQ ID NO: 192) |
| Anti-TNF-NMT16 fAb w/ | EVQLVQSGAE VKKPGASVKV SCKASGYIFT |
| YTE Fc | NYPIHWVRQA PGQGLEWMGF IDPGGGYDEP |
| NMT16 Fv-VH | DERFRDRVTM TADTSTSTAY MELSSLRSED |
| IgG1Fc-Full CH | TAVYYCARRG GGYYLDYWGQ GTTVTVSSAS |
| | TKEVQLVQSG AEVKKPGASV KVSCKASGYI |
| | FTNYPIHWVR QAPGQGLEWM GFIDPGGGYD |
| | EPDERFRDRV TMTADTSTST AYMELSSLRS |
| | EDTAVYYCAR RGGGYYLDYW GQGTTVTVSS ASTK |
| | (SEQ ID NO: 193) |
| NMT16 Fv-VL | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN RGEC (SEQ |
| | ID NO: 194) |
| Anti-TNF Fv-VH | EVQLVESGGG LVQPGRSLRL SCAASGFTFD |
| IgG1 Fc-Full CH | DYAMHWVRQA PGKGLEWVSA ITWNSGHIDY |
| | ADSVEGRFTI SRDNAKNSLY LQMNSLRAED |
| | TAVYYCAKVS YLSTASSLDY WGQGTLVTVS |
| | SASTKGPSVF PLAPSSKSTS GGTAALGCLV KDYFPEPVTV |
| | SWNSGALTSG VHTFPAVLQS SGLYSLSSVV TVPSSSLGTQ |
| | TYICNVNHKP SNTKVDKKVE PKSCDKTHTC |
| | PPCPAPELLG GPSVFLFPPK PKDTLYITRE PEVTCVVVDV |
| | SHEDPEVKFN WYVDGVEVHN AKTKPREEQY |
| | NSTYRVVSVL TVLHQDWLNG KEYKCKVSNK |
| | ALPAPIEKTI SKAKGQPREP QVYTLPPSRD ELTKNQVSLT |
| | CLVKGFYPSD IAVEWESNGQ PENNYKTTPP |
| | VLDSDGSFFL YSKLTVDKSR WQQGNVFSCS |
| | VMHEALHNHY TQKSLSLSPG K (SEQ ID NO: 195) |
| Anti-TNF Fv-VL | DIQMTQSPSS LSASVGDRVT ITCRASQGIR |
| Fc-CL | NYLAWYQQKP GKAPKLLIYA ASTLQSGVPS |
| | RFSGSGSGTD FTLTISSLQP EDVATYYCQR YNRAPYTFGQ |
| | GTKVEIKRTV AAPSVFIFPP SDEQLKSGTA SVVCLLNNFY |
| | PREAKVQWKV DNALQSGNSQ ESVTEQDSKD |
| | STYSLSSTLT LSKADYEKHK VYACEVTHQG |
| | LSSPVTKSFN RGEC (SEQ ID NO: 196) |
| Anti-VEGF-NMT16 fAb | EVQLVQSGAE VKKPGASVKV SCKASGYIFT |
| w/ Fc | NYPIHWVRQA PGQGLEWMGF IDPGGGYDEP |
| NMT16 Fv-VH | DERFRDRVTM TADTSTSTAY MELSSLRSED |
| IgG1 YTE Fc-Full CH | TAVYYCARRG GGYYLDYWGQ GTTVTVSSAS |
| | TKEVQLVQSG AEVKKPGASV KVSCKASGYI |
| | FTNYPIHWVR QAPGQGLEWM GFIDPGGGYD |
| | EPDERFRDRV TMTADTSTST AYMELSSLRS |
| | EDTAVYYCAR RGGGYYLDYW GQGTTVTVSS ASTK |
| | (SEQ ID NO: 197) |
| NMT16 Fv-VL | DIQMTQSPSS LSASVGDRVT ITCRASQDIS FFLNWYQQKP |
| Fc-CL | GKAPKLLIYY TSRYHSGVPS RFSGSGSGTD FTLTISSLQP |
| | EDFATYYCQH GNTLPWTFGQ GTKLEIKRTV AAPSVFIFPP |
| | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN RGEC (SEQ |
| | ID NO: 198) |
| Anti-VEGF Fv-VH | EVQLVESGGG LVQPGGSLRL SCAASGYTFT |
| IgG1 Fc-Full CH | NYGMNWVRQA PGKGLEWVGW INTYTGEPTY |
| | AADFKRRFTF SLDTSKSTAY LQMNSLRAED |
| | TAVYYCAKYP HYYGSSHWYF DVWGQGTLVT |
| | VSSASTKGPS VFPLAPSSKS TSGGTAALGC LVKDYFPEPV |
| | TVSWNSGALT SGVHTFPAVL QSSGLYSLSS VVTVPSSSLG |
| | TQTYICNVNH KPSNTKVDKK VEPKSCDKTH |
| | TCPPCPAPEL LGGPSVFLFP PKPKDTLYIT REPEVTCVVV |
| | DVSHEDPEVK FNWYVDGVEV HNAKTKPREE |
| | QYNSTYRVVS VLTVLHQDWL NGKEYKCKVS |
| | NKALPAPIEK TISKAKGQPR EPQVYTLPPS RDELTKNQVS |
| | LTCLVKGFYP SDIAVEWESN GQPENNYKTT |
| | PPVLDSDGSF FLYSKLTVDK SRWQQGNVFS |
| | CSVMHEALHN HYTQKSLSLS PGK (SEQ ID NO: 199) |
| Anti-VEGF Fv-VL | DIQMTQSPSS LSASVGDRVT ITCSASQDIS NYLNWYQQKP |
| Fc-CL | GKAPKVLIYF TSSLHSGVPS RFSGSGSGTD FTLTISSLQP |
| | EDFATYYCQQ YSTVPWTFGQ GTKVEIKRTV AAPSVFIFPP |
| | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN RGEC (SEQ |
| | ID NO: 200) |

### Example 8

### NMT15-NMT22 Binding Potency and Functional Assays

### Binding Affinity to Properdin

Polystyrene microtiter plates were coated with human (2.0 ag/50 al per well) properdin in phosphate buffered saline (PBS) overnight at 4° C. After aspirating the properdin solution, the wells were blocked with PBS containing 1% bovine serum albumin (BSA) (Sigma-Aldrich, St. Louis, Mo.) for 2 hours at room temperature. Wells without peptide or properdin coating served as background controls. Aliquots of the test antibody in blocking solution were added to the properdin coated wells and allowed to incubate for 1 hour to allow binding to occur. Following a 1 hour incubation at room temperature, the plate was rinsed with PBS five times and incubated with 1:2000 diluted anti-properdin polyclonal antibody.

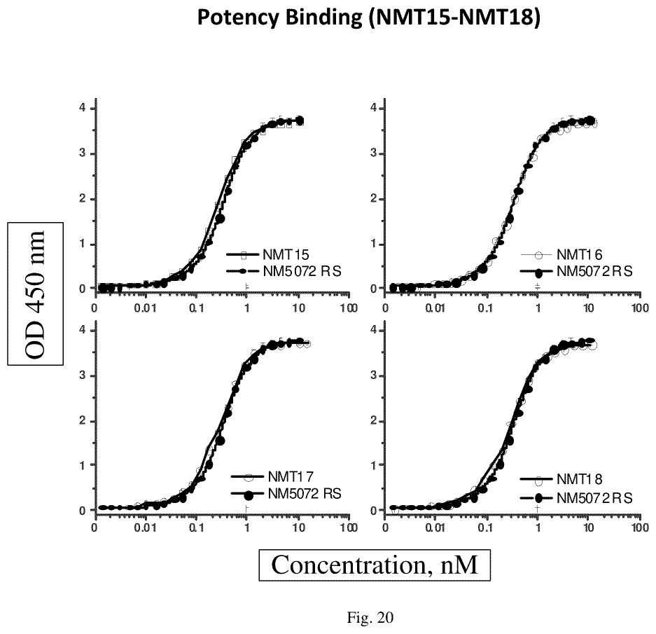

As shown in FIG. 20, NMT15, NMT16, NMT17, and NMT18 bind to properdin with high affinity. The affinity values of NMT15, NMT16, NMT17, and NMT18 were similar to NMT5072.

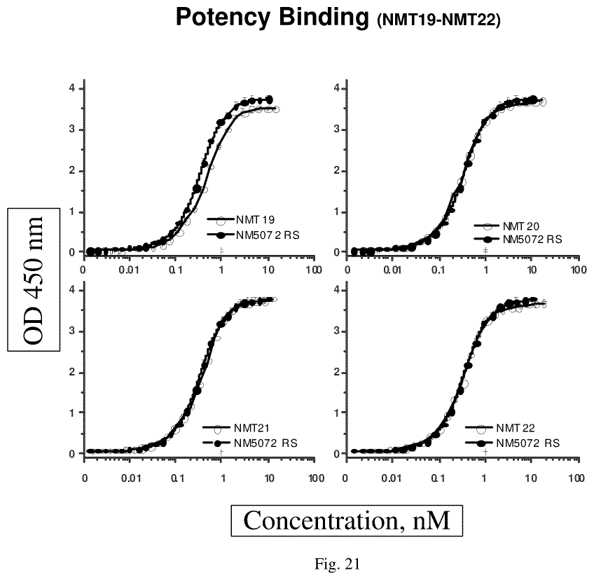

As shown in FIG. 21, NMT19, NMT20, NMT21, and NMT22 bind to properdin with high affinity. The affinity values of NMT19, NMT20, NMT21, and NMT22 were similar to NMT5072.

### Inhibition of AP Hemolysis

This cellular assay is based on the formation of terminal complement complex on the surface of the rRBC. As a result, the rRBC are lysed. The evidence of lysed cells is reflected in progressive decrease in light scatter at 700 nm. rRBC are incubated in normal human serum in AP buffer. The surface of rRBC triggers the activation of AP in normal human serum. AP cascade begins and leads to the formation of C5b-9 complex on the surface of the rRBC. Agents that inhibit the activation are expected to inhibit cellular lysis.

To evaluate the effect of the test antibody on AP activation, various concentrations of the test antibody in AP buffer were incubated with normal human serum (10% NHS) at 37° C. with a fixed number of rabbit erythrocytes (Covance) in a temperature controlled ELISA plate reader capable of reading at 700 nm A progressive decrease in light scatter (due to lysis of intact cells) was measured at 700 nm as a function of time. The data were recorded and analyzed with a SpectraMax 190 plate reader and SoftMax Pro software. For calculation total inhibition was calculated at each concentration of the antibody and the results were expressed as a % of unlysed controls. Data at each concentration was plotted in a sigmoidal plot with MicroCal Origin Software.

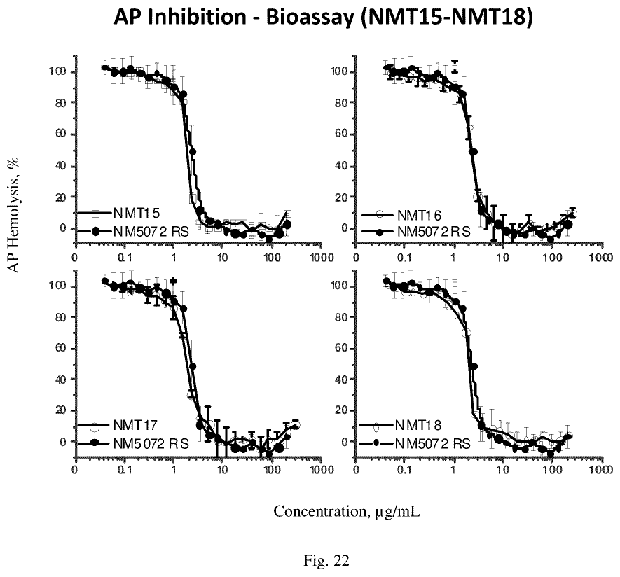

FIG. 22 demonstrates the potent activity of NMT15, NMT16, NMT17, and NMT18 in inhibiting erythrocyte lysis. NMT15, NMT16, NMT17, and NMT18 were able to inhibit lysis at a similar concentration as NM5072.

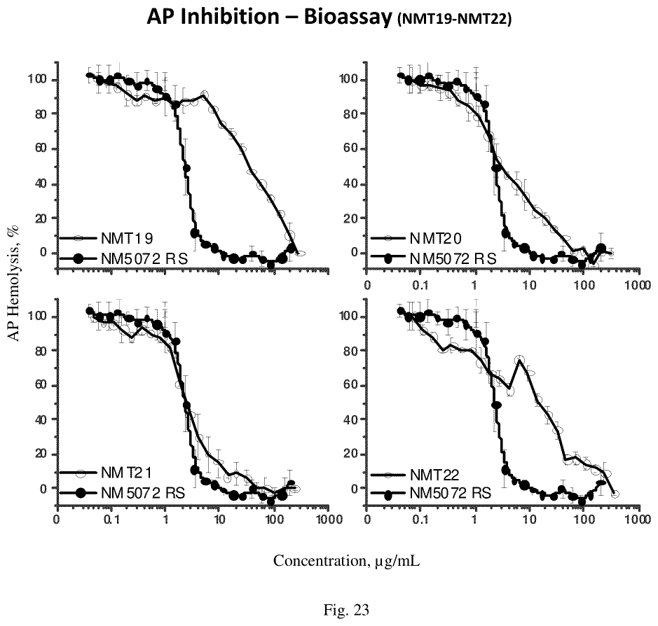

FIG. 23 demonstrates the potent activity of NMT19, NMT20, NMT21, and NMT22 in inhibiting erythrocyte lysis. NMT21 was able to inhibit lysis at a similar concentration as NM5072.

### Inhibition of MAC Formation

C5b-9 Formation Assay: Microtiter wells were coated with LPS (2 μg/50 upper well) in PBS overnight at 4° C. Uncoated wells served as background controls. After aspirating the LPS solution, wells were treated with 1% BSA in phosphate buffered saline, pH 7.4 (PBS) for 2-hours. Following a 2 hour incubation, wells were rinsed with PBS and incubated with various concentrations of the antibody in AP buffer containing 10% Normal Human Serum (NHS). Following a 2-hour incubation at 37° C. to allow AP activation to occur, deposited MAC(C5b-9) was detected with 1:2000 diluted mouse anti-human soluble neo-05b-9 monoclonal antibody. All dilutions of the antibody were made in blocking solution and all antibody incubations were done for 1 hour at room temperature. The primary antibody was detected with goat anti mouse monoclonal. Following each incubation the plate was rinsed five times with PBS. The plate was developed with TMB and the blue color reaction was quenched with 1M phosphoric acid.

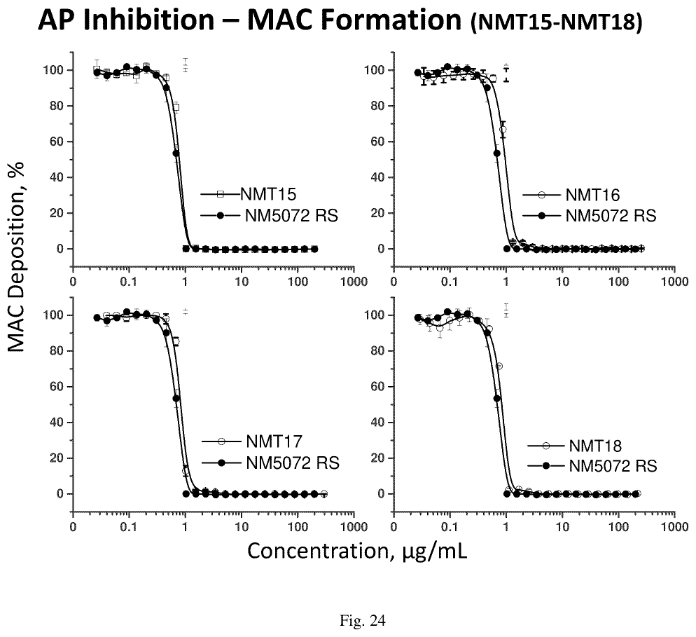

FIG. 24 illustrates plots showing inhibition of MAC Formation by NMT15, NMT16, NMT17, and NMT18. NMT15, NMT16, NMT17, and NMT18 were able to inhibit MAC formation and deposition at a similar concentration as NM5072.

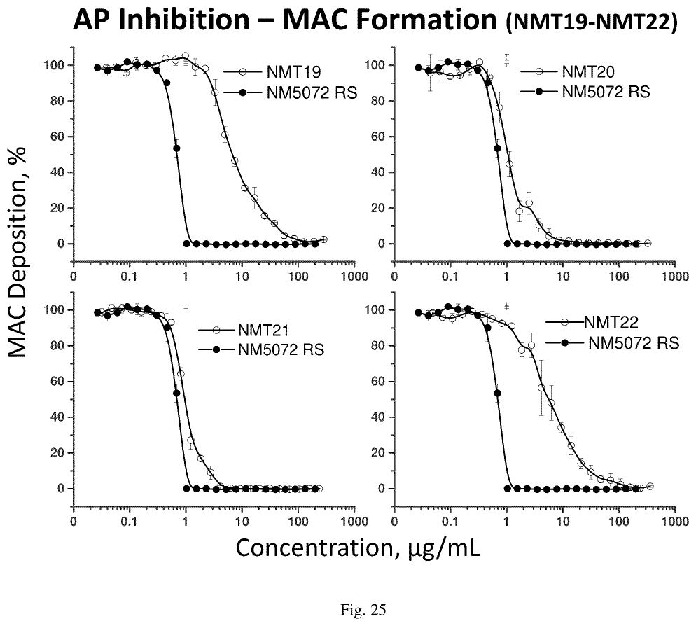

FIG. 25 illustrates plots showing inhibition of MAC Formation by NMT19, NMT20, NMT21, and NMT22. NMT20 and NMT21 were able to MAC formation and deposition at a similar concentration as NM5072.

### Inhibition of Properdin-C3b Binding

AP activation generates C3a and C3b as a result of C3 cleavage by the C3 convertase of the alternative complement pathway. Alternative complement pathway is activated in normal human serum by lip polysaccharide from Salmonella Typhosa under conditions that allow the activation of the alternative complement pathway. We have utilized this assay to demonstrate whether anti-properdin antibody of this invention would inhibit the formation and deposition of C3b. Deposition of C3b initiates the start of the alternative complement pathway. As a way of mechanism, activated and deposited C3b provides high affinity binding to properdin. Properdin-C3b complexes bind factor B and the complex is cleaved by factor D to generate PC3bBb, an alternative pathway C3 convertase. As the alternative pathway proceeds, C5b-9 complexes are formed and deposited.

The formation and deposition of C3b is inhibited. Because C3b formation and deposition is inhibited, the deposition of other components, such as properdin, is also inhibited.

In a typical assay, polystyrene microtiter plate wells were coated with LPS (Lip polysaccharide from Salmonella Typhosa) at 2 μg/50 μl in PBS overnight. The wells were incubated with BSA in PBS to block the unoccupied sites in the wells. Following a 2-hour blocking at room temperature and rinsing with PBS, normal human serum (10%) in AP buffer was mixed with varying concentrations of the anti-properdin antibody and derived fragments. The mixture was incubated onto LPS coated wells. The plate was incubated for 2 hours at 37° C. to allow complement AP activation to occur. Following incubation, the plates were extensively washed with PBS, and components of the C3 convertase were detected with the appropriate antibodies. We detected C3b with rabbit anti-human C3c at 1:2000 in blocking solution, properdin was detected with goat anti-human P, Bb was detected with goat anti-human factor Bb at 1:500 in blocking solution and C5b-9 was detected with HRPO-conjugated neo-anti-human C5b-9 at 1:2000 in blocking solution. Plates were incubated with their respective antibodies for 1-hour at room temperature. Following the incubation, the plates were rinsed with PBS and the bound antibodies were detected with peroxidase labeled goat anti-rabbit at 1:2000 for C3b and peroxidase labeled rabbit anti-goat at 1:2000 in blocking solution for P detection. All plates were developed with TMB following extensive washing with PBS. The blue color was quenched with 1 M orthophosphoric acid. The presence of C3b, P and Bb and MAC together are indicative of AP C3 convertase formation. The antibodies of the present invention are shown to inhibit C3b formation and therefore deposition. This data provides direct evidence that anti-properdin monoclonal antibodies prevent C3 convertase formation and thus AP activation.

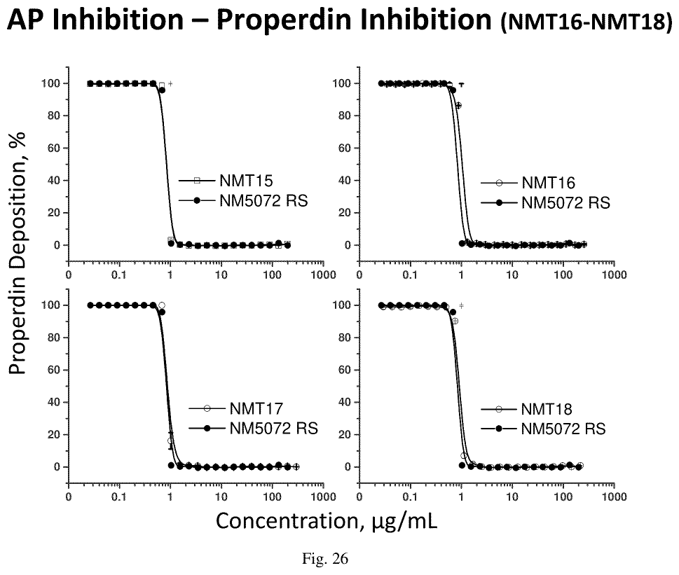

FIG. 26 illustrates plots showing inhibition of deposition of properdin by NMT15, NMT16, NMT17, and NMT18. NMT15, NMT16, NMT17, and NMT18 were able to inhibit properdin binding to C3b and deposition of properdin at a similar concentration as NM5072.

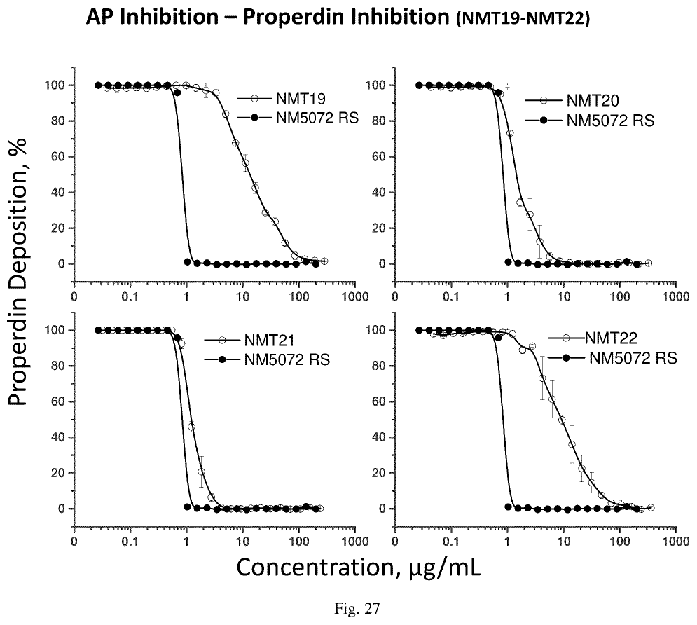

FIG. 27 illustrates plots showing inhibition of deposition of properdin by NMT15, NMT19, NMT20, NMT21, and NMT22. NMT20 and NMT21 were able to inhibit properdin binding to C3b and deposition of properdin at a similar concentration as NM5072.

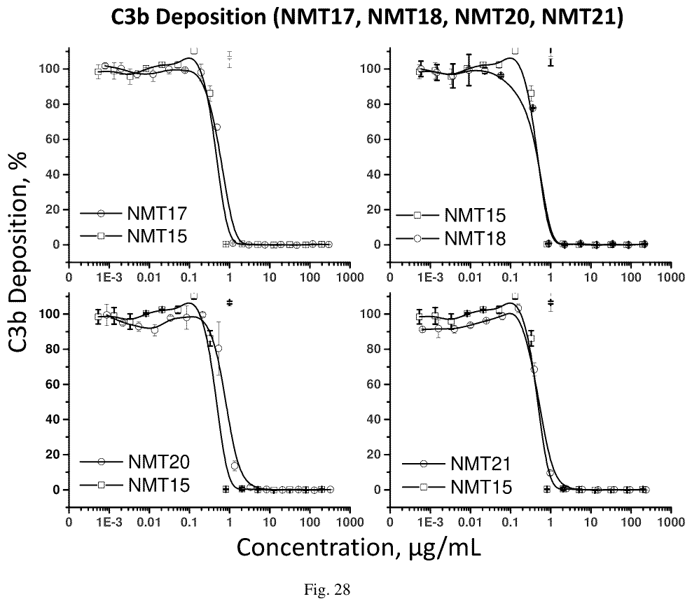

FIG. 28 illustrates plots showing inhibition of AP mediated C3b Formation and Deposition by NMT15, NMT17, NMT18, NMT20, and NMT21. NMT15, NMT17, NMT18, NMT20, and NMT21 were able to inhibit properdin binding to C3b and C3b deposition at a similar concentrations.

### Inhibition of Classical Pathway Activation

To test the activity of the antibodies for CP inhibition, antibody-sensitized, sheep erythrocytes (sRBC) were incubated in 1% normal human serum in CP buffer (Ca²⁺/Mg²⁺) These sRBCs activate the CP, which induces lysis of cell membranes. Lysis of the cell membranes results in a gradual decrease in light scattered by cells. When an alternative pathway specific antibody of the present invention was incubated with sRBCs at 37° C. in 1% NHS with a buffer containing Ca²⁺ and Mg²⁺ (“the CP buffer”) no effect on hemolysis was observed within the time period beginning with the start of hemolysis and concluding with maximal hemolysis. This implies that the antibodies not affect CP hemolytic activity in NHS and is not expected to compromise the CP's expected contribution to host defense against pathogens.

Antibodies described herein irrespective of the target antigen against which they have been raised, do not inhibit the classical pathway. In a typical assay, antibody sensitized sheep erythrocytes are incubated with Normal Human Serum, with CP buffer containing Ca2+. These conditions allow for selective activation of the classical pathway. Mechanistically, the antigen-Antibody complex on the surface of the sheep cells activates the classical complement pathway which causes erythrocyte lysis.

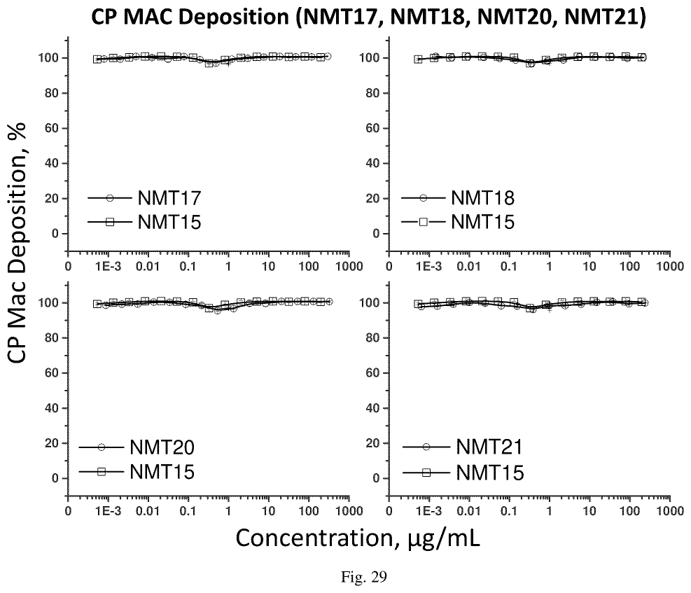

FIG. 29 illustrates plots showing CP mediated MAC Formation and Deposition by NMT15, NMT17, NMT18, NMT20, and NMT21. NMT15, NMT17, NMT18, NMT20, and NMT21 did not inhibit CP mediated MAC formation and deposition.

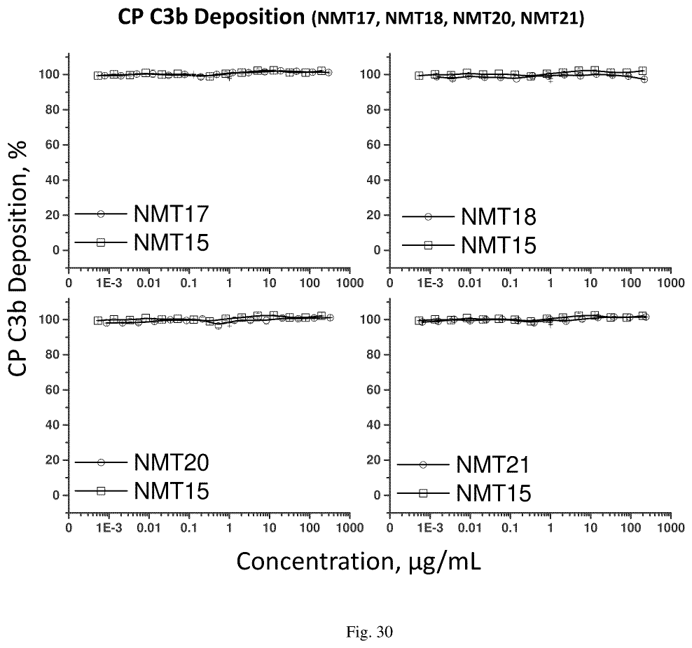

FIG. 30 illustrates plots showing CP mediated C3b Formation and Deposition by NMT15, NMT17, NMT18, NMT20, and NMT21. NMT15, NMT17, NMT18, NMT20, and NMT21 did not inhibit CP mediated C3b Formation and Deposition.

Table 12 provides a summary of the binding potency and functional assays for NMT15-NMT22.

TABLE 12

| | Binding Affinity | AP Hemolysis | MAC | Properdin |
|---|---|---|---|---|
| Variant | (pM) | (μg/mL) | (μg/Ml) | (μg/mL) |
| NMT#15 | 270 | 5 | 1 | 2 |
| NMT#16 | 315 | 13 | 3 | 4 |
| NMT#17 | 283 | 10 | 5 | 4 |
| NMT#18 | 289 | 20 | 9 | 3 |
| NMT#19 | 486 | — | — | — |
| NMT#20 | 297 | 65 | 25 | 30 |
| NMT#21 | 353 | 50 | 8 | 8 |
| NMT#22 | 335 | — | — | — |

### Example 9

### NMT23-NMT28 Binding Affinity and Functional Assays

### Binding Affinity to Properdin

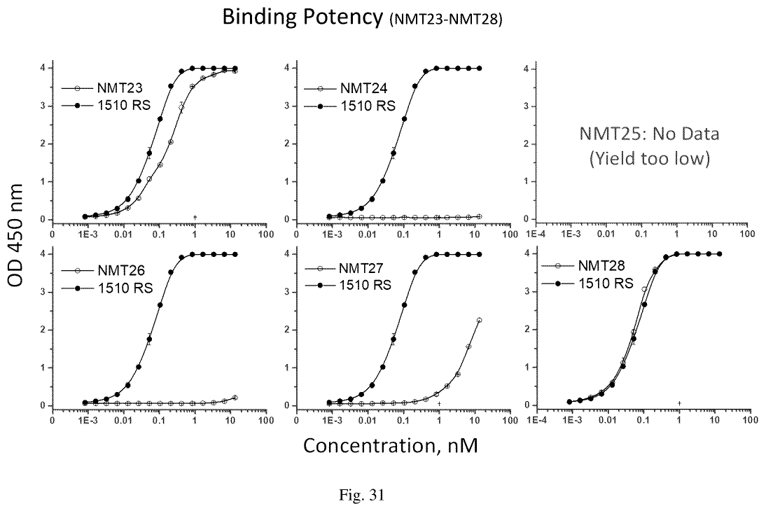

FIG. 31 illustrates plots showing binding Affinity of NMT23, NMT24, NMT25, NMT26, NMT27, and NMT28 to properdin. The properdin affinity values of NMT23 and NMT 28 were similar to NMT1510.

### Inhibition of MAC Formation

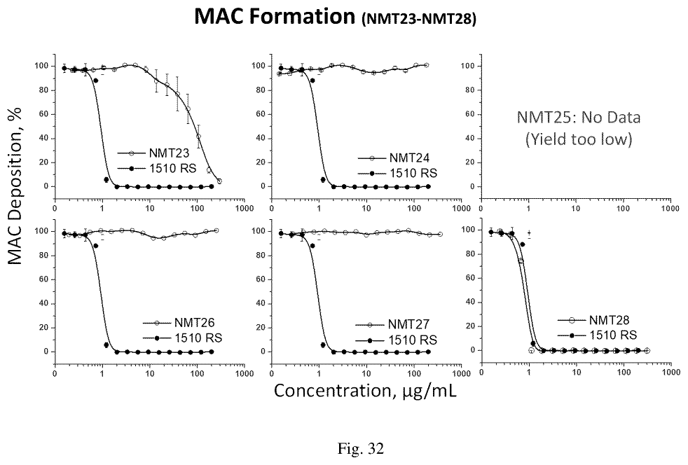

FIG. 32 illustrates plots showing inhibition of MAC Formation and Deposition by NMT23, NMT24, NMT25, NMT26, NMT27, and NMT28. NMT28 was able to inhibit MAC formation and deposition at a similar concentration as NMT1510.

### Inhibition of AP Hemolysis

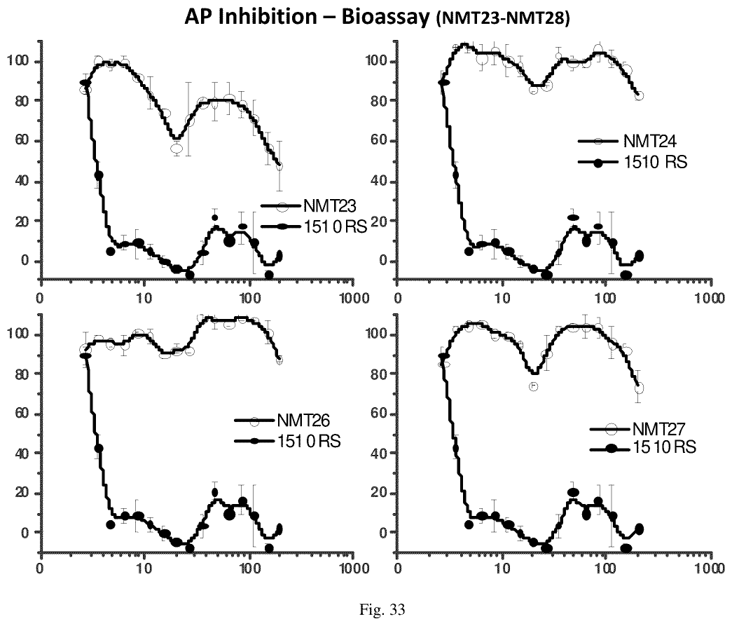

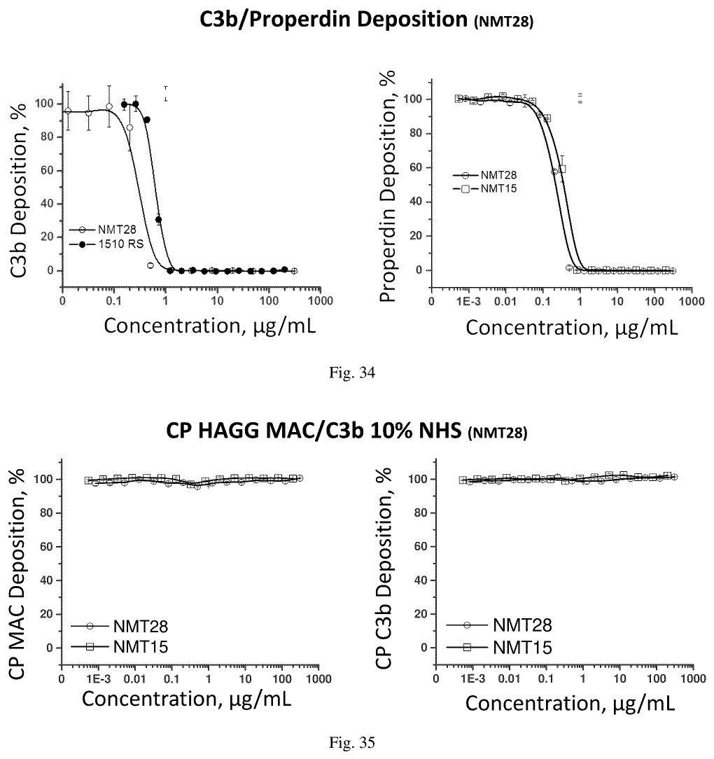

FIG. 33 illustrates plots showing inhibition of AP Hemolysis by NMT23, NMT24, NMT26, and NMT27. NMT15, NMT16, NMT17, and NMT28 were not able to inhibit lysis at a similar concentration as NMT1510.

### Inhibition of Properdin-C3b Binding

FIG. 34 illustrates plots showing inhibition of AP mediated C3 convertase formation and Deposition of properdin and C3b by NMT28. NMT28 was able to inhibit properdin binding to C3b and deposition of properdin and C3b at a similar concentration as NMT1510.

### Inhibition of Classical Pathway Activation

FIG. 35 illustrates plots showing CP mediated MAC Formation and Deposition by NMT28. NMT28 did not inhibit CP mediated MAC formation and deposition.

Table 13 provides a summary of the binding potency and functional assays for NMT23-NMT28.

TABLE 13

| | Binding | AP Hemolysis | MAC |
|---|---|---|---|
| Variants | Affinity | (μg/mL) | (μg/mL) |
| NMT#23 | 176 | — | — |
| NMT#24 | — | — | — |
| NMT#25 | — | — | — |
| NMT#26 | — | — | — |
| NMT#27 | — | — | — |
| NMT#28 | 53 | 8 | 3 |

NMT17, NMT18, NMT28 with Modified Fc Regions Binding Potency and Functional Assays Binding Affinity to Properdin

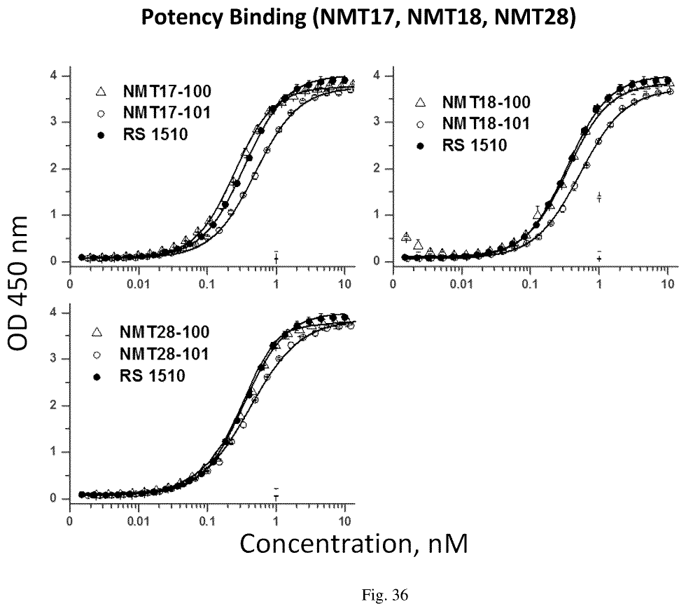

FIG. 36 illustrates plots showing binding Affinity of NMT17-100, NMT17-101, NMT18-100, NMT18-101, NMT28-100, and NMT28-101 to Properdin. Properdin binding affinity values of NMT17-100, NMT17-101, NMT18-100, NMT18-101, NMT28-100, and NMT28-101 were similar to NMT1510.

### Inhibition of AP Hemolysis

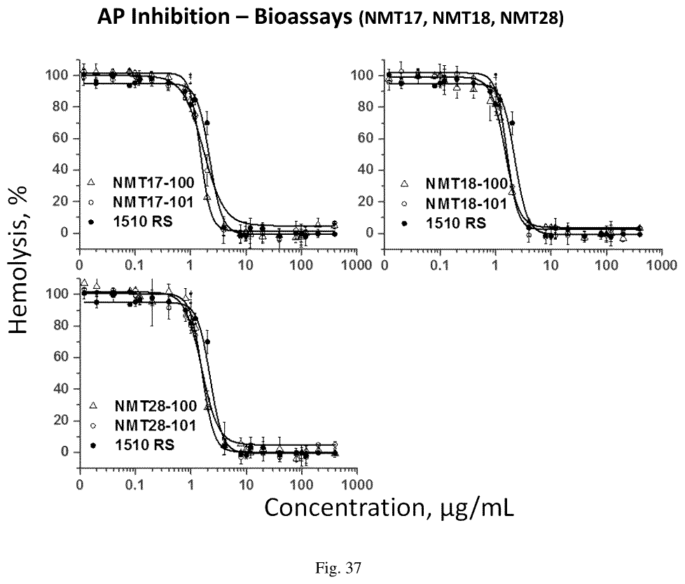

FIG. 37 illustrates plots showing inhibition of AP Hemolysis by NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit lysis at a similar concentration as NMT1510.

### Inhibition of MAC Formation

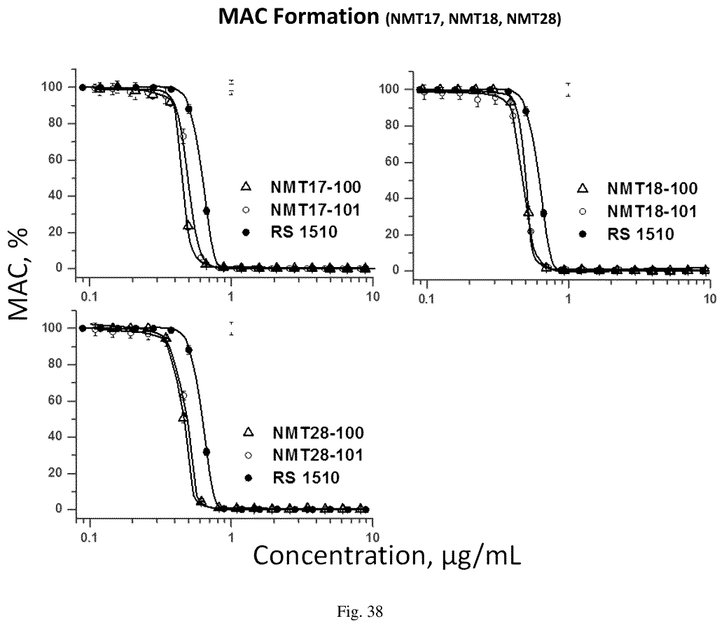

FIG. 38 illustrates plots showing inhibition of Mac Formation by NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit MAC formation and deposition at a similar concentration as NMT1510.

### Inhibition of Properdin-C3b Binding

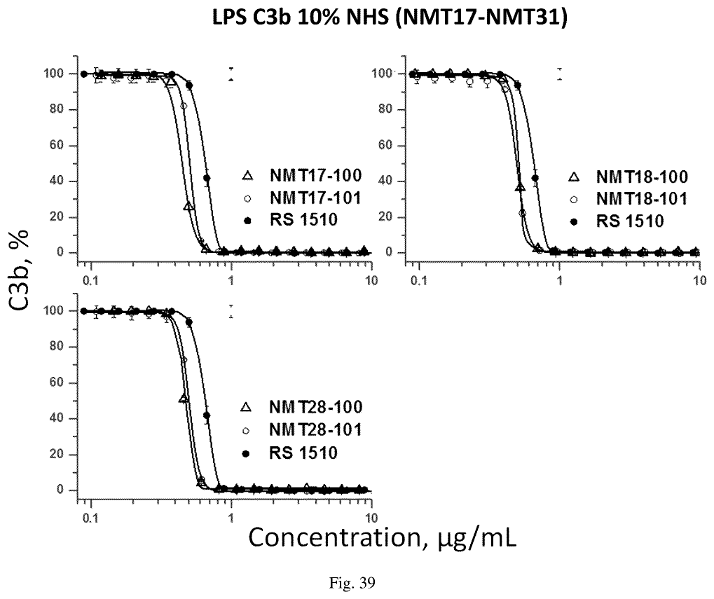

FIG. 39 illustrates plots showing inhibition of AP mediated C3b Formation and Deposition by NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit properdin binding to C3b and deposition C3b at a similar concentration as NMT1510.

Table 14 provides a summary of the binding potency and functional assays for NMT17-100, NMT17-101, NMT18-100, NMT18-101, NMT28-100, and NMT28-101.

TABLE 14

| | Affinity | AP Hemolysis | MAC | C3b |
|---|---|---|---|---|
| Construct | (pM) | (μg/mL) | (μg/mL) | (μg/mL) |
| NMT17-100 | 263 | 5 | 1 | 1 |
| NMT17-101 | 499 | 5 | 1 | 1 |
| NMT18-100 | 322 | 4 | 1 | 1 |
| NMT18-101 | 590 | 5 | 3 | 1 |
| NMT28-100 | 318 | 5 | 3 | 1 |
| NMT28-101 | 425 | 4 | 3 | 1 |

### Example 10

NMT17, NMT18, NMT28 with N297 and Xtend Fc Regions Binding Potency and Functional Assays

### Binding Affinity to Properdin

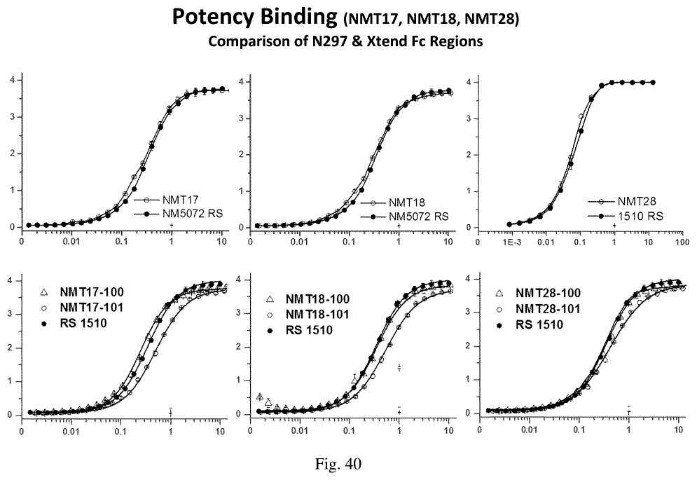

FIG. 40 illustrates plots showing binding Affinity Comparison of N297 and Xtend Fc regions—NMT17, NMT18, NMT28, NMT17-100, NMT17-101, NMT18-100, NMT18-101, NMT28-100, and NMT28-101 to Properdin. Properdin binding affinity values of NMT17-100, NMT17-101, NMT18-100, NMT18-101, NMT28-100, and NMT28-101 were similar to NMT1510.

### Inhibition of AP Hemolysis

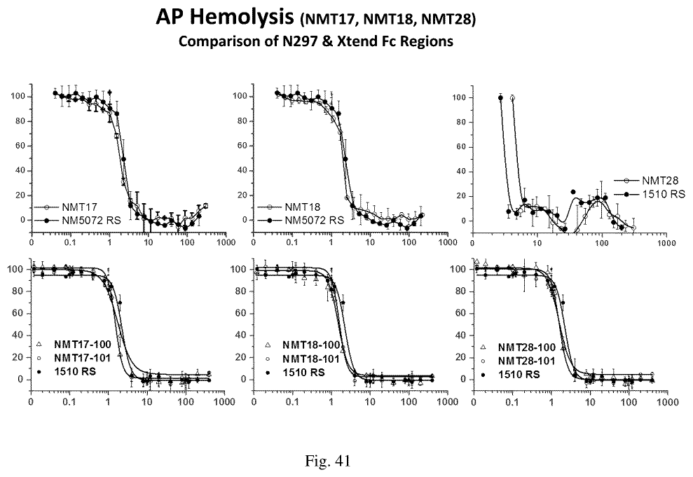

FIG. 41 illustrates plots showing inhibition of AP-Hemolysis Comparison of N297 and Xtend Fc regions—NMT17, NMT18, NMT28, NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit lysis at a similar concentration as NMT1510.

### Inhibition of MAC Formation

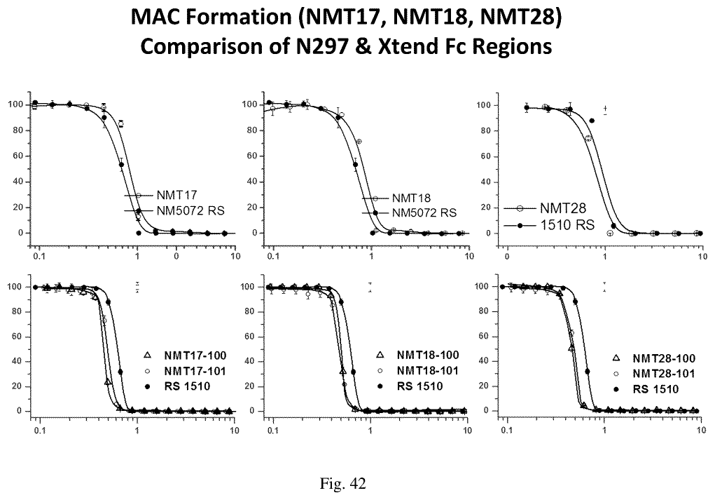

FIG. 42 illustrates plots showing inhibition of Mac Formation by Comparison of N297 and Xtend Fc regions—NMT17, NMT18, NMT28, NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit MAC formation and deposition at a similar concentration as NMT1510.

### Inhibition of Properdin-C3b Binding

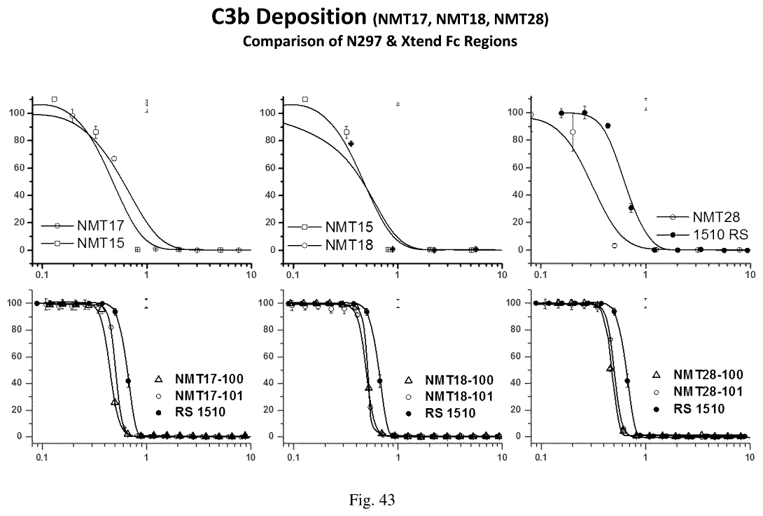

FIG. 43 illustrates plots showing inhibition of C3b Formation and Deposition—Comparison of N297 and Xtend Fc regions—NMT17, NMT18, NMT28, NMT17-100, NMT17-101, NMT18-100, NMT18-101, NMT28-100, and NMT28-101. NMT17-100, NMT17-101, NMT18-100, NMT18-101, NMT28-100, and NMT28-101 were able to inhibit properdin binding to C3b and deposition C3b at a similar concentration as NMT1510.

### Example 11

### NMT29-NMT31 Binding Potency and Functional Assays

### Binding Affinity to Properdin

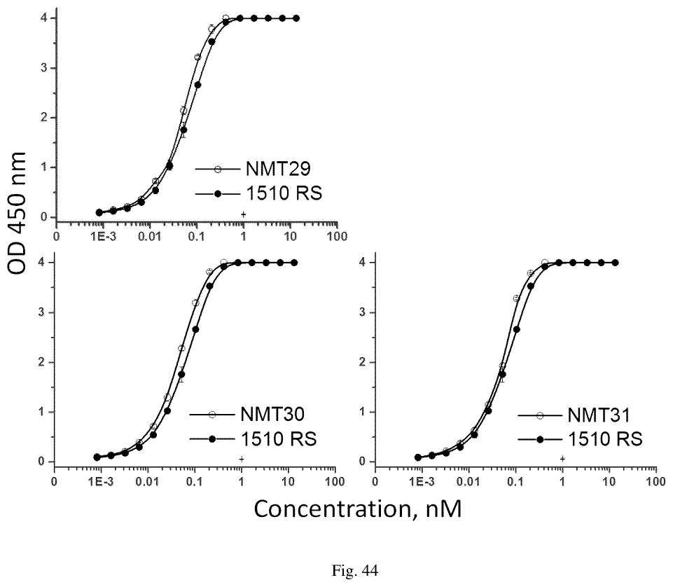

FIG. 44 illustrates plots showing binding Potency to Properdin—Comparison of N297 and Xtend Fc regions—NMT29, NMT30, NMT31. The properdin binding affinity values of NMT29, NMT30, and NMT31 were similar to NMT1510.

### Inhibition of MAC Formation

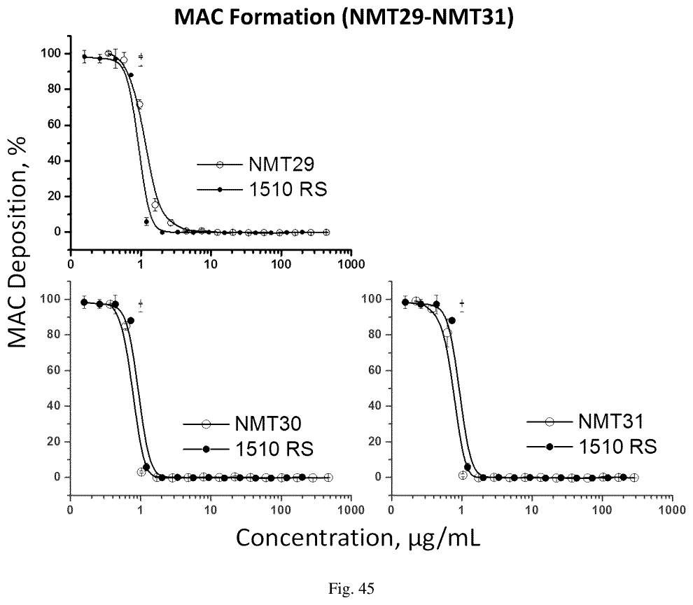

FIG. 45 illustrates plots showing formation of AP mediated MAC—Comparison of N297 and Xtend Fc regions—NMT29, NMT30, NMT31. NMT29, NMT30, NMT31 were able to inhibit MAC formation and deposition at a similar concentration as NMT1510.

### Inhibition of AP Hemolysis

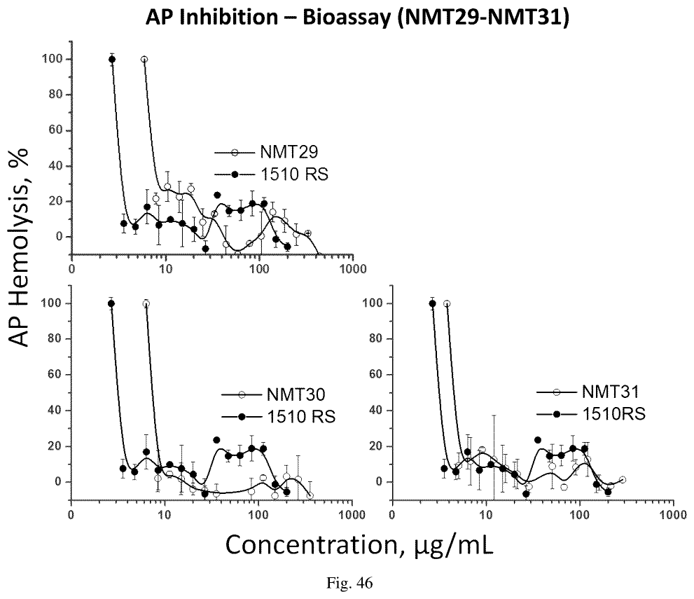

FIG. 46 illustrates plots showing inhibition of AP-mediated Hemolysis—Comparison of N297 and Xtend Fc regions—NMT29, NMT30, NMT31. NMT29, NMT30, NMT31 were able to inhibit lysis at a similar concentration as NMT1510.

### Inhibition of Properdin-C3b Binding

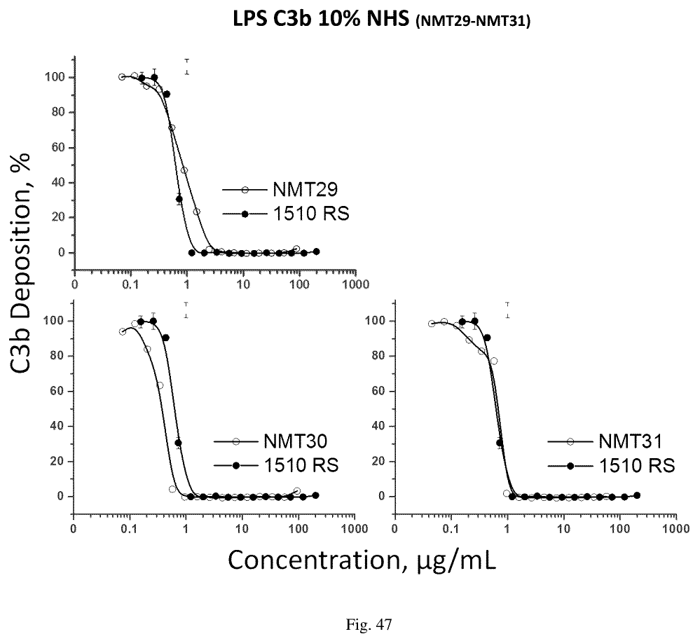

FIG. 47 illustrates plots showing inhibition of AP-mediated C3b Formation & Deposition—Comparison of N297 and Xtend Fc regions—NMT29, NMT30, NMT31. NMT29, NMT30, NMT31 were able to inhibit properdin binding to C3b and deposition of C3b at a similar concentration as NMT1510.

### Example 12

NMT30 and NMT31 with Modified Fc Regions Binding Potency and Functional Assays
Binding Affinity to properdin.

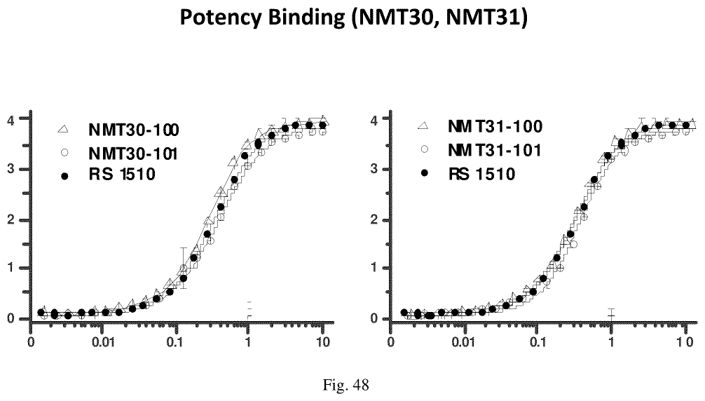

FIG. 48 illustrates plots showing binding Potency to Properdin of NMT30-100, NMT30-101, NMT31-100, and NMT31-101. Properdin binding affinity values of NMT30-100, NMT30-101, NMT31-100, and NMT31-101 were similar to NMT1510.

### Inhibition of AP Hemolysis

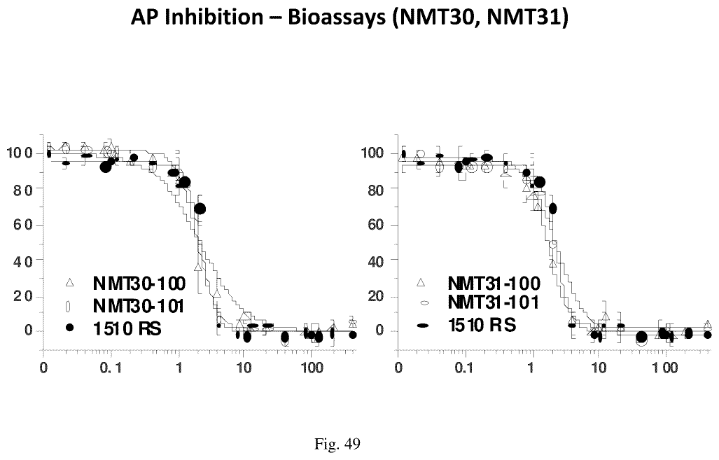

FIG. 49 illustrates plots showing inhibition of AP Hemolysis of NMT30-100, NMT30-101, NMT31-100, and NMT31-101. NMT30-100, NMT30-101, NMT31-100, and NMT31-101 were able to inhibit lysis at a similar concentration as NMT1510.

### Inhibition of MAC Formation

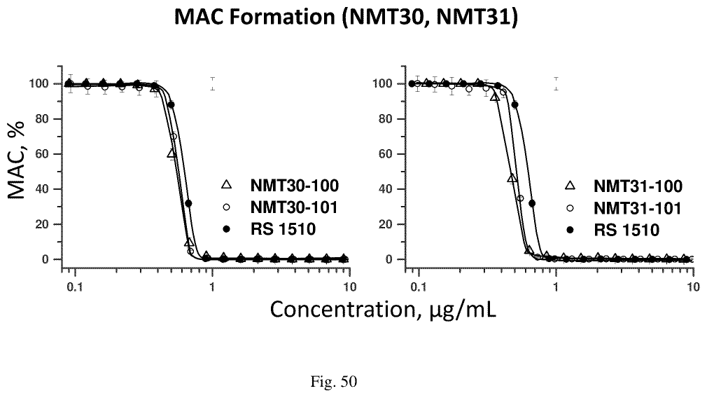

FIG. 50 illustrates plots showing inhibition of AP mediated MAC Formation by NMT30-100, NMT30-101, NMT31-100, and NMT31-101. NMT30-100, NMT30-101, NMT31-100, and NMT31-101 were able to inhibit MAC formation and deposition at a similar concentration as NMT1510.

### Inhibition of Properdin-C3b Binding

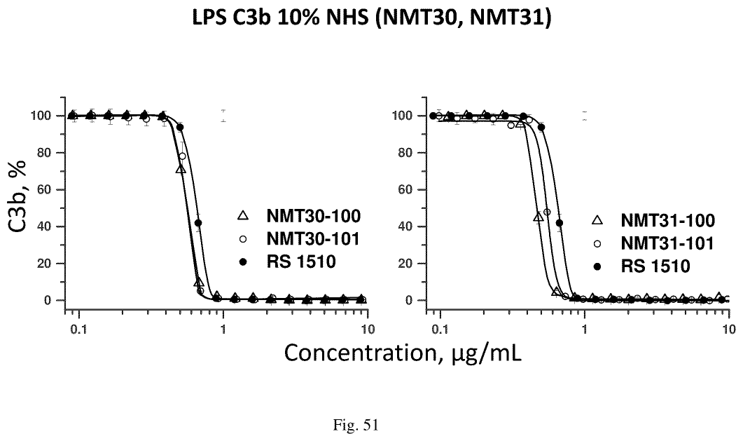

FIG. 51 illustrates plots showing inhibition of AP mediated C3b Formation and Deposition by NMT30-100, NMT30-101, NMT31-100, and NMT31-101. NMT30-100, NMT30-101, NMT31-100, and NMT31-101 were able to inhibit properdin binding to C3b and deposition C3b at a similar concentration as NMT1510.

### Example 13

NMT30 and NMT31 with N297 and Xtend Fc Regions Binding Potency and Functional Assays

### Binding Affinity to Properdin

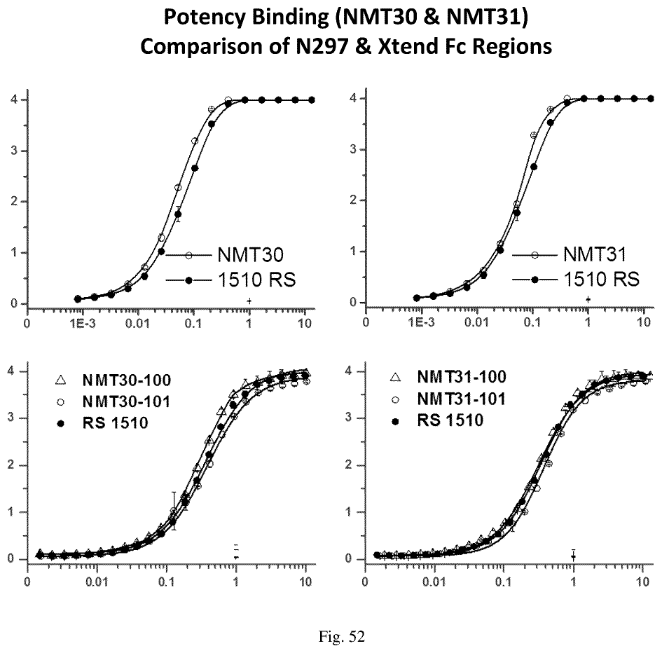

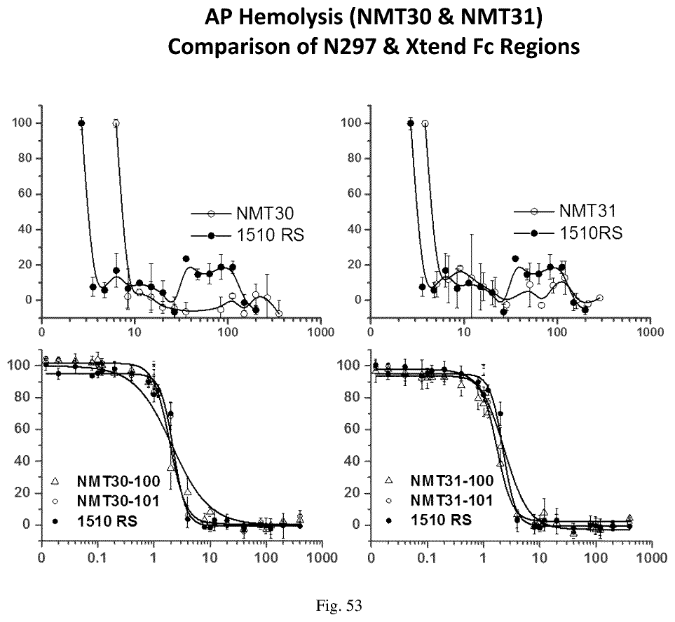

FIG. 52 illustrates plots showing potency Binding to Properdin—Comparison of N297 and Xtend Fc regions—NMT30, NMT31, NMT30-100, NMT30-101, NMT31-100, and NMT31-101. Properdin binding affinity values of NMT30-100, NMT30-101, NMT31-100, and NMT31-101 were similar to NMT1510.

### Inhibition of AP Hemolysis

FIG. 53 illustrates plots showing inhibition of AP Hemolysis—Comparison of N297 and Xtend Fc regions—NMT30, NMT31, NMT30-100, NMT30-101, NMT31-100, and NMT31-101. NMT30-100, NMT30-101, NMT31-100, and NMT31-101 were able to inhibit lysis at a similar concentration as NMT1510.

### Inhibition of MAC Formation

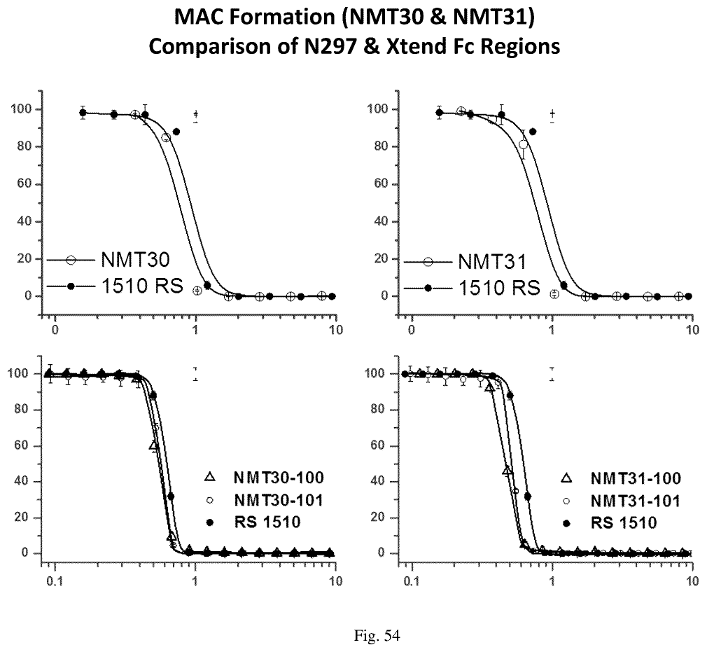

FIG. 54 illustrates plots showing inhibition of MAC Formation—Comparison of N297 and Xtend Fc regions—NMT30, NMT31, NMT30-100, NMT30-101, NMT31-100, and NMT31-101. NMT30-100, NMT30-101, NMT31-100, and NMT31-101 were able to inhibit MAC formation and deposition at a similar concentration as NMT1510.

### Inhibition of Properdin-C3b Binding

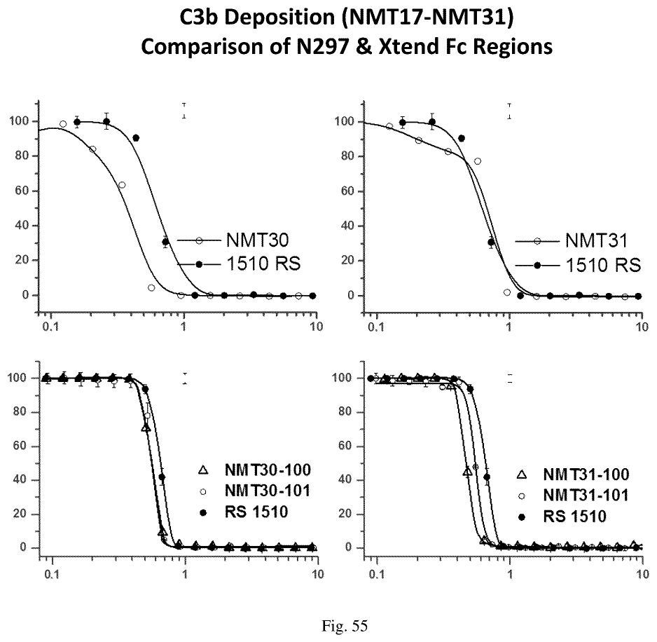

FIG. 55 illustrates plots showing inhibition of C3b Formation and Deposition—Comparison of N297 and Xtend Fc regions—NMT30, NMT31, NMT30-100, NMT30-101, NMT31-100, and NMT31-101. NMT30-100, NMT30-101, NMT31-100, and NMT31-101 were able to inhibit properdin binding to C3b and deposition C3b at a similar concentration as NMT1510.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: An isolated monospecific or bispecific antibody or antigen binding fragment thereof comprising:
at least one of:
a)
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEXX₄X₅X₆X₇X₈X₉ (SEQ ID NO: 202), wherein X₁ is D, E, S, or A; X₂ is H or Y; X₃ is P, S, or Y; X₄ is A or D; X₅ is D, R, or Q; X₆ is K, R, or S; X₇ is F or V; X₅ is E, K, Q, or R; and X₉ is D or G; and
a CDR-H3 comprising the amino acid sequence of RGGGYYLDY (SEQ ID NO: 203);
b)
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y;
c)
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX-₂PALKS (SEQ ID NO: 212), wherein X₁ is F, H, W, or Y; and X₂ is A, N, Q, or S; and
a CDR-H3 comprising the amino acid sequence of IGX₁GYYSFDY (SEQ ID NO: 213), wherein X₁ is A, D, E, or S;
d)
a CDR-L1 comprising the amino acid sequence of X₁ASQDVSDAVA (SEQ ID NO: 216), wherein X₁ is K or R;
a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y;
e)
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222); and
a CDR-H3 comprising the amino acid sequence of SVX₁GIX₁SYX₁ AAFX₂L (SEQ ID NO: 223), wherein X₁ is A, D, E, or S; and X₂ is A, N, Q, or S;
f)
a CDR-L1 comprising the amino acid sequence of X₁ASDX₂IYSLLA (SEQ ID NO: 229), wherein X₁ is Q or R; X₂ is A, N, Q, or S;
a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S;
g)
a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M; and
a CDR-H3 comprising the amino acid sequence of SAX₁GSX₁SYX₁AYFTL (SEQ ID NO: 227), wherein X₁ is A, D, E, or S;
h)
a CDR-L1 comprising the amino acid sequence of X₁ASDX₂IYSX₂LA (SEQ ID NO: 233), wherein X₁ is Q or R; X₂ is A, N, Q, or S;
a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y;
i)
a CDR-H1 comprising the amino acid sequence of GRISSIIHMA (SEQ ID NO: 237), wherein X₁ is F, H, W, or Y,
a CDR-H2 comprising the amino acid sequence of RX₁GTTX₁YAX-₂SX₁X₃G (SEQ ID NO: 238), wherein X₁ is I or V; X₂ is A, D, E, or S; and X₃ is A or K; and
a CDR-H3 comprising the amino acid sequence of LQYEX₁HGGAX₂Y (SEQ ID NO: 239), wherein X₁ is A or K; and X₂ is A, D, E, or S;
j)
a CDR-H1 comprising the amino acid sequence of GRIFEX₁X₂MMA (SEQ ID NO: 241), wherein X₁ is I or V; and X₂ is A, D, N, Q, or S,
a CDR-H2 comprising the amino acid sequence of RX₁GTTTYAX₂SX₁X₃G (SEQ ID NO: 242), wherein X₁ is I or V; X₂ is A, D, E, or S; and X₃ is A or K; and
a CDR-H3 comprising the amino acid sequence of LQYX₁RYGGAEY (SEQ ID NO: 243), wherein X₁ is A, D, E, or S; or
k)
or a heavy chain variable region and/or light chain variable region that competitively inhibits binding of an isolated monospecific or bispecific antibody or antigen binding fragment thereof comprising at least one of a), b), c), d), e), f), g), h), i), or j) to monomeric properdin.

2: The isolated monospecific or bispecific antibody or antigen binding fragment thereof of claim 1, comprising:
a CDR-H1 comprising the amino acid sequence of GYIFTX₁YPIH (SEQ ID NO: 201), wherein X₁ is N, Q, S, A, or D,
a CDR-H2 comprising the amino acid sequence of FIX₁PGGGX₂DEXX₄X₅X₆X₇X₈X₉ (SEQ ID NO: 202), wherein X₁ is D, E, S, or A; X₂ is H or Y; X₃ is P, S, or Y; X₄ is A or D; X₅ is D, R, or Q; X₆ is K, R, or S; X₇ is F or V; X₅ is E, K, Q, or R; and X₉ is D or G; and
a CDR-H3 comprising the amino acid sequence of RGGGYYLDY (SEQ ID NO: 203);
a CDR-L1 comprising the amino acid sequence of RASQDISFFLN (SEQ ID NO: 206),
a CDR-L2 comprising the amino acid sequence of X₁X₂SX₃YHS (SEQ ID NO: 207), wherein X₁ is G or Y; X₂ is A or T; and X₃ is R or S; and
a CDR-L3 comprising the amino acid sequence of QHGX₁TLPX₂T (SEQ ID NO: 208), wherein X₁ is A, D, N, Q, or S; and X₂ is F, H, R, W, or Y.

3: The isolated monospecific or bispecific antibody or antigen binding fragment thereof of claim 1, comprising:
a CDR-H1 comprising the amino acid sequence of GFSLSTSGX₁GVG (SEQ ID NO: 211), wherein X₁ is I, K, M, or V,
a CDR-H2 comprising the amino acid sequence of HIX₁X₁DDVKSYX-2PALKS (SEQ ID NO: 212), wherein X₁ is F, H, W, or Y; and X₂ is A, N, Q, or S;
a CDR-H3 comprising the amino acid sequence of IGX₁GYYSFDY (SEQ ID NO: 213), wherein X₁ is A, D, E, or S;
a CDR-L1 comprising the amino acid sequence of X₁ASQDVSDAVA (SEQ ID NO: 216), wherein X₁ is K or R;
a CDR-L2 comprising the amino acid sequence of SPSYRYT (SEQ ID NO: 217); and
a CDR-L3 comprising the amino acid sequence of QQHYSTPX₁TF (SEQ ID NO: 218), wherein X₁ is F, H, W, or Y.

4: The isolated monospecific or bispecific antibody or antigen binding fragment thereof of claim 1, comprising:
a CDR-H1 comprising the amino acid sequence of GFSFSSGYX₁IF (SEQ ID NO: 221), wherein X₁ is F, H, W, or Y;
a CDR-H2 comprising the amino acid sequence of GIYSGSSGTTY (SEQ ID NO: 222);
a CDR-H3 comprising the amino acid sequence of SVX₁GIX₁SYX₁ AAFX₂L (SEQ ID NO: 223), wherein X₁ is A, D, E, or S; and X₂ is A, N, Q, or S;
a CDR-L1 comprising the amino acid sequence of X₁ASDX₂IYSLLA (SEQ ID NO: 229), wherein X₁ is Q or R; X₂ is A, N, Q, or S;
a CDR-L2 comprising the amino acid sequence of RASTLAS (SEQ ID NO: 230); and
a CDR-L3 comprising the amino acid sequence of QQHYDYX₁YLDVA (SEQ ID NO: 231), wherein X₁ is A, N, Q, or S.

5: The isolated monospecific or bispecific antibody or antigen binding fragment thereof of claim 1, comprising:
a CDR-H1 comprising the amino acid sequence of GFSFSSSYX₁IF (SEQ ID NO: 225), wherein X₁ is F, H, W, or Y,
a CDR-H2 comprising the amino acid sequence of GIYSSSGRX₁Y (SEQ ID NO: 226), wherein X₁ is I, K, L, or M;
a CDR-H3 comprising the amino acid sequence of SAX₁GSX₁SYX₁AYFTL (SEQ ID NO: 227), wherein X₁ is A, D, E, or S;
a CDR-L1 comprising the amino acid sequence of X₁ASDX₂IYSX₂LA (SEQ ID NO: 233), wherein X₁ is Q or R; X₂ is A, N, Q, or S;
a CDR-L2 comprising the amino acid sequence of RASTLAS (SEQ ID NO: 234); and
a CDR-L3 comprising the amino acid sequence of QQHX₁DYDYIDVA (SEQ ID NO: 235), wherein X₁ is F, H, W, or Y.

6: The isolated monospecific or bispecific antibody or antigen binding fragment thereof of claim 1, comprising:
a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107; or
a heavy chain variable region that competitively inhibits binding of an isolated antibody or antigen binding fragment comprising at least one of a heavy chain variable region that includes the 3 CDRs of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

7: The isolated monospecific or bispecific antibody or antigen binding fragment thereof of claim 6, comprising a heavy chain variable region that includes an amino acid sequence at least about 70%, at least about 80%, or at least about 90% identical to the amino acid sequence of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 40, SEQ ID NO: 41, SEQ ID NO: 42, SEQ ID NO: 43, SEQ ID NO: 44, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 71, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, or SEQ ID NO: 107.

8: The isolated monospecific or bispecific antibody or antigen binding fragment thereof of claim 1, comprising:
a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72; or
a light chain variable region that competitively inhibits binding of an isolated antibody or antigen binding fragment comprising at least one of a light chain variable region that includes the 3 CDRs of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

9: The isolated monospecific or bispecific antibody or antigen binding fragment thereof of claim 8, comprising a light chain variable region that includes an amino acid sequence at least about 70%, at least about 80%, or at least about 90% identical to the amino acid sequence of one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 45, SEQ ID NO: 46, SEQ ID NO: 47, SEQ ID NO: 48, SEQ ID NO: 49, SEQ ID NO: 50, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 72.

10: The isolated monospecific or bispecific antibody or antigen binding fragment thereof of claim 1, comprising at least one of the following:
a) a heavy chain variable region that includes the 3 CDRs of SEQ ID NO: 1 and a light chain variable region that includes the 3 CDRs of SEQ ID NO: 24;
b) a heavy chain variable region that includes the 3 CDRs of SEQ ID NO: 2 and a light chain variable region that includes the 3 CDRs of SEQ ID NO: 25;
c) a heavy chain variable region that includes the 3 CDRs of SEQ ID NO: 3 and a light chain variable region that includes the 3 CDRs of SEQ ID NO: 26;
d) a heavy chain variable region that includes the 3 CDRs of SEQ ID NO: 4 and a light chain variable region that includes the 3 CDRs of SEQ ID NO: 27;
e) a heavy chain variable region that includes the 3 CDRs of SEQ ID NO: 5 and a light chain variable region that includes the 3 CDRs of SEQ ID NO: 28;
f) a heavy chain variable region that includes the 3 CDRs of SEQ ID NO: 6 and a light chain variable region that includes the 3 CDRs of SEQ ID NO: 29;
g) a heavy chain variable region that includes the 3 CDRs of SEQ ID NO: 7 and a light chain variable region that includes the 3 CDRs of SEQ ID NO: 30;
h) a heavy chain variable region that includes the 3 CDRs of SEQ ID NO: 8 and a light chain variable region that includes the 3 CDRs of SEQ ID NO: 31;
i) a heavy chain variable region that includes the 3 CDRs of SEQ ID NO: 9 and a light chain variable region that includes the 3 CDRs of SEQ ID NO: 32;
j) a heavy chain variable region that includes the 3 CDRs of SEQ ID NO: 10 and a light chain variable region that includes the 3 CDRs of SEQ ID NO: 33;
k) a heavy chain variable region that includes the 3 CDRs of SEQ ID NO: 11 and a light chain variable region that includes the 3 CDRs of SEQ ID NO: 34;
l) a heavy chain variable region that includes the 3 CDRs of SEQ ID NO: 12 and a light chain variable region that includes the 3 CDRs of SEQ ID NO: 35;
m) a heavy chain variable region that includes the 3 CDRs of SEQ ID NO: 13 and a light chain variable region that includes the 3 CDRs of SEQ ID NO: 36;
n) a heavy chain variable region that includes the 3 CDRs of SEQ ID NO: 14 and a light chain variable region that includes the 3 CDRs of SEQ ID NO: 37;
o) a heavy chain variable region that includes the 3 CDRs of SEQ ID NO: 15 and a light chain variable region that includes the 3 CDRs of SEQ ID NO: 38 or SEQ ID NO: 39;
p) a heavy chain variable region that includes the 3 CDRs of SEQ ID NO: 16 and a light chain variable region that includes the 3 CDRs of SEQ ID NO: 38 or SEQ ID NO: 39;
q) a heavy chain variable region that includes the 3 CDRs of SEQ ID NO: 17 and a light chain variable region that includes the 3 CDRs of SEQ ID NO: 38 or SEQ ID NO: 39;
r) a heavy chain variable region that includes the 3 CDRs of SEQ ID NO: 18 and a light chain variable region that includes the 3 CDRs of SEQ ID NO: 38 or SEQ ID NO: 39;
s) a heavy chain variable region that includes the 3 CDRs of SEQ ID NO: 19 and a light chain variable region that includes the 3 CDRs of SEQ ID NO: 38 or SEQ ID NO: 39;
t) a heavy chain variable region that includes the 3 CDRs of SEQ ID NO: 20 and a light chain variable region that includes the 3 CDRs of SEQ ID NO: 38 or SEQ ID NO: 39;
u) a heavy chain variable region that includes the 3 CDRs of SEQ ID NO: 21 and a light chain variable region that includes the 3 CDRs of SEQ ID NO: 38 or SEQ ID NO: 39;
v) a heavy chain variable region that includes the 3 CDRs of SEQ ID NO: 22 and a light chain variable region that includes the 3 CDRs of SEQ ID NO: 38 or SEQ ID NO: 39;
w) a heavy chain variable region that includes the 3 CDRs of SEQ ID NO: 23 and a light chain variable region that includes the 3 CDRs of SEQ ID NO: 38 or SEQ ID NO: 39;
x) a heavy chain variable region that includes the 3 CDRs of SEQ ID NO: 40 and a light chain variable region that includes the 3 CDRs of SEQ ID NO: 45;
y) a heavy chain variable region that includes the 3 CDRs of SEQ ID NO: 41 and a light chain variable region that includes the 3 CDRs of SEQ ID NO: 46;
z) a heavy chain variable region that includes the 3 CDRs of SEQ ID NO: 42 and a light chain variable region that includes the 3 CDRs of SEQ ID NO: 47;
aa) a heavy chain variable region that includes the 3 CDRs of SEQ ID NO: 43 and a light chain variable region that includes the 3 CDRs of SEQ ID NO: 48, SEQ ID NO: 49, or SEQ ID NO: 50;
bb) a heavy chain variable region that includes the 3 CDRs of SEQ ID NO: 44 and a light chain variable region that includes the 3 CDRs of SEQ ID NO: 48, SEQ ID NO: 49, or SEQ ID NO: 50;
cc) a heavy chain variable region that includes the 3 CDRs of SEQ ID NO: 51 and a light chain variable region that includes the 3 CDRs of SEQ ID NO: 61, SEQ ID NO: 62, SEQ ID NO: 63, SEQ ID NO: 64, or SEQ ID NO: 65;
dd) a heavy chain variable region that includes the 3 CDRs of SEQ ID NO: 52 and a light chain variable region that includes the 3 CDRs of SEQ ID NO: 61, SEQ ID NO: 62, SEQ ID NO: 63, SEQ ID NO: 64, or SEQ ID NO: 65;
ee) a heavy chain variable region that includes the 3 CDRs of SEQ ID NO: 53 and a light chain variable region that includes the 3 CDRs of SEQ ID NO: 61, SEQ ID NO: 62, SEQ ID NO: 63, SEQ ID NO: 64, or SEQ ID NO: 65;
ff) a heavy chain variable region that includes the 3 CDRs of SEQ ID NO: 54 and a light chain variable region that includes the 3 CDRs of SEQ ID NO: 61, SEQ ID NO: 62, SEQ ID NO: 63, SEQ ID NO: 64, or SEQ ID NO: 65;
gg) a heavy chain variable region that includes the 3 CDRs of SEQ ID NO: 55 and a light chain variable region that includes the 3 CDRs of SEQ ID NO: 61, SEQ ID NO: 62, SEQ ID NO: 63, SEQ ID NO: 64, or SEQ ID NO: 65;
hh) a heavy chain variable region that includes the 3 CDRs of SEQ ID NO: 56 and a light chain variable region that includes the 3 CDRs of SEQ ID NO: 66, SEQ ID NO: 67, SEQ ID NO: 68, SEQ ID NO: 69, or SEQ ID NO: 70;
ii) a heavy chain variable region that includes the 3 CDRs of SEQ ID NO: 57 and a light chain variable region that includes the 3 CDRs of SEQ ID NO: 66, SEQ ID NO: 67, SEQ ID NO: 68, SEQ ID NO: 69, or SEQ ID NO: 70;
jj) a heavy chain variable region that includes the 3 CDRs of SEQ ID NO: 58 and a light chain variable region that includes the 3 CDRs of SEQ ID NO: 66, SEQ ID NO: 67, SEQ ID NO: 68, SEQ ID NO: 69, or SEQ ID NO: 70;
kk) a heavy chain variable region that includes the 3 CDRs of SEQ ID NO: 59 and a light chain variable region that includes the 3 CDRs of SEQ ID NO: 66, SEQ ID NO: 67, SEQ ID NO: 68, SEQ ID NO: 69, or SEQ ID NO: 70; or
II) a heavy chain variable region that includes the 3 CDRs of SEQ ID NO: 60 and a light chain variable region that includes the 3 CDRs of SEQ ID NO: 66, SEQ ID NO: 67, SEQ ID NO: 68, SEQ ID NO: 69, or SEQ ID NO: 70.

11: The isolated monospecific or bispecific antibody or antigen binding fragment thereof of claim 10, comprising at least one of the following:
a) a heavy chain that includes an amino acid sequence at least about 70%, at least about 80%, or at least about 90% identical to the amino acid sequence of SEQ ID NO: 1 and a light chain that includes an amino acid sequence at least about 70%, at least about 80%, or at least about 90% identical to the amino acid sequence of SEQ ID NO: 24;
b) a heavy chain that includes an amino acid sequence at least about 70%, at least about 80%, or at least about 90% identical to the amino acid sequence of SEQ ID NO: 2 and a light chain that includes an amino acid sequence at least about 70%, at least about 80%, or at least about 90% identical to the amino acid sequence of SEQ ID NO: 25;
c) a heavy chain variable that includes an amino acid sequence at least about 70%, at least about 80%, or at least about 90% identical to the amino acid sequence of SEQ ID NO: 3 and a light chain that includes an amino acid sequence at least about 70%, at least about 80%, or at least about 90% identical to the amino acid sequence of SEQ ID NO: 26;
d) a heavy chain that includes an amino acid sequence at least about 70%, at least about 80%, or at least about 90% identical to the amino acid sequence of SEQ ID NO: 4 and a light chain that includes an amino acid sequence at least about 70%, at least about 80%, or at least about 90% identical to the amino acid sequence of SEQ ID NO: 27;
e) a heavy chain that includes an amino acid sequence at least about 70%, at least about 80%, or at least about 90% identical to the amino acid sequence of SEQ ID NO: 5 and a light chain that includes an amino acid sequence at least about 70%, at least about 80%, or at least about 90% identical to the amino acid sequence of SEQ ID NO: 28;
f) a heavy chain that includes an amino acid sequence at least about 70%, at least about 80%, or at least about 90% identical to the amino acid sequence of SEQ ID NO: 6 and a light chain that includes an amino acid sequence at least about 70%, at least about 80%, or at least about 90% identical to the amino acid sequence of SEQ ID NO: 29;
g) a heavy chain that includes an amino acid sequence at least about 70%, at least about 80%, or at least about 90% identical to the amino acid sequence of SEQ ID NO: 7 and a light chain that includes an amino acid sequence at least about 70%, at least about 80%, or at least about 90% identical to the amino acid sequence of SEQ ID NO: 30;
h) a heavy chain that includes an amino acid sequence at least about 70%, at least about 80%, or at least about 90% identical to the amino acid sequence of SEQ ID NO: 8 and a light chain that includes an amino acid sequence at least about 70%, at least about 80%, or at least about 90% identical to the amino acid sequence of SEQ ID NO: 31;
i) a heavy chain that includes an amino acid sequence at least about 70%, at least about 80%, or at least about 90% identical to the amino acid sequence of SEQ ID NO: 9 and a light chain that includes an amino acid sequence at least about 70%, at least about 80%, or at least about 90% identical to the amino acid sequence of SEQ ID NO: 32;
j) a heavy chain that includes an amino acid sequence at least about 70%, at least about 80%, or at least about 90% identical to the amino acid sequence of SEQ ID NO: 10 and a light chain that includes an amino acid sequence at least about 70%, at least about 80%, or at least about 90% identical to the amino acid sequence of SEQ ID NO: 33;
k) a heavy chain that includes an amino acid sequence at least about 70%, at least about 80%, or at least about 90% identical to the amino acid sequence of SEQ ID NO: 11 and a light chain that includes an amino acid sequence at least about 70%, at least about 80%, or at least about 90% identical to the amino acid sequence of SEQ ID NO: 34;
l) a heavy chain that includes an amino acid sequence at least about 70%, at least about 80%, or at least about 90% identical to the amino acid sequence of SEQ ID NO: 12 and a light chain that includes an amino acid sequence at least about 70%, at least about 80%, or at least about 90% identical to the amino acid sequence of SEQ ID NO: 35;
m) a heavy chain that includes an amino acid sequence at least about 70%, at least about 80%, or at least about 90% identical to the amino acid sequence of SEQ ID NO: 13 and a light chain that includes an amino acid sequence at least about 70%, at least about 80%, or at least about 90% identical to the amino acid sequence of SEQ ID NO: 36;
n) a heavy chain that includes an amino acid sequence at least about 70%, at least about 80%, or at least about 90% identical to the amino acid sequence of SEQ ID NO: 14 and a light chain that includes an amino acid sequence at least about 70%, at least about 80%, or at least about 90% identical to the amino acid sequence of SEQ ID NO: 37;
o) a heavy chain that includes an amino acid sequence at least about 70%, at least about 80%, or at least about 90% identical to the amino acid sequence of SEQ ID NO: 15 and a light chain that includes an amino acid sequence at least about 70%, at least about 80%, or at least about 90% identical to the amino acid sequence of SEQ ID NO: 38 or SEQ ID NO: 39;
p) a heavy chain that includes an amino acid sequence at least about 70%, at least about 80%, or at least about 90% identical to the amino acid sequence of SEQ ID NO: 16 and a light chain that includes an amino acid sequence at least about 70%, at least about 80%, or at least about 90% identical to the amino acid sequence of SEQ ID NO: 38 or SEQ ID NO: 39;
q) a heavy chain that includes an amino acid sequence at least about 70%, at least about 80%, or at least about 90% identical to the amino acid sequence of SEQ ID NO: 17 and a light chain that includes an amino acid sequence at least about 70%, at least about 80%, or at least about 90% identical to the amino acid sequence of SEQ ID NO: 38 or SEQ ID NO: 39;
r) a heavy chain that includes an amino acid sequence at least about 70%, at least about 80%, or at least about 90% identical to the amino acid sequence of SEQ ID NO: 18 and a light chain that includes an amino acid sequence at least about 70%, at least about 80%, or at least about 90% identical to the amino acid sequence of SEQ ID NO: 38 or SEQ ID NO: 39;
s) a heavy chain that includes an amino acid sequence at least about 70%, at least about 80%, or at least about 90% identical to the amino acid sequence of SEQ ID NO: 19 and a light chain that includes an amino acid sequence at least about 70%, at least about 80%, or at least about 90% identical to the amino acid sequence of SEQ ID NO: 38 or SEQ ID NO: 39;
t) a heavy chain that includes an amino acid sequence at least about 70%, at least about 80%, or at least about 90% identical to the amino acid sequence of SEQ ID NO: 20 and a light chain that includes an amino acid sequence at least about 70%, at least about 80%, or at least about 90% identical to the amino acid sequence of SEQ ID NO: 38 or SEQ ID NO: 39;
u) a heavy chain that includes an amino acid sequence at least about 70%, at least about 80%, or at least about 90% identical to the amino acid sequence of SEQ ID NO: 21 and a light chain that includes an amino acid sequence at least about 70%, at least about 80%, or at least about 90% identical to the amino acid sequence of SEQ ID NO: 38 or SEQ ID NO: 39;
v) a heavy chain that includes an amino acid sequence at least about 70%, at least about 80%, or at least about 90% identical to the amino acid sequence of SEQ ID NO: 22 and a light chain that includes an amino acid sequence at least about 70%, at least about 80%, or at least about 90% identical to the amino acid sequence of SEQ ID NO: 38 or SEQ ID NO: 39;
w) a heavy chain that includes an amino acid sequence at least about 70%, at least about 80%, or at least about 90% identical to the amino acid sequence of SEQ ID NO: 23 and a light chain that includes an amino acid sequence at least about 70%, at least about 80%, or at least about 90% identical to the amino acid sequence of SEQ ID NO: 38 or SEQ ID NO: 39;
x) a heavy chain that includes an amino acid sequence at least about 70%, at least about 80%, or at least about 90% identical to the amino acid sequence of SEQ ID NO: 40 and a light chain that includes an amino acid sequence at least about 70%, at least about 80%, or at least about 90% identical to the amino acid sequence of SEQ ID NO: 45;
y) a heavy chain that includes an amino acid sequence at least about 70%, at least about 80%, or at least about 90% identical to the amino acid sequence of SEQ ID NO: 41 and a light chain that includes an amino acid sequence at least about 70%, at least about 80%, or at least about 90% identical to the amino acid sequence of SEQ ID NO: 46;
z) a heavy chain that includes an amino acid sequence at least about 70%, at least about 80%, or at least about 90% identical to the amino acid sequence of SEQ ID NO: 42 and a light chain that includes an amino acid sequence at least about 70%, at least about 80%, or at least about 90% identical to the amino acid sequence of SEQ ID NO: 47;
aa) a heavy chain that includes an amino acid sequence at least about 70%, at least about 80%, or at least about 90% identical to the amino acid sequence of SEQ ID NO: 43 and a light chain that includes an amino acid sequence at least about 70%, at least about 80%, or at least about 90% identical to the amino acid sequence of SEQ ID NO: 48, SEQ ID NO: 49, or SEQ ID NO: 50;
bb) a heavy chain that includes an amino acid sequence at least about 70%, at least about 80%, or at least about 90% identical to the amino acid sequence of SEQ ID NO: 44 and a light chain that includes an amino acid sequence at least about 70%, at least about 80%, or at least about 90% identical to the amino acid sequence of SEQ ID NO: 48, SEQ ID NO: 49, or SEQ ID NO: 50;
cc) a heavy chain that includes an amino acid sequence at least about 70%, at least about 80%, or at least about 90% identical to the amino acid sequence of SEQ ID NO: 51 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
dd) a heavy chain that includes an amino acid sequence at least about 70%, at least about 80%, or at least about 90% identical to the amino acid sequence of SEQ ID NO: 52 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ee) a heavy chain that includes an amino acid sequence at least about 70%, at least about 80%, or at least about 90% identical to the amino acid sequence of SEQ ID NO: 53 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
ff) a heavy chain that includes an amino acid sequence at least about 70%, at least about 80%, or at least about 90% identical to the amino acid sequence of SEQ ID NO: 54 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
gg) a heavy chain that includes an amino acid sequence at least about 70%, at least about 80%, or at least about 90% identical to the amino acid sequence of SEQ ID NO: 55 and a light chain that includes an amino acid sequence at least about 70%, at least about 80%, or at least about 90% identical to the amino acid sequence of SEQ ID NO: 61, SEQ ID NO: 62, SEQ ID NO: 63, SEQ ID NO: 64, or SEQ ID NO: 65;
hh) a heavy chain that includes an amino acid sequence at least about 70%, at least about 80%, or at least about 90% identical to the amino acid sequence of SEQ ID NO: 56 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
ii) a heavy chain that includes an amino acid sequence at least about 70%, at least about 80%, or at least about 90% identical to the amino acid sequence of SEQ ID NO: 57 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
jj) a heavy chain that includes an amino acid sequence at least about 70%, at least about 80%, or at least about 90% identical to the amino acid sequence of SEQ ID NO: 58 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70;
kk) a heavy chain that includes an amino acid sequence at least about 70%, at least about 80%, or at least about 90% identical to the amino acid sequence of SEQ ID NO: 59 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70; or
ll) a heavy chain that includes an amino acid sequence at least about 70%, at least about 80%, or at least about 90% identical to the amino acid sequence of SEQ ID NO: 60 and a light chain that includes an amino acid sequence at least about 70%, at least about 80%, or at least about 90% identical to the amino acid sequence of SEQ ID NO: 66, SEQ ID NO: 67, SEQ ID NO: 68, SEQ ID NO: 69, or SEQ ID NO: 70.

12: The isolated monospecific or bispecific antibody or antigen binding fragment thereof of claim 1, further comprising a constant chain region and wherein the antibody or antigen binding fragment including the constant chain region has enhanced in vivo half-live and/or reduced immunogenicity compared to the antibody or antigen binding fragment thereof without the constant chain region.

13: The isolated monospecific or bispecific antibody or antigen binding fragment thereof of claim 12, wherein the constant chain region includes an amino acid sequence at least about 70%, at least about 80%, or at least about 90% identical to the amino acid sequence of SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, or SEQ ID NO: 139,

14: The isolated monospecific or bispecific antibody or antigen binding fragment thereof of claim 1, comprising at least one of the following:
a heavy chain that includes an amino acid sequence at least about 70%, at least about 80%, or at least about 90% identical to the amino acid sequence of SEQ ID NO: 156 and a light chain that includes an amino acid sequence at least about 70%, at least about 80%, or at least about 90% identical to the amino acid sequence of SEQ ID NO: 157;
a heavy chain that includes an amino acid sequence at least about 70%, at least about 80%, or at least about 90% identical to the amino acid sequence of SEQ ID NO: 166 and a light chain that includes an amino acid sequence at least about 70%, at least about 80%, or at least about 90% identical to the amino acid sequence of SEQ ID NO: 167;
a heavy chain that includes an amino acid sequence at least about 70%, at least about 80%, or at least about 90% identical to the amino acid sequence of SEQ ID NO: 170 and a light chain that includes an amino acid sequence at least about 70%, at least about 80%, or at least about 90% identical to the amino acid sequence of SEQ ID NO: 171;
a heavy chain that includes an amino acid sequence at least about 70%, at least about 80%, or at least about 90% identical to the amino acid sequence of SEQ ID NO: 178 and a light chain that includes an amino acid sequence at least about 70%, at least about 80%, or at least about 90% identical to the amino acid sequence of SEQ ID NO: 179;
a heavy chain that includes an amino acid sequence at least about 70%, at least about 80%, or at least about 90% identical to the amino acid sequence of SEQ ID NO: 182;
a heavy chain that includes an amino acid sequence at least about 70%, at least about 80%, or at least about 90% identical to the amino acid sequence of SEQ ID NO: 184 and a light chain that includes an amino acid sequence at least about 70%, at least about 80%, or at least about 90% identical to the amino acid sequence of SEQ ID NO: 185;
a heavy chain that includes an amino acid sequence at least about 70%, at least about 80%, or at least about 90% identical to the amino acid sequence of SEQ ID NO: 187 and a light chain that includes an amino acid sequence at least about 70%, at least about 80%, or at least about 90% identical to the amino acid sequence of SEQ ID NO: 188;
a heavy chain that includes an amino acid sequence at least about 70%, at least about 80%, or at least about 90% identical to the amino acid sequence of SEQ ID NO: 191;
a heavy chain that includes an amino acid sequence at least about 70%, at least about 80%, or at least about 90% identical to the amino acid sequence of SEQ ID NO: 193 and a light chain that includes an amino acid sequence at least about 70%, at least about 80%, or at least about 90% identical to the amino acid sequence of SEQ ID NO: 194; or
a heavy chain that includes an amino acid sequence at least about 70%, at least about 80%, or at least about 90% identical to the amino acid sequence of SEQ ID NO: 197 and a light chain that includes an amino acid sequence at least about 70%, at least about 80%, or at least about 90% identical to the amino acid sequence of SEQ ID NO: 198.

15: The isolated monospecific or bispecific antibody or antigen binding fragment thereof of claim 1 further comprising a peptide linker.

16: The isolated monospecific or bispecific antibody or antigen binding fragment thereof of claim 15, wherein the peptide linker comprises the amino acid sequence of SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 245, or SEQ ID NO: 246.

17: The isolated monospecific or bispecific antibody or antigen binding fragment thereof of any of claim 1, wherein the antibody or antigen binding fragment thereof is humanized.

18: The isolated monospecific or bispecific antibody or antigen binding fragment thereof of any of claim 1, wherein the antibody or antigen binding fragment thereof binds to human properdin.

19: The isolated monospecific or bispecific antibody or antigen binding fragment thereof of claim 1, wherein the antibody or antigen binding fragment thereof inhibits alternative complement pathway activation in a mammal without inhibiting classical complement pathway activation.

20: The isolated monospecific or bispecific antibody or antigen binding fragment thereof of claim 1, comprising a first heavy chain variable region that binds to properdin and a second heavy chain variable region that binds to a different epitope than the first heavy chain variable region.

21-30. (canceled)
